(19) United States
(12) Patent Application Publication
(10) Pub. No.: US 2021/0395256 A1
(43) Pub. Date: Dec. 23, 2021

(54) TYROSINE KINASE INHIBITORS, COMPOSITIONS AND METHODS THERE OF

(71) Applicant: Betta Pharmaceuticals Co., Ltd., Zhejiang (CN)

(72) Inventors: Bang FU, Beijing (CN); Yinlong LI, Beijing (CN); Wei REN, Beijing (CN); Jie CHEN, Beijing (CN); Xiangyong LIU, Beijing (CN); Jiabing WANG, Beijing (CN); Lieming DING, Zhejiang (CN)

(21) Appl. No.: 17/311,105

(22) PCT Filed: Dec. 6, 2019
(86) PCT No.: PCT/CN2019/123719

§ 371 Date: Jun. 4, 2021

(30) Foreign Application Priority Data
Dec. 7, 2018 (CN) PCT/CN2018/119895
May 9, 2019 (CN) PCT/CN2019/086204

(51) Int. Cl. C07D 487/04; C07D 519/00

(52) U.S. Cl. CPC C07D 487/04; C07D 519/00

(57) ABSTRACT

The present invention relates to compounds of Formula I, methods of using the compounds as Trk inhibitors, and pharmaceutical compositions comprising such compounds. The compounds are useful in treating, preventing or ameliorating diseases or disorders such as cancer or infections.

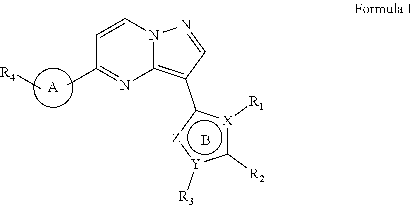

### FIELD OF THE INVENTION

The present application is concerned with pharmaceutically active compounds. The disclosure provides compounds as well as their compositions and methods of use. The compounds inhibit tropomyosin-related kinases (Trks) and are useful in the treatment of various diseases including infectious diseases and cancer.

### BACKGROUND OF THE INVENTION

Tropomyosin-related kinases (Trks) are a group of receptor tyrosine kinases which are regulated by neurotrophins, including 3 members TrkA, TrkB and TrkC, encoded by the genes NTRK1, NTRK2 and NTRK3 respectively. Many cellular functions, for example, cell proliferation, cell differentiation, metabolism and apoptosis are mediated by Trks through phosphorylation and regulation of their downstream signal pathway members. Gene fusions involving NTRK genes result in continuous activation or overexpression of these kinases, which increase the risk of tumor genesis.

Trk plays an important physiological role in the development of nerves, including the growth and function maintenance of neuronal axons, the development of memory and the protection of neurons from injury, etc. Also, it is showed that Trk uncommonly expresses in normal tissues or cancer, while fusion drives abnormally high expression and activation of Trk kinase domain. Trk fusions are found in diverse cancer histologies with low fusion frequency, such as thyroid cancer, lung cancer, colon cancer, and melanoma. It is estimated that 1,500-5,000 patients harbor Trk fusion-positive cancers in the United States annually.

In recent years, Trk fusion protein is becoming a valid cancer target, the small molecule inhibitor for Trk with most rapid development is Loxo Oncology's larotrectinib which is highly potent against Trk in clinical development. Earlier application, WO2010048314, WO2011006074, WO2016097869, and WO2018077246 disclosed a series of Trk inhibitors. Accordingly, there is still a great demand for Trk inhibitors which have more potent activity, and better liver microsomes metabolic stability. Additionally, in view of the importance of Trk's physiological functions, there is great demand for Trk inhibitors which can inhibit not only Trk A, B, and C but also mutated forms of Trk A, B and C (for example the G595R, G667C, A608D, F589L, G623R) which are reported in patients receiving first generation Trk kinase inhibitors. In this invention, applicant discovered potent small molecules that can have activity as Trk inhibitors, and thus may be useful for therapeutic administration to fight against cancer and/or infectious diseases. These small molecules are expected to be useful as pharmaceuticals with desirable stability, solubility, bioavailability, therapeutic index and toxicity values that are crucial to become efficient medicines to promote human health.

### SUMMARY OF INVENTION

The present invention relates to compounds that are used as Trk inhibitors. Trk inhibitors are useful in the treatment of cancers and infectious diseases.

The compounds of the invention have the general structures as Formula I. A compound of Formula I, or an isomeride, a stereoisomer, tautomer, pharmaceutically acceptable salt, prodrug, chelate, non-covalent complex, or solvate thereof,

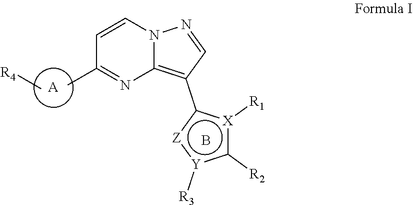

wherein,

ring A is C₅₋₆ heterocyclic ring, wherein the C₅₋₆ heterocyclic ring optionally comprising 1, 2 or 3 hetero atoms independently selected from N, S, or O;

ring B is 5-membered aromatic heterocycle;

X and Z are each independently selected from C, N, O, or S;

Y is C or N;

R₁ is absent, H, or —C₁₋₈ alkyl;

R₂ is H, —C₀₋₄ alkyl-COOR₁₀, —C₀₋₄ alkyl-NH—COOR₁₀, —C₀₋₄ alkyl-O(CO)R₁₀, —C₀₋₄ alkyl-O(CO)—C₁₋₄ alkyl-NHCO—R₁₀, —C₁₋₄ alkyl-NH₂, —C₀₋₄ alkyl-OH, —C₀₋₄ alkyl-C₃₋₁₀ carbocyclic ring, or —C₀₋₄ alkyl-C₃₋₁₀ heterocyclic ring, —C₀₋₄ alkyl-C₆₋₁₀ aryl ring, or —C₀₋₄ alkyl-C₅₋₁₀ heteroaryl ring, wherein the —C₀₋₄ alkyl-COOR₁₀, —C₀₋₄ alkyl-NH—COOR₁₀, —C₀₋₄ alkyl-O(CO)R₁₀, —C₀₋₄ alkyl-O(CO)—C₁₋₄ alkyl-NHCO—R₁₀, —C₁₋₄ alkyl-NH₂, —C₀₋₄ alkyl-OH, —C₀₋₄ alkyl-C₃₋₁₀ carbocyclic ring, —O₀₋₄ alkyl-C₃₋₁₀ heterocyclic ring, —C₀₋₄ alkyl-C₆₋₁₀ aryl ring, or —C₀₋₄ alkyl-C₅₋₁₀ heteroaryl ring is optionally substituted with —C₁₋₈ alkyl, —C₂₋₈ alkynyl, —C₁₋₈ haloalkyl, —C₁₋₈ alkyl-OH, halogen, OH, CN, NH₂, —C₀₋₄ alkyl-COOR₁₀, —C₆₋₁₀ aryl ring, —O—C₆₋₁₀ aryl ring, substituted or unsubstituted —C₃₋₁₀ carbocyclic ring, or substituted or unsubstituted —C₃₋₁₀ heterocyclic ring;

R₃ is absent, C₃₋₁₀ heterocyclic ring; or

R₂ and R₃ together with the atoms to which they are attached to form a 5- to 6-membered carbocyclic ring, heterocyclic ring, aryl ring, or heteroaryl ring, wherein the 5- to 6-membered carbocyclic ring, heterocyclic ring, aryl ring, or heteroaryl ring is optionally substituted with halogen, OH, CN, NH₂, —CONHOH, —CONH₂, —C₀₋₄ alkyl-COOR₁₀, —C₀₋₄ alkyl-O(CO)OR₁₀, —C₁₋₈ alkoxy, —C₁₋₈ haloalkoxy, —C₁₋₈ alkoxy-C₁₋₈ alkoxy, —C₁₋₈ alkylthio, —C₁₋₈ haloalkylthio, —C₁₋₈ alkyl, —C₁₋₈ haloalkyl, —C₀₋₄ alkyl-OH, —O—CH₂—CN, —C₀₋₄ alkyl-O—C₃₋₁₀ heterocyclic ring, substituted or unsubstituted —C₃₋₁₀ carbocyclic ring or substituted or unsubstituted —C₃₋₁₀ heterocyclic ring, or the 5- to 6-membered carbocyclic ring, heterocyclic ring, aryl ring, or heteroaryl ring forms a ring structure with other substituted or unsubstituted carbocyclic ring, substituted or unsubstituted heterocyclic ring, substituted or unsubstituted aryl ring, or substituted or unsubstituted heteroaryl ring;

R₄ is (i) phenyl optionally substituted with one or more substituents independently selected from halogen, —C₁₋₄ alkyl, —C₁₋₄ haloalkyl, C₁₋₄ alkoxyl, or (ii) a C₅₋₆ heteroaryl ring having a ring heteroatom selected from N, S, or O, wherein the C₅₋₆ heteroaryl ring is optionally substituted with one or more halogen atoms;

R₁₀ is H, or —C₁₋₈ alkyl;

wherein the heterocyclic ring or the heteroaryl ring optionally has 1, 2 or 3 heteroatoms independently selected from N, S, O or B.

In some embodiments of Formula I, ring A is

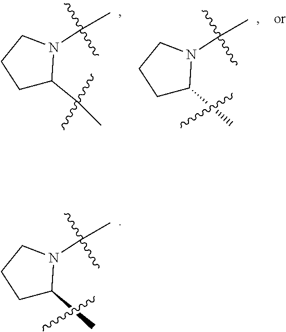

In some embodiments of Formula I, X is independently selected from O, S or N.

In some embodiments of Formula I, Y is C.

In some embodiments of Formula I, Z is N.

In some embodiments of Formula I, R₄ is

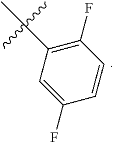 [SMILES: *c1cc(F)ccc1F]

In some embodiments of Formula I, the compound is of Formula II or an isomeride, a stereoisomer, tautomer, pharmaceutically acceptable salt, prodrug, chelate, non-covalent complex, or solvate thereof,

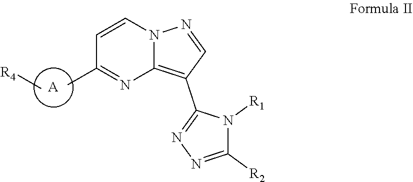

wherein,

ring A is C₅₋₆ heterocyclic ring, wherein the C₅₋₆ heterocyclic ring optionally comprising 1, 2 or 3 hetero atoms independently selected from N, S, or O;

R₁ is H, or —C₁₋₈ alkyl;

R₂ is H, —C₀₋₄ alkyl-COOR₁₀, —₀₋₄ alkyl-NH—COOR₁₀, —C₀₋₄ alkyl-O(CO)R₁₀, —C₀₋₄ alkyl-O(CO)—C₁₋₄ alkyl-NHCO—R₁₀, —C₁₋₄ alkyl-NH₂, —C₀₋₄ alkyl-OH, —C₁₋₄ alkyl-C₃₋₁₀ carbocyclic ring, or —C₀₋₄ alkyl-C₃₋₁₀ heterocyclic ring, —C₀₋₄ alkyl-C₆₋₁₀ aryl ring, or —C₀₋₄ alkyl-C₅₋₁₀ heteroaryl ring, wherein the —C₀₋₄ alkyl-COOR₁₀, —C₀₋₄ alkyl-NH—COOR₁₀, —C₀₋₄ alkyl-O(CO)R₁₀, —C₀₋₄ alkyl-O(CO)—C₁₋₄ alkyl-NHCO—R₁₀, —C₁₋₄ alkyl-NH₂, —C₀₋₄ alkyl-OH, —C₁₋₄ alkyl-C₃₋₁₀ carbocyclic ring, —C₀₋₄ alkyl-C₃₋₁₀ heterocyclic ring, —C₀₋₄ alkyl-C₆₋₁₀ aryl ring, or —C₀₋₄ alkyl-C₅₋₁₀ heteroaryl ring is optionally substituted with —C₁₋₈alkyl, —C₂₋₈ alkynyl, —C₁₋₈ haloalkyl, —C₁₋₈ alkyl-OH, halogen, OH, CN, NH₂, —C₀₋₄ alkyl-COOR₁₀, —C₆₋₁₀ aryl ring, —O—C₆₋₁₀ aryl ring, substituted or unsubstituted —C₃₋₁₀ carbocyclic ring or substituted or unsubstituted —C₃₋₁₀ heterocyclic ring;

R₄ is (i) phenyl optionally substituted with one or more substituents independently selected from halogen, —C₁₋₄ alkyl, —C₁₋₄ haloalkyl, C₁₋₄ alkoxyl, or (ii) a C₅₋₆ heteroaryl ring having a ring heteroatom selected from N, S, or O, wherein the C₅₋₆ heteroaryl ring is optionally substituted with one or more halogen atoms;

R₁₀ is H, or —C₁₋₈ alkyl;

wherein the heterocyclic ring or the heteroaryl ring optionally has 1, 2 or 3 heteroatoms independently selected from N, S, O or B.

In some embodiments of Formula II, ring A is

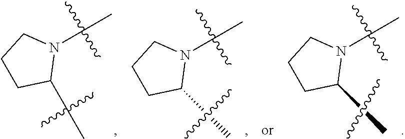

In some embodiments of Formula II, R₁ is independently selected from H or CH₃.

In some embodiments of Formula II, R₄ is

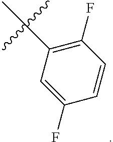 [SMILES: *c1cc(F)ccc1F]

In some embodiments of Formula II, R₂ is independently selected from

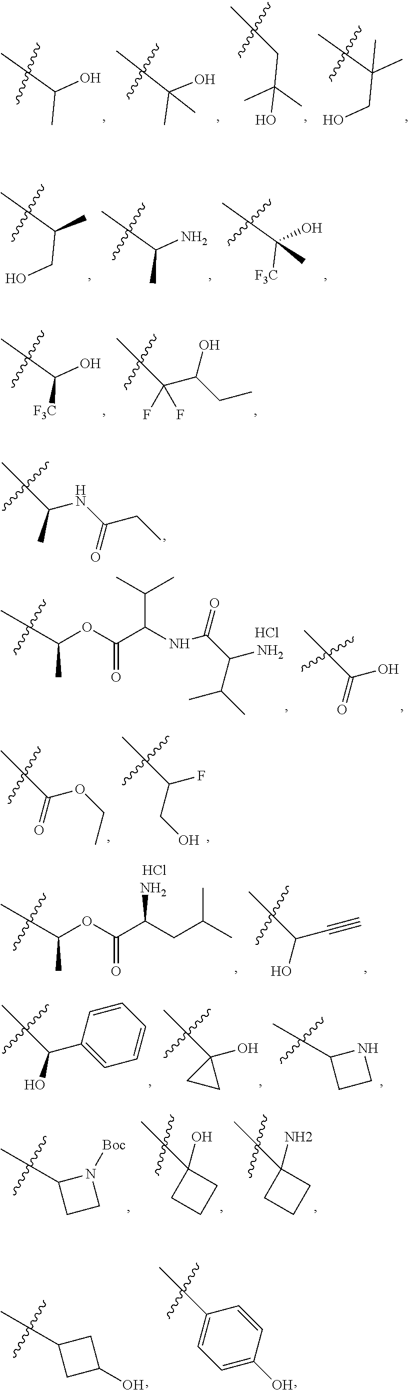

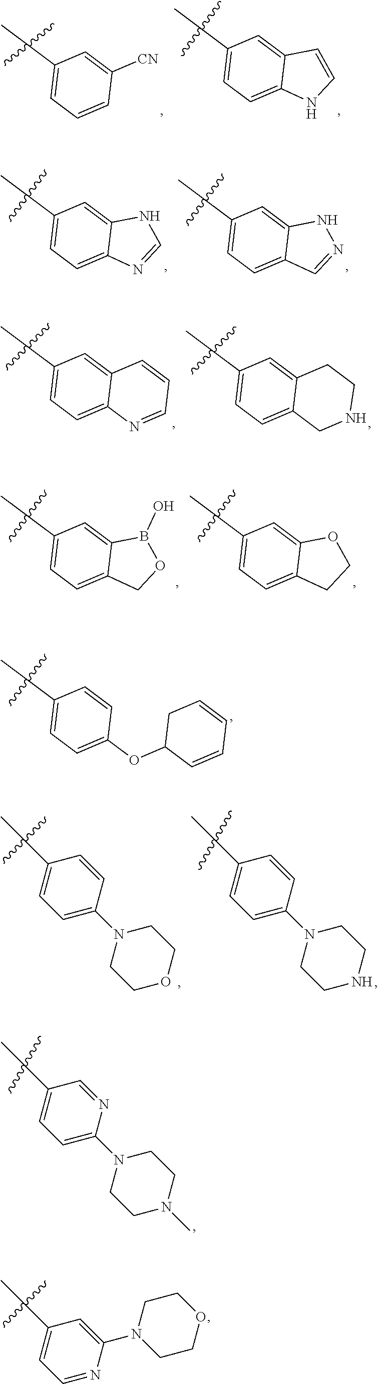

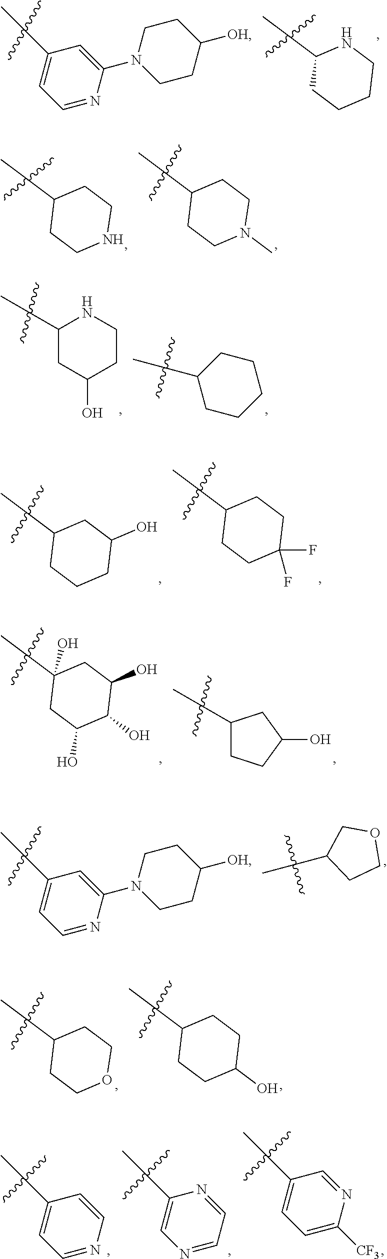

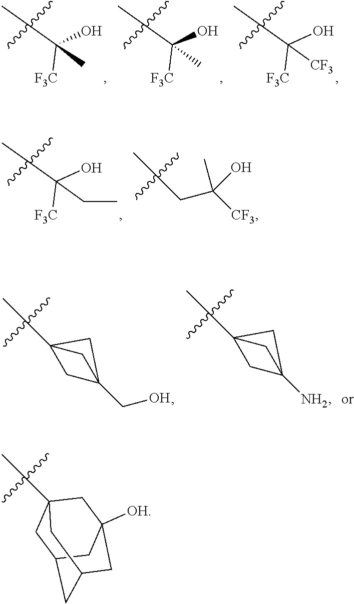

In some embodiments of Formula II, R₂ is

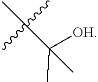 [SMILES: *C(C)(C)O]

In some embodiments of Formula I, the compound is of Formula III or an isomeride, a stereoisomer, tautomer, pharmaceutically acceptable salt, prodrug, chelate, non-covalent complex, or solvate thereof:

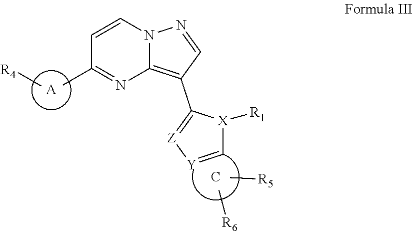

wherein,

ring A is C₅₋₆ heterocyclic ring, wherein the C₅₋₆ heterocyclic ring optionally comprising 1, 2 or 3 hetero atoms independently selected from N, S, or O;

ring C is a 5- to 6-membered carbocyclic ring, or heterocyclic ring, aryl ring, or heteroaryl ring;

X and Z are each independently selected from C, N, O, or S;

Y is C or N;

R₁ is absent, H, or —C₁₋₈ alkyl;

R₄ is (i) phenyl optionally substituted with one or more substituents independently selected from halogen, —C₁₋₄ alkyl, —C₁₋₄ haloalkyl, C₁₋₄alkoxyl, or (ii) a C₅₋₆ heteroaryl ring having a ring heteroatom selected from N, S, or O, wherein the C₅₋₆ heteroaryl ring is optionally substituted with one or more halogen atoms;

R₅ and R₆ are each independently selected from H, OH, NH₂, CN, —COON, —CONHOH, —CONH₂, halogen, —C₁₋₈alkyl, —C₀₋₄ alkyl-COOR₁₀, —C₀₋₄ alkyl-O(CO)OR₁₀, —C₁₋₈ alkoxy, —C₁₋₈ haloalkoxy, —C₁₋₈ alkoxy-C₁₋₈ alkoxy, —C₁₋₈ alkylthio, —C₁₋₈ haloalkylthio, —C₁₋₈ alkyl, —C₁₋₈ haloalkyl, —C₀₋₄ alkyl-OH, —O—CH₂—CN, —C₀₋₄ alkyl-O—C₃₋₁₀ heterocyclic ring, substituted or unsubstituted —C₃₋₁₀ carbocyclic ring or substituted or unsubstituted —C₃₋₁₀ heterocyclic ring;

or R₅ and R₆ together with the atoms to which they are attached to form a 5 to 12-membered carbocyclic ring, heterocyclic ring, aryl ring, or heteroaryl ring, wherein the 5 to 12-membered carbocyclic ring, heterocyclic ring, aryl ring, or heteroaryl ring is optionally substituted with halogen;

R₁₀ is H, or —C₁₋₈ alkyl;

wherein the heterocyclic ring or the heteroaryl ring optionally has 1, 2 or 3 heteroatoms independently selected from N, S, or O.

In some embodiments of Formula III, ring A is

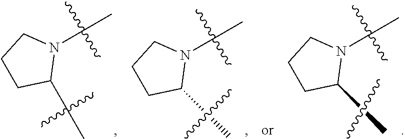

In some embodiments of Formula III, ring C is 6-membered aromatic ring.

In some embodiments of Formula III, ring C is phenyl, pyridyl, pyridazinyl, or pyrimidinyl.

In some embodiments of Formula III, ring C is phenyl.

In some embodiments of Formula III, X is selected from O, S, or N.

In some embodiments of Formula III, X is N.

In some embodiments of Formula III, Y is C.

In some embodiments of Formula III, Z is N.

In some embodiments of Formula III, R₁ is absent, H, or CH₃.

In some embodiments of Formula III, R₄ is

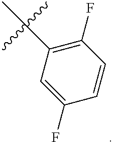 [SMILES: *c1cc(F)ccc1F]

In some embodiments of Formula III, R₅ and R₆ are each independently selected from H, OH, NH₂, F, Cl, Br—CN, —CF₃, —OCF₃, CH₃, —O—CH₃, —S—CH₃, —CH₂OH, —COOH,

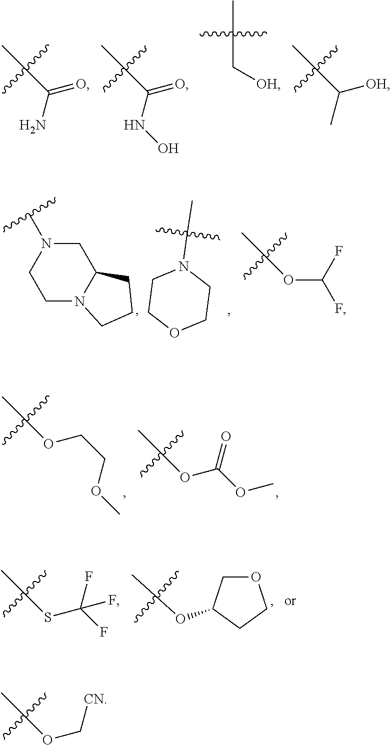

In some embodiments of Formula III, R₅ and R₆ are both —O—CH₃.

In some embodiments of Formula III, R₅ and R₆ together with the atoms to which they are attached from

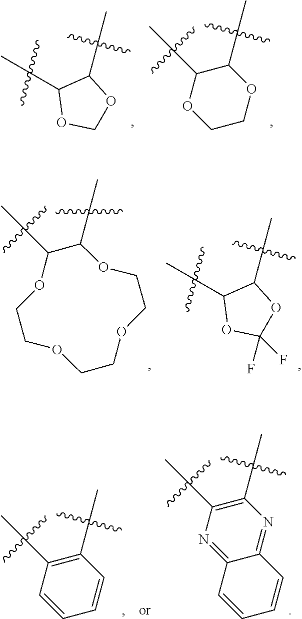 [SMILES: *C1OCOC1* ; *C1OCCOC1* ; *C1OCCOCCOCCOC1* ; *C1OC(F)(F)OC1* ; *c1ccccc1* ; *c1nc2ccccc2nc1*]

In some embodiments of Formula I, the compound is of Formula IV or an isomeride, a stereoisomer, tautomer, pharmaceutically acceptable salt, prodrug, chelate, non-covalent complex, or solvate thereof,

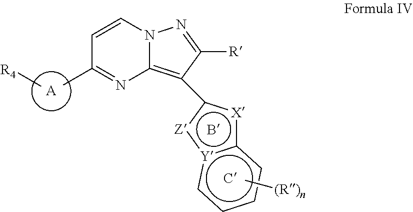

wherein,

Ring A is C₅₋₆ heterocyclic ring, wherein the C₅₋₆ heterocyclic ring optionally comprising 1, 2 or 3 hetero atoms independently selected from N, S, or O;

R₄ is (i) phenyl optionally substituted with one or more substituents independently selected from halogen, —C₁₋₄ alkyl, —C₁₋₄ haloalkyl, —C₁₋₄ alkoxyl, or (ii) a C₅₋₆ heteroaryl ring having a ring heteroatom selected from N, S, or O, wherein the C₅₋₆ heteroaryl ring is optionally substituted with one or more halogen atoms;

R′ is H, NH₂, or —C₁₋₄ alkyl;

Ring B′ is a 5-membered aromatic heterocyclic ring optionally comprising 1, 2 or 3 hetero atoms independently selected from N, S, or O;

Ring C′ is a phenyl, 6-membered heterocyclic ring, or 6-membered heteroaryl ring;

X′ and Z′ are each independently selected from C, N, O, or S;

Y′ is C or N;

R″ is —C(O)—C₁₋₄ alkyl, —SO—C₁₋₄ alkyl, —SO₂—C₁₋₄ alkyl, —NR₇(CH₂)ₘNR₈R₉, —(CH₂)ₘC₄₋₁₀ heterocyclyl; or NH₂, —C(O)OH, —C(O)NH₂, —C₁₋₄ alkyl, —C₁₋₄ alkoxyl, —C(O)—C₁₋₄ alkyl, —C(O)O—C₁₋₄ alkyl, —OC(O)O—C₁₋₄ alkyl, —S—C₁₋₄ alkyl, —SO₂—C₁₋₄ alkyl, —SO₂—C₁₋₄ alkyl, —OC₄₋₆heterocyclyl, —NR₇(CH₂)ₘNR₈R₉, —(CH₂)ₘC₄₋₁₀ heterocyclyl optionally substituted with one or more substituents independently selected from OH, CN, NH₂, —C(O)OH, halogen, —C₁₋₄ alkyl or —C₁₋₄ alkoxyl; or

any two R″ together with the atoms to which they are attached form a 5- to 12-membered ring;

R₇, R₈ and R₉ are each independently selected from H, or —C₁₋₄ alkyl;

m and n are each independently selected from 0, 1, 2, 3 or 4.

In some embodiments of Formula IV, Ring A is

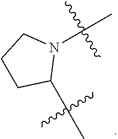 [SMILES: *C1CCCN1*]

In some embodiments of Formula IV, R′ is selected from H.

In some embodiments of Formula IV, R₄ is

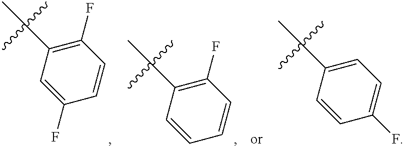

In some embodiments of Formula IV, Ring B′ is selected from imidazole, oxazole, thiazole, triazole or pyrrole.

In some embodiments of Formula IV, Ring B′ is selected from

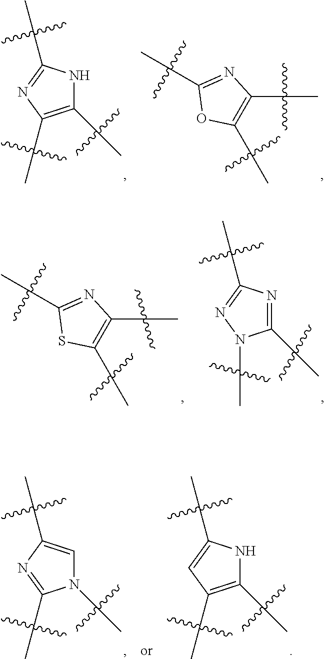 [SMILES: *c1nc(*)c(*)[nH]1 ; *c1nc(*)c(*)o1 ; *c1nc(*)c(*)s1 ; *c1nc(*)n(*)n1 ; *c1cn(*)c(*)n1 ; *c1cc(*)c(*)[nH]1]

In some embodiments of Formula IV, Ring C′ is selected from phenyl, pyridine, pyrazine, pyrimidine, pyridazine, piperidine, or tetrahydropyran.

In some embodiments of Formula IV, Ring C′ is selected from

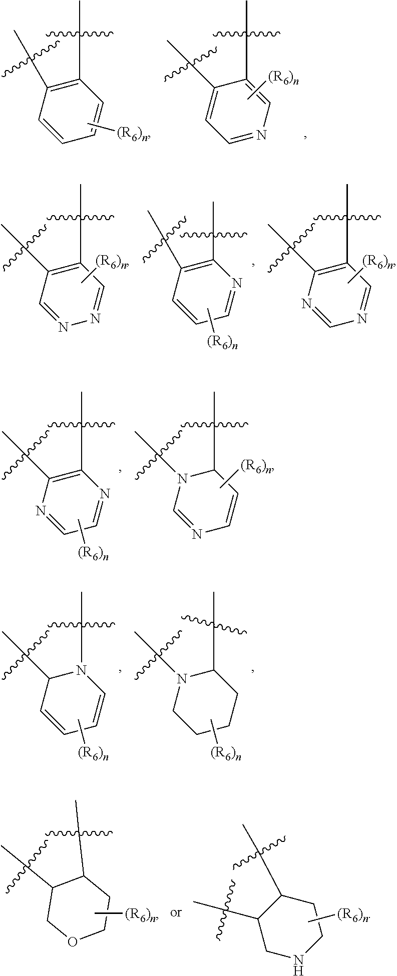

In some embodiments of Formula IV, R″ is selected from

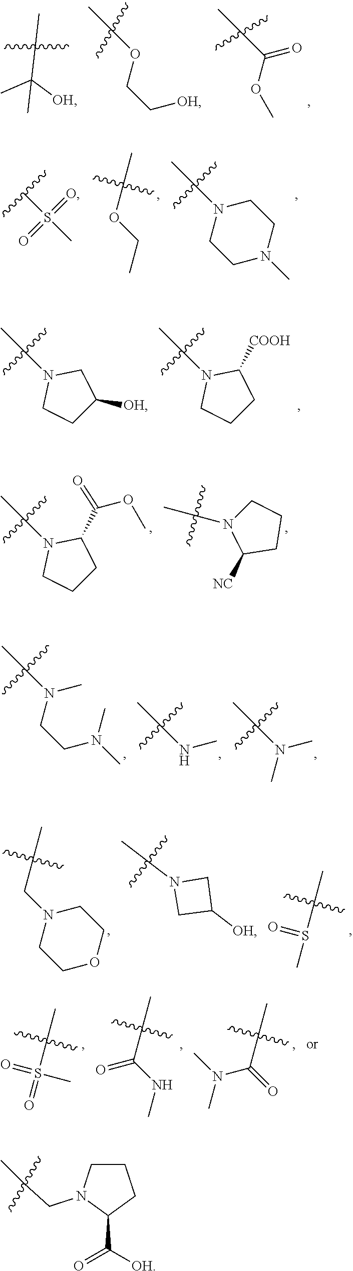

In some embodiments of Formula IV, two R″ with the atoms to which they are attached form

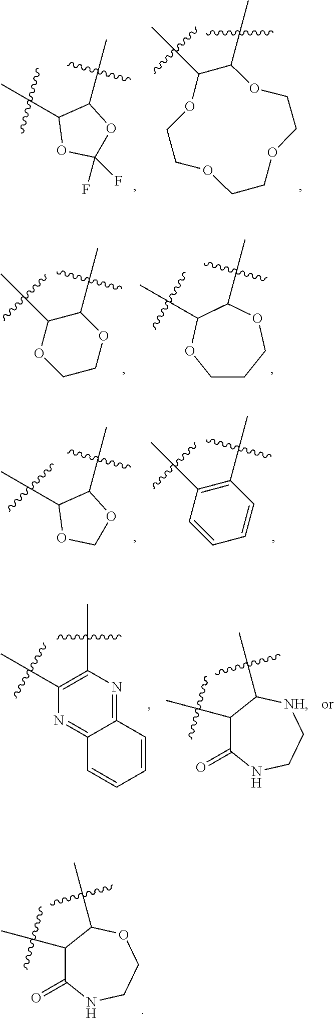

In some embodiments of Formula I, wherein the compound is of Formula V or an isomeride, pharmaceutically acceptable salt thereof,

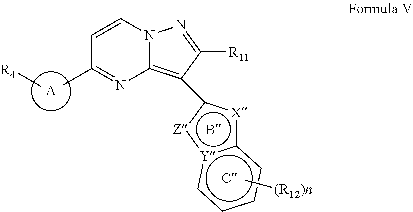

wherein,

Ring A is C₅₋₆ heterocyclic ring, wherein the C₅₋₆ heterocyclic ring optionally comprising 1, 2 or 3 hetero atoms independently selected from N, S, or O;

R₄ is (i) phenyl optionally substituted with one or more substituents independently selected from halogen, —C₁₋₄ alkyl, —C₁₋₄ haloalkyl, —C₁₋₄ alkoxyl, or (ii) a C₅₋₆ heteroaryl ring having a ring heteroatom selected from N, S, or O, wherein the C₅₋₆ heteroaryl ring is optionally substituted with one or more halogen atoms;

R₁₁ is H, NH₂, or —C₁₋₄ alkyl;

Ring B″ is a 5-membered aromatic heterocyclic ring optionally comprising 1, 2 or 3 hetero atoms independently selected from N, S, or O;

Ring C″ is a phenyl, 6-membered heterocyclic ring, or 6-membered heteroaryl ring;

X″ and Z″ are each independently selected from C, N, O, or S;

Y″ is C or N;

R₁₂ is selected from H, OH, CN, NH₂, —C(O)OH, —C(O)NH₂, halogen, —Cₘ alkyl, —C₁₋₄ alkoxyl, —C(O)—C₁₋₄ alkyl, —C(O)O—C₁₋₄ alkyl, —OC(O)O—C₁₋₄ alkyl, —S—C₁₋₄ alkyl, —SO—C₁₋₄ alkyl, —SO₂—C₁₋₄ alkyl, —OC₄₋₆heterocyclyl, —NR′₇(CH₂)ₘNR_{8′}R′₉, —(CH₂)ₘC₄₋₁₀heterocyclyl; wherein NH₂, —C(O)OH, —C(O)NH₂, halogen, —C₁₋₄ alkyl, —C₁₋₄ alkoxyl, —C(O)—C₁₋₄ alkyl, —C(O)O—C₁₋₄ alkyl, —OC(O)O—C₁₋₄ alkyl, —S—C₁₋₄ alkyl, —SO—C₁₋₄ alkyl, —SO₂—C₁₋₄ alkyl, —OC₄₋₆heterocyclyl, —NR₇′(CH₂)ₘNR₈′R₉′, —(CH₂)ₘC₄₋₁₀heterocyclyl optionally substituted with one or more substituents independently selected from OH, CN, NH₂, —C(O)OH, halogen, —C₁₋₄ alkyl or —C₁₋₄ alkoxyl; or

any two R₁₂ together with the atoms to which they are attached form a 5- to 12-membered ring;

R₇′, R₈′ and R₉′ are each independently selected from H, or —C₁₋₄ alkyl;

m and n are each independently selected from 0, 1, 2, 3 or 4.

In some embodiments of Formula V, wherein Ring A is

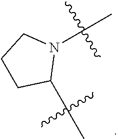 [SMILES: *C1CCCN1*]

In some embodiments of Formula V, wherein R₁₁ is selected from H, NH₂ or CH₃.

In some embodiments of Formula V, wherein R₄ is

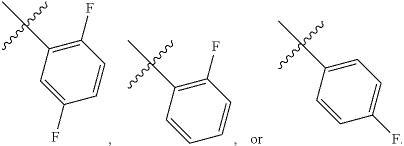

In some embodiments of Formula V, wherein Ring B″ is selected from imidazole, oxazole, thiazole, triazole or pyrrole.

In some embodiments of Formula V, wherein Ring B″ is selected from

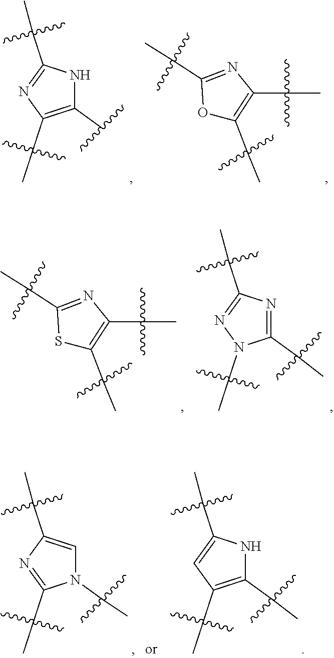 [SMILES: *c1nc(*)c(*)[nH]1 ; *c1nc(*)c(*)o1 ; *c1nc(*)c(*)s1 ; *c1nc(*)n(*)n1 ; *c1cn(*)c(*)n1 ; *c1cc(*)c(*)[nH]1]

In some embodiments of Formula V, wherein Ring C″ is selected from phenyl, pyridine, pyrazine, pyrimidine, pyridazine, piperidine or tetrahydropyran.

In some embodiments of Formula V, wherein Ring C″ is selected from

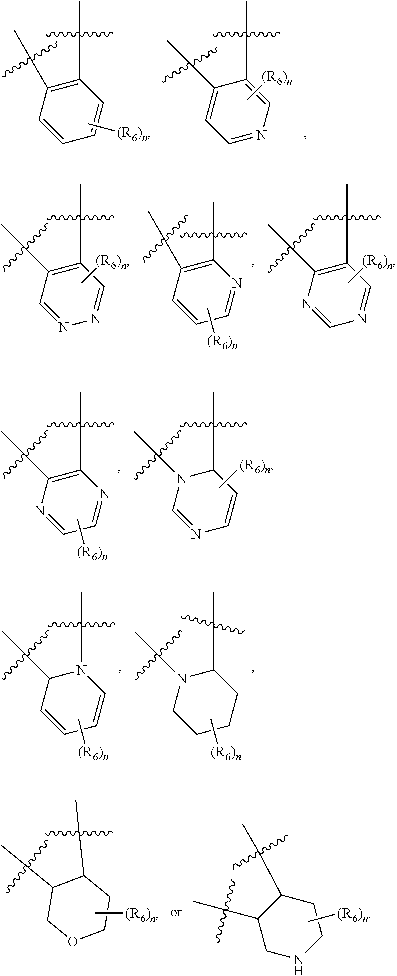

In some embodiments of Formula V, wherein R₁₂ is selected from H, —OH, F, Cl, Br, —CH₃, —NH₂, —COOH, —CN,

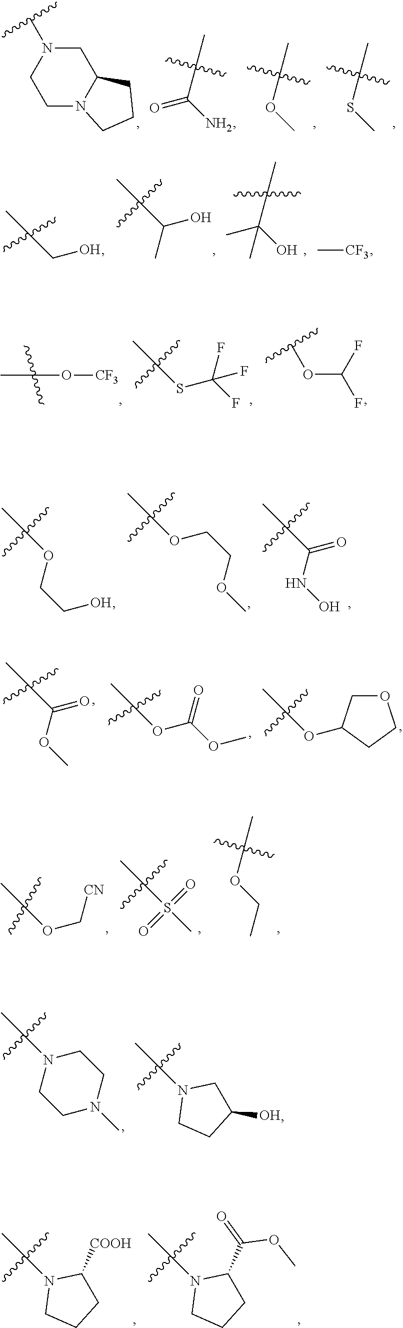

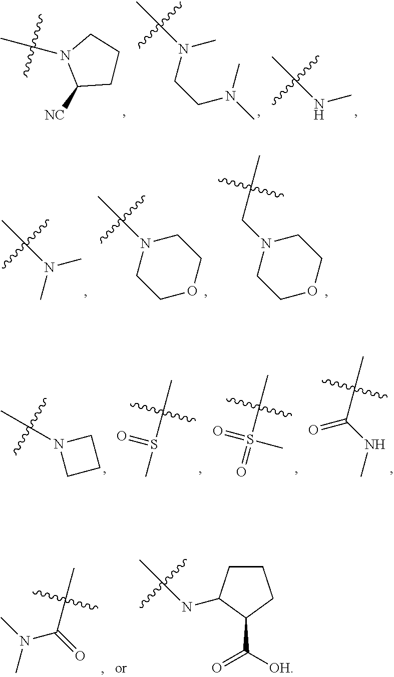

In some embodiments of Formula V, wherein two R₁₂ with the atoms to which they are attached form

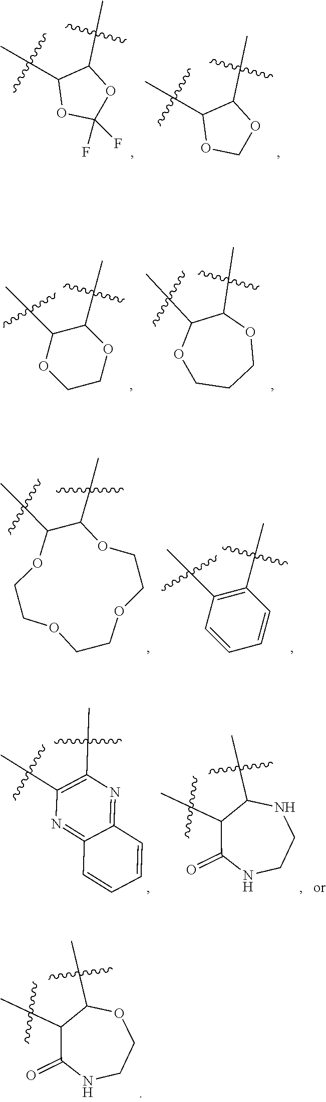

The present invention further provides some preferred technical solutions with regard to compound of Formula I, Formula II, Formula III, Formula IV, or Formula V, or an isomeride, pharmaceutically acceptable salt or solvate thereof, wherein the compound is:
1) (R)-4-(4-(5-(5-(2-(2,5-difluorophenyl)pyrrolidin-1-yl)pyrazolo[1,5-a]pyrimidin-3-yl)-4H-1,2,4-triazol-3-yl)phenyl)morpholine;
2) (R)-1-(4-(5-(5-(2-(2,5-difluorophenyl)pyrrolidin-1-yl)pyrazolo[1,5-a]pyrimidin-3-yl)-4H-1,2,4-triazol-3-yl)pyridin-2-yl)piperidin-4-ol;
3) 5-((R)-2-(2,5-difluorophenyl)pyrrolidin-1-yl)-3-(5-(tetrahydrofuran-3-yl)-4H-1,2,4-triazol-3-yl)pyrazolo[1,5-a]pyrimidine;
4) (S)-1-(5-(5-((R)-2-(2,5-difluorophenyl)pyrrolidin-1-yl)pyrazolo[1,5-a]pyrimidin-3-yl)-4H-1,2,4-triazol-3-yl)ethan-1-ol;
5) (1S,4s)-4-(5-(5-((R)-2-(2,5-difluorophenyl)pyrrolidin-1-yl)pyrazolo[1,5-a]pyrimidin-3-yl)-4H-1,2,4-triazol-3-yl)cyclohexan-1-ol;
6) (R)-4-(4-(5-(5-(2-(2,5-difluorophenyl)pyrrolidin-1-yl)pyrazolo[1,5-a]pyrimidin-3-yl)-4H-1,2,4-triazol-3-yl)pyridin-2-yl)morpholine;
7) (R)-2-(5-(5-(2-(2,5-difluorophenyl)pyrrolidin-1-yl)pyrazolo[1,5-a]pyrimidin-3-yl)-4H-1,2,4-triazol-3-yl)propan-2-ol;
8) (R)-5-(2-(2,5-difluorophenyl)pyrrolidin-1-yl)-3-(5-(pyridin-4-yl)-4H-1,2,4-triazol-3-yl)pyrazolo[1,5-a]pyrimidine;
9) (R)-4-(5-(5-(2-(2,5-difluorophenyl)pyrrolidin-1-yl)pyrazolo[1,5-a]pyrimidin-3-yl)-4H-1,2,4-triazol-3-yl)phenol;
10) (R)-5-(2-(2,5-difluorophenyl)pyrrolidin-1-yl)-3-(5-(pyrazin-2-yl)-4H-1,2,4-triazol-3-yl)pyrazolo[1,5-a]pyrimidine;
11) (S)-1-(5-(5-((R)-2-(2,5-difluorophenyl)pyrrolidin-1-yl)pyrazolo[1,5-a]pyrimidin-3-yl)-4H-1,2,4-triazol-3-yl)ethan-1-amine;
12) methyl ((S)-1-(5-(5-((R)-2-(2,5-difluorophenyl)pyrrolidin-1-yl)pyrazolo[1,5-a]pyrimidin-3-yl)-4H-1,2,4-triazol-3-yl)ethyl)carbamate;
13) (R)-3-(5-(5-(2-(2,5-difluorophenyl)pyrrolidin-1-yl)pyrazolo[1,5-a]pyrimidin-3-yl)-4H-1,2,4-triazol-3-yl)benzonitrile;
14) (R)-5-(2-(2,5-difluorophenyl)pyrrolidin-1-yl)-3-(5-(6-(trifluoromethyl)pyridin-3-yl)-4H-1,2,4-triazol-3-yl)pyrazolo[1,5-a]pyrimidine;
15) 3-(5-(azetidin-2-yl)-4H-1,2,4-triazol-3-yl)-5-((R)-2-(2,5-difluorophenyl)pyrrolidin-1-yl)pyrazolo[1,5-a]pyrimidine;
16) ethyl (R)-5-(5-(2-(2,5-difluorophenyl)pyrrolidin-1-yl)pyrazolo[1,5-a]pyrimidin-3-yl)-4H-1,2,4-triazole-3-carboxylate;
17) (R)-5-(5-(2-(2,5-difluorophenyl)pyrrolidin-1-yl)pyrazolo[1,5-a]pyrimidin-3-yl)-4H-1,2,4-triazole-3-carboxylic acid;
18) (3S)-3-(5-(5-((R)-2-(2,5-difluorophenyl)pyrrolidin-1-yl)pyrazolo[1,5-a]pyrimidin-3-yl)-4H-1,2,4-triazol-3-yl)cyclohexan-1-ol;
19) (3 S)-3-(5-(5-((R)-2-(2,5-difluorophenyl)pyrrolidin-1-yl)pyrazolo[1,5-a]pyrimidin-3-yl)-4H-1,2,4-triazol-3-yl)cyclopentan-1-ol;
20) tert-butyl 2-(5-(5-((R)-2-(2,5-difluorophenyl)pyrrolidin-1-yl)pyrazolo[1,5-a]pyrimidin-3-yl)-4H-1,2,4-triazol-3-yl)azetidine-1-carboxylate;
21) (R)-1-(4-(5-(5-(2-(2,5-difluorophenyl)pyrrolidin-1-yl)pyrazolo[1,5-a]pyrimidin-3-yl)-4-methyl-4H-1,2,4-triazol-3-yl)pyridin-2-yl)piperidin-4-ol;
22) (R)-5-(2-(2,5-difluorophenyl)pyrrolidin-1-yl)-3-(5-(piperidin-4-yl)-4H-1,2,4-triazol-3-yl)pyrazolo[1,5-a]pyrimidine;
23) (R)-1-(5-(5-(2-(2,5-difluorophenyl)pyrrolidin-1-yl)pyrazolo[1,5-a]pyrimidin-3-yl)-4H-1,2,4-triazol-3-yl)cyclobutan-1-ol;
24) (R)-1-(5-(5-(2-(2,5-difluorophenyl)pyrrolidin-1-yl)pyrazolo[1,5-a]pyrimidin-3-yl)-4H-1,2,4-triazol-3-yl)cyclobutan-1-amine;
25) (S)-2-(5-(5-((R)-2-(2,5-difluorophenyl)pyrrolidin-1-yl)pyrazolo[1,5-a]pyrimidin-3-yl)-4H-1,2,4-triazol-3-yl)-1,1,1-trifluoropropan-2-ol;
26) (R)-2-(5-(5-((R)-2-(2,5-difluorophenyl)pyrrolidin-1-yl)pyrazolo[1,5-a]pyrimidin-3-yl)-4H-1,2,4-triazol-3-yl)-1,1,1-trifluoropropan-2-ol;
27) (R)-2-(5-(5-(2-(2,5-difluorophenyl)pyrrolidin-1-yl)pyrazolo[1,5-a]pyrimidin-3-yl)-4H-1,2,4-triazol-3-yl)-1,1,1,3,3,3-hexafluoropropan-2-ol;
28) 2-(5-(5-((R)-2-(2,5-difluorophenyl)pyrrolidin-1-yl)pyrazolo[1,5-a]pyrimidin-3-yl)-4H-1,2,4-triazol-3-yl)-1,1,1-trifluorobutan-2-ol;
29) 3-(5-(5-((R)-2-(2,5-difluorophenyl)pyrrolidin-1-yl)pyrazolo[1,5-a]pyrimidin-3-yl)-4H-1,2,4-triazol-3-yl)-1,1,1-trifluoro-2-methylpropan-2-ol;
30) (R)-1-(5-(5-(2-(2,5-difluorophenyl)pyrrolidin-1-yl)pyrazolo[1,5-a]pyrimidin-3-yl)-4H-1,2,4-triazol-3-yl)-2-methylpropan-2-ol;
31) (R)-3-(5-(5-(2-(2,5-difluorophenyl)pyrrolidin-1-yl)pyrazolo[1,5-a]pyrimidin-3-yl)-4H-1,2,4-triazol-3-yl)cyclobutan-1-ol;
32) (R)-5-(2-(2,5-difluorophenyl)pyrrolidin-1-yl)-3-(5-(tetrahydro-2H-pyran-4-yl)-4H-1,2,4-triazol-3-yl)pyrazolo[1,5-a]pyrimidine;
33) (R)-2-(5-(5-(2-(2,5-difluorophenyl)pyrrolidin-1-yl)pyrazolo[1,5-a]pyrimidin-3-yl)-4H-1,2,4-triazol-3-yl)-2-methylpropan-1-ol;
34) (R)-1-(5-(5-((R)-2-(2,5-difluorophenyl)pyrrolidin-1-yl)pyrazolo[1,5-a]pyrimidin-3-yl)-4H-1,2,4-triazol-3-yl)ethan-1-ol;
35) 2-(5-(5-((R)-2-(2,5-difluorophenyl)pyrrolidin-1-yl)pyrazolo[1,5-a]pyrimidin-3-yl)-4H-1,2,4-triazol-3-yl)piperidin-4-ol;
36) (R)-6-(5-(5-(2-(2,5-difluorophenyl)pyrrolidin-1-yl)pyrazolo[1,5-a]pyrimidin-3-yl)-4H-1,2,4-triazol-3-yl)-1,2,3,4-tetrahydroisoquinoline;
37) (1R,3r)-3-(5-(5-((R)-2-(2,5-difluorophenyl)pyrrolidin-1-yl)pyrazolo[1,5-a]pyrimidin-3-yl)-4H-1,2,4-triazol-3-yl)adamantan-1-ol;
38) (R)-5-(2-(2,5-difluorophenyl)pyrrolidin-1-yl)-3-(5-(1-methylpiperidin-4-yl)-4H-1,2,4-triazol-3-yl)pyrazolo[1,5-a]pyrimidine;
39) (R)-2-(5-(5-((R)-2-(2,5-difluorophenyl)pyrrolidin-1-yl)pyrazolo[1,5-a]pyrimidin-3-yl)-4H-1,2,4-triazol-3-yl)propan-1-ol;
40) (R)-5-(2-(2,5-difluorophenyl)pyrrolidin-1-yl)-3-(5-(4-(piperazin-1-yl)phenyl)-4H-1,2,4-triazol-3-yl)pyrazolo[1,5-a]pyrimidine;
41) (R)-3-(5-(4,4-difluorocyclohexyl)-4H-1,2,4-triazol-3-yl)-5-(2-(2,5-difluorophenyl)pyrrolidin-1-yl)pyrazolo[1,5-a]pyrimidine;
42) (R)-(5-(5-((R)-2-(2,5-difluorophenyl)pyrrolidin-1-yl)pyrazolo[1,5-a]pyrimidin-3-yl)-4H-1,2,4-triazol-3-yl)(phenyl)methanol;
43) (R)-(3-(5-(5-(2-(2,5-difluorophenyl)pyrrolidin-1-yl)pyrazolo[1,5-a]pyrimidin-3-yl)-4H-1,2,4-triazol-3-yl)bicyclo[1.1.1]pentan-1-yl)methanol;
44) (R)-3-(5-(5-(2-(2,5-difluorophenyl)pyrrolidin-1-yl)pyrazolo[1,5-a]pyrimidin-3-yl)-4H-1,2,4-triazol-3-yl)bicyclo[1.1.1]pentan-1-amine;
45) (R)-6-(5-(5-(2-(2,5-difluorophenyl)pyrrolidin-1-yl)pyrazolo[1,5-a]pyrimidin-3-yl)-4H-1,2,4-triazol-3-yl)benzo[c][1,2]oxaborol-1(3H)-ol;
46) 1-(5-(5-((R)-2-(2,5-difluorophenyl)pyrrolidin-1-yl)pyrazolo[1,5-a]pyrimidin-3-yl)-4H-1,2,4-triazol-3-yl)-1,1-difluorobutan-2-ol;
47) 1-(5-(5-((R)-2-(2,5-difluorophenyl)pyrrolidin-1-yl)pyrazolo[1,5-a]pyrimidin-3-yl)-4H-1,2,4-triazol-3-yl)-2,2,2-trifluoroethan-1-ol;
48) 1-(5-(5-((R)-2-(2,5-difluorophenyl)pyrrolidin-1-yl)pyrazolo[1,5-a]pyrimidin-3-yl)-4H-1,2,4-triazol-3-yl)prop-2-yn-1-ol;
49) 3-(5-(5-((R)-2-(2,5-difluorophenyl)pyrrolidin-1-yl)pyrazolo[1,5-a]pyrimidin-3-yl)-4H-1,2,4-triazol-3-yl)morpholine;
50) (R)-3-(5-(1H-indol-5-yl)-4H-1,2,4-triazol-3-yl)-5-(2-(2,5-difluorophenyl)pyrrolidin-1-yl)pyrazolo[1,5-a]pyrimidine;
51) (S)-1-(5-(5-((R)-2-(2,5-difluorophenyl)pyrrolidin-1-yl)pyrazolo[1,5-a]pyrimidin-3-yl)-4H-1,2,4-triazol-3-yl)ethyl L-leucinate hydrochloride;
52) 2-(5-(5-((R)-2-(2,5-difluorophenyl)pyrrolidin-1-yl)pyrazolo[1,5-a]pyrimidin-3-yl)-4H-1,2,4-triazol-3-yl)-2-fluoroethan-1-ol;
53) (R)-1-(5-(5-(2-(2,5-difluorophenyl)pyrrolidin-1-yl)pyrazolo[1,5-a]pyrimidin-3-yl)-4H-1,2,4-triazol-3-yl)cyclopropan-1-ol;
54) (R)-5-(2-(2,5-difluorophenyl)pyrrolidin-1-yl)-3-(5-(6-(4-methylpiperazin-1-yl)pyridin-3-yl)-4H-1,2,4-triazol-3-yl)pyrazolo[1,5-a]pyrimidine;
55) (S)-1-(5-(5-((R)-2-(2,5-difluorophenyl)pyrrolidin-1-yl)pyrazolo[1,5-a]pyrimidin-3-yl)-4H-1,2,4-triazol-3-yl)ethyl L-valylvalinate hydrochloride;
56) (R)-6-(5-(5-(2-(2,5-difluorophenyl)pyrrolidin-1-yl)pyrazolo[1,5-a]pyrimidin-3-yl)-4H-1,2,4-triazol-3-yl)quinoline;
57) (R)-3-(5-(1H-benzo[d]imidazol-6-yl)-4H-1,2,4-triazol-3-yl)-5-(2-(2,5-difluorophenyl)pyrrolidin-1-yl)pyrazolo[1,5-a]pyrimidine;
58) (R)-5-(2-(2,5-difluorophenyl)pyrrolidin-1-yl)-3-(5-(4-phenoxyphenyl)-4H-1,2,4-triazol-3-yl)pyrazolo[1,5-a]pyrimidine;
59) (R)-3-(5-(1H-indazol-6-yl)-4H-1,2,4-triazol-3-yl)-5-(2-(2,5-difluorophenyl)pyrrolidin-1-yl)pyrazolo[1,5-a]pyrimidine;
60) (1R,2S,3R,5S)-5-(5-(5-((R)-2-(2,5-difluorophenyl)pyrrolidin-1-yl)pyrazolo[1,5-a]pyrimidin-3-yl)-4H-1,2,4-triazol-3-yl)cyclohexane-1,2,3,5-tetraol;
61) (R)-5-(2-(2,5-difluorophenyl)pyrrolidin-1-yl)-3-(5-(2,3-dihydrobenzofuran-6-yl)-4H-1,2,4-triazol-3-yl)pyrazolo[1,5-a]pyrimidine;
62) 5-((R)-2-(2,5-difluorophenyl)pyrrolidin-1-yl)-3-(6-((R)-hexahydropyrrolo[1,2-a]pyrazin-2(1H)-yl)-1H-benzo[d]imidazol-2-yl)pyrazolo[1,5-a]pyrimidine;
63) 2-(5-((R)-2-(2,5-difluorophenyl)pyrrolidin-1-yl)pyrazolo[1,5-a]pyrimidin-3-yl)-6-((R)-hexahydropyrrolo[1,2-a]pyrazin-2(1H)-yl)benzo[d]thiazole;
64) (R)-4-(2-(5-(2-(2,5-difluorophenyl)pyrrolidin-1-yl)pyrazolo[1,5-a]pyrimidin-3-yl)-1H-imidazo[4,5-c]pyridin-6-yl)morpholine;
65) (R)-2-(5-(2-(2,5-difluorophenyl)pyrrolidin-1-yl)pyrazolo[1,5-a]pyrimidin-3-yl)-1H-imidazo[4,5-c]pyridine;
66) 1-(2-(5-((R)-2-(2,5-difluorophenyl)pyrrolidin-1-yl)pyrazolo[1,5-a]pyrimidin-3-yl)-5-methoxy-1H-benzo[d]imidazol-6-yl)ethan-1-ol;
67) (R)-(2-(5-(2-(2,5-difluorophenyl)pyrrolidin-1-yl)pyrazolo[1,5-a]pyrimidin-3-yl)-5-methoxy-1H-benzo[d]imidazol-6-yl)methanol;
68) 1-(2-(5-((R)-2-(2,5-difluorophenyl)pyrrolidin-1-yl)pyrazolo[1,5-a]pyrimidin-3-yl)benzo[d]oxazol-6-yl)ethan-1-ol;
69) (R)-(2-(5-(2-(2,5-difluorophenyl)pyrrolidin-1-yl)pyrazolo[1,5-a]pyrimidin-3-yl)benzo[d]oxazol-6-yl)methanol;
70) 1-(2-(5-((R)-2-(2,5-difluorophenyl)pyrrolidin-1-yl)pyrazolo[1,5-a]pyrimidin-3-yl)-3H-imidazo[4,5-c]pyridin-6-yl)ethan-1-ol;
71) (R)-5-(2-(2,5-difluorophenyl)pyrrolidin-1-yl)-3-(5-(trifluoromethoxy)-1H-benzo[d]imidazol-2-yl)pyrazolo[1,5-a]pyrimidine;
72) (R)-2-(5-(2-(2,5-difluorophenyl)pyrrolidin-1-yl)pyrazolo[1,5-a]pyrimidin-3-yl)-6-(trifluoromethyl)-3H-imidazo[4,5-c]pyridine;
73) (R)-2-(5-(2-(2,5-difluorophenyl)pyrrolidin-1-yl)pyrazolo[1,5-a]pyrimidin-3-yl)oxazolo[4,5-c]pyridine;
74) (R)-5-(2-(2,5-difluorophenyl)pyrrolidin-1-yl)-3-(6-fluoro-1H-benzo[d]imidazol-2-yl)pyrazolo[1,5-a]pyrimidine;
75) (R)-2-(5-(2-(2,5-difluorophenyl)pyrrolidin-1-yl)pyrazolo[1,5-a]pyrimidin-3-yl)thiazolo[4,5-c]pyridine;
76) (R)-2-(5-(2-(2,5-difluorophenyl)pyrrolidin-1-yl)pyrazolo[1,5-a]pyrimidin-3-yl)-1H-imidazo[4,5-d]pyridazine;
77) (R)-5-(2-(2,5-difluorophenyl)pyrrolidin-1-yl)-3-(6-methoxy-1H-benzo[d]imidazol-2-yl)pyrazolo[1,5-a]pyrimidine;
78) (R)-5-(2-(2,5-difluorophenyl)pyrrolidin-1-yl)-3-(5,6-dimethoxy-1H-benzo[d]imidazol-2-yl)pyrazolo[1,5-a]pyrimidine;
79) (R)-6-(5-(2-(2,5-difluorophenyl)pyrrolidin-1-yl)pyrazolo[1,5-a]pyrimidin-3-yl)-2,2-difluoro-5H-[1,3]dioxolo[4′,5′:4,5]benzo[1,2-d]imidazole;
80) (R)-2-(5-(2-(2,5-difluorophenyl)pyrrolidin-1-yl)pyrazolo[1,5-a]pyrimidin-3-yl)-6-(trifluoromethoxy)benzo[d]oxazole;
81) (R)-3-(6-(difluoromethoxy)-1H-benzo[d]imidazol-2-yl)-5-(2-(2,5-difluorophenyl)pyrrolidin-1-yl)pyrazolo[1,5-a]pyrimidine;
82) (R)-2-(5-(2-(2,5-difluorophenyl)pyrrolidin-1-yl)pyrazolo[1,5-a]pyrimidin-3-yl)-6,7,9,10,12,13-hexahydro-1H-[1,4,7,10]tetraoxacyclododecino[2′,3′:4,5]benzo[1,2-d]imidazole;
83) (R)-2-(5-(2-(2,5-difluorophenyl)pyrrolidin-1-yl)pyrazolo[1,5-a]pyrimidin-3-yl)-1-methyl-6,7-dihydro-1H-[1,4]dioxino[2′,3′:4,5]benzo[1,2-d]imidazole;
84) (R)-2-(5-(2-(2,5-difluorophenyl)pyrrolidin-1-yl)pyrazolo[1,5-a]pyrimidin-3-yl)-5-methoxy-3H-imidazo[4,5-b]pyridine;
85) (R)-2-(5-(2-(2,5-difluorophenyl)pyrrolidin-1-yl)pyrazolo[1,5-a]pyrimidin-3-yl)-6-methoxy-1H-imidazo[4,5-c]pyridine;
86) (R)-2-(5-(2-(2,5-difluorophenyl)pyrrolidin-1-yl)pyrazolo[1,5-a]pyrimidin-3-yl)-6-methyl-1H-imidazo[4,5-c]pyridine;
87) (R)-8-(5-(2-(2,5-difluorophenyl)pyrrolidin-1-yl)pyrazolo[1,5-a]pyrimidin-3-yl)-7H-purin-6-amine;
88) (R)-8-(5-(2-(2,5-difluorophenyl)pyrrolidin-1-yl)pyrazolo[1,5-a]pyrimidin-3-yl)-7H-purin-6-ol;
89) (R)-2-(5-(2-(2,5-difluorophenyl)pyrrolidin-1-yl)pyrazolo[1,5-a]pyrimidin-3-yl)-N-hydroxy-5-methoxy-1H-benzo[d]imidazole-6-carboxamide;
90) (R)-2-(5-(2-(2,5-difluorophenyl)pyrrolidin-1-yl)pyrazolo[1,5-a]pyrimidin-3-yl)-5-methoxy-1H-benzo[d]imidazole-6-carboxylic acid;
91) (R)-2-(5-(2-(2,5-difluorophenyl)pyrrolidin-1-yl)pyrazolo[1,5-a]pyrimidin-3-yl)-5-methoxy-1H-benzo[d]imidazole-6-carboxamide;
92) (R)-3-(5-chloro-6-(trifluoromethoxy)-1H-benzo[d]imidazol-2-yl)-5-(2-(2,5-difluorophenyl)pyrrolidin-1-yl)pyrazolo[1,5-a]pyrimidine;
93) 1-(2-(5-((R)-2-(2,5-difluorophenyl)pyrrolidin-1-yl)pyrazolo[1,5-a]pyrimidin-3-yl)-6-fluorobenzo[d]oxazol-5-yl)ethan-1-ol;
94) (R)-(2-(5-(2-(2,5-difluorophenyl)pyrrolidin-1-yl)pyrazolo[1,5-a]pyrimidin-3-yl)-6-fluorobenzo[d]oxazol-5-yl)methanol;
95) (R)-(2-(5-(2-(2,5-difluorophenyl)pyrrolidin-1-yl)pyrazolo[1,5-a]pyrimidin-3-yl)-3H-imidazo[4,5-c]pyridin-6-yl)methanol;
96) (R)-2-(5-(2-(2,5-difluorophenyl)pyrrolidin-1-yl)pyrazolo[1,5-a]pyrimidin-3-yl)-6,7-dihydro-1H-[1,4]dioxino[2′,3′:4,5]benzo[1,2-d]imidazole;
97) (R)-3-(7-chloro-1H-benzo[d]imidazol-2-yl)-5-(2-(2,5-difluorophenyl)pyrrolidin-1-yl)pyrazolo[1,5-a]pyrimidine;
98) (R)-3-(7-chloro-5-fluoro-1H-benzo[d]imidazol-2-yl)-5-(2-(2,5-difluorophenyl)pyrrolidin-1-yl)pyrazolo[1,5-a]pyrimidine;
99) (R)-2-(5-(2-(2,5-difluorophenyl)pyrrolidin-1-yl)pyrazolo[1,5-a]pyrimidin-3-yl)-7-methyl-1H-imidazo[4,5-c]pyridine;
100) (R)-2-(5-(2-(2,5-difluorophenyl)pyrrolidin-1-yl)pyrazolo[1,5-a]pyrimidin-3-yl)-4-methoxybenzo[d]oxazole;
101) (R)-3-(5,6-bis(2-methoxyethoxy)-1H-benzo[d]imidazol-2-yl)-5-(2-(2,5-difluorophenyl)pyrrolidin-1-yl)pyrazolo[1,5-a]pyrimidine;
102) (R)-6,7-dichloro-2-(5-(2-(2,5-difluorophenyl)pyrrolidin-1-yl)pyrazolo[1,5-a]pyrimidin-3-yl)-1H-imidazo[4,5-b]pyridine;
103) (R)-2-(5-(2-(2,5-difluorophenyl)pyrrolidin-1-yl)pyrazolo[1,5-a]pyrimidin-3-yl)-4-methyl-3H-imidazo[4,5-c]pyridine;
104) (R)-3-(4,7-dichloro-1H-benzo[d]imidazol-2-yl)-5-(2-(2,5-difluorophenyl)pyrrolidin-1-yl)pyrazolo[1,5-a]pyrimidine;
105) (R)-3-(5,6-dichloro-1H-benzo[d]imidazol-2-yl)-5-(2-(2,5-difluorophenyl)pyrrolidin-1-yl)pyrazolo[1,5-a]pyrimidine;
106) (R)-2-(5-(2-(2,5-difluorophenyl)pyrrolidin-1-yl)pyrazolo[1,5-a]pyrimidin-3-yl)-5-methyl-3H-imidazo[4,5-b]pyridine;
107) (R)-2-(5-(2-(2,5-difluorophenyl)pyrrolidin-1-yl)pyrazolo[1,5-a]pyrimidin-3-yl)-1H-benzo[d]imidazole-6-carbonitrile;
108) (R)-2-(5-(2-(2,5-difluorophenyl)pyrrolidin-1-yl)pyrazolo[1,5-a]pyrimidin-3-yl)-5-fluoro-3H-imidazo[4,5-b]pyridine;
109) (R)-3-(5,6-bis(difluoromethoxy)-1H-benzo[d]imidazol-2-yl)-5-(2-(2,5-difluorophenyl)pyrrolidin-1-yl)pyrazolo[1,5-a]pyrimidine;
110) (R)-2-(5-(2-(2,5-difluorophenyl)pyrrolidin-1-yl)pyrazolo[1,5-a]pyrimidin-3-yl)-6-(trifluoromethyl)-1H-imidazo[4,5-b]pyridine;
111) (R)-2-(5-(2-(2,5-difluorophenyl)pyrrolidin-1-yl)pyrazolo[1,5-a]pyrimidin-3-yl)-5,7-difluorobenzo[d]oxazole;
112) (R)-3-(5-chloro-6-methoxy-1H-benzo[d]imidazol-2-yl)-5-(2-(2,5-difluorophenyl)pyrrolidin-1-yl)pyrazolo[1,5-a]pyrimidine;
113) (R)-5-(2-(2,5-difluorophenyl)pyrrolidin-1-yl)-3-(7-(trifluoromethoxy)-1H-benzo[d]imidazol-2-yl)pyrazolo[1,5-a]pyrimidine;
114) (R)-2-(5-(2-(2,5-difluorophenyl)pyrrolidin-1-yl)pyrazolo[1,5-a]pyrimidin-3-yl)-5-(trifluoromethyl)-3H-imidazo[4,5-b]pyridine;
115) (R)-2-(5-(2-(2,5-difluorophenyl)pyrrolidin-1-yl)pyrazolo[1,5-a]pyrimidin-3-yl)-1H-benzo[d]imidazole-5,6-diyl dimethyl bis(carbonate);
116) (R)-5-(2-(2,5-difluorophenyl)pyrrolidin-1-yl)-3-(6-((trifluoromethyl)thio)-1H-benzo[d]imidazol-2-yl)pyrazolo[1,5-a]pyrimidine;
117) (R)-2-(5-(2-(2,5-difluorophenyl)pyrrolidin-1-yl)pyrazolo[1,5-a]pyrimidin-3-yl)-1H-benzo[d]imidazole-5,6-diol;
118) 5-((R)-2-(2,5-difluorophenyl)pyrrolidin-1-yl)-3-(5-(((R)-tetrahydrofuran-3-yl)oxy)-6-(((S)-tetrahydrofuran-3-yl)oxy)-1H-benzo[d]imidazol-2-yl)pyrazolo[1,5-a]pyrimidine;
119) (R)-2,2′-(2-(5-(2-(2,5-difluorophenyl)pyrrolidin-1-yl)pyrazolo[1,5-a]pyrimidin-3-yl)-1H-benzo[d]imidazole-5,6-diyl)bis(oxy))diacetonitrile;
120) (R)-2-(5-(2-(2,5-difluorophenyl)pyrrolidin-1-yl)pyrazolo[1,5-a]pyrimidin-3-yl)-5,6-dimethoxybenzo[d]oxazole;
121) (R)-2-(5-(2-(2,5-difluorophenyl)pyrrolidin-1-yl)pyrazolo[1,5-a]pyrimidin-3-yl)-1H-imidazo[4,5-b]quinoxaline;
122) (R)-2-(5-(2-(2,5-difluorophenyl)pyrrolidin-1-yl)pyrazolo[1,5-a]pyrimidin-3-yl)-7-methyl-3H-imidazo[4,5-b]pyridine;
123) (R)-2-(5-(2-(2,5-difluorophenyl)pyrrolidin-1-yl)pyrazolo[1,5-a]pyrimidin-3-yl)-5-fluoro-1H-benzo[d]imidazole-6-carbonitrile;
124) (R)-2-(5-(2-(2,5-difluorophenyl)pyrrolidin-1-yl)pyrazolo[1,5-a]pyrimidin-3-yl)-1-methyl-1H-imidazo[4,5-c]pyridine;
125) (R)-2-(5-(2-(2,5-difluorophenyl)pyrrolidin-1-yl)pyrazolo[1,5-a]pyrimidin-3-yl)-5-methoxy-1H-benzo[d]imidazole-6-carbonitrile;
126) (R)-2-(5-(2-(2,5-difluorophenyl)pyrrolidin-1-yl)pyrazolo[1,5-a]pyrimidin-3-yl)-5-(methylthio)-1H-benzo[d]imidazole-6-carbonitrile;
127) (R)-5-(2-(2,5-difluorophenyl)pyrrolidin-1-yl)-3-(7-fluoro-6-methoxy-1H-benzo[d]imidazol-2-yl)pyrazolo[1,5-a]pyrimidine;
128) (R)-2-(5-(2-(2,5-difluorophenyl)pyrrolidin-1-yl)pyrazolo[1,5-a]pyrimidin-3-yl)-1H-imidazo[4,5-b]pyrazine;
129) (R)-6-bromo-2-(5-(2-(2,5-difluorophenyl)pyrrolidin-1-yl)pyrazolo[1,5-a]pyrimidin-3-yl)-1H-imidazo[4,5-b]pyrazine;
130) (R)-2-(5-(2-(2,5-difluorophenyl)pyrrolidin-1-yl)pyrazolo[1,5-a]pyrimidin-3-yl)-1H-imidazo[4,5-b]phenazine;
131) (R)-6-(5-(2-(2,5-difluorophenyl)pyrrolidin-1-yl)pyrazolo[1,5-a]pyrimidin-3-yl)-[1,3]dioxolo[4′,5′:4,5]benzo[1,2-d]oxazole;
132) 2-(5-((R)-2-(2,5-difluorophenyl)pyrrolidin-1-yl)pyrazolo[1,5-a]pyrimidin-3-yl)-5,6,7,8-tetrahydro-[1,2,4]triazolo[1,5-a]pyridin-6-ol;
133) (R)-2-(5-(2-(2,5-difluorophenyl)pyrrolidin-1-yl)pyrazolo[1,5-a]pyrimidin-3-yl)-1H-indole-5-carbonitrile;
134) (R)-2-(5-(2-(2,5-difluorophenyl)pyrrolidin-1-yl)pyrazolo[1,5-a]pyrimidin-3-yl)-7,8-dihydro-1H,6H-[1,4]dioxepino[2′,3′:4,5]benzo[1,2-d]imidazole;
135) (R)-(2-(5-(2-(2,5-difluorophenyl)pyrrolidin-1-yl)pyrazolo[1,5-a]pyrimidin-3-yl)-3H-imidazo[4,5-c]pyridin-6-yl)methanol;
136) (R)-3-(5,6-difluoro-1H-benzo[d]imidazol-2-yl)-5-(2-(2,5-difluorophenyl)pyrrolidin-1-yl)pyrazolo[1,5-a]pyrimidine;
137) methyl (R)-2-(5-(2-(2,5-difluorophenyl)pyrrolidin-1-yl)pyrazolo[1,5-a]pyrimidin-3-yl)-4,5-difluoro-1H-benzo[d]imidazole-6-carboxylate;
138) (R)-2-(5-(2-(2,5-difluorophenyl)pyrrolidin-1-yl)pyrazolo[1,5-a]pyrimidin-3-yl)-4,5-difluoro-1H-benzo[d]imidazole-6-carboxylic acid;
139) (R)-5-(2-(2,5-difluorophenyl)pyrrolidin-1-yl)-3-(5-fluoro-6-(trifluoromethyl)-1H-benzo[d]imidazol-2-yl)pyrazolo[1,5-a]pyrimidine;
140) (R)-2-(5-(2-(2,5-difluorophenyl)pyrrolidin-1-yl)pyrazolo[1,5-a]pyrimidin-3-yl)-6-ethoxy-1H-benzo[d]imidazole-5-carbonitrile;
141) (R)-2-(5-(2-(2,5-difluorophenyl)pyrrolidin-1-yl)pyrazolo[1,5-a]pyrimidin-3-yl)-6-fluoro-1H-benzo[d]imidazole-5-carboxylic acid;
142) (R)-2-(5-(2-(2,5-difluorophenyl)pyrrolidin-1-yl)pyrazolo[1,5-a]pyrimidin-3-yl)-6-(methylamino)-1H-benzo[d]imidazole-5-carbonitrile;
143) (R)-2-(5-(2-(2,5-difluorophenyl)pyrrolidin-1-yl)pyrazolo[1,5-a]pyrimidin-3-yl)-6-morpholino-1H-benzo[d]imidazole-5-carbonitrile;
144) (R)-2-(5-(2-(2,5-difluorophenyl)pyrrolidin-1-yl)pyrazolo[1,5-a]pyrimidin-3-yl)-6-(dimethylamino)-1H-benzo[d]imidazole-5-carbonitrile;
145) (R)-2-(5-(2-(2,5-difluorophenyl)pyrrolidin-1-yl)pyrazolo[1,5-a]pyrimidin-3-yl)-6-(3-hydroxyazetidin-1-yl)-1H-benzo[d]imidazole-5-carbonitrile;
146) (R)-2-(5-(2-(2,5-difluorophenyl)pyrrolidin-1-yl)pyrazolo[1,5-a]pyrimidin-3-yl)-5,6,7,8-tetrahydroimidazo[4′,5′:4,5]benzo[1,2-e][1,4]diazepin-9(3H)-one;
147) (R)-2-(5-(2-(2,5-difluorophenyl)pyrrolidin-1-yl)pyrazolo[1,5-a]pyrimidin-3-yl)-7,8-dihydro-3H-imidazo[4′,5′:4,5]benzo[1,2-f][1,4]oxazepin-9(6H)-one;
148) (R)-2-(5-(2-(2,5-difluorophenyl)pyrrolidin-1-yl)pyrazolo[1,5-a]pyrimidin-3-yl)-1H-benzo[d]imidazole-5,6-dicarbonitrile;
149) (R)-2-(5-(2-(2,5-difluorophenyl)pyrrolidin-1-yl)pyrazolo[1,5-a]pyrimidin-3-yl)-6-hydroxy-1H-benzo[d]imidazole-5-carbonitrile;
150) (R)-2-(5-(2-(2,5-difluorophenyl)pyrrolidin-1-yl)pyrazolo[1,5-a]pyrimidin-3-yl)-6-(2-hydroxyethoxy)-1H-benzo[d]imidazole-5-carbonitrile;
151) (R)-6-bromo-2-(5-(2-(2,5-difluorophenyl)pyrrolidin-1-yl)pyrazolo[1,5-a]pyrimidin-3-yl)-1H-benzo[d]imidazole-5-carbonitrile;
152) methyl (R)-5-cyano-2-(5-(2-(2,5-difluorophenyl)pyrrolidin-1-yl)pyrazolo[1,5-a]pyrimidin-3-yl)-1H-benzo[d]imidazole-6-carboxylate;
153) (R)-5-cyano-2-(5-(2-(2,5-difluorophenyl)pyrrolidin-1-yl)pyrazolo[1,5-a]pyrimidin-3-yl)-1H-benzo[d]imidazole-6-carboxylic acid;
154) (R)-5-cyano-2-(5-(2-(2,5-difluorophenyl)pyrrolidin-1-yl)pyrazolo[1,5-a]pyrimidin-3-yl)-1H-benzo[d]imidazole-6-carboxamide;
155) methyl (R)-2-(5-(2-(2,5-difluorophenyl)pyrrolidin-1-yl)pyrazolo[1,5-a]pyrimidin-3-yl)-6-methoxy-1H-benzo[d]imidazole-5-carboxylate;
156) (R)-6-(difluoromethoxy)-2-(5-(2-(2,5-difluorophenyl)pyrrolidin-1-yl)pyrazolo[1,5-a]pyrimidin-3-yl)-1H-benzo[d]imidazole-5-carbonitrile;
157) (R)-2-(5-(2-(2,5-difluorophenyl)pyrrolidin-1-yl)pyrazolo[1,5-a]pyrimidin-3-yl)-5-(trifluoromethyl)-1H-benzo[d]imidazole-6-carbonitrile;
158) methyl (R)-2-(5-(2-(2,5-difluorophenyl)pyrrolidin-1-yl)pyrazolo[1,5-a]pyrimidin-3-yl)-6-fluoro-1H-benzo[d]imidazole-7-carboxylate;
159) (R)-2-(5-(2-(2,5-difluorophenyl)pyrrolidin-1-yl)pyrazolo[1,5-a]pyrimidin-3-yl)-6-methyl-1H-benzo[d]imidazole-5-carbonitrile;
160) (R)-2-(5-(2-(2,5-difluorophenyl)pyrrolidin-1-yl)pyrazolo[1,5-a]pyrimidin-3-yl)-6-methoxy-N-methyl-1H-benzo[d]imidazole-5-carboxamide;
161) (R)-2-(5-(2-(2,5-difluorophenyl)pyrrolidin-1-yl)pyrazolo[1,5-a]pyrimidin-3-yl)-6-methoxy-N,N-dimethyl-1H-benzo[d]imidazole-5-carboxamide;
162) (R)-4-((2-(5-(2-(2,5-difluorophenyl)pyrrolidin-1-yl)pyrazolo[1,5-a]pyrimidin-3-yl)-1H-benzo[d]imidazol-5-yl)methyl)morpholine;
163) (R)-2-(5-(2-(2,5-difluorophenyl)pyrrolidin-1-yl)pyrazolo[1,5-a]pyrimidin-3-yl)-6-(4-methylpiperazin-1-yl)-1H-benzo[d]imidazole-5-carbonitrile;
164) 2-(5-((R)-2-(2,5-difluorophenyl)pyrrolidin-1-yl)pyrazolo[1,5-a]pyrimidin-3-yl)-6-((S)-3-hydroxypyrrolidin-1-yl)-1H-benzo[d]imidazole-5-carbonitrile;
165) 6-((S)-2-cyanopyrrolidin-1-yl)-2-(5-((R)-2-(2,5-difluorophenyl)pyrrolidin-1-yl)pyrazolo[1,5-a]pyrimidin-3-yl)-1H-benzo[d]imidazole-5-carbonitrile;
166) methyl (5-cyano-2-(5-((R)-2-(2,5-difluorophenyl)pyrrolidin-1-yl)pyrazolo[1,5-a]pyrimidin-3-yl)-1H-benzo[d]imidazol-6-yl)-L-prolinate;
167) (5-cyano-2-(5-((R)-2-(2,5-difluorophenyl)pyrrolidin-1-yl)pyrazolo[1,5-a]pyrimidin-3-yl)-1H-benzo[d]imidazol-6-yl)-L-proline;
168) (R)-2-(5-(2-(2,5-difluorophenyl)pyrrolidin-1-yl)pyrazolo[1,5-a]pyrimidin-3-yl)-6-((2-(dimethylamino)ethyl)(methyl)amino)-1H-benzo[d]imidazole-5-carbonitrile;
169) (R)-2-(5-(2-(2,5-difluorophenyl)pyrrolidin-1-yl)pyrazolo[1,5-a]pyrimidin-3-yl)-6-(2-methoxyethoxy)-1H-benzo[d]imidazole-5-carbonitrile;
170) (R)-5-(2-(2,5-difluorophenyl)pyrrolidin-1-yl)-3-(6-(methylsulfonyl)-1H-benzo[d]imidazol-2-yl)pyrazolo[1,5-a]pyrimidine;
171) 2-(5-((R)-2-(2,5-difluorophenyl)pyrrolidin-1-yl)pyrazolo[1,5-a]pyrimidin-3-yl)-5-(methylsulfinyl)-1H-benzo[d]imidazole-6-carbonitrile;
172) (R)-2-(5-(2-(2,5-difluorophenyl)pyrrolidin-1-yl)pyrazolo[1,5-a]pyrimidin-3-yl)-5-(methylsulfonyl)-1H-benzo[d]imidazole-6-carbonitrile;
173) (R)-2-(5-(2-(2,5-difluorophenyl)pyrrolidin-1-yl)pyrazolo[1,5-a]pyrimidin-3-yl)-5-(methylsulfonyl)-1H-benzo[d]imidazole-6-carboxamide;
174) (R)-2-(5-(2-(2,5-difluorophenyl)pyrrolidin-1-yl)pyrazolo[1,5-a]pyrimidin-3-yl)-6-methoxybenzo[d]oxazole-5-carbonitrile;
175) methyl (R)-2-(5-(2-(2,5-difluorophenyl)pyrrolidin-1-yl)pyrazolo[1,5-a]pyrimidin-3-yl)-4-fluorobenzo[d]oxazole-7-carboxylate;
176) (R)-2-(5-(2-(2,5-difluorophenyl)pyrrolidin-1-yl)pyrazolo[1,5-a]pyrimidin-3-yl)-6-(trifluoromethoxy)benzo[d]oxazole-5-carbonitrile;
177) (R)-2-(5-(2-(2,5-difluorophenyl)pyrrolidin-1-yl)pyrazolo[1,5-a]pyrimidin-3-yl)-6-hydroxybenzo[d]oxazole-5-carbonitrile;
178) methyl (R)-2-(5-(2-(2,5-difluorophenyl)pyrrolidin-1-yl)pyrazolo[1,5-a]pyrimidin-3-yl)-5-methoxybenzo[d]oxazole-6-carboxy late;
179) (R)-6-(difluoromethoxy)-2-(5-(2-(2,5-difluorophenyl)pyrrolidin-1-yl)pyrazolo[1,5-a]pyrimidin-3-yl)-5-methylbenzo[d]oxazole;
180) ((2-(5-((R)-2-(2,5-difluorophenyl)pyrrolidin-1-yl)pyrazolo[1,5-a]pyrimidin-3-yl)-6-methoxybenzo[d]oxazol-5-yl)methyl)-L-proline;
181) (R)-2-(5-(2-(2,5-difluorophenyl)pyrrolidin-1-yl)pyrazolo[1,5-a]pyrimidin-3-yl)-5,8-dimethoxy-[1,2,4]triazolo[1,5-c]pyrimidine;
182) (R)-2-(5-(2-(2,5-difluorophenyl)pyrrolidin-1-yl)pyrazolo[1,5-a]pyrimidin-3-yl)-6,7-dimethoxy-[1,2,4]triazolo[1,5-a]pyridine;
183) (R)-5-(2-(2,5-difluorophenyl)pyrrolidin-1-yl)-3-(6-fluoro-1H-indol-2-yl)pyrazolo[1,5-a]pyrimidine;
184) methyl (R)-2-(5-(2-(2,5-difluorophenyl)pyrrolidin-1-yl)pyrazolo[1,5-a]pyrimidin-3-yl)-1H-indole-5-carboxylate;
185) (R)-2-(5-(2-(2,5-difluorophenyl)pyrrolidin-1-yl)pyrazolo[1,5-a]pyrimidin-3-yl)-1H-indole-5-carboxylic acid;
186) (R)-2-(5-(2-(2,5-difluorophenyl)pyrrolidin-1-yl)pyrazolo[1,5-a]pyrimidin-3-yl)-1H-indol-6-ol;
187) (S)-2-(5-((R)-2-(2,5-difluorophenyl)pyrrolidin-1-yl)pyrazolo[1,5-a]pyrimidin-3-yl)-5,6,7,8-tetrahydro-[1,2,4]triazolo[1,5-a]pyridin-7-ol;
188) (R)-2-(5-(2-(2,5-difluorophenyl)pyrrolidin-1-yl)pyrazolo[1,5-a]pyrimidin-3-yl)-3,4,6,7-tetrahydropyrano[3,4-d]imidazole;
189) (R)-2-(5-(2-(2,5-difluorophenyl)pyrrolidin-1-yl)pyrazolo[1,5-a]pyrimidin-3-yl)-4,5,6,7-tetrahydrothiazolo[4,5-c]pyridine;
190) (R)-2-(5-(2-(2,5-difluorophenyl)pyrrolidin-1-yl)pyrazolo[1,5-a]pyrimidin-3-yl)-4,5,6,7-tetrahydrothiazolo[5,4-c]pyridine;
191) (R)-2-(5-(2-(2,5-difluorophenyl)pyrrolidin-1-yl)pyrazolo[1,5-a]pyrimidin-3-yl)-6,7-dihydrothiazolo[5,4-c]pyridine-5(4H)-carboxamide;
192) (R)-2-(5-(2-(2,5-difluorophenyl)pyrrolidin-1-yl)pyrazolo[1,5-a]pyrimidin-3-yl)-6,7-dihydro-4H-pyrano[4,3-d]thiazole;
193) (R)-5-(2-(2,5-difluorophenyl)pyrrolidin-1-yl)-3-(5,6-dimethoxy-1H-benzo[d]imidazol-2-yl)pyrazolo[1,5-a]pyrimidin-2-amine;
194) (R)-2-(2-amino-5-(2-(2,5-difluorophenyl)pyrrolidin-1-yl)pyrazolo[1,5-a]pyrimidin-3-yl)-5-methoxy-1H-benzo[d]imidazole-6-carbonitrile;
195) (R)-2-(5-(2-(2-fluorophenyl)pyrrolidin-1-yl)pyrazolo[1,5-a]pyrimidin-3-yl)-6-methoxy-1H-benzo[d]imidazole-5-carbonitrile;
196) (R)-2-(5-(2-(3-fluorophenyl)pyrrolidin-1-yl)pyrazolo[1,5-a]pyrimidin-3-yl)-6-methoxy-1H-benzo[d]imidazole-5-carbonitrile;
197) (S)-2-(5-(2-(2,5-difluorophenyl)pyrrolidin-1-yl)pyrazolo[1,5-a]pyrimidin-3-yl)-6-methoxy-1H-benzo[d]imidazole-5-carbonitrile;
198) (R)-2-(5-(2-(4-fluorophenyl)pyrrolidin-1-yl)pyrazolo[1,5-a]pyrimidin-3-yl)-6-methoxy-1H-benzo[d]imidazole-5-carbonitrile;
199) (R)-5-(2-(2,5-difluorophenyl)pyrrolidin-1-yl)-3-(6,7-dimethoxyimidazo[1,2-a]pyridin-2-yl)pyrazolo[1,5-a]pyrimidine; or
200) (R)-3-(5-(2-(2,5-difluorophenyl)pyrrolidin-1-yl)pyrazolo[1,5-a]pyrimidin-3-yl)-5,6-dihydro-8H-[1,2,4]triazolo[3,4-c][1,4]oxazine.

The present invention also provides a pharmaceutical composition comprising a compound of any one of the present invention, or a pharmaceutically acceptable salt or a stereoisomer thereof, and at least one pharmaceutically acceptable carrier or excipient.

The present invention additionally provided a method of inhibiting Trk, including wildtype TrkA, TrkB and TrkC, the TrkA G595R, the TrkA G667C, the TrkA A608D, the TrkA F589L, and the TrkC G623R, said method comprising administering to a patient a compound of any one of the present invention or a pharmaceutically acceptable salt or an isomeride thereof.

The present invention further provides a method of treating a disease associated with inhibition of Trk, including wildtype TrkA, TrkB and TrkC, the TrkA G595R, the TrkA G667C, the TrkA A608D, the TrkA F589L, and the TrkC G623R, said method comprising administering to a patient in need thereof a therapeutically effective amount of a compound in any one in the present invention, or a pharmaceutically acceptable salt or an isomeride thereof. Wherein the disease is mammary analogue secretory carcinoma (MASC) of the salivary glands, infantile fibrosarcoma, spitz tumors, colon cancer, gastric cancer, thyroid cancer (such as papillary thyroid cancer), lung cancer, leukemia, pancreatic cancer, melanoma (such as multiple melanoma), brain cancer (such pontine glioma), renal cancer (such as congenital mesoblastic nephroma), prostate cancer, ovarian cancer or breast cancer (such as secretory breast carcinoma).

The present invention provided a method of inhibiting Trk in a patient, said method comprising administering to the patient in need thereof a therapeutically effective amount of a compound of the present invention, or a pharmaceutically acceptable salt or an isomeride thereof.

The present invention also provides a use of the present compound or its pharmaceutical composition for the preparation of a medicament.

In some embodiments, wherein the medicament is used for the treatment or prevention of cancer.

In some embodiments, wherein the disease is mammary analogue secretory carcinoma (MASC) of the salivary glands, infantile fibrosarcoma, spitz tumors, colon cancer, gastric cancer, thyroid cancer (such as papillary thyroid cancer), lung cancer, leukemia, pancreatic cancer, melanoma (such as multiple melanoma), brain cancer (such pontine glioma), renal cancer (such as congenital mesoblastic nephroma), prostate cancer, ovarian cancer or breast cancer (such as secretory breast carcinoma).

In some embodiments, wherein the medicament is used as an inhibitor of Trk.

In some embodiments, wherein the Trk is wildtype TrkA, TrkB, TrkC, or the TrkA G595R, the TrkA G667C, the TrkA A608D, the TrkA F589L, or the TrkC G623R.

The present invention also provides a method of enhancing, stimulating and/or increasing the immune response in a patient, said method comprising administering to the patient in need thereof a therapeutically effective amount of a compound of the present invention, or a pharmaceutically acceptable salt or an isomeride thereof.

The general chemical terms used in the formula above have their usual meanings. For example, the term “halogen”, as used herein, unless otherwise indicated, means fluoro, chloro, bromo or iodo. The preferred halogen groups include F, Cl and Br.

As used herein, unless otherwise indicated, alkyl includes saturated monovalent hydrocarbon radicals having straight, branched or cyclic moieties. For example, alkyl radicals include methyl, ethyl, propyl, isopropyl, cyclopropyl, n-butyl, isobutyl, sec-butyl, t-butyl, cyclobutyl, n-pentyl, 3-(2-methyl) butyl, 2-pentyl, 2-methylbutyl, neopentyl, cyclopentyl, n-hexyl, 2-hexyl, 2-methylpentyl and cyclohexyl. Similarly, C₁₋₈, as in C₁₋₈ alkyl is defined to identify the group as having 1, 2, 3, 4, 5, 6, 7 or 8 carbon atoms in a linear or branched arrangement.

Alkenyl and alkynyl groups include straight, branched chain or cyclic alkenes and alkynes. Likewise, “C₂₋₈ alkenyl” and “C₂₋₈ alkynyl” means an alkenyl or alkynyl radicals having 2, 3, 4, 5, 6, 7 or 8 carbon atoms in a linear or branched arrangement.

Alkoxy radicals are oxygen ethers formed from the previously described straight, branched chain or cyclic alkyl groups.

The term “aryl”, as used herein, unless otherwise indicated, refers to an unsubstituted or substituted mono- or polycyclic ring system containing carbon ring atoms. The preferred aryls are mono cyclic or bicyclic 6-10 membered aromatic ring systems. Phenyl and naphthyl are preferred aryls. The most preferred aryl is phenyl.

The term “heterocyclyl”, as used herein, unless otherwise indicated, represents an unsubstituted or substituted stable three to eight membered monocyclic saturated ring system which consists of carbon atoms and from one to three heteroatoms selected from N, O or S, and wherein the nitrogen or sulfur heteroatoms may optionally be oxidized, and the nitrogen heteroatom may optionally be quaternized. The heterocyclyl group may be attached at any heteroatom or carbon atom which results in the creation of a stable structure. Examples of such heterocyclyl groups include, but are not limited to azetidinyl, pyrrolidinyl, piperidinyl, piperazinyl, oxopiperazinyl, oxopiperidinyl, oxoazepinyl, azepinyl, tetrahydrofuranyl, dioxolanyl, tetrahydroimidazolyl, tetrahydrothiazolyl, tetrahydrooxazolyl, tetrahydropyranyl, morpholinyl, thiomorpholinyl, thiamorpholinyl sulfoxide, thiamorpholinyl sulfone and oxadiazolyl.

The term “heteroaryl”, as used herein, unless otherwise indicated, represents an unsubstituted or substituted stable five or six membered monocyclic aromatic ring system or an unsubstituted or substituted nine or ten membered benzo-fused heteroaromatic ring system or bicyclic heteroaromatic ring system which consists of carbon atoms and from one to four heteroatoms selected from N, O or S, and wherein the nitrogen or sulfur heteroatoms may optionally be oxidized, and the nitrogen heteroatom may optionally be quaternized. The heteroaryl group may be attached at any heteroatom or carbon atom which results in the creation of a stable structure. Examples of heteroaryl groups include, but are not limited to thienyl, furanyl, imidazolyl, isoxazolyl, oxazolyl, pyrazolyl, pyrrolyl, thiazolyl, thiadiazolyl, triazolyl, pyridyl, pyridazinyl, indolyl, azaindolyl, indazolyl, benzimidazolyl, benzofuranyl, benzothienyl, benzisoxazolyl, benzoxazolyl, benzopyrazolyl, benzothiazolyl, benzothiadiazolyl, benzotriazolyl adeninyl, quinolinyl or isoquinolinyl. The term “alkenyloxy” refers to the group —O-alkenyl, where alkenyl is defined as above.

The term “alknyloxy” refers to the group —O-alknyl, where alknyl is defined as above.

The term “cycloalkyl” to a cyclic saturated alkyl chain having from 3 to 12 carbon atoms, for example, cyclopropyl, cyclobutyl, cyclopentyl, or cyclohexyl.

The term “substituted” refers to a group in which one or more hydrogen atoms are each independently replaced with the same or different substituent(s). Typical substituents include, but are not limited to, halogen (F, Cl, Br or I), C₁₋₈ alkyl, C₃₋₁₂ cycloalkyl, —OR′, SR′, ═O, ═S, —C(O)R¹, —C(S)R¹, ═NR¹, —C(O)OR¹, —C(S)OR¹, —NR¹R², —C(O)NR′R², cyano, nitro, —S(O)₂R¹, —OS(O₂)OR¹, —OS(O)₂R¹, —OP(O)(OR¹)(OR²); wherein R¹ and R² is independently selected from —H, lower alkyl, lower haloalkyl. In some embodiments, the substituent(s) is independently selected from the group consisting of —F, —Cl, —Br, —I, —OH, trifluoromethoxy, ethoxy, propyloxy, iso-propyloxy, n-butyloxy, isobutyloxy, t-butyloxy, —SCH₃, —SC₂H₅, formaldehyde group, —C(OCH₃), cyano, nitro, CF₃, —OCF₃, amino, dimethylamino, methyl thio, sulfonyl and acetyl.

The term “composition”, as used herein, is intended to encompass a product comprising the specified ingredients in the specified amounts, as well as any product which results, directly or indirectly, from combinations of the specified ingredients in the specified amounts. Accordingly, pharmaceutical compositions containing the compounds of the present invention as the active ingredient as well as methods of preparing the instant compounds are also part of the present invention. Furthermore, some of the crystalline forms for the compounds may exist as polymorphs and as such are intended to be included in the present invention. In addition, some of the compounds may form solvates with water (i.e., hydrates) or common organic solvents and such solvates are also intended to be encompassed within the scope of this invention.

Examples of substituted alkyl group include, but not limited to, 2-aminoethyl, 2-hydroxyethyl, pentachloroethyl, trifluoromethyl, methoxymethyl, pentafluoroethyl and piperazinylmethyl.

Examples of substituted alkoxy groups include, but not limited to, aminomethoxy, trifluoromethoxy, 2-diethylaminoethoxy, 2-ethoxycarbonylethoxy, 3-hydroxypropoxy.

The compounds of the present invention may also be present in the form of pharmaceutically acceptable salts. For use in medicine, the salts of the compounds of this invention refer to non-toxic “pharmaceutically acceptable salts”. The pharmaceutically acceptable salt forms include pharmaceutically acceptable acidic/anionic or basic/cationic salts. The pharmaceutically acceptable acidic/anionic salt generally takes a form in which the basic nitrogen is protonated with an inorganic or organic acid. Representative organic or inorganic acids include hydrochloric, hydrobromic, hydriodic, perchloric, sulfuric, nitric, phosphoric, acetic, propionic, glycolic, lactic, succinic, maleic, fumaric, malic, tartaric, citric, benzoic, mandelic, methanesulfonic, hydroxyethanesulfonic, benzenesulfonic, oxalic, pamoic, 2-naphthalenesulfonic, p-toluenesulfonic, cyclohexanesulfamic, salicylic, saccharinic or trifluoroacetic. Pharmaceutically acceptable basic/cationic salts include, and are not limited to aluminum, calcium, chloroprocaine, choline, diethanolamine, ethylenediamine, lithium, magnesium, potassium, sodium and zinc.

The present invention includes within its scope the prodrugs of the compounds of this invention. In general, such prodrugs will be functional derivatives of the compounds that are readily converted in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the subject. Conventional procedures for the selection and preparation of suitable prodrug derivatives are described, for example, in “Design of Prodrugs”, ed. H. Bundgaard, Elsevier, 1985.

It is intended that the definition of any substituent or variable at a particular location in a molecule be independent of its definitions elsewhere in that molecule. It is understood that substituents and substitution patterns on the compounds of this invention can be selected by one of ordinary skill in the art to provide compounds that are chemically stable and that can be readily synthesized by techniques know in the art as well as those methods set forth herein.

The present invention includes compounds described herein can contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof.

The above Formula I are shown without a definitive stereochemistry at certain positions. The present invention includes all stereoisomers of Formula I and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

When a tautomer of the compound of Formula I exists, the present invention includes any possible tautomers and pharmaceutically acceptable salts thereof, and mixtures thereof, except where specifically stated otherwise.

The present invention includes all isomerides of Formula III and Formula IV and pharmaceutically acceptable salts thereof. Further, mixtures of isomerides as well as isolated specific isomerides are also included. During the course of the synthetic procedures known to those skilled in the art used to prepare the present invention, two isomerides may be obtained by ring-closure reactions. For example, the carboxyl of Compound 7-4 reacts with one of two amino groups of Compound 163-3, then it may result two isomerides which are Compound 163 and its isomeride, and their mixture also may be available. Wherein, isomeride may be stereoisomer, or tautomer.

When the compound of Formula I and pharmaceutically acceptable salts thereof exist in the form of solvates or polymorphic forms, the present invention includes any possible solvates and polymorphic forms. A type of a solvent that forms the solvate is not particularly limited so long as the solvent is pharmacologically acceptable. For example, water, ethanol, propanol, acetone or the like can be used.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N′,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, formic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, formic, hydrochloric, maleic, phosphoric, sulfuric and tartaric acids, particularly preferred are formic and hydrochloric acid. Since the compounds of Formula I are intended for pharmaceutical use they are preferably provided in substantially pure form, for example at least 60% pure, more suitably at least 75% pure, especially at least 98% pure (% are on a weight for weight basis).

The pharmaceutical compositions of the present invention comprise a compound represented by Formula I (or a pharmaceutically acceptable salt thereof) as an active ingredient, a pharmaceutically acceptable carrier and optionally other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds represented by Formula I, or a prodrug, or a metabolite, or pharmaceutically acceptable salts thereof, of this invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, the compound represented by Formula I, or a pharmaceutically acceptable salt thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound, or a pharmaceutically acceptable salt, of Formula I. The compounds of Formula I, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include such as lactose, terra alba, sucrose, talc, gelatin, agar, pectin, acacia, magnesium stearate, and stearic acid. Examples of liquid carriers include such as sugar syrup, peanut oil, olive oil, and water. Examples of gaseous carriers include such as carbon dioxide and nitrogen. In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably containing from about 0.05 mg to about 5 g of the active ingredient. For example, a formulation intended for the oral administration to humans may contain from about 0.5 mg to about 5 g of active agent, compounded with an appropriate and convenient amount of carrier material which may vary from about 5 to about 95 percent of the total composition. Unit dosage forms will generally contain between from about 1 mg to about 2 g of the active ingredient, typically 25 mg, 50 mg, 100 mg, 200 mg, 300 mg, 400 mg, 500 mg, 600 mg, 800 mg, or 1000 mg.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, utilizing a compound represented by Formula I of this invention, or a pharmaceutically acceptable salt thereof, via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including antioxidants) and the like. Furthermore, other adjuvants can be included to render the formulation isotonic with the blood of the intended recipient. Compositions containing a compound described by Formula I, or pharmaceutically acceptable salts thereof, may also be prepared in powder or liquid concentrate form.

Generally, dosage levels on the order of from about 0.01 mg/kg to about 150 mg/kg of body weight per day are useful in the treatment of the above-indicated conditions, or alternatively about 0.5 mg to about 7 g per patient per day. For example, colon cancer, rectal cancer, mantle cell lymphoma, multiple myeloma, breast cancer, prostate cancer, glioblastoma, squamous cell esophageal cancer, liposarcoma, T-cell lymphoma melanoma, pancreatic cancer, or lung cancer, may be effectively treated by the administration of from about 0.01 to 50 mg of the compound per kilogram of body weight per day, or alternatively about 0.5 mg to about 3.5 g per patient per day.

It is understood, however, that lower or higher doses than those recited above may be required. Specific dose level and treatment regimens for any particular subject will depend upon a variety of factors, including the activity of the specific compound employed, the age, body weight, general health, sex, diet, time of administration, route of administration, rate of excretion, drug combination, the severity and course of the particular disease undergoing therapy, the subject disposition to the disease, and the judgment of the treating physician.

These and other aspects will become apparent from the following written description of the invention.

The following Examples are provided to better illustrate the present invention. All parts and percentages are by weight and all temperatures are degrees Celsius, unless explicitly stated otherwise.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results. The compounds of the Examples have been found to inhibit Trk according to at least one assay described herein.

### EXAMPLES

Experimental procedures for compounds of the invention are provided below.

The following abbreviations have been used in the examples:
AcOH: Acetic acid;
DCM: Dichloromethane;
DIBAL-H: Diisobutylaluminium hydride;
DIEA: N,N-Diisopropylethylamine;
DMF: Dimethylformamide;
DMAP: 4-Dimethylaminopyridine;
DMSO: Dimethyl sulfoxide;
EA: Ethyl acetate;
EDTA: Ethylenediaminetetraacetic acid;
HATU: Hexafluorophosphate Azabenzotriazole Tetramethyl Uronium;
HEPES: 4-(2-hydroxyethyl)-1-piperazineethanesulfonic acid;
LCMS: Liquid chromatography-mass spectrometry;
h or hrs: hour or hours;
PE: Petroleum ether;
MeOH: Methanol;
min: minute;
NCS: N-Chlorosuccinimide; rt or R.T: room temperature;
TFA: Trifluoroacetic acid;
THF: Tetrahydrofuran;
TLC: Preparative thin layer chromatography;

(21\1:2 mol·L⁻¹, etc.).

### Example 7 Synthesis of Compound 7

### (R)-2-(5-(5-(2-(2,5-difluorophenyl)pyrrolidin-1-yl)pyrazolo[1,5-a]pyrimidin-3-yl)-4H-1,2,4-triazol-3-yl)propan-2-ol

### Step 1: Preparation of ethyl (R)-5-(2-(2-chloro-5-fluorophenyl)pyrrolidin-1-yl)pyrazolo[1,5-a]pyrimidine-3-carboxylate

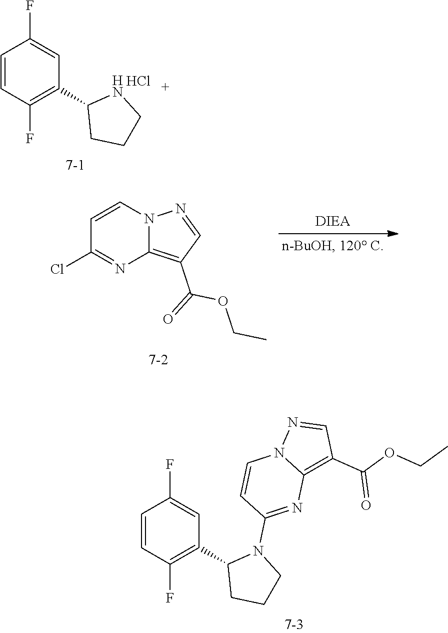 [SMILES: Fc1ccc(F)c([C@H]2CCCN2)c1 ; Cl ; CCOC(=O)c1cnn2ccc(Cl)nc12 ; CCOC(=O)c1cnn2ccc(N3CCC[C@@H]3c3cc(F)ccc3F)nc12]

To a solution of (R)-2-(2,5-difluorophenyl)pyrrolidine hydrochloride (76 g) in 1-BuOH (1 L) was added ethyl 5-chloropyrazolo[1,5-a]pyrimidine-3-carboxylate (78 g) and DIEA (89 g). The mixture was heated to 120° C. for 14 h. Monitored by LCMS until the reaction was completed.

The mixture was concentrated under reduced pressure to remove 1-BuOH, the residue was poured into ice-water and extracted with EA (300 mL*3), combined the organic layers, washed with brine and dried over Na₂SO₄. Concentrated in vacuo and the residue was washed with Hexane (500 mL) to afford the desired product ethyl (R)-5-(2-(2-chloro-5-fluorophenyl)pyrrolidin-1-yl)pyrazolo[1,5-a]pyrimidine-3-carboxylate (122 g, 95%) as a white solid.

### Step 2: Preparation of (R)-5-(2-(2,5-difluorophenyl)pyrrolidin-1-yl)pyrazolo[1,5-a]pyrimidine-3-carboxylic acid

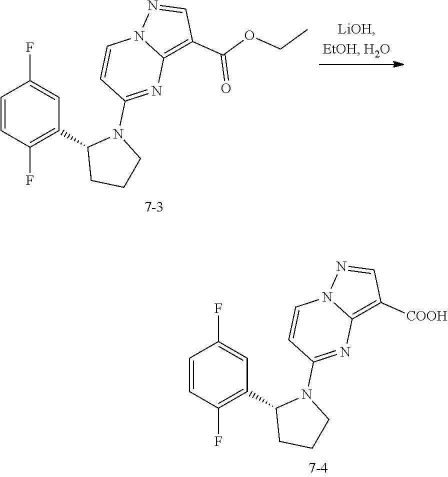 [SMILES: CCOC(=O)c1cnn2ccc(N3CCC[C@@H]3c3cc(F)ccc3F)nc12 ; O=C(O)c1cnn2ccc(N3CCC[C@@H]3c3cc(F)ccc3F)nc12]

To a solution of ethyl (R)-5-(2-(2-chloro-5-fluorophenyl)pyrrolidin-1-yl)pyrazolo[1,5-a]pyrimidine-3-carboxylate (122 g) in EtOH (1 L) was added LiOH aqueous solution (1M, 1 L). The reaction mixture was heated to 80° C. for 8 h. Monitored by LCMS until the reaction was completed.

The mixture was concentrated in vacuo to remove EtOH, the residue was added water (1 L) and acidified with HCl (1M) to pH=4-5, filtered and the solid was washed with water, dried in vacuo to afford desired product (R)-5-(2-(2,5-difluorophenyl)pyrrolidin-1-yl)pyrazolo[1,5-a]pyrimidine-3-carboxylic acid (110 g, 98%) as a white solid.

### Step 3: Preparation of (R)-5-(2-(2,5-difluorophenyl)pyrrolidin-1-yl)pyrazolo[1,5-a]pyrimidine-3-carboxamide

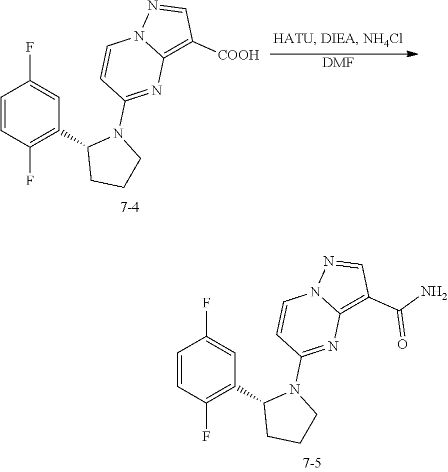

To a solution of (R)-5-(2-(2,5-difluorophenyl)pyrrolidin-1-yl)pyrazolo[1,5-a]pyrimidine-3-carboxylic acid (110 g) in DMF (1 L) was added HATU (146 g), DIEA (82 g) and NH₄Cl (85 g). the mixture was stirred at room temperature for 8 h. Monitored by LCMS until the reaction was completed.

The reaction mixture was poured into water (3 L) and extracted with EA (1 L*5), combined the organic layers and washed with brine (1 L*3), dried over Na₂SO₄. Concentrated under reduced pressure to afford desired product (R)-5-(2-(2,5-difluorophenyl)pyrrolidin-1-yl)pyrazolo[1,5-a]pyrimidine-3-carboxamide (105 g, 96%) as a yellow solid

### Step 4: Preparation of (R)-5-(2-(2,5-difluorophenyl)pyrrolidin-1-yl)pyrazolo[1,5-a]pyrimidine-3-carbothioamide

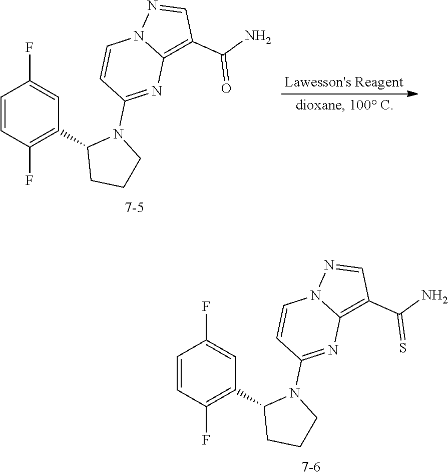 [SMILES: NC(=O)c1cnn2ccc(N3CCC[C@@H]3c3cc(F)ccc3F)nc12 ; NC(=S)c1cnn2ccc(N3CCC[C@@H]3c3cc(F)ccc3F)nc12]

To a solution of (R)-5-(2-(2,5-difluorophenyl)pyrrolidin-1-yl)pyrazolo[1,5-a]pyrimidine-3-carboxamide (105 g) in dioxane (1 L) was added Lawesson's Reagent (210 g), the mixture was heated to 100° C. for 3 h. Monitored by LCMS until the reaction was completed.

The reaction mixture was cooled down to room temperature and filtered, the solid was washed with Dioxane, the filtrate was concentrated and the residue was purified by combi flash (DCM:MeOH=100%:0% to 95%:5%) to afford desired product (R)-5-(2-(2,5-difluorophenyl)pyrrolidin-1-yl)pyrazolo[1,5-a]pyrimidine-3-carbothioamide (85 g, 78%) as a yellow solid.

### Step 5: Preparation of methyl (R)-5-(2-(2,5-difluorophenyl)pyrrolidin-1-yl)pyrazolo[1,5-a]pyrimidine-3-carbimidothioate

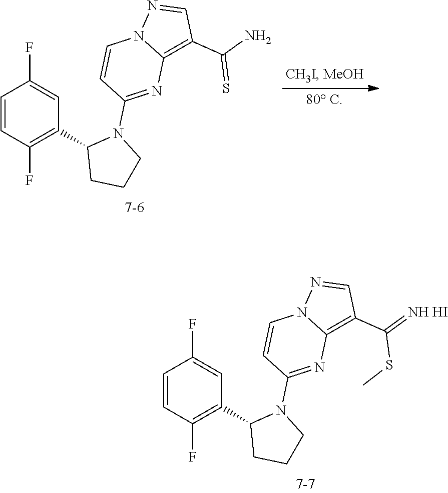

To a solution of (R)-5-(2-(2,5-difluorophenyl)pyrrolidin-1-yl)pyrazolo[1,5-a]pyrimidine-3-carbothioamide (78 g) in MeOH (800 mL) was added CH₃I (46 g), the mixture was heated to 80° C. for 2 h. Monitored by LCMS until the reaction was completed.

The reaction mixture was concentrated in vacuo and the residue was purified by combi flash (DCM:MeOH=100%:O % to 90%:10%) to afford desired product methyl (R)-5-(2-(2,5-difluorophenyl)pyrrolidin-1-yl)pyrazolo[1,5-a]pyrimidine-3-carbimidothioate (90 g, 83%) as a yellow solid

### Step 6: Preparation of (R)-2-(5-(5-(2-(2,5-difluorophenyl)pyrrolidin-1-yl)pyrazolo[1,5-a]pyrimidin-3-yl)-4H-1,2,4-triazol-3-yl)propan-2-ol (Compound 7)

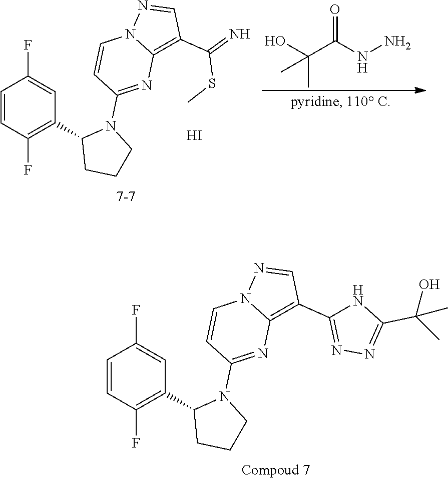

To a solution of methyl (R)-5-(2-(2,5-difluorophenyl)pyrrolidin-1-yl)pyrazolo[1,5-a]pyrimidine-3-carbimidothioate hydroiodide (5 g) in pyridine (50 mL) was added 2-hydroxy-2-methylpropanehydrazide (2.37 g), the mixture was heated to 110° C. overnight. Monitored by LCMS until the reaction was completed.

The reaction mixture was concentrated under reduced pressure to remove pyridine. The residue was purified by combi flash (DCM:MeOH=100%:O % to 90%:10%) to yield 3.48 g (61% yield) of the title compound. MS(ES⁺): m/z=426.2 (M+H)⁺

¹H NMR (500 MHz, CD₃OD) δ 8.62-8.30 (m, 2H), 7.19 (s, 1H), 7.13-6.88 (m, 2H), 6.65 and 6.11 (1H, s+s), 5.70 and 5.35 (1H, s+s), 4.27-3.71 (m, 2H), 2.57 (s, 1H), 2.31-1.95 (m, 3H), 1.63 (s, 6H).

### Example 42 Synthesis of Compound 42

### (S)-(5-(5-((R)-2-(2,5-difluorophenyl)pyrrolidin-1-yl)pyrazolo[1,5-a]pyrimidin-3-yl)-4H-1,2,4-triazol-3-yl)(phenyl)methanol

### Step 1: Preparation of (S)-2-hydroxy-2-phenylacetohydrazide

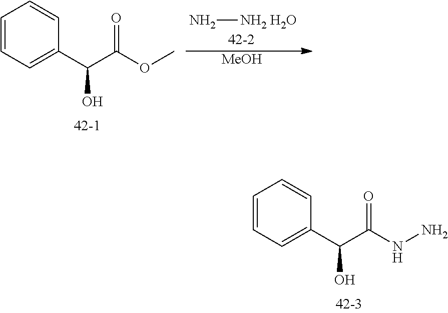

To a solution of methyl (S)-2-hydroxy-2-phenylacetate (166 mg) in MeOH (10 mL) was added hydrazine hydrate (200 mg), the mixture was heated to 80° C. and stirred overnight. Monitored by LCMS until the reaction was completed.

The reaction mixture was concentrated in vacuo to afford desired product (S)-2-hydroxy-2-phenylacetohydrazide (100 mg, 60%) as a yellow oil.

### Step 2: Preparation of (5)-(5-(5-((R)-2-(2,5-difluorophenyl)pyrrolidin-1-yl)pyrazolo[1,5-a]pyrimidin-3-yl)-4H-1,2,4-triazol-3-yl)(phenyl)methanol

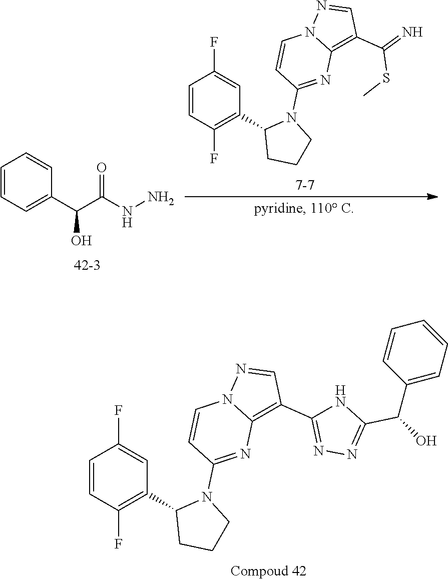 [SMILES: NNC(=O)[C@@H](O)c1ccccc1 ; CSC(=N)c1cnn2ccc(N3CCC[C@@H]3c3cc(F)ccc3F)nc12 ; O[C@@H](c1ccccc1)c1nnc(-c2cnn3ccc(N4CCC[C@@H]4c4cc(F)ccc4F)nc23)[nH]1]

To a solution of (S)-2-hydroxy-2-phenylacetohydrazide (100 mg) in pyridine (10 mL) was added methyl (R)-5-(2-(2,5-difluorophenyl)pyrrolidin-1-yl)pyrazolo[1,5-a]pyrimidine-3-carbimidothioate (100 mg), the mixture was heated to 110° C. overnight. Monitored by LCMS until the reaction was completed.

The reaction mixture was concentrated in vacuo and the residue was purified by combi flash (DCM:MeOH=100%:O % to 90%:10%) to yield 60 mg (44.7%, yield) of the title compound. MS(ES⁺): m/z=474.5 (M+H)⁺

¹H NMR (500 MHz, CD₃OD) δ 8.65-8.32 (m, 2H), 7.38-7.18 (m, 6H), 7.12-6.83 (m, 2H), 6.63 and 6.11 (1H, s+s), 5.86 (s, 1H), 5.71 and 5.33 (1H, s+s), 4.27-3.71 (m, 2H), 2.57 (s, 1H), 2.30-1.93 (m, 3H).

### Example 45 Synthesis of Compound 45

### (R)-6-(5-(5-(2-(2,5-difluorophenyl)pyrrolidin-1-yl)pyrazolo[1,5-a]pyrimidin-3-yl)-4H-1,2,4-triazol-3-yl)benzo[c][1,2]oxaborol-1(3H)-ol

### Step 1: Preparation of tert-butyl 2-(1-hydroxy-1,3-dihydrobenzo[1,2]oxaborole-6-carbonyl)hydrazine-1-carboxylate

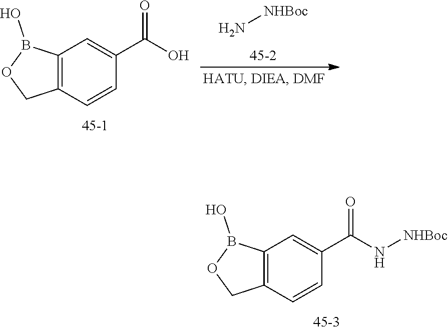 [SMILES: O=C(O)c1ccc2c(c1)B(O)OC2 ; CC(C)(C)OC(=O)NN ; CC(C)(C)OC(=O)NNC(=O)c1ccc2c(c1)B(O)OC2]

To a solution of 1-hydroxy-1,3-dihydrobenzo[c][1,2]oxaborole-6-carboxylic acid (178 g) in DMF (10 mL) was added HATU (572 mg), DIEA (259 mg) and tert-butyl hydrazinecarboxylate (158 mg), the mixture was stirred at room temperature overnight. Monitored by LCMS until the reaction was completed.

The reaction mixture was poured into water (50 mL) and extracted with EA (30 mL*3), combined the organic layers, washed with brine, dried over Na₂SO₄.Concentrated in vacuo, the residue was purified by combi flash (PE:EA=100%:0% to 50%:50%) to afford desired product tert-butyl 2-(1-hydroxy-1,3-di hydrobenzo[c][1,2]oxaborole-6-carbonyl)hydrazine-1-carboxy late (120 mg, 41%) as a white solid

### Step 2: Preparation of 1-hydroxy-1,3-dihydrobenzo[c][1,2]oxaborole-6-carbohydrazide hydrochloride

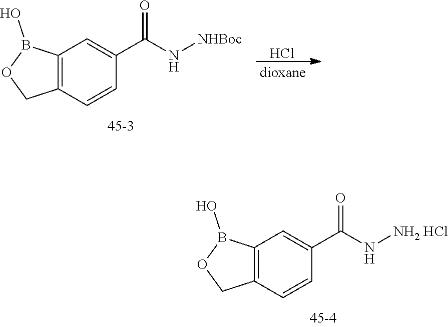

To tert-butyl 2-(1-hydroxy-1,3-dihydrobenzo[c][1,2]oxaborole-6-carbonyl)hydrazine-1-carboxylate (120 mg) was added HCl in dioxane (4M), the mixture was stirred for 2 h. Monitored by LCMS until the reaction was completed.

The reaction mixture was concentrated in vacuo to afford desired product 1-hydroxy-1,3-dihydrobenzo[c][1,2]oxaborole-6-carbohydrazide hydrochloride (90 mg, 97%) as a yellow solid

### Step 3: Preparation of (R)-6-(5-(5-(2-(2,5-difluorophenyl)pyrrolidin-1-yl)pyrazolo[1,5-a]pyrimidin-3-yl)-4H-1,2,4-triazol-3-yl)benzo[c][1,2]oxaborol-1(3H)-ol (Compound 45)

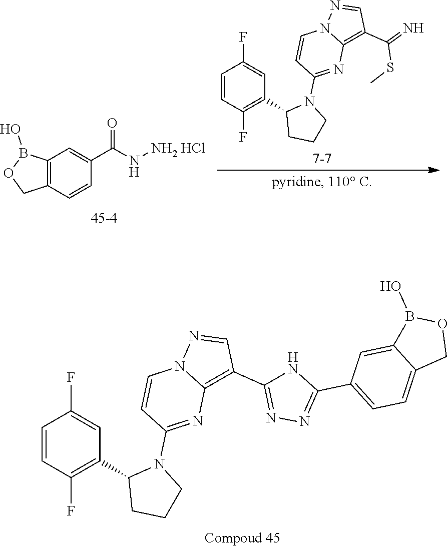

To a solution of 1-hydroxy-1,3-dihydrobenzo[c][1,2]oxaborole-6-carbohydrazide hydrochloride (90 mg) in pyridine (10 mL) was added methyl (R)-5-(2-(2,5-difluorophenyl)pyrrolidin-1-yl)pyrazolo[1,5-a]pyrimidine-3-carbimidothioate (100 mg), the mixture was heated to 110° C. overnight. Monitored by LCMS until the reaction was completed.

The reaction mixture was concentrated in vacuo and the residue was purified by combi flash (DCM:MeOH=100%:O % to 90%:10%) to yield 40 mg (2%, yield) of the title compound. MS(ES⁺): m/z=500.3 (M+H)⁺.

¹H NMR (500 MHz, CD₃OD) δ 8.71-8.42 (m, 3H), 7.70 (s, 1H), 7.47 (d, J=8.1 Hz, 1H), 7.20 (s, 1H), 7.12-6.85 (m, 2H), 6.61 and 6.10 (1H, s+s), 5.71 and 5.37 (1H, s+s), 5.12 (s, 2H), 4.29-3.74 (m, 2H), 2.56 (s, 1H), 2.33-1.92 (m, 3H).

Prepare the following examples (shown in Table 1) essentially as described for Example 45 using the corresponding starting materials. For example, prepare the following Example 1 (shown in Table 1) essentially as described for Example 45 using

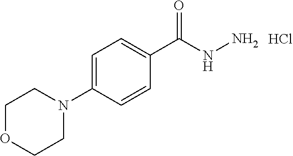 [SMILES: NNC(=O)c1ccc(N2CCOCC2)cc1 ; Cl]

instead of

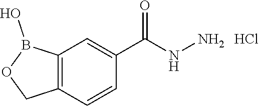 [SMILES: NNC(=O)c1ccc2c(c1)B(O)OC2 ; Cl]

and other starting materials are either commercially available or made by known procedures in the reported literature or as illustrated.

TABLE 1

| | | | Physical Data |
|---|---|---|---|
| EX No. | Chemical Name | Structure | (MS) (M + H)⁺ |
| 1 | (R)-4-(4-(5-(5-(2-(2,5- difluorophenyl)pyrrolidin- 1-yl)pyrazolo[1,5- a]pyrimidin-3-yl)-4H- 1,2,4-triazol-3- yl)phenyl)morpholine | 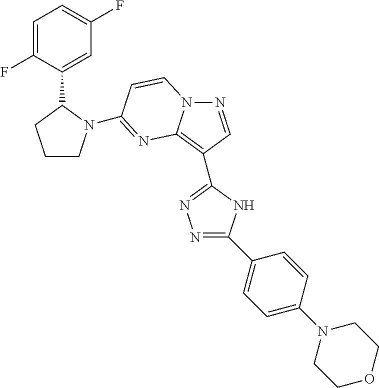 | 529.6 |
| 2 | (R)-1-(4-(5-(5-(2-(2,5- difluorophenyl)pyrrolidin- 1-yl)pyrazolo[1,5- a]pyrimidin-3-yl)-4H- 1,2,4-triazol-3- yl)pyridin-2- yl)piperidin-4-ol | 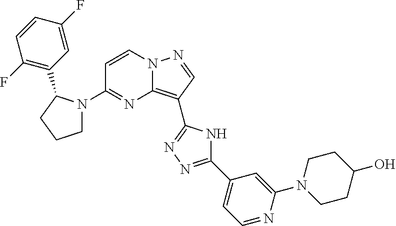 [SMILES: OC1CCN(c2cc(-c3nnc(-c4cnn5ccc(N6CCC[C@@H]6c6cc(F)ccc6F)nc45)[nH]3)ccn2)CC1] | 544.6 |
| 3 | 5-((R)-2-(2,5- difluorophenyl)pyrrolidin- 1-yl)-3-(5- (tetrahydrofuran-3-yl)- 4H-1,2,4-triazol-3- yl)pyrazolo[1,5- a]pyrimidine | 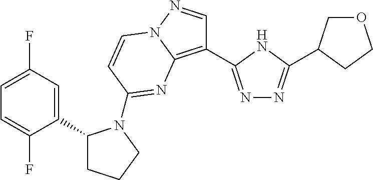 [SMILES: Fc1ccc(F)c([C@H]2CCCN2c2ccn3ncc(-c4nnc(C5CCOC5)[nH]4)c3n2)c1] | 438.5 |
| 4 | (S)-1-(5-(5-((R)-2-(2,5- difluorophenyl)pyrrolidin- 1-yl)pyrazolo[1,5- a]pyrimidin-3-yl)-4H- 1,2,4-triazol-3-yl)ethan- 1-ol | 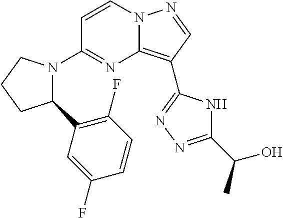 [SMILES: C[C@H](O)c1nnc(-c2cnn3ccc(N4CCC[C@@H]4c4cc(F)ccc4F)nc23)[nH]1] | 412.4 |
| 5 | (1S,4s)-4-(5-(5-((R)-2- (2,5-difluorophenyl) pyrrolidin-1-yl) pyrazolo[1,5- a]pyrimidin-3-yl)-4H- 1,2,4-triazol-3- yl)cyclohexan-1-ol | 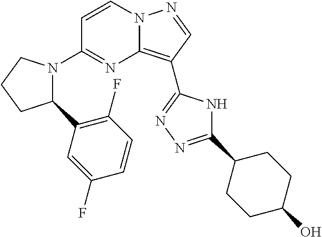 [SMILES: O[C@H]1CC[C@@H](c2nnc(-c3cnn4ccc(N5CCC[C@@H]5c5cc(F)ccc5F)nc34)[nH]2)CC1] | 466.5 |
| 6 | (R)-4-(4-(5-(5-(2-(2,5- difluorophenyl)pyrrolidin- 1-yl)pyrazolo[1,5- a]pyrimidin-3-yl)-4H- 1,2,4-triazol-3- yl)pyridin-2- yl)morpholine | 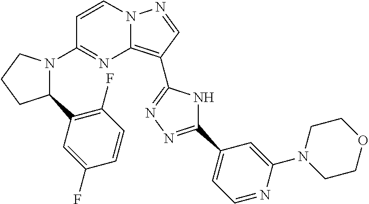 | 530.6 |
| 7 | (R)-2-(5-(5-(2-(2,5- difluorophenyl)pyrrolidin- 1-yl)pyrazolo[1,5- a]pyrimidin-3-yl)-4H- 1,2,4-triazol-3- yl)propan-2-ol | 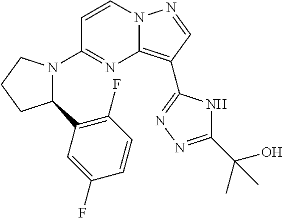 [SMILES: CC(C)(O)c1nnc(-c2cnn3ccc(N4CCC[C@@H]4c4cc(F)ccc4F)nc23)[nH]1] | 426.2 |
| 8 | (R)-5-(2-(2,5- difluorophenyl)pyrrolidin- 1-yl)-3-(5-(pyridin-4- yl)-4H-1,2,4-triazol-3- yl)pyrazolo[1,5- a]pyrimidine | 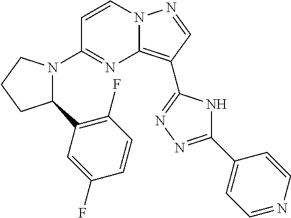 [SMILES: Fc1ccc(F)c([C@H]2CCCN2c2ccn3ncc(-c4nnc(-c5ccncc5)[nH]4)c3n2)c1] | 445.2 |
| 9 | (R)-4-(5-(5-(2-(2,5- difluorophenyl)pyrrolidin- 1-yl)pyrazolo[1,5- a]pyrimidin-3-yl)-4H- 1,2,4-triazol-3-yl)phenol | 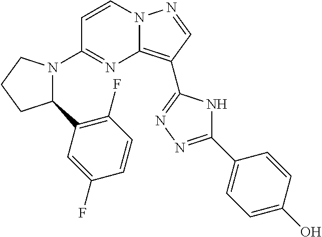 [SMILES: Oc1ccc(-c2nnc(-c3cnn4ccc(N5CCC[C@@H]5c5cc(F)ccc5F)nc34)[nH]2)cc1] | 460.2 |
| 10 | (R)-5-(2-(2,5- difluorophenyl)pyrrolidin- 1-yl)-3-(5-(pyrazin-2- yl)-4H-1,2,4-triazol-3- yl)pyrazolo[1,5- a]pyrimidine | 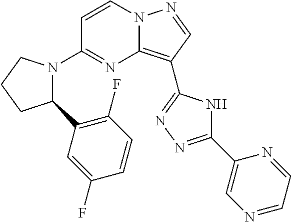 [SMILES: Fc1ccc(F)c([C@H]2CCCN2c2ccn3ncc(-c4nnc(-c5cnccn5)[nH]4)c3n2)c1] | 446.2 |
| 11 | (S)-1-(5-(5-((R)-2-(2,5- difluorophenyl)pyrrolidin- 1-yl)pyrazolo[1,5- a]pyrimidin-3-yl)-4H- 1,2,4-triazol-3-yl)ethan- 1-amine | 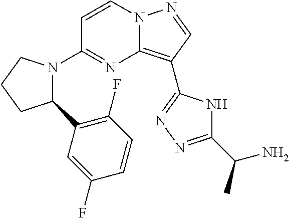 [SMILES: C[C@H](N)c1nnc(-c2cnn3ccc(N4CCC[C@@H]4c4cc(F)ccc4F)nc23)[nH]1] | 411.2 |
| 12 | methyl ((S)-1-(5-(5-((R)- 2-(2,5-difluorophenyl) pyrrolidin-1-yl) pyrazolo[1,5- a]pyrimidin-3-yl)-4H- 1,2,4-triazol-3- yl)ethyl)carbamate | 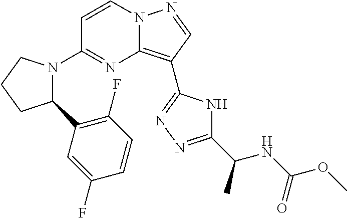 [SMILES: COC(=O)N[C@@H](C)c1nnc(-c2cnn3ccc(N4CCC[C@@H]4c4cc(F)ccc4F)nc23)[nH]1] | 469.2 |
| 13 | (R)-3-(5-(5-(2-(2,5- difluorophenyl)pyrrolidin- 1-yl)pyrazolo[1,5- a]pyrimidin-3-yl)-4H- 1,2,4-triazol-3- yl)benzonitrile | 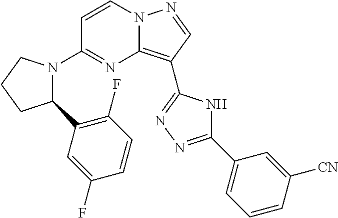 [SMILES: N#Cc1cccc(-c2nnc(-c3cnn4ccc(N5CCC[C@@H]5c5cc(F)ccc5F)nc34)[nH]2)c1] | 469.2 |
| 14 | (R)-5-(2-(2,5- difluorophenyl)pyrrolidin- 1-yl)-3-(5-(6- (trifluoromethyl)pyridin- 3-yl)-4H-1,2,4-triazol-3- yl)pyrazolo[1,5- a]pyrimidine | 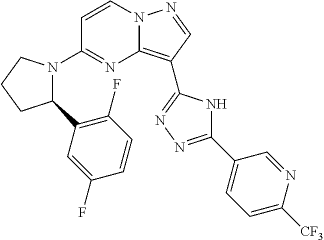 [SMILES: Fc1ccc(F)c([C@H]2CCCN2c2ccn3ncc(-c4nnc(-c5ccc(C(F)(F)F)nc5)[nH]4)c3n2)c1] | 513.2 |
| 15 | 3-(5-(azetidin-2-yl)-4H- 1,2,4-triazol-3-yl)-5- ((R)-2-(2,5- difluorophenyl)pyrrolidin- 1-yl)pyrazolo[1,5- a]pyrimidine | 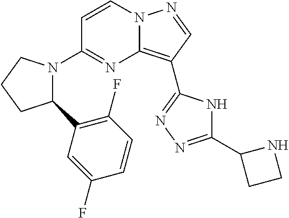 [SMILES: Fc1ccc(F)c([C@H]2CCCN2c2ccn3ncc(-c4nnc(C5CCN5)[nH]4)c3n2)c1] | 423.2 |
| 16 | ethyl (R)-5-(5-(2-(2,5- difluorophenyl)pyrrolidin- 1-yl)pyrazolo[1,5- a]pyrimidin-3-yl)-4H- 1,2,4-triazole-3- carboxylate | 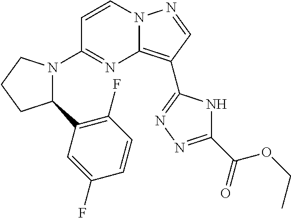 [SMILES: CCOC(=O)c1nnc(-c2cnn3ccc(N4CCC[C@@H]4c4cc(F)ccc4F)nc23)[nH]1] | 440.2 |
| 17 | (R)-5-(5-(2-(2,5- difluorophenyl)pyrrolidin- 1-yl)pyrazolo[1,5- a]pyrimidin-3-yl)-4H- 1,2,4-triazole-3- carboxylic acid | 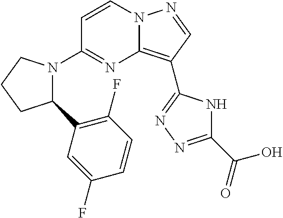 [SMILES: O=C(O)c1nnc(-c2cnn3ccc(N4CCC[C@@H]4c4cc(F)ccc4F)nc23)[nH]1] | 412.1 |
| 18 | (3S)-3-(5-(5-((R)-2-(2,5- difluorophenyl)pyrrolidin- 1-yl)pyrazolo[1,5- a]pyrimidin-3-yl)-4H- 1,2,4-triazol-3- yl)cyclohexan-1-ol | 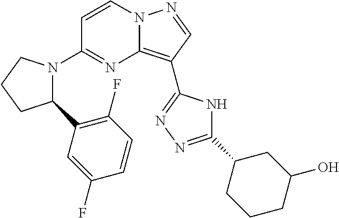 [SMILES: OC1CCC[C@H](c2nnc(-c3cnn4ccc(N5CCC[C@@H]5c5cc(F)ccc5F)nc34)[nH]2)C1] | 466.2 |
| 19 | (3S)-3-(5-(5-((R)-2-(2,5- difluorophenyl)pyrrolidin- 1-yl)pyrazolo[1,5- a]pyrimidin-3-yl)-4H- 1,2,4-triazol-3- yl)cyclopentan-1-ol | 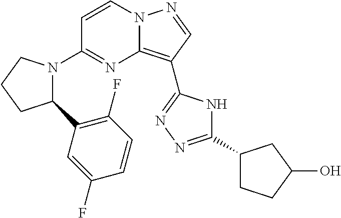 [SMILES: OC1CC[C@H](c2nnc(-c3cnn4ccc(N5CCC[C@@H]5c5cc(F)ccc5F)nc34)[nH]2)C1] | 452.2 |
| 20 | tert-butyl 2-(5-(5-((R)-2- (2,5-difluorophenyl) pyrrolidin-1-yl) pyrazolo[1,5- a]pyrimidin-3-yl)-4H- 1,2,4-triazol-3- yl)azetidine-1- carboxylate | 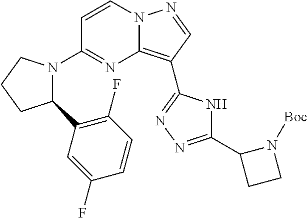 [SMILES: CC(C)(C)OC(=O)N1CCC1c1nnc(-c2cnn3ccc(N4CCC[C@@H]4c4cc(F)ccc4F)nc23)[nH]1] | 523.2 |
| 21 | (R)-1-(4-(5-(5-(2-(2,5- difluorophenyl)pyrrolidin- 1-yl)pyrazolo[1,5- a]pyrimidin-3-yl)-4- methyl-4H-1,2,4-triazol- 3-yl)pyridin-2- yl)piperidin-4-ol | 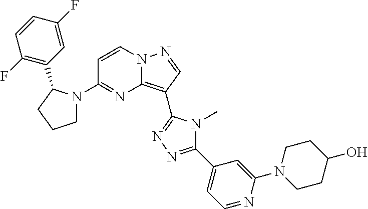 [SMILES: Cn1c(-c2ccnc(N3CCC(O)CC3)c2)nnc1-c1cnn2ccc(N3CCC[C@@H]3c3cc(F)ccc3F)nc12] | 558.3 |
| 22 | (R)-5-(2-(2,5- difluorophenyl)pyrrolidin- 1-yl)-3-(5-(piperidin- 4-yl)-4H-1,2,4-triazol-3- yl)pyrazolo[1,5- a]pyrimidine | 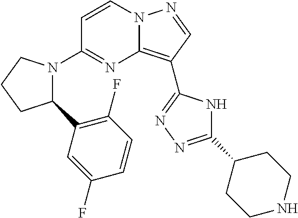 | 451.2 |
| 23 | (R)-1-(5-(5-(2-(2,5- difluorophenyl)pyrrolidin- 1-yl)pyrazolo[1,5- a]pyrimidin-3-yl)-4H- 1,2,4-triazol-3- yl)cyclobutan-1-ol | 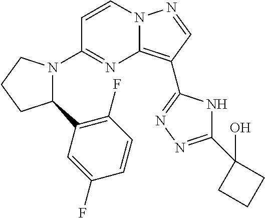 [SMILES: OC1(c2nnc(-c3cnn4ccc(N5CCC[C@@H]5c5cc(F)ccc5F)nc34)[nH]2)CCC1] | 438.2 |
| 24 | (R)-1-(5-(5-(2-(2,5- difluorophenyl)pyrrolidin- 1-yl)pyrazolo[1,5- a]pyrimidin-3-yl)-4H- 1,2,4-triazol-3- yl)cyclobutan-1-amine | 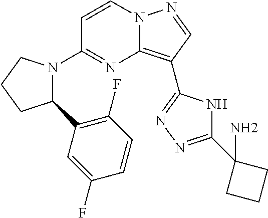 [SMILES: NC1(c2nnc(-c3cnn4ccc(N5CCC[C@@H]5c5cc(F)ccc5F)nc34)[nH]2)CCC1] | 437.2 |
| 25 | (S)-2-(5-(5-((R)-2-(2,5- difluorophenyl)pyrrolidin- 1-yl)pyrazolo[1,5- a]pyrimidin-3-yl)-4H- 1,2,4-triazol-3-yl)-1,1,1- trifluoropropan-2-ol | 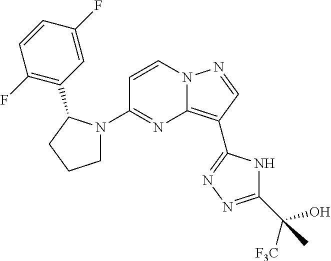 [SMILES: C[C@](O)(c1nnc(-c2cnn3ccc(N4CCC[C@@H]4c4cc(F)ccc4F)nc23)[nH]1)C(F)(F)F] | 480.2 |
| 26 | (R)-2-(5-(5-((R)-2-(2,5- difluorophenyl)pyrrolidin- 1-yl)pyrazolo[1,5- a]pyrimidin-3-yl)-4H- 1,2,4-triazol-3-yl)-1,1,1- trifluoropropan-2-ol | 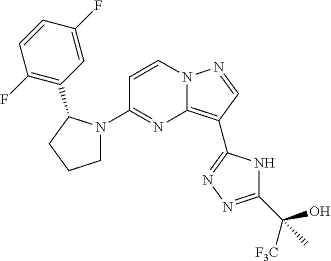 [SMILES: C[C@@](O)(c1nnc(-c2cnn3ccc(N4CCC[C@@H]4c4cc(F)ccc4F)nc23)[nH]1)C(F)(F)F] | 480.2 |
| 27 | (R)-2-(5-(5-(2-(2,5- difluorophenyl)pyrrolidin- 1-yl)pyrazolo[1,5- a]pyrimidin-3-yl)-4H- 1,2,4-triazol-3-yl)- 1,1,1,3,3,3- hexafluoropropan-2-ol | 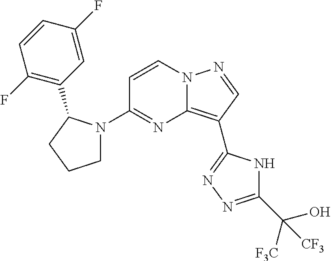 [SMILES: OC(c1nnc(-c2cnn3ccc(N4CCC[C@@H]4c4cc(F)ccc4F)nc23)[nH]1)(C(F)(F)F)C(F)(F)F] | 534.1 |
| 28 | 2-(5-(5-((R)-2-(2,5- difluorophenyl)pyrrolidin- 1-yl)pyrazolo[1,5- a]pyrimidin-3-yl)-4H- 1,2,4-triazol-3-yl)-1,1,1- trifluorobutan-2-ol | 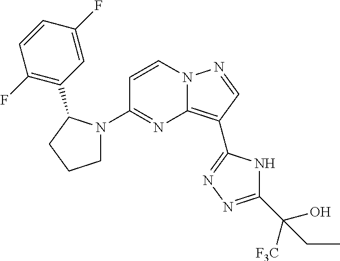 [SMILES: CCC(O)(c1nnc(-c2cnn3ccc(N4CCC[C@@H]4c4cc(F)ccc4F)nc23)[nH]1)C(F)(F)F] | 494.2 |
| 29 | 3-(5-(5-((R)-2-(2,5- difluorophenyl)pyrrolidin- 1-yl)pyrazolo[1,5- a]pyrimidin-3-yl)-4H- 1,2,4-triazol-3-yl)-1,1,1- trifluoro-2- methylpropan-2-ol | 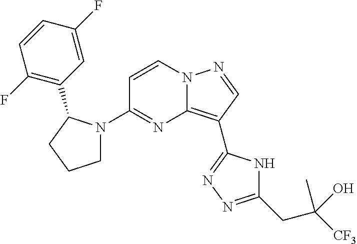 [SMILES: CC(O)(Cc1nnc(-c2cnn3ccc(N4CCC[C@@H]4c4cc(F)ccc4F)nc23)[nH]1)C(F)(F)F] | 494.2 |
| 30 | (R)-1-(5-(5-(2-(2,5- difluorophenyl)pyrrolidin- 1-yl)pyrazolo[1,5- a]pyrimidin-3-yl)-4H- 1,2,4-triazol-3-yl)-2- methylpropan-2-ol | 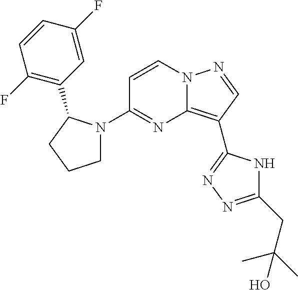 [SMILES: CC(C)(O)Cc1nnc(-c2cnn3ccc(N4CCC[C@@H]4c4cc(F)ccc4F)nc23)[nH]1] | 440.2 |
| 31 | (R)-3-(5-(5-(2-(2,5- difluorophenyl)pyrrolidin- 1-yl)pyrazolo[1,5- a]pyrimidin-3-yl)-4H- 1,2,4-triazol-3- yl)cyclobutan-1-ol | 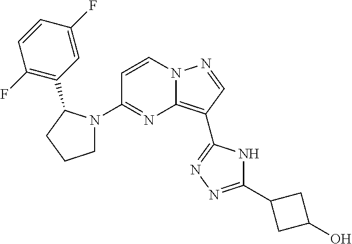 [SMILES: OC1CC(c2nnc(-c3cnn4ccc(N5CCC[C@@H]5c5cc(F)ccc5F)nc34)[nH]2)C1] | 438.2 |
| 32 | (R)-5-(2-(2,5- difluorophenyl)pyrrolidin- 1-yl)-3-(5-(tetrahydro- 2H-pyran-4-yl)-4H- 1,2,4-triazol-3- yl)pyrazolo[1,5- a]pyrimidine | 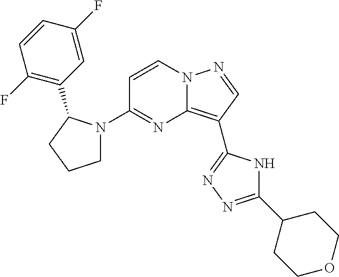 [SMILES: Fc1ccc(F)c([C@H]2CCCN2c2ccn3ncc(-c4nnc(C5CCOCC5)[nH]4)c3n2)c1] | 452.2 |
| 33 | (R)-2-(5-(5-(2-(2,5- difluorophenyl)pyrrolidin- 1-yl)pyrazolo[1,5- a]pyrimidin-3-yl)-4H- 1,2,4-triazol-3-yl)-2- methylpropan-1-ol | 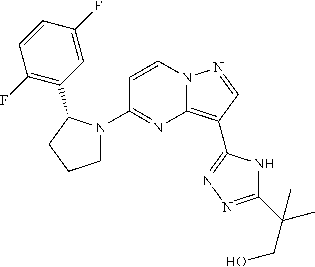 [SMILES: CC(C)(CO)c1nnc(-c2cnn3ccc(N4CCC[C@@H]4c4cc(F)ccc4F)nc23)[nH]1] | 440.2 |
| 34 | (R)-1-(5-(5-((R)-2-(2,5- difluorophenyl)pyrrolidin- 1-yl)pyrazolo[1,5- a]pyrimidin-3-yl)-4H- 1,2,4-triazol-3-yl)ethan- 1-ol | 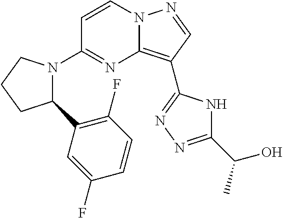 [SMILES: C[C@@H](O)c1nnc(-c2cnn3ccc(N4CCC[C@@H]4c4cc(F)ccc4F)nc23)[nH]1] | 412.4 |
| 35 | 2-(5-(5-((R)-2-(2,5- difluorophenyl)pyrrolidin- 1-yl)pyrazolo[1,5- a]pyrimidin-3-yl)-4H- 1,2,4-triazol-3- yl)piperidin-4-ol | 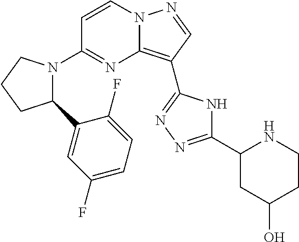 [SMILES: OC1CCNC(c2nnc(-c3cnn4ccc(N5CCC[C@@H]5c5cc(F)ccc5F)nc34)[nH]2)C1] | 467.5 |
| 36 | (R)-6-(5-(5-(2-(2,5- difluorophenyl)pyrrolidin- 1-yl)pyrazolo[1,5- a]pyrimidin-3-yl)-4H- 1,2,4-triazol-3-yl)- 1,2,3,4- tetrahydroisoquinoline | 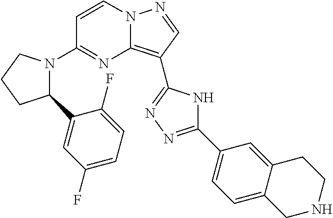 [SMILES: Fc1ccc(F)c([C@H]2CCCN2c2ccn3ncc(-c4nnc(-c5ccc6c(c5)CCNC6)[nH]4)c3n2)c1] | 499.5 |
| 37 | (1R,3r)-3-(5-(5-((R)-2- (2,5- difluorophenyl)pyrrolidin- 1-yl)pyrazolo[1,5- a]pyrimidin-3-yl)-4H- 1,2,4-triazol-3- yl)adamantan-1-ol | 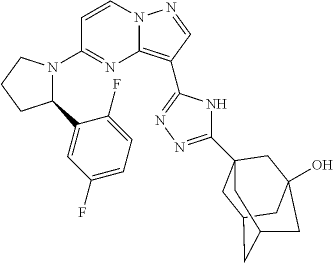 [SMILES: OC12CC3CC(C1)CC(c1nnc(-c4cnn5ccc(N6CCC[C@@H]6c6cc(F)ccc6F)nc45)[nH]1)(C3)C2] | 518.6 |
| 38 | (R)-5-(2-(2,5- difluorophenyl)pyrrolidin- 1-yl)-3-(5-(1- methylpiperidin-4-yl)- 4H-1,2,4-triazol-3- yl)pyrazolo[1,5- a]pyrimidine | 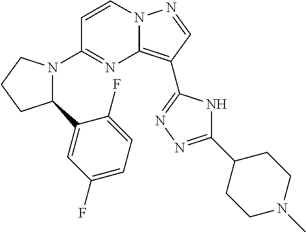 [SMILES: CN1CCC(c2nnc(-c3cnn4ccc(N5CCC[C@@H]5c5cc(F)ccc5F)nc34)[nH]2)CC1] | 465.5 |
| 39 | (R)-2-(5-(5-((R)-2-(2,5- difluorophenyl)pyrrolidin- 1-yl)pyrazolo[1,5- a]pyrimidin-3-yl)-4H- 1,2,4-triazol-3- yl)propan-1-ol | 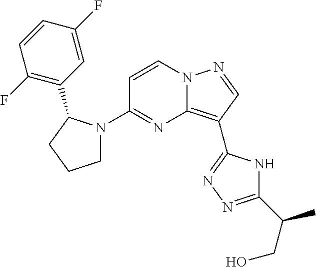 [SMILES: C[C@@H](CO)c1nnc(-c2cnn3ccc(N4CCC[C@@H]4c4cc(F)ccc4F)nc23)[nH]1] | 426.2 |
| 40 | (R)-5-(2-(2,5- difluorophenyl)pyrrolidin- 1-yl)-3-(5-(4- (piperazin-1-yl)phenyl)- 4H-1,2,4-triazol-3- yl)pyrazolo[1,5- a]pyrimidine | 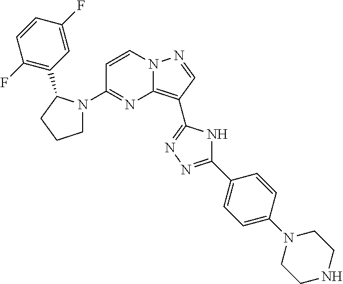 [SMILES: Fc1ccc(F)c([C@H]2CCCN2c2ccn3ncc(-c4nnc(-c5ccc(N6CCNCC6)cc5)[nH]4)c3n2)c1] | 528.2 |
| 41 | (R)-3-(5-(4,4- difluorocyclohexyl)-4H- 1,2,4-triazol-3-yl)-5-(2- (2,5-difluorophenyl) pyrrolidin-1-yl) pyrazolo[1,5-a] pyrimidine | 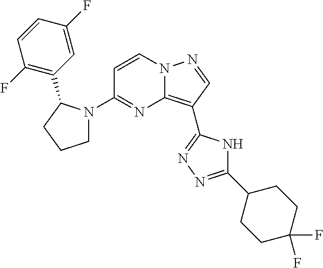 [SMILES: Fc1ccc(F)c([C@H]2CCCN2c2ccn3ncc(-c4nnc(C5CCC(F)(F)CC5)[nH]4)c3n2)c1] | 486.5 |
| 42 | (R)-(5-(5-((R)-2-(2,5- difluorophenyl)pyrrolidin- 1-yl)pyrazolo[1,5- a]pyrimidin-3-yl)-4H- 1,2,4-triazol-3- yl)(phenyl)methanol | 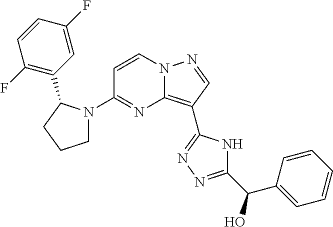 [SMILES: O[C@H](c1ccccc1)c1nnc(-c2cnn3ccc(N4CCC[C@@H]4c4cc(F)ccc4F)nc23)[nH]1] | 474.5 |
| 43 | (R)-(3-(5-(5-(2-(2,5- difluorophenyl)pyrrolidin- 1-yl)pyrazolo[1,5- a]pyrimidin-3-yl)-4H- 1,2,4-triazol-3- yl)bicyclo[1.1.1]pentan- 1-yl)methanol | 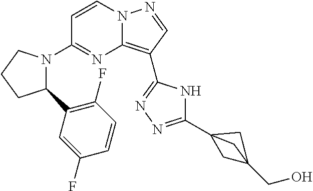 [SMILES: OCC12CC(c3nnc(-c4cnn5ccc(N6CCC[C@@H]6c6cc(F)ccc6F)nc45)[nH]3)(C1)C2] | 464.5 |
| 44 | (R)-3-(5-(5-(2-(2,5- difluorophenyl)pyrrolidin- 1-yl)pyrazolo[1,5- a]pyrimidin-3-yl)-4H- 1,2,4-triazol-3- yl)bicyclo[1.1.1]pentan- 1-amine | 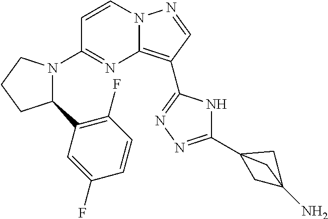 [SMILES: NC12CC(c3nnc(-c4cnn5ccc(N6CCC[C@@H]6c6cc(F)ccc6F)nc45)[nH]3)(C1)C2] | 485.9 |
| 45 | (R)-6-(5-(5-(2-(2,5- difluorophenyl)pyrrolidin- 1-yl)pyrazolo[1,5- a]pyrimidin-3-yl)-4H- 1,2,4-triazol-3- yl)benzo[c][1,2]oxaborol- 1(3H)-ol | 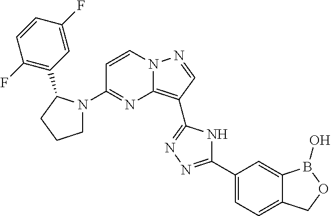 [SMILES: OB1OCc2ccc(-c3nnc(-c4cnn5ccc(N6CCC[C@@H]6c6cc(F)ccc6F)nc45)[nH]3)cc21] | 500.3 |
| 46 | 1-(5-(5-((R)-2-(2,5- difluorophenyl)pyrrolidin- 1-yl)pyrazolo[1,5- a]pyrimidin-3-yl)-4H- 1,2,4-triazol-3-yl)-1,1- difluorobutan-2-ol | 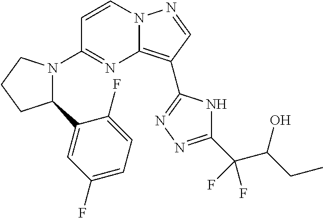 [SMILES: CCC(O)C(F)(F)c1nnc(-c2cnn3ccc(N4CCC[C@@H]4c4cc(F)ccc4F)nc23)[nH]1] | 476.4 |
| 47 | 1-(5-(5-((R)-2-(2,5- difluorophenyl)pyrrolidin- 1-yl)pyrazolo[1,5- a]pyrimidin-3-yl)-4H- 1,2,4-triazol-3-yl)-2,2,2- trifluoroethan-1-ol | 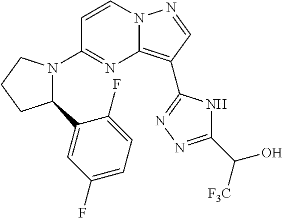 [SMILES: OC(c1nnc(-c2cnn3ccc(N4CCC[C@@H]4c4cc(F)ccc4F)nc23)[nH]1)C(F)(F)F] | 466.4 |
| 48 | 1-(5-(5-((R)-2-(2,5- difluorophenyl)pyrrolidin- 1-yl)pyrazolo[1,5- a]pyrimidin-3-yl)-4H- 1,2,4-triazol-3-yl)prop- 2-yn-1-ol | 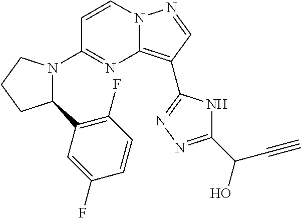 [SMILES: C#CC(O)c1nnc(-c2cnn3ccc(N4CCC[C@@H]4c4cc(F)ccc4F)nc23)[nH]1] | 422.4 |
| 49 | 3-(5-(5-((R)-2-(2,5- difluorophenyl)pyrrolidin- 1-yl)pyrazolo[1,5- a]pyrimidin-3-yl)-4H- 1,2,4-triazol-3- yl)morpholine | 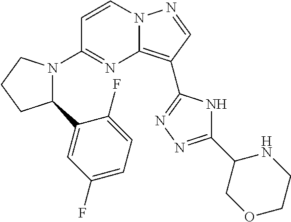 [SMILES: Fc1ccc(F)c([C@H]2CCCN2c2ccn3ncc(-c4nnc(C5COCCN5)[nH]4)c3n2)c1] | 453.5 |
| 50 | (R)-3-(5-(1H-indol-5- yl)-4H-1,2,4-triazol-3- yl)-5-(2-(2,5- difluorophenyl)pyrrolidin- 1-yl)pyrazolo[1,5- a]pyrimidine | 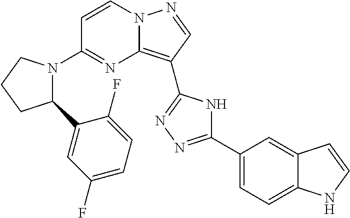 [SMILES: Fc1ccc(F)c([C@H]2CCCN2c2ccn3ncc(-c4nnc(-c5ccc6[nH]ccc6c5)[nH]4)c3n2)c1] | 483.5 |
| 51 | (S)-1-(5-(5-((R)-2-(2,5- difluorophenyl)pyrrolidin- 1-yl)pyrazolo[1,5- a]pyrimidin-3-yl)-4H- 1,2,4-triazol-3-yl)ethyl L-leucinate hydrochloride | 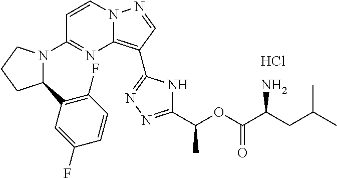 [SMILES: CC(C)C[C@H](N)C(=O)O[C@@H](C)c1nnc(-c2cnn3ccc(N4CCC[C@@H]4c4cc(F)ccc4F)nc23)[nH]1 ; Cl] | 562.0 |
| 52 | 2-(5-(5-((R)-2-(2,5- difluorophenyl)pyrrolidin- 1-yl)pyrazolo[1,5- a]pyrimidin-3-yl)-4H- 1,2,4-triazol-3-yl)-2- fluoroethan-1-ol | 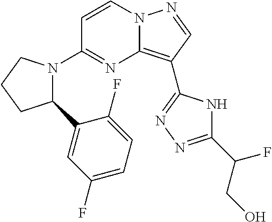 [SMILES: OCC(F)c1nnc(-c2cnn3ccc(N4CCC[C@@H]4c4cc(F)ccc4F)nc23)[nH]1] | 430.4 |
| 53 | (R)-1-(5-(5-(2-(2,5- difluorophenyl)pyrrolidin- 1-yl)pyrazolo[1,5- a]pyrimidin-3-yl)-4H- 1,2,4-triazol-3- yl)cyclopropan-1-ol | 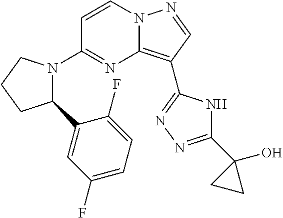 [SMILES: OC1(c2nnc(-c3cnn4ccc(N5CCC[C@@H]5c5cc(F)ccc5F)nc34)[nH]2)CC1] | 424.4 |
| 54 | (R)-5-(2-(2,5- difluorophenyl)pyrrolidin- 1-yl)-3-(5-(6-(4- methylpiperazin-1- yl)pyridin-3-yl)-4H- 1,2,4-triazol-3- yl)pyrazolo[1,5- a]pyrimidine | 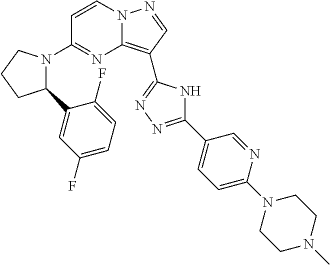 [SMILES: CN1CCN(c2ccc(-c3nnc(-c4cnn5ccc(N6CCC[C@@H]6c6cc(F)ccc6F)nc45)[nH]3)cn2)CC1] | 543.6 |
| 55 | (S)-1-(5-(5-((R)-2-(2,5- difluorophenyl)pyrrolidin- 1-yl)pyrazolo[1,5- a]pyrimidin-3-yl)-4H- 1,2,4-triazol-3-yl)ethyl L-valylvalinate hydrochloride | 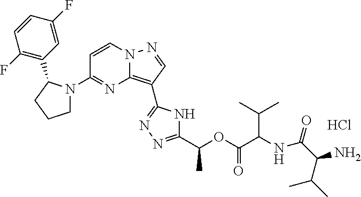 [SMILES: CC(C)C(NC(=O)[C@@H](N)C(C)C)C(=O)O[C@@H](C)c1nnc(-c2cnn3ccc(N4CCC[C@@H]4c4cc(F)ccc4F)nc23)[nH]1 ; Cl] | 647.1 |
| 56 | (R)-6-(5-(5-(2-(2,5- difluorophenyl)pyrrolidin- 1-yl)pyrazolo[1,5- a]pyrimidin-3-yl)-4H- 1,2,4-triazol-3- yl)quinoline | 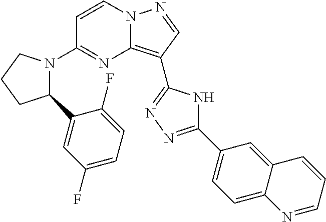 [SMILES: Fc1ccc(F)c([C@H]2CCCN2c2ccn3ncc(-c4nnc(-c5ccc6ncccc6c5)[nH]4)c3n2)c1] | 495.5 |
| 57 | (R)-3-(5-(1H- benzo[d]imidazol-6-yl)- 4H-1,2,4-triazol-3-yl)-5- (2-(2,5- difluorophenyl)pyrrolidin- 1-yl)pyrazolo[1,5- a]pyrimidine | 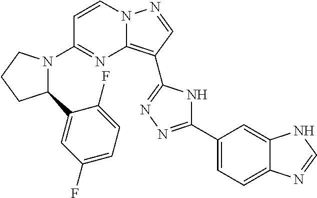 [SMILES: Fc1ccc(F)c([C@H]2CCCN2c2ccn3ncc(-c4nnc(-c5ccc6nc[nH]c6c5)[nH]4)c3n2)c1] | 484.5 |
| 58 | (R)-5-(2-(2,5- difluorophenyl)pyrrolidin- 1-yl)-3-(5-(4- phenoxyphenyl)-4H- 1,2,4-triazol-3- yl)pyrazolo[1,5- a]pyrimidine | 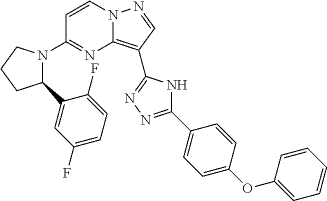 [SMILES: Fc1ccc(F)c([C@H]2CCCN2c2ccn3ncc(-c4nnc(-c5ccc(Oc6ccccc6)cc5)[nH]4)c3n2)c1] | 536.6 |
| 59 | (R)-3-(5-(1H-indazol-6- yl)-4H-1,2,4-triazol-3- yl)-5-(2-(2,5- difluorophenyl)pyrrolidin- 1-yl)pyrazolo[1,5- a]pyrimidine | 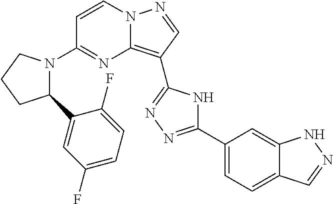 [SMILES: Fc1ccc(F)c([C@H]2CCCN2c2ccn3ncc(-c4nnc(-c5ccc6cn[nH]c6c5)[nH]4)c3n2)c1] | 484.5 |
| 60 | (1R,2S,3R,5S)-5-(5-(5- ((R)-2-(2,5- difluorophenyl)pyrrolidin- 1-yl)pyrazolo[1,5- a]pyrimidin-3-yl)-4H- 1,2,4-triazol-3- yl)cyclohexane-1,2,3,5- tetraol | 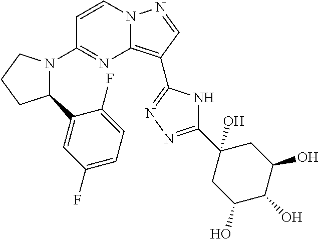 [SMILES: O[C@@H]1C[C@](O)(c2nnc(-c3cnn4ccc(N5CCC[C@@H]5c5cc(F)ccc5F)nc34)[nH]2)C[C@@H](O)[C@H]1O] | 514.5 |
| 61 | (R)-5-(2-(2,5- difluorophenyl)pyrrolidin- 1-yl)-3-(5-(2,3- dihydrobenzofuran-6- yl)-4H-1,2,4-triazol-3- yl)pyrazolo[1,5- a]pyrimidine | 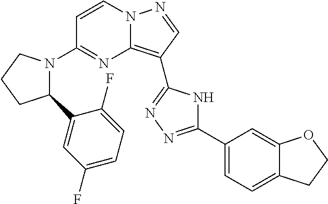 [SMILES: Fc1ccc(F)c([C@H]2CCCN2c2ccn3ncc(-c4nnc(-c5ccc6c(c5)OCC6)[nH]4)c3n2)c1] | 486.5 |

### Example 94 Synthesis of Compound 94

### (R)-(2-(5-(2-(2,5-difluorophenyl)pyrrolidin-yl)pyrazolo[1,5-a]pyrimidin-3-yl)-6-fluorobenzo[d]oxazol-5-yl)methanol

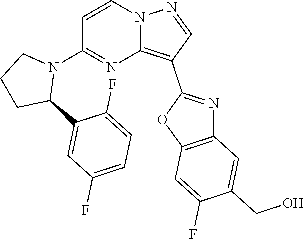 [SMILES: OCc1cc2nc(-c3cnn4ccc(N5CCC[C@@H]5c5cc(F)ccc5F)nc34)oc2cc1F]

### Step 1: Preparation of (R)-methyl 2-(5-(2-(2,5-difluorophenyl)pyrrolidin-1-yl)pyrazolo[1,5-a]pyrimidin-3-yl)-6-fluorobenzo[d]oxazole-5-carboxylate

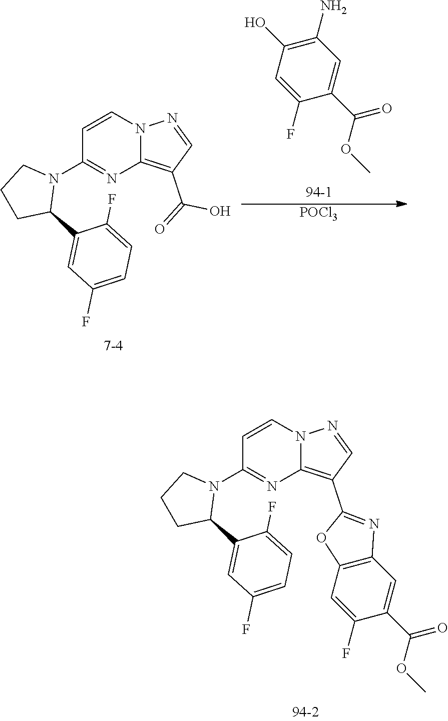 [SMILES: O=C(O)c1cnn2ccc(N3CCC[C@@H]3c3cc(F)ccc3F)nc12 ; COC(=O)c1cc(N)c(O)cc1F ; "POCl3" ; COC(=O)c1cc2nc(-c3cnn4ccc(N5CCC[C@@H]5c5cc(F)ccc5F)nc34)oc2cc1F]

To a solution of (R)-5-(2-(2,5-difluorophenyl)pyrrolidin-1-yl)pyrazolo[1,5-a]pyrimidine-3-carboxylic acid (365.8 mg) in POCl₃ (5 mL) was added methyl 5-amino-2-fluoro-4-hydroxybenzoate (203.6 mg) at 100° C. for 3 h. The reaction was detected by TLC and LCMS. Then the mixture was concentrated in vacuo and the residue was adjusted pH=8, the residue was purified by combi flash (DCM:MeOH=100%:O % to 93%:7%) to afford crude product (R)-methyl 2-(5-(2-(2,5-difluorophenyl)pyrrolidin-1-yl)pyrazolo[1,5-a]pyrimidin-3-yl)-6-fluorobenzo[d]oxazole-5-carboxylate (193.6 mg, 37%) as a yellow solid.

### Step 2: Preparation of (R)-(2-(5-(2-(2,5-difluorophenyl)pyrrolidin-1-yl)pyrazolo[1,5-a]pyrimidin-3-yl)-6-fluorobenzo[d]oxazol-5-yl)methanol

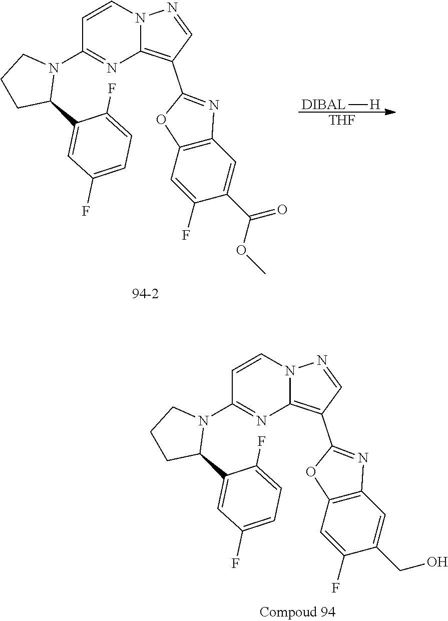

To a solution of (R)-methyl 2-(5-(2-(2,5-difluorophenyl)pyrrolidin-1-yl)pyrazolo[1,5-a]pyrimidin-3-yl)-6-fluorobenzo[d]oxazole-5-carboxylate (193.6 mg) in THF (3 mL) was added DIBAL-H (1 mL) at 0° C. for 1 h. The reaction was detected by TLC and LCMS. The mixture was added saturated NH₄Cl solution (3 mL) and acetic ether. The mixture was extracted by acetic ether (3*15 mL), the organic layer was dried by Na₂SO₄, then the mixture was concentrated in vacuo and the residue was purified by combi flash (DCM:MeOH=100%:O % to 95%:5%) to yield 56.3 mg (31%, yield) of the title compound. MS(ES⁺): m/z=466.4 (M+H)⁺

¹H NMR (500 MHz, CD₃OD) δ 8.61-8.28 (m, 2H), 7.74 (s, 1H), 7.19 (s, 1H), 7.16-6.90 (m, 314), 6.60 and 6.12 (1H, s+s), 5.73 and 5.36 (1H, s+s), 4.61 (s, 2H), 4.30-3.68 (m, 2H), 2.57 (s, 1H), 2.31-1.95 (m, 3H).

Prepare the following examples (shown in Table 2) essentially as described for Example 94 using the corresponding starting materials.

TABLE 2

| | | | Physical Data |
|---|---|---|---|
| EX No. | Chemical Name | Structure | (MS) (M + H)⁺ |
| 68 | 1-(2-(5-((R)-2-(2,5- difluorophenyl)pyrrolidin-1- yl)pyrazolo[1,5-a]pyrimidin-3- yl)benzo[d]oxazol-6-yl)ethan-1-ol | 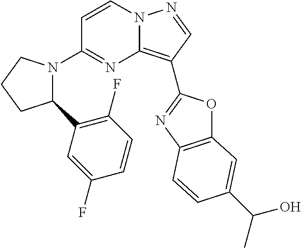 [SMILES: CC(O)c1ccc2nc(-c3cnn4ccc(N5CCC[C@@H]5c5cc(F)ccc5F)nc34)oc2c1] | 462.5 |
| 69 | (R)-(2-(5-(2-(2,5- difluorophenyl)pyrrolidin-1- yl)pyrazolo[1,5-a]pyrimidin-3- yl)benzo[d]oxazol-6-yl)methanol | 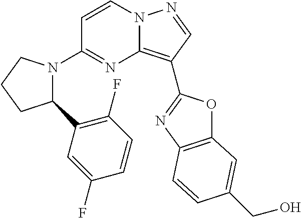 [SMILES: OCc1ccc2nc(-c3cnn4ccc(N5CCC[C@@H]5c5cc(F)ccc5F)nc34)oc2c1] | 448.4 |
| 73 | (R)-2-(5-(2-(2,5- difluorophenyl)pyrrolidin-1- yl)pyrazolo[1,5-a]pyrimidin-3- yl)oxazolo[4,5-c]pyridine | 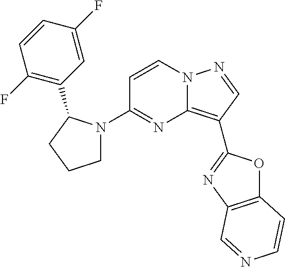 [SMILES: Fc1ccc(F)c([C@H]2CCCN2c2ccn3ncc(-c4nc5cnccc5o4)c3n2)c1] | 419.1 |
| 80 | (R)-2-(5-(2-(2,5- difluorophenyl)pyrrolidin-1- yl)pyrazolo[1,5-a]pyrimidin- 3-yl)-6-(trifluoromethoxy) benzo[d]oxazole | 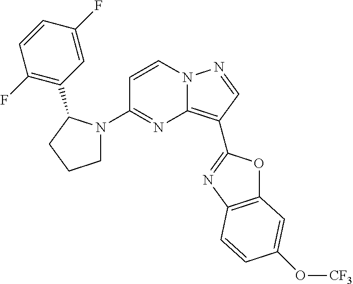 [SMILES: Fc1ccc(F)c([C@H]2CCCN2c2ccn3ncc(-c4nc5ccc(OC(F)(F)F)cc5o4)c3n2)c1] | 502.4 |
| 93 | 1-(2-(5-((R)-2-(2,5- difluorophenyl)pyrrolidin-1- yl)pyrazolo[1,5-a]pyrimidin-3-yl)- 6-fluorobenzo[d]oxazol-5- yl)ethan-1-ol | 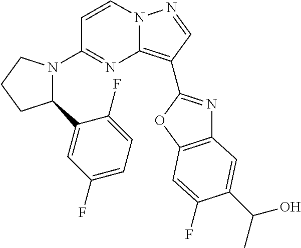 [SMILES: CC(O)c1cc2nc(-c3cnn4ccc(N5CCC[C@@H]5c5cc(F)ccc5F)nc34)oc2cc1F] | 480.5 |
| 94 | (R)-(2-(5-(2-(2,5- difluorophenyl)pyrrolidin-1- yl)pyrazolo[1,5-a]pyrimidin- 3-yl)-6-fluorobenzo[d]oxazol-5- yl)methanol | 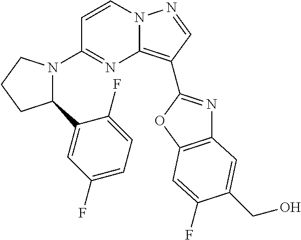 [SMILES: OCc1cc2nc(-c3cnn4ccc(N5CCC[C@@H]5c5cc(F)ccc5F)nc34)oc2cc1F] | 466.4 |
| 100 | (R)-2-(5-(2-(2,5- difluorophenyl)pyrrolidin-1- yl)pyrazolo[1,5-a]pyrimidin-3-yl)- 4-methoxybenzo[d]oxazole | 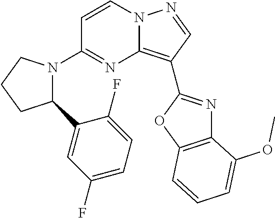 [SMILES: COc1cccc2oc(-c3cnn4ccc(N5CCC[C@@H]5c5cc(F)ccc5F)nc34)nc12] | 448.5 |
| 111 | (R)-2-(5-(2-(2,5- difluorophenyl)pyrrolidin-1- yl)pyrazolo[1,5-a]pyrimidin-3-yl)- 5,7-difluorobenzo[d]oxazole | 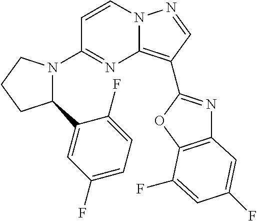 [SMILES: Fc1ccc(F)c([C@H]2CCCN2c2ccn3ncc(-c4nc5cc(F)cc(F)c5o4)c3n2)c1] | 454.4 |
| 120 | (R)-2-(5-(2-(2,5- difluorophenyl)pyrrolidin-1- yl)pyrazolo[1,5-a]pyrimidin-3-yl)- 5,6-dimethoxybenzo[d]oxazole | 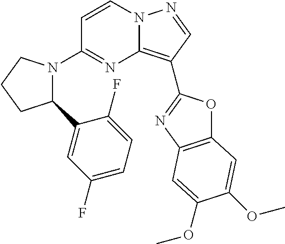 [SMILES: COc1cc2nc(-c3cnn4ccc(N5CCC[C@@H]5c5cc(F)ccc5F)nc34)oc2cc1OC] | 478.5 |
| 131 | (R)-6-(5-(2-(2,5- difluorophenyl)pyrrolidin-1- yl)pyrazolo[1,5-a]pyrimidin-3-yl)- [1,3]dioxolo[4′,5′:4,5]benzo[1,2- d]oxazole | 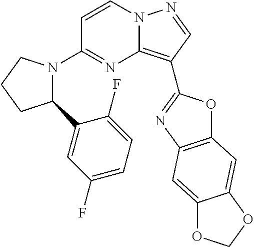 [SMILES: Fc1ccc(F)c([C@H]2CCCN2c2ccn3ncc(-c4nc5cc6c(cc5o4)OCO6)c3n2)c1] | 462.4 |
| 174 | (R)-2-(5-(2-(2,5- difluorophenyl)pyrrolidin-1- yl)pyrazolo[1,5-a]pyrimidin-3-yl)- 6-methoxybenzo[d]oxazole-5- carbonitrile | 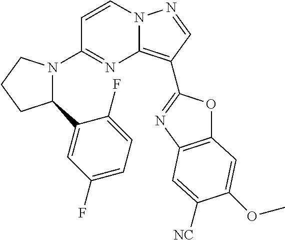 [SMILES: COc1cc2oc(-c3cnn4ccc(N5CCC[C@@H]5c5cc(F)ccc5F)nc34)nc2cc1C#N] | 472.6 |
| 175 | methyl (R)-2-(5-(2-(2,5- difluorophenyl)pyrrolidin-1- yl)pyrazolo[1,5-a]pyrimidin-3-yl)- 4-fluorobenzo[d]oxazole-7- carboxylate | 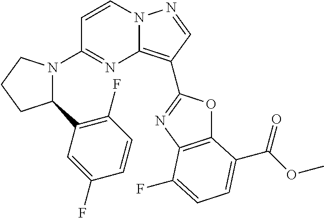 [SMILES: COC(=O)c1ccc(F)c2nc(-c3cnn4ccc(N5CCC[C@@H]5c5cc(F)ccc5F)nc34)oc12] | 493.5 |
| 176 | (R)-2-(5-(2-(2,5- difluorophenyl)pyrrolidin-1- yl)pyrazolo[1,5-a]pyrimidin- 3-yl)-6-(trifluoromethoxy) benzo[d]oxazole-5-carbonitrile | 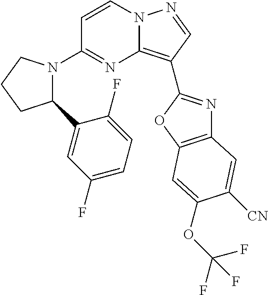 [SMILES: N#Cc1cc2nc(-c3cnn4ccc(N5CCC[C@@H]5c5cc(F)ccc5F)nc34)oc2cc1OC(F)(F)F] | 526.5 |
| 177 | (R)-2-(5-(2-(2,5- difluorophenyl)pyrrolidin-1- yl)pyrazolo[1,5-a]pyrimidin-3-yl)- 6-hydroxybenzo[d]oxazole-5- carbonitrile | 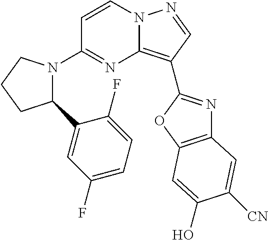 [SMILES: N#Cc1cc2nc(-c3cnn4ccc(N5CCC[C@@H]5c5cc(F)ccc5F)nc34)oc2cc1O] | 458.5 |
| 178 | methyl (R)-2-(5-(2-(2,5- difluorophenyl)pyrrolidin-1- yl)pyrazolo[1,5-a]pyrimidin-3-yl)- 5-methoxybenzo[d]oxazole-6- carboxylate | 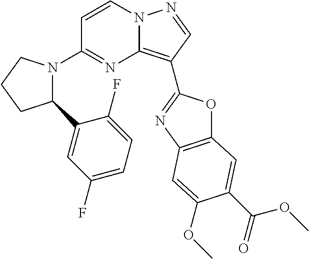 [SMILES: COC(=O)c1cc2oc(-c3cnn4ccc(N5CCC[C@@H]5c5cc(F)ccc5F)nc34)nc2cc1OC] | 505.6 |
| 179 | (R)-6-(difluoromethoxy)-2-(5-(2- (2,5-difluorophenyl)pyrrolidin-1- yl)pyrazolo[1,5-a]pyrimidin-3-yl)- 5-methylbenzo[d]oxazole | 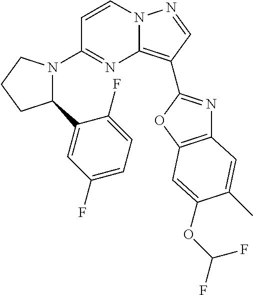 [SMILES: Cc1cc2nc(-c3cnn4ccc(N5CCC[C@@H]5c5cc(F)ccc5F)nc34)oc2cc1OC(F)F] | 497.6 |
| 180 | ((2-(5-((R)-2-(2,5- difluorophenyl)pyrrolidin-1- yl)pyrazolo[1,5-a]pyrimidin-3-yl)- 6-methoxybenzo[d]oxazol-5- yl)methyl)-L-proline | 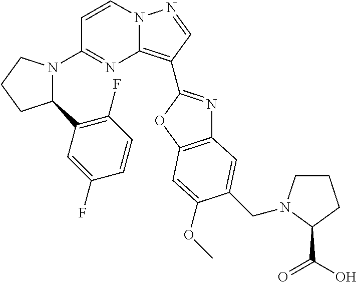 [SMILES: COc1cc2oc(-c3cnn4ccc(N5CCC[C@@H]5c5cc(F)ccc5F)nc34)nc2cc1CN1CCC[C@H]1C(=O)O] | 574.7 |
| 181 | (R)-2-(5-(2-(2,5- difluorophenyl)pyrrolidin-1- yl)pyrazolo[1,5-a]pyrimidin-3-yl)- 5,8-dimethoxy-[1,2,4]triazolo[1,5- c]pyrimidine | 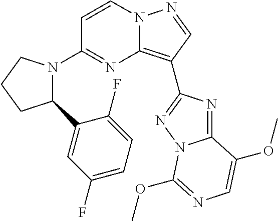 [SMILES: COc1cnc(OC)n2nc(-c3cnn4ccc(N5CCC[C@@H]5c5cc(F)ccc5F)nc34)nc12] | 478.6 |
| 182 | (R)-2-(5-(2-(2,5- difluorophenyl)pyrrolidin-1- yl)pyrazolo[1,5-a]pyrimidin-3-yl)- 6,7-dimethoxy-[1,2,4]triazolo[1,5- a]pyridine | 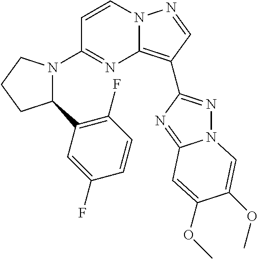 [SMILES: COc1cc2nc(-c3cnn4ccc(N5CCC[C@@H]5c5cc(F)ccc5F)nc34)nn2cc1OC] | 477.6 |

### Example 101 Synthesis of Compound 101

### (R)-3-(5,6-bis(2-methoxyethoxy)-1H-benzo[d]imidazol-2-yl)-5-(2-(2,5-difluorophenyl)pyrrolidin-1-yl)pyrazolo[1,5-a]pyrimidine

### Step 1: Preparation of 4,5-bis(2-methoxyethoxy)-2-nitrobenzoic acid

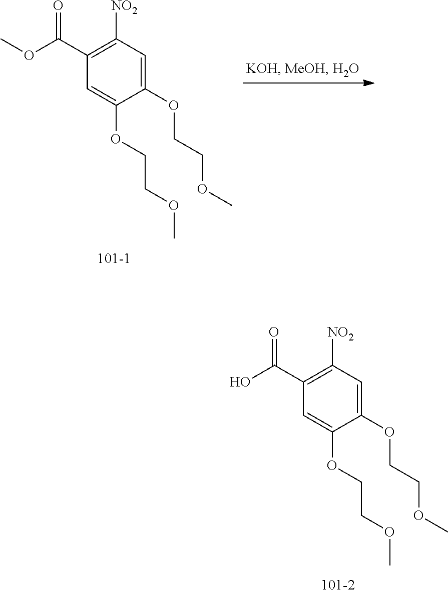

To a solution of methyl 4,5-bis(2-methoxyethoxy)-2-nitrobenzoate (986.5 mg) in MeOH (15 mL) at R.T was added H₂O (3 mL) and KOH (526.7 mg) for 6 h. The reaction was detected by TLC and LCMS. Then the mixture was concentrated in vacuo and the residue was adjusted pH=6, the residue was purified by combi flash (DCM:MeOH=100%:O % to 93%:7%) to afford crude product 4,5-bis(2-methoxyethoxy)-2-nitrobenzoic acid (726.5 mg, 77%) as a yellow solid.

### Step 2: Preparation of tert-butyl (4,5-bis(2-methoxyethoxy)-2-nitrophenyl)carbamate

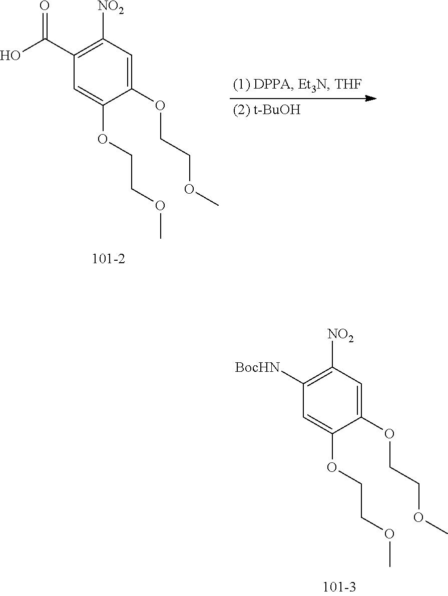 [SMILES: COCCOc1cc(C(=O)O)c([N+](=O)[O-])cc1OCCOC ; COCCOc1cc(NC(=O)OC(C)(C)C)c([N+](=O)[O-])cc1OCCOC]

To a solution of 4,5-bis(2-methoxyethoxy)-2-nitrobenzoic acid (722.8 mg) in THF (15 mL) was added Et₃N (687.3 mg) and DPPA (628.1 mg) at R.T for 12 h. The reaction was detected by TLC and LCMS. Then the mixture was concentrated in vacuo and the residue was added t-BuOH (10 mL) at 80° C. for 6 h. The reaction was detected by TLC and LCMS. Then the mixture was concentrated in vacuo and the residue was purified by combi flash (PE:EA=100%:O % to 66%:34%) to afford crude product 4,5-bis(2-methoxyethoxy)-2-nitrophenyl)carbamate (586.2 mg, 73%) as a yellow solid.

### Step 3: Preparation of 4,5-bis(2-methoxyethoxy)-2-nitroaniline

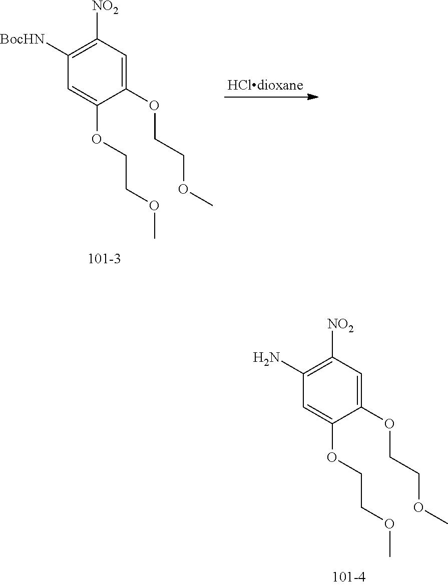 [SMILES: COCCOc1cc(NC(=O)OC(C)(C)C)c([N+](=O)[O-])cc1OCCOC ; COCCOc1cc(N)c([N+](=O)[O-])cc1OCCOC]

To a solution of 4,5-bis(2-methoxyethoxy)-2-nitrophenyl)carbamate (580.2 mg) in dioxane (2 mL) was added HCl dioxane (8 mL), the reaction was stirred at R.T for 13 h. The reaction was detected by TLC and LCMS. Then the mixture was concentrated in vacuo and the residue was adjusted pH=8, and the crude product 4,5-bis(2-methoxyethoxy)-2-nitroaniline (381.7 mg, 89%) was a yellow solid.

### Step 4: Preparation of 4,5-bis(2-methoxyethoxy)benzene-1,2-diamine

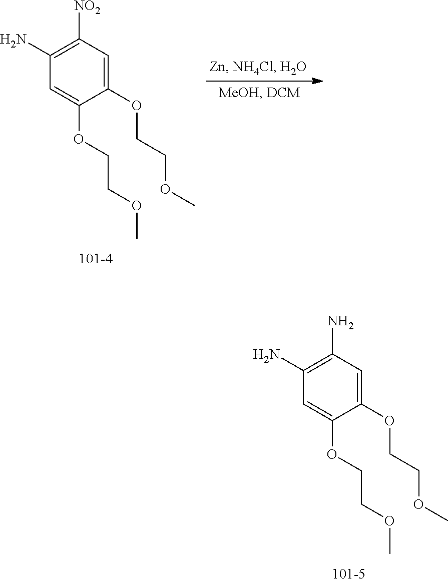 [SMILES: COCCOc1cc(N)c([N+](=O)[O-])cc1OCCOC ; COCCOc1cc(N)c(N)cc1OCCOC]

To a solution of 4,5-bis(2-methoxyethoxy)-2-nitroaniline (380.2 mg) in MeOH (6 mL) was added Zn powder (418.7 mg), NH₄Cl (406.2 mg), H₂O (2 mL), DCM (4 mL) at R.T for 6 h. The reaction was detected by TLC and LCMS. Then the mixture was concentrated in vacuo and the residue was purified by combi flash (DCM:MeOH=100%:O % to 93%:7%) to afford crude product 4,5-bis(2-methoxyethoxy)benzene-1,2-diamine (257.6 mg, 76%) as a yellow solid.

### Step 5: Preparation of (R)-3-(5,6-bis(2-methoxyethoxy)-1H-benzo[d]imidazol-2-yl)-5-(2-(2,5-difluorophenyl)pyrrolidin-1-yl)pyrazolo[1,5-a]pyrimidine (Compound 101)

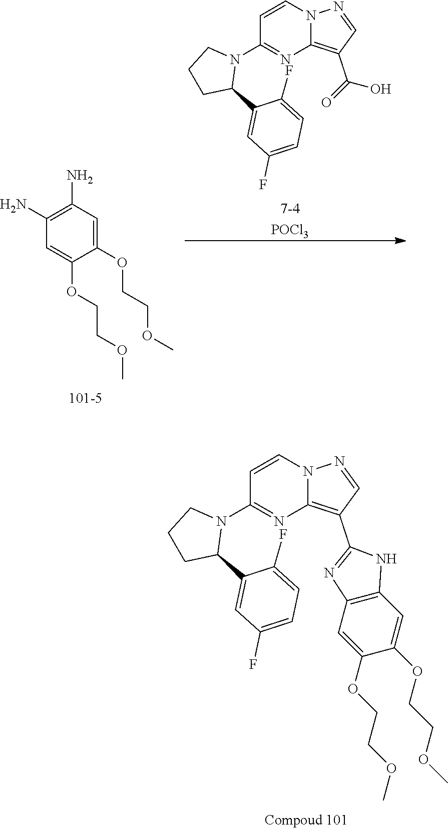

To a solution of 4,5-bis(2-methoxyethoxy)benzene-1,2-diamine (85.9 mg) in POCl₃ (3 mL) was added (R)-5-(2-(2,5-difluorophenyl)pyrrolidin-1-yl)pyrazolo[1,5-a]pyrimidine-3-carboxylic acid (112.6 mg), the mixture was stirred at 100° C. for 6 h. The reaction was detected by TLC and LCMS. Then the mixture was concentrated in vacuo and the residue was adjusted pH=8, the residue was purified by combi flash (DCM:MeOH=100%:O % to 93%:7%) to yield 22.6 mg (12%, yield) of the title compound. MS(ES⁺): m/z=565.6 (M+H)⁺.

¹H NMR (500 MHz, CD₃OD) δ 8.56-8.24 (m, 2H), 7.15 (s, 1H), 7.12-6.87 (m, 4H), 6.63 and 6.12 (1H, s+s), 5.62 and 5.27 (1H, s+s), 4.27-3.71 (m, 6H), 4.87-3.81 (m, 4H), 3.30 (s, 6H), 2.56 (s, 1H), 2.30-1.94 (m, 3H).

Prepare the following examples (shown in Table 3) essentially as described for Example 101 using the corresponding starting materials. For example, prepare the following Example 62 (shown in Table 3) essentially as described for Example 101 using

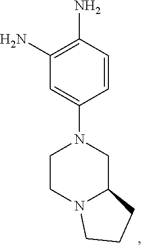 [SMILES: Nc1ccc(N2CCN3CCC[C@@H]3C2)cc1N]

instead of

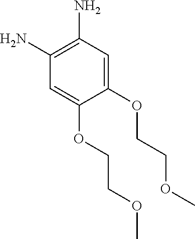 [SMILES: COCCOc1cc(N)c(N)cc1OCCOC]

and other starting materials are either commercially available or made by known procedures in the reported literature or as illustrated.

TABLE 3

| | | | Physical |
|---|---|---|---|
| | | | Data |
| EX | | | (MS) |
| No. | Chemical Name | Structure | (M + H)⁺ |
| 62 | 5-((R)-2-(2,5- difluorophenyl)pyrrolidin-1-yl)- 3-(6-((R)-hexahydropyrrolo[1,2- a]pyrazin-2(1H)-yl)-1H- benzo[d]imidazol-2- yl)pyrazolo[1,5-a]pyrimidine | 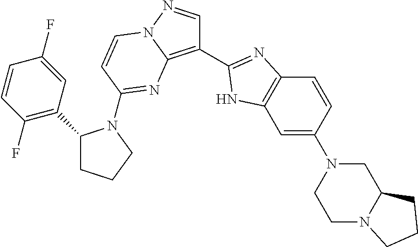 [SMILES: Fc1ccc(F)c([C@H]2CCCN2c2ccn3ncc(-c4nc5ccc(N6CCN7CCC[C@@H]7C6)cc5[nH]4)c3n2)c1] | 541.6 |
| 64 | (R)-4-(2-(5-(2-(2,5- difluorophenyl)pyrrolidin-1- yl)pyrazolo[1,5-a]pyrimidin-3- yl)-1H-imidazo[4,5-c]pyridin-6- yl)morpholine | 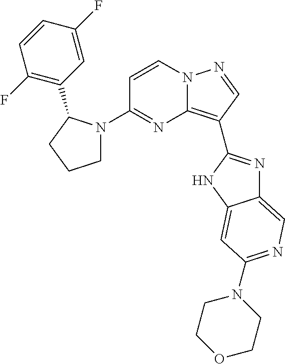 [SMILES: Fc1ccc(F)c([C@H]2CCCN2c2ccn3ncc(-c4nc5cnc(N6CCOCC6)cc5[nH]4)c3n2)c1] | 503.5 |
| 65 | (R)-2-(5-(2-(2,5- difluorophenyl)pyrrolidin-1- yl)pyrazolo[1,5-a]pyrimidin-3- yl)-1H-imidazo[4,5-c]pyridine | 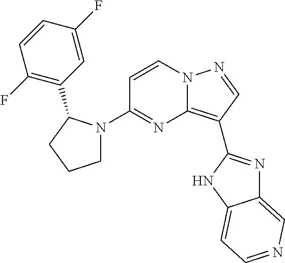 [SMILES: Fc1ccc(F)c([C@H]2CCCN2c2ccn3ncc(-c4nc5cnccc5[nH]4)c3n2)c1] | 418.2 |
| 66 | 1-(2-(5-((R)-2-(2,5- difluorophenyl)pyrrolidin-1- yl)pyrazolo[1,5-a]pyrimidin-3- yl)-5-methoxy-1H- benzo[d]imidazol-6-yl)ethan-1-ol | 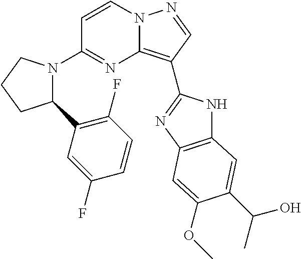 [SMILES: COc1cc2nc(-c3cnn4ccc(N5CCC[C@@H]5c5cc(F)ccc5F)nc34)[nH]c2cc1C(C)O] | 491.5 |
| 67 | (R)-(2-(5-(2-(2,5- difluorophenyl)pyrrolidin-1- yl)pyrazolo[1,5-a]pyrimidin-3- yl)-5-methoxy-1H- benzo[d]imidazol-6-yl)methanol | 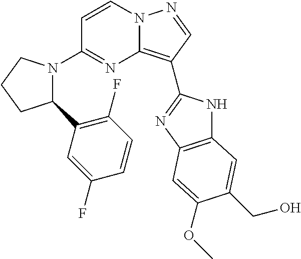 [SMILES: COc1cc2nc(-c3cnn4ccc(N5CCC[C@@H]5c5cc(F)ccc5F)nc34)[nH]c2cc1CO] | 477.5 |
| 70 | 1-(2-(5-((R)-2-(2,5- difluorophenyl)pyrrolidin-1- yl)pyrazolo[1,5-a]pyrimidin-3- yl)-3H-imidazo[4,5-c]pyridin-6- yl)ethan-1-ol | 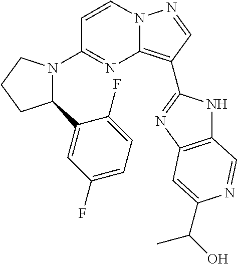 [SMILES: CC(O)c1cc2nc(-c3cnn4ccc(N5CCC[C@@H]5c5cc(F)ccc5F)nc34)[nH]c2cn1] | 462.5 |
| 71 | (R)-5-(2-(2,5- difluorophenyl)pyrrolidin-1-yl)- 3-(5-(trifluoromethoxy)-1H- benzo[d]imidazol-2- yl)pyrazolo[1,5-a]pyrimidine | 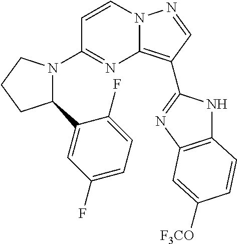 [SMILES: Fc1ccc(F)c([C@H]2CCCN2c2ccn3ncc(-c4nc5cc(OC(F)(F)F)ccc5[nH]4)c3n2)c1] | 501.4 |
| 72 | (R)-2-(5-(2-(2,5- difluorophenyl)pyrrolidin-1- yl)pyrazolo[1,5-a]pyrimidin-3- yl)-6-(trifluoromethyl)-3H- imidazo[4,5-c]pyridine | 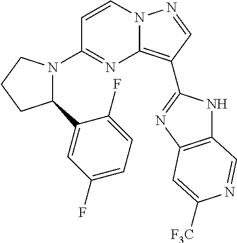 [SMILES: Fc1ccc(F)c([C@H]2CCCN2c2ccn3ncc(-c4nc5cc(C(F)(F)F)ncc5[nH]4)c3n2)c1] | 486.4 |
| 74 | (R)-5-(2-(2,5- difluorophenyl)pyrrolidin-1-yl)- 3-(6-fluoro-1H- benzo[d]imidazol-2- yl)pyrazolo[1,5-a]pyrimidine | 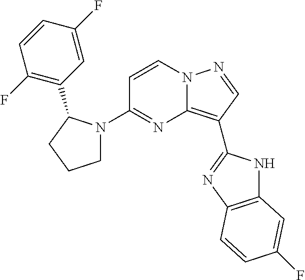 [SMILES: Fc1ccc(F)c([C@H]2CCCN2c2ccn3ncc(-c4nc5ccc(F)cc5[nH]4)c3n2)c1] | 435.2 |
| 76 | (R)-2-(5-(2-(2,5- difluorophenyl)pyrrolidin-1- yl)pyrazolo[1,5-a]pyrimidin-3- yl)-1H-imidazo[4,5-d]pyridazine | 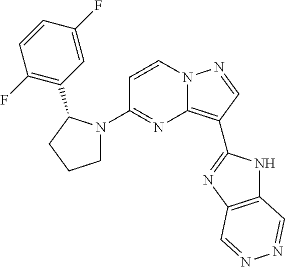 [SMILES: Fc1ccc(F)c([C@H]2CCCN2c2ccn3ncc(-c4nc5cnncc5[nH]4)c3n2)c1] | 419.4 |
| 77 | (R)-5-(2-(2,5- difluorophenyl)pyrrolidin-1-yl)- 3-(6-methoxy-1H- benzo[d]imidazol-2- yl)pyrazolo[1,5-a]pyrimidine | 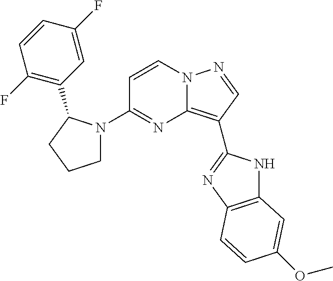 [SMILES: COc1ccc2nc(-c3cnn4ccc(N5CCC[C@@H]5c5cc(F)ccc5F)nc34)[nH]c2c1] | 447.5 |
| 78 | (R)-5-(2-(2,5- difluorophenyl)pyrrolidin-1-yl)- 3-(5,6-dimethoxy-1H- benzo[d]imidazol-2- yl)pyrazolo[1,5-a]pyrimidine | 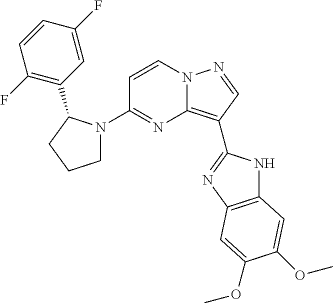 [SMILES: COc1cc2nc(-c3cnn4ccc(N5CCC[C@@H]5c5cc(F)ccc5F)nc34)[nH]c2cc1OC] | 477.5 |
| 79 | (R)-6-(5-(2-(2,5- difluorophenyl)pyrrolidin-1- yl)pyrazolo[1,5-a]pyrimidin-3- yl)-2,2-difluoro-5H- [1,3]dioxolo[4′,5′:4,5]benzo[1,2- d]imidazole | 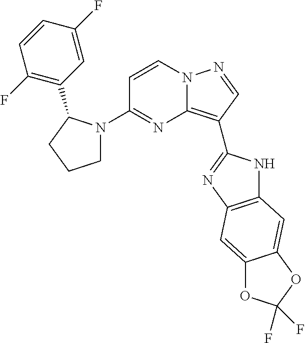 [SMILES: Fc1ccc(F)c([C@H]2CCCN2c2ccn3ncc(-c4nc5cc6c(cc5[nH]4)OC(F)(F)O6)c3n2)c1] | 497.4 |
| 81 | (R)-3-(6-(difluoromethoxy)-1H- benzo[d]imidazol-2-yl)-5-(2- (2,5-difluorophenyl)pyrrolidin-1- yl)pyrazolo[1,5-a]pyrimidine | 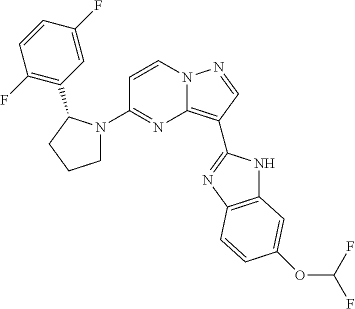 [SMILES: Fc1ccc(F)c([C@H]2CCCN2c2ccn3ncc(-c4nc5ccc(OC(F)F)cc5[nH]4)c3n2)c1] | 483.4 |
| 82 | (R)-2-(5-(2-(2,5- difluorophenyl)pyrrolidin-1- yl)pyrazolo[1,5-a]pyrimidin-3- yl)-6,7,9,10,12,13-hexahydro- 1H-[1,4,7,10]tetraoxacyclo- dodecino[2′,3′:4,5]benzo [1,2-d]imidazole | 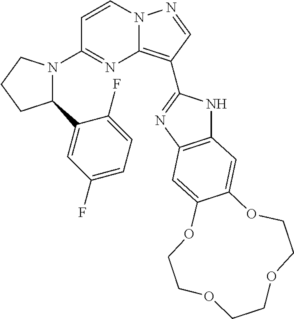 [SMILES: Fc1ccc(F)c([C@H]2CCCN2c2ccn3ncc(-c4nc5cc6c(cc5[nH]4)OCCOCCOCCO6)c3n2)c1] | 563.6 |
| 83 | (R)-2-(5-(2-(2,5- difluorophenyl)pyrrolidin-1- yl)pyrazolo[1,5-a]pyrimidin-3- yl)-1-methyl-6,7-dihydro-1H- [1,4]dioxino[2′,3′:4,5]benzo[1,2- d]imidazole | 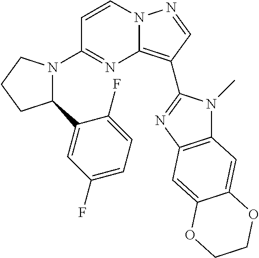 [SMILES: Cn1c(-c2cnn3ccc(N4CCC[C@@H]4c4cc(F)ccc4F)nc23)nc2cc3c(cc21)OCCO3] | 489.5 |
| 84 | (R)-2-(5-(2-(2,5- difluorophenyl)pyrrolidin-1- yl)pyrazolo[1,5-a]pyrimidin-3- yl)-5-methoxy-3H-imidazo[4,5- b]pyridine | 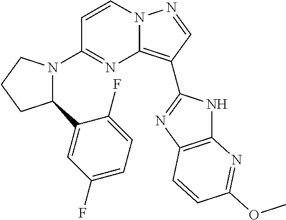 [SMILES: COc1ccc2nc(-c3cnn4ccc(N5CCC[C@@H]5c5cc(F)ccc5F)nc34)[nH]c2n1] | 448.4 |
| 85 | (R)-2-(5-(2-(2,5- difluorophenyl)pyrrolidin-1- yl)pyrazolo[1,5-a]pyrimidin-3- yl)-6-methoxy-1H-imidazo[4,5- c]pyridine | 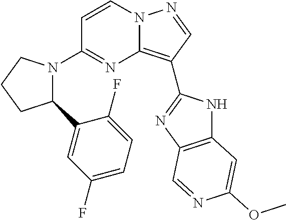 [SMILES: COc1cc2[nH]c(-c3cnn4ccc(N5CCC[C@@H]5c5cc(F)ccc5F)nc34)nc2cn1] | 448.4 |
| 86 | (R)-2-(5-(2-(2,5- difluorophenyl)pyrrolidin-1- yl)pyrazolo[1,5-a]pyrimidin-3- yl)-6-methy1-1H-imidazo[4,5- c]pyridine | 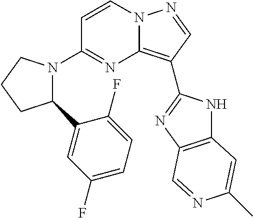 [SMILES: Cc1cc2[nH]c(-c3cnn4ccc(N5CCC[C@@H]5c5cc(F)ccc5F)nc34)nc2cn1] | 432.4 |
| 87 | (R)-8-(5-(2-(2,5- difluorophenyl)pyrrolidin-1- yl)pyrazolo[1,5-a]pyrimidin-3- yl)-7H-purin-6-amine | 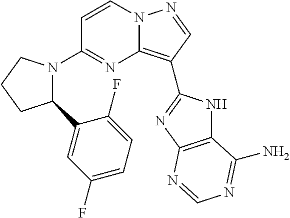 [SMILES: Nc1ncnc2nc(-c3cnn4ccc(N5CCC[C@@H]5c5cc(F)ccc5F)nc34)[nH]c12] | 434.4 |
| 88 | (R)-8-(5-(2-(2,5- difluorophenyl)pyrrolidin-1- yl)pyrazolo[1,5-a]pyrimidin-3- yl)-7H-purin-6-ol | 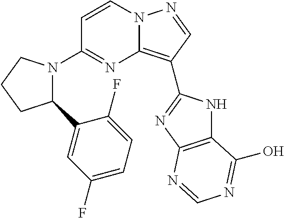 [SMILES: Oc1ncnc2nc(-c3cnn4ccc(N5CCC[C@@H]5c5cc(F)ccc5F)nc34)[nH]c12] | 435.4 |
| 89 | (R)-2-(5-(2-(2,5- difluorophenyl)pyrrolidin-1- yl)pyrazolo[1,5-a]pyrimidin-3- yl)-N-hydroxy-5-methoxy-1H- benzo[d]imidazole-6- carboxamide | 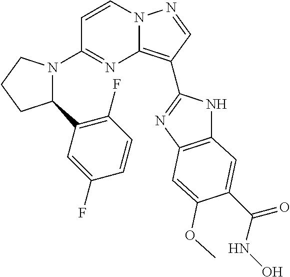 [SMILES: COc1cc2nc(-c3cnn4ccc(N5CCC[C@@H]5c5cc(F)ccc5F)nc34)[nH]c2cc1C(=O)NO] | 506.5 |
| 90 | (R)-2-(5-(2-(2,5- difluorophenyl)pyrrolidin-1- yl)pyrazolo[1,5-a]pyrimidin-3- yl)-5-methoxy-1H- benzo[d]imidazole-6-carboxylic acid | 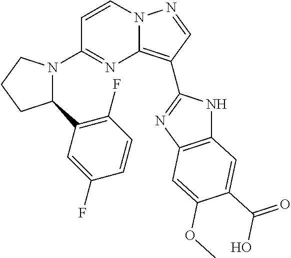 [SMILES: COc1cc2nc(-c3cnn4ccc(N5CCC[C@@H]5c5cc(F)ccc5F)nc34)[nH]c2cc1C(=O)O] | 491.5 |
| 91 | (R)-2-(5-(2-(2,5- difluorophenyl)pyrrolidin-1- yl)pyrazolo[1,5-a]pyrimidin-3- yl)-5-methoxy-1H- benzo[d]imidazole-6- carboxamide | 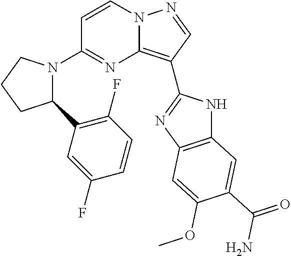 [SMILES: COc1cc2nc(-c3cnn4ccc(N5CCC[C@@H]5c5cc(F)ccc5F)nc34)[nH]c2cc1C(N)=O] | 490.5 |
| 92 | (R)-3-(5-chloro-6- (trifluoromethoxy)-1H- benzo[d]imidazol-2-yl)-5-(2- (2,5-difluorophenyl)pyrrolidin-1- yl)pyrazolo[1,5-a]pyrimidine | 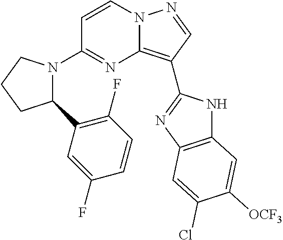 [SMILES: Fc1ccc(F)c([C@H]2CCCN2c2ccn3ncc(-c4nc5cc(Cl)c(OC(F)(F)F)cc5[nH]4)c3n2)c1] | 535.9 |
| 95 | (R)-(2-(5-(2-(2,5- difluorophenyl)pyrrolidin-1- yl)pyrazolo[1,5-a]pyrimidin-3- yl)-3H-imidazo[4,5-c]pyridin-6- yl)methanol | 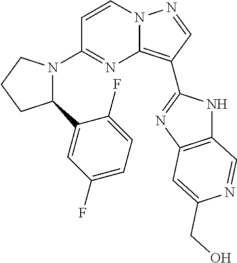 [SMILES: OCc1cc2nc(-c3cnn4ccc(N5CCC[C@@H]5c5cc(F)ccc5F)nc34)[nH]c2cn1] | 448.4 |
| 96 | (R)-2-(5-(2-(2,5- difluorophenyl)pyrrolidin-1- yl)pyrazolo[1,5-a]pyrimidin-3- yl)-6,7-dihydro-1H- [1,4]dioxino[2′,3′:4,5]benzo [1,2-d]imidazole | 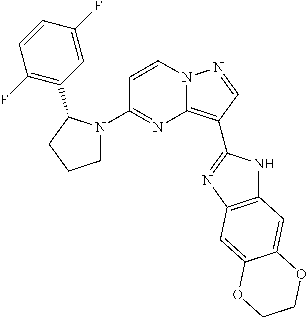 [SMILES: Fc1ccc(F)c([C@H]2CCCN2c2ccn3ncc(-c4nc5cc6c(cc5[nH]4)OCCO6)c3n2)c1] | 475.5 |
| 97 | (R)-3-(7-chloro-1H- benzo[d]imidazol-2-yl)-5-(2- (2,5-difluorophenyl)pyrrolidin-1- yl)pyrazolo[1,5-a]pyrimidine | 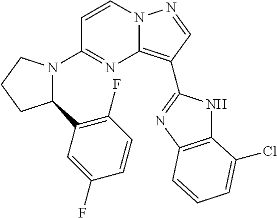 [SMILES: Fc1ccc(F)c([C@H]2CCCN2c2ccn3ncc(-c4nc5cccc(Cl)c5[nH]4)c3n2)c1] | 451.9 |
| 98 | (R)-3-(7-chloro-5-fluoro-1H- benzo[d]imidazol-2-yl)-5-(2- (2,5-difluorophenyl)pyrrolidin-1- yl)pyrazolo[1,5-a]pyrimidine | 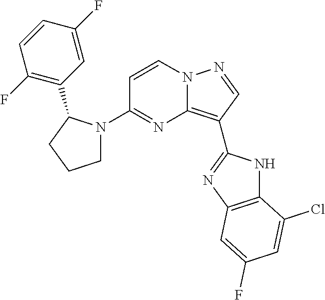 [SMILES: Fc1ccc(F)c([C@H]2CCCN2c2ccn3ncc(-c4nc5cc(F)cc(Cl)c5[nH]4)c3n2)c1] | 469.9 |
| 99 | (R)-2-(5-(2-(2,5- difluorophenyl)pyrrolidin-1- yl)pyrazolo[1,5-a]pyrimidin-3- yl)-7-methyl-1H-imidazo[4,5- c]pyridine | 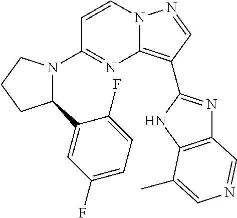 [SMILES: Cc1cncc2nc(-c3cnn4ccc(N5CCC[C@@H]5c5cc(F)ccc5F)nc34)[nH]c12] | 432.5 |
| 101 | (R)-3-(5,6-bis(2- methoxyethoxy)-1H- benzo[d]imidazol-2-yl)-5-(2- (2,5-difluorophenyl)pyrrolidin-1- yl)pyrazolo[1,5-a]pyrimidine | 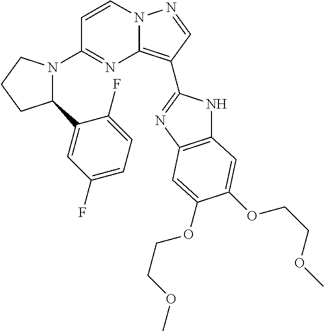 [SMILES: COCCOc1cc2nc(-c3cnn4ccc(N5CCC[C@@H]5c5cc(F)ccc5F)nc34)[nH]c2cc1OCCOC] | 565.6 |
| 102 | (R)-6,7-dichloro-2-(5-(2-(2,5- difluorophenyl)pyrrolidin-1- yl)pyrazolo[1,5-a]pyrimidin-3- yl)-1H-imidazo[4,5-b]pyridine | 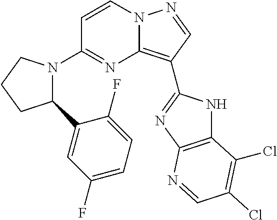 [SMILES: Fc1ccc(F)c([C@H]2CCCN2c2ccn3ncc(-c4nc5ncc(Cl)c(Cl)c5[nH]4)c3n2)c1] | 487.3 |
| 103 | (R)-2-(5-(2-(2,5- difluorophenyl)pyrrolidin-1- yl)pyrazolo[1,5-a]pyrimidin-3- yl)-4-methyl-3H-imidazo[4,5- c]pyridine | 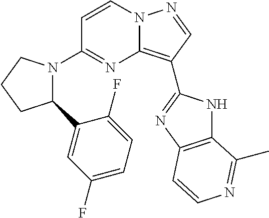 [SMILES: Cc1nccc2nc(-c3cnn4ccc(N5CCC[C@@H]5c5cc(F)ccc5F)nc34)[nH]c12] | 432.5 |
| 104 | (R)-3-(4,7-dichloro-1H- benzo[d]imidazol-2-yl)-5-(2- (2,5-difluorophenyl)pyrrolidin-1- yl)pyrazolo[1,5-a]pyrimidine | 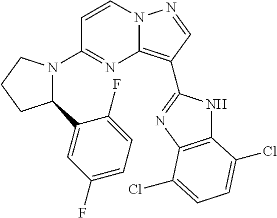 [SMILES: Fc1ccc(F)c([C@H]2CCCN2c2ccn3ncc(-c4nc5c(Cl)ccc(Cl)c5[nH]4)c3n2)c1] | 486.3 |
| 105 | (R)-3-(5,6-dichloro-1H- benzo[d]imidazol-2-yl)-5-(2- (2,5-difluorophenyl)pyrrolidin-1- yl)pyrazolo[1,5-a]pyrimidine | 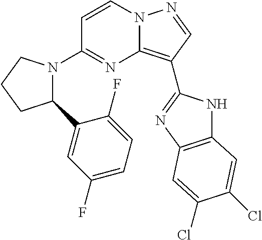 [SMILES: Fc1ccc(F)c([C@H]2CCCN2c2ccn3ncc(-c4nc5cc(Cl)c(Cl)cc5[nH]4)c3n2)c1] | 486.3 |
| 106 | (R)-2-(5-(2-(2,5- difluorophenyl)pyrrolidin-1- yl)pyrazolo[1,5-a]pyrimidin-3- yl)-5-methyl-3H-imidazo[4,5- b]pyridine | 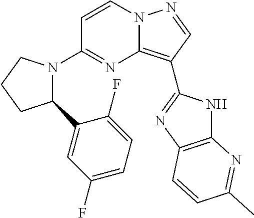 [SMILES: Cc1ccc2nc(-c3cnn4ccc(N5CCC[C@@H]5c5cc(F)ccc5F)nc34)[nH]c2n1] | 432.5 |
| 107 | (R)-2-(5-(2-(2,5- difluorophenyl)pyrrolidin-1- yl)pyrazolo[1,5-a]pyrimidin-3- yl)-1H-benzo[d]imidazole-6- carbonitrile | 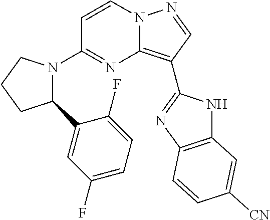 [SMILES: N#Cc1ccc2nc(-c3cnn4ccc(N5CCC[C@@H]5c5cc(F)ccc5F)nc34)[nH]c2c1] | 442.5 |
| 108 | (R)-2-(5-(2-(2,5- difluorophenyl)pyrrolidin-1- yl)pyrazolo[1,5-a]pyrimidin-3- yl)-5-fluoro-3H-imidazo[4,5- b]pyridine | 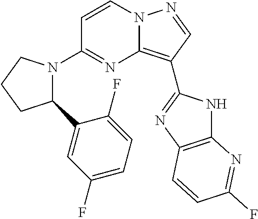 [SMILES: Fc1ccc(F)c([C@H]2CCCN2c2ccn3ncc(-c4nc5ccc(F)nc5[nH]4)c3n2)c1] | 436.4 |
| 109 | (R)-3-(5,6-bis(difluoromethoxy)- 1H-benzo[d]imidazol-2-yl)-5-(2- (2,5-difluorophenyl)pyrrolidin-1- yl)pyrazolo[1,5-a]pyrimidine | 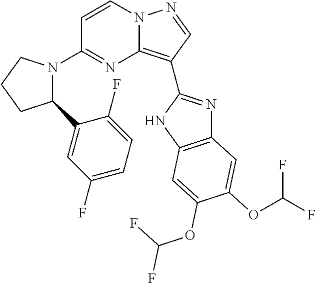 [SMILES: Fc1ccc(F)c([C@H]2CCCN2c2ccn3ncc(-c4nc5cc(OC(F)F)c(OC(F)F)cc5[nH]4)c3n2)c1] | 549.5 |
| 110 | (R)-2-(5-(2-(2,5- difluorophenyl)pyrrolidin-1- yl)pyrazolo[1,5-a]pyrimidin-3- yl)-6-(trifluoromethyl)-1H- imidazo[4,5-b]pyridine | 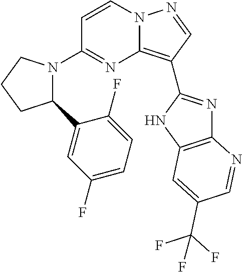 [SMILES: Fc1ccc(F)c([C@H]2CCCN2c2ccn3ncc(-c4nc5ncc(C(F)(F)F)cc5[nH]4)c3n2)c1] | 486.4 |
| 112 | (R)-3-(5-chloro-6-methoxy-1H- benzo[d]imidazol-2-yl)-5-(2- (2,5-difluorophenyl)pyrrolidin-1- yl)pyrazolo[1,5-a]pyrimidine | 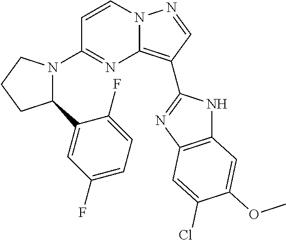 [SMILES: COc1cc2[nH]c(-c3cnn4ccc(N5CCC[C@@H]5c5cc(F)ccc5F)nc34)nc2cc1Cl] | 481.9 |
| 113 | (R)-5-(2-(2,5- difluorophenyl)pyrrolidin-1-yl)- 3-(7-(trifluoromethoxy)-1H- benzo[d]imidazol-2- yl)pyrazolo[1,5-a]pyrimidine | 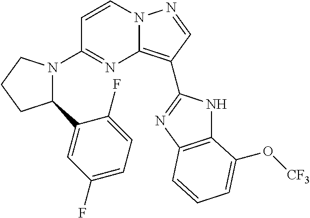 [SMILES: Fc1ccc(F)c([C@H]2CCCN2c2ccn3ncc(-c4nc5cccc(OC(F)(F)F)c5[nH]4)c3n2)c1] | 501.4 |
| 114 | (R)-2-(5-(2-(2,5- difluorophenyl)pyrrolidin-1- yl)pyrazolo[1,5-a]pyrimidin-3- yl)-5-(trifluoromethyl)-3H- imidazo[4,5-b]pyridine | 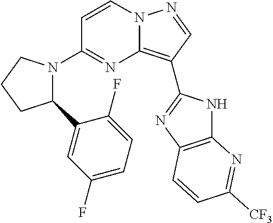 [SMILES: Fc1ccc(F)c([C@H]2CCCN2c2ccn3ncc(-c4nc5ccc(C(F)(F)F)nc5[nH]4)c3n2)c1] | 486.4 |
| 115 | (R)-2-(5-(2-(2,5- difluorophenyl)pyrrolidin-1- yl)pyrazolo[1,5-a]pyrimidin-3- yl)-1H-benzo[d]imidazole-5,6- diyl dimethyl bis(carbonate) | 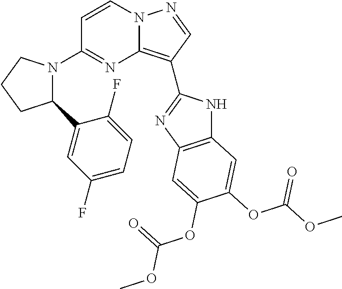 [SMILES: COC(=O)Oc1cc2nc(-c3cnn4ccc(N5CCC[C@@H]5c5cc(F)ccc5F)nc34)[nH]c2cc1OC(=O)OC] | 565.5 |
| 116 | (R)-5-(2-(2,5- difluorophenyl)pyrrolidin-1-yl)- 3-(6-((trifluoromethyl)thio)-1H- benzo[d]imidazol-2- yl)pyrazolo[1,5-a]pyrimidine | 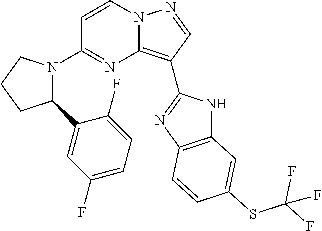 [SMILES: Fc1ccc(F)c([C@H]2CCCN2c2ccn3ncc(-c4nc5ccc(SC(F)(F)F)cc5[nH]4)c3n2)c1] | 517.5 |
| 117 | (R)-2-(5-(2-(2,5- difluorophenyl)pyrrolidin-1- yl)pyrazolo[1,5-a]pyrimidin-3- yl)-1H-benzo[d]imidazole-5,6- diol | 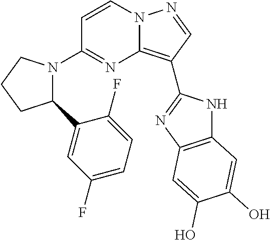 [SMILES: Oc1cc2nc(-c3cnn4ccc(N5CCC[C@@H]5c5cc(F)ccc5F)nc34)[nH]c2cc1O] | 449.4 |
| 118 | 5-((R)-2-(2,5- difluorophenyl)pyrrolidin-1-yl)- 3-(5-(((R)-tetrahydrofuran-3- yl)oxy)-6-(((S)-tetrahydrofuran- 3-yl)oxy)-1H-benzo[d]imidazol- 2-yl)pyrazolo[1,5-a]pyrimidine | 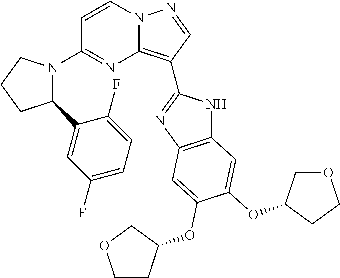 [SMILES: Fc1ccc(F)c([C@H]2CCCN2c2ccn3ncc(-c4nc5cc(O[C@@H]6CCOC6)c(O[C@H]6CCOC6)cc5[nH]4)c3n2)c1] | 589.6 |
| 119 | (R)-2,2′-((2-(5-(2-(2,5- difluorophenyl)pyrrolidin-1- yl)pyrazolo[1,5-a]pyrimidin-3- yl)-1H-benzo[d]imidazole-5,6- diyl)bis(oxy))diacetonitrile | 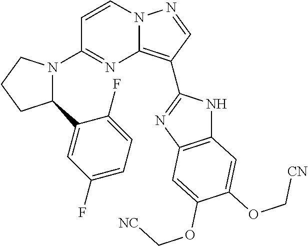 [SMILES: N#CCOc1cc2nc(-c3cnn4ccc(N5CCC[C@@H]5c5cc(F)ccc5F)nc34)[nH]c2cc1OCC#N] | 527.5 |
| 121 | (R)-2-(5-(2-(2,5- difluorophenyl)pyrrolidin-1- yl)pyrazolo[1,5-a]pyrimidin-3- yl)-1H-imidazo[4,5- b]quinoxaline | 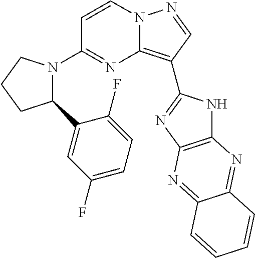 [SMILES: Fc1ccc(F)c([C@H]2CCCN2c2ccn3ncc(-c4nc5nc6ccccc6nc5[nH]4)c3n2)c1] | 469.5 |
| 122 | (R)-2-(5-(2-(2,5- difluorophenyl)pyrrolidin-1- yl)pyrazolo[1,5-a]pyrimidin-3- yl)-7-methyl-3H-imidazo[4,5- b]pyridine | 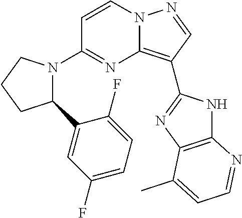 [SMILES: Cc1ccnc2[nH]c(-c3cnn4ccc(N5CCC[C@@H]5c5cc(F)ccc5F)nc34)nc12] | 432.5 |
| 123 | (R)-2-(5-(2-(2,5- difluorophenyl)pyrrolidin-1- yl)pyrazolo[1,5-a]pyrimidin-3- yl)-5-fluoro-1H- benzo[d]imidazole-6-carbonitrile | 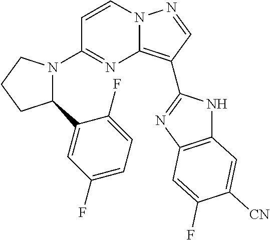 [SMILES: N#Cc1cc2[nH]c(-c3cnn4ccc(N5CCC[C@@H]5c5cc(F)ccc5F)nc34)nc2cc1F] | 460.4 |
| 124 | (R)-2-(5-(2-(2,5- difluorophenyl)pyrrolidin-1- yl)pyrazolo[1,5-a]pyrimidin-3- yl)-1-methyl-1H-imidazo[4,5- c]pyridine | 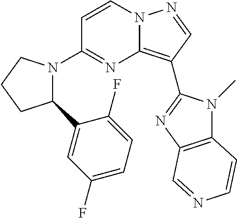 [SMILES: Cn1c(-c2cnn3ccc(N4CCC[C@@H]4c4cc(F)ccc4F)nc23)nc2cnccc21] | 432.5 |
| 125 | (R)-2-(5-(2-(2,5- difluorophenyl)pyrrolidin-1- yl)pyrazolo[1,5-a]pyrimidin-3- yl)-5-methoxy-1H- benzo[d]imidazole-6-carbonitrile | 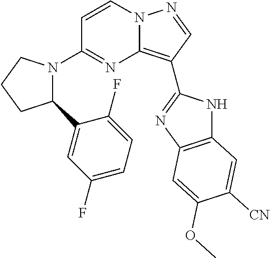 [SMILES: COc1cc2nc(-c3cnn4ccc(N5CCC[C@@H]5c5cc(F)ccc5F)nc34)[nH]c2cc1C#N] | 472.16 |
| 126 | (R)-2-(5-(2-(2,5- difluorophenyl)pyrrolidin-1- yl)pyrazolo[1,5-a]pyrimidin-3- yl)-5-(methylthio)-1H- benzo[d]imidazole-6-carbonitrile | 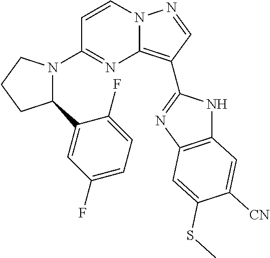 [SMILES: CSc1cc2nc(-c3cnn4ccc(N5CCC[C@@H]5c5cc(F)ccc5F)nc34)[nH]c2cc1C#N] | 488.5 |
| 127 | (R)-5-(2-(2,5- difluorophenyl)pyrrolidin-1-yl)- 3-(7-fluoro-6-methoxy-1H- benzo[d]imidazol-2- yl)pyrazolo[1,5-a]pyrimidine | 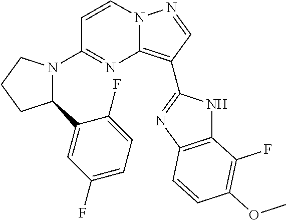 [SMILES: COc1ccc2nc(-c3cnn4ccc(N5CCC[C@@H]5c5cc(F)ccc5F)nc34)[nH]c2c1F] | 465.5 |
| 128 | (R)-2-(5-(2-(2,5- difluorophenyl)pyrrolidin-1- yl)pyrazolo[1,5-a]pyrimidin-3- yl)-1H-imidazo[4,5-b]pyrazine | 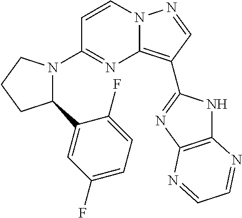 [SMILES: Fc1ccc(F)c([C@H]2CCCN2c2ccn3ncc(-c4nc5nccnc5[nH]4)c3n2)c1] | 419.4 |
| 129 | (R)-6-bromo-2-(5-(2-(2,5- difluorophenyl)pyrrolidin-1- yl)pyrazolo[1,5-a]pyrimidin-3- yl)-1H-imidazo[4,5-b]pyrazine | 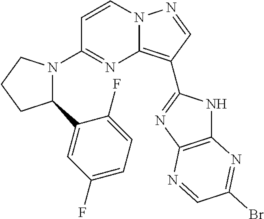 [SMILES: Fc1ccc(F)c([C@H]2CCCN2c2ccn3ncc(-c4nc5ncc(Br)nc5[nH]4)c3n2)c1] | 498.3 |
| 130 | (R)-2-(5-(2-(2,5- difluorophenyl)pyrrolidin-1- yl)pyrazolo[1,5-a]pyrimidin-3- yl)-1H-imidazo[4,5-b]phenazine | 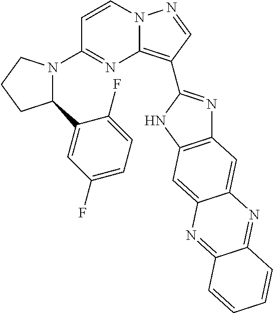 [SMILES: Fc1ccc(F)c([C@H]2CCCN2c2ccn3ncc(-c4nc5cc6nc7ccccc7nc6cc5[nH]4)c3n2)c1] | 519.5 |
| 134 | (R)-2-(5-(2-(2,5- difluorophenyl)pyrrolidin-1- yl)pyrazolo[1,5-a]pyrimidin-3- yl)-7,8-dihydro-1H,6H- [1,4]dioxepino[2′,3′:4,5]benzo [1,2-d]imidazole | 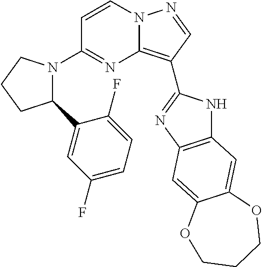 [SMILES: Fc1ccc(F)c([C@H]2CCCN2c2ccn3ncc(-c4nc5cc6c(cc5[nH]4)OCCCO6)c3n2)c1] | 488.6 |
| 135 | (R)-(2-(5-(2-(2,5- difluorophenyl)pyrrolidin-1- yl)pyrazolo[1,5-a]pyrimidin-3- yl)-3H-imidazo[4,5-c]pyridin-6- yl)methanol | 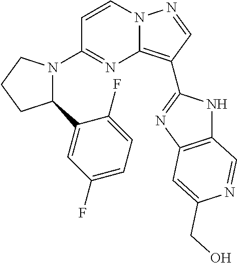 [SMILES: OCc1cc2nc(-c3cnn4ccc(N5CCC[C@@H]5c5cc(F)ccc5F)nc34)[nH]c2cn1] | 477.5 |
| 136 | (R)-3-(5,6-difluoro-1H- benzo[d]imidazol-2-yl)-5-(2- (2,5-difluorophenyl)pyrrolidin-1- yl)pyrazolo[1,5-a]pyrimidine | 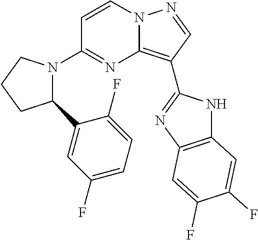 [SMILES: Fc1ccc(F)c([C@H]2CCCN2c2ccn3ncc(-c4nc5cc(F)c(F)cc5[nH]4)c3n2)c1] | 452.5 |
| 137 | methyl (R)-2-(5-(2-(2,5- difluorophenyl)pyrrolidin-1- yl)pyrazolo[1,5-a]pyrimidin-3- yl)-4,5-difluoro-1H-benzo [d]imidazole-6-carboxylate | 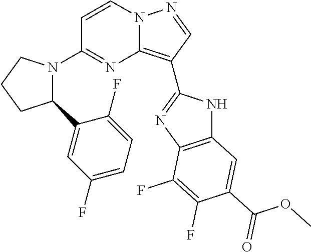 [SMILES: COC(=O)c1cc2[nH]c(-c3cnn4ccc(N5CCC[C@@H]5c5cc(F)ccc5F)nc34)nc2c(F)c1F] | 510.5 |
| 138 | (R)-2-(5-(2-(2,5- difluorophenyl)pyrrolidin-1- yl)pyrazolo[1,5-a]pyrimidin-3- yl)-4,5-difluoro-1H- benzo[d]imidazole-6-carboxylic acid | 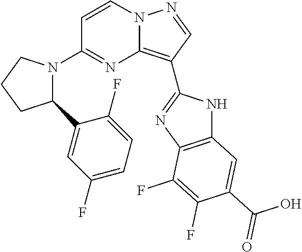 [SMILES: O=C(O)c1cc2[nH]c(-c3cnn4ccc(N5CCC[C@@H]5c5cc(F)ccc5F)nc34)nc2c(F)c1F] | 496.5 |
| 139 | (R)-5-(2-(2,5- difluorophenyl)pyrrolidin-1-yl)- 3-(5-fluoro-6-(trifluoromethyl)- 1H-benzo[d]imidazol-2- yl)pyrazolo[1,5-a]pyrimidine | 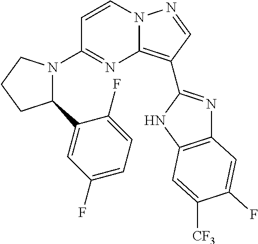 [SMILES: Fc1ccc(F)c([C@H]2CCCN2c2ccn3ncc(-c4nc5cc(F)c(C(F)(F)F)cc5[nH]4)c3n2)c1] | 502.5 |
| 140 | (R)-2-(5-(2-(2,5- difluorophenyl)pyrrolidin-1- yl)pyrazolo[1,5-a]pyrimidin-3- yl)-6-ethoxy-1H- benzo[d]imidazole-5-carbonitrile | 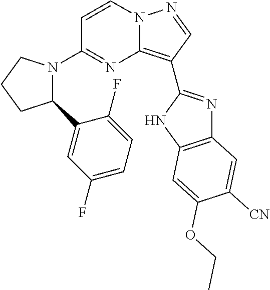 [SMILES: CCOc1cc2[nH]c(-c3cnn4ccc(N5CCC[C@@H]5c5cc(F)ccc5F)nc34)nc2cc1C#N] | 485.6 |
| 141 | (R)-2-(5-(2-(2,5- difluorophenyl)pyrrolidin-1- yl)pyrazolo[1,5-a]pyrimidin-3- yl)-6-fluoro-1H- benzo[d]imidazole-5-carboxylic acid | 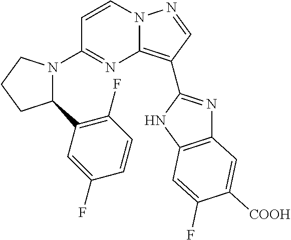 [SMILES: O=C(O)c1cc2nc(-c3cnn4ccc(N5CCC[C@@H]5c5cc(F)ccc5F)nc34)[nH]c2cc1F] | 478.5 |
| 142 | (R)-2-(5-(2-(2,5- difluorophenyl)pyrrolidin-1- yl)pyrazolo[1,5-a]pyrimidin-3- yl)-6-(methylamino)-1H- benzo[d]imidazole-5-carbonitrile | 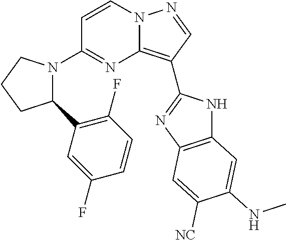 [SMILES: CNc1cc2[nH]c(-c3cnn4ccc(N5CCC[C@@H]5c5cc(F)ccc5F)nc34)nc2cc1C#N] | 470.6 |
| 143 | (R)-2-(5-(2-(2,5- difluorophenyl)pyrrolidin-1- yl)pyrazolo[1,5-a]pyrimidin-3- yl)-6-morpholino-1H- benzo[d]imidazole-5-carbonitrile | 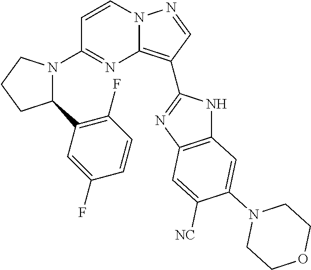 [SMILES: N#Cc1cc2nc(-c3cnn4ccc(N5CCC[C@@H]5c5cc(F)ccc5F)nc34)[nH]c2cc1N1CCOCC1] | 526.6 |
| 144 | (R)-2-(5-(2-(2,5- difluorophenyl)pyrrolidin-1- yl)pyrazolo[1,5-a]pyrimidin-3- yl)-6-(dimethylamino)-1H- benzo[d]imidazole-5-carbonitrile | 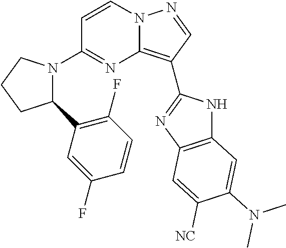 [SMILES: CN(C)c1cc2[nH]c(-c3cnn4ccc(N5CCC[C@@H]5c5cc(F)ccc5F)nc34)nc2cc1C#N] | 484.6 |
| 145 | (R)-2-(5-(2-(2,5- difluorophenyl)pyrrolidin-1- yl)pyrazolo[1,5-a]pyrimidin-3- yl)-6-(3-hydroxyazetidin-1-yl)- 1H-benzo[d]imidazole-5- carbonitrile | 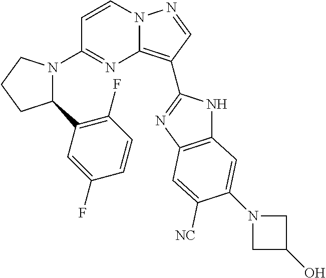 [SMILES: N#Cc1cc2nc(-c3cnn4ccc(N5CCC[C@@H]5c5cc(F)ccc5F)nc34)[nH]c2cc1N1CC(O)C1] | 512.6 |
| 146 | (R)-2-(5-(2-(2,5- difluorophenyl)pyrrolidin-1- yl)pyrazolo[1,5-a]pyrimidin-3- yl)-5,6,7,8- tetrahydroimidazo[4′,5′:4,5] benzo[1,2-e][1,4]diazepin- 9(3H)-one | 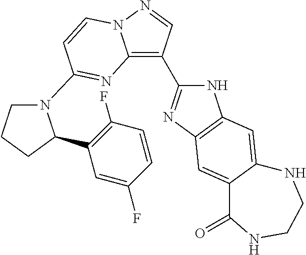 [SMILES: O=C1NCCNc2cc3[nH]c(-c4cnn5ccc(N6CCC[C@@H]6c6cc(F)ccc6F)nc45)nc3cc21] | 500.6 |
| 147 | (R)-2-(5-(2-(2,5- difluorophenyl)pyrrolidin-1- yl)pyrazolo[1,5-a]pyrimidin-3- yl)-7,8-dihydro-3H- imidazo[4′,5′:4,5]benzo[1,2- f][1,4]oxazepin-9(6H)-one | 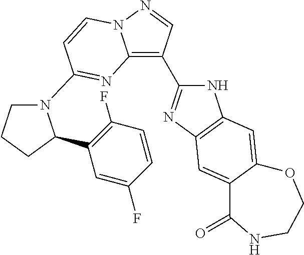 [SMILES: O=C1NCCOc2cc3[nH]c(-c4cnn5ccc(N6CCC[C@@H]6c6cc(F)ccc6F)nc45)nc3cc21] | 501.6 |
| 148 | (R)-2-(5-(2-(2,5- difluorophenyl)pyrrolidin-1- yl)pyrazolo[1,5-a]pyrimidin-3- yl)-1H-benzo[d]imidazole-5,6- dicarbonitrile | 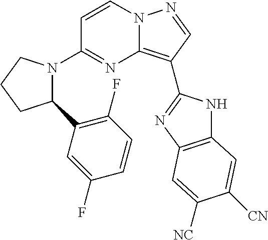 [SMILES: N#Cc1cc2nc(-c3cnn4ccc(N5CCC[C@@H]5c5cc(F)ccc5F)nc34)[nH]c2cc1C#N] | 466.5 |
| 149 | (R)-2-(5-(2-(2,5- difluorophenyl)pyrrolidin-1- yl)pyrazolo[1,5-a]pyrimidin-3- yl)-6-hydroxy-1H- benzo[d]imidazole-5-carbonitrile | 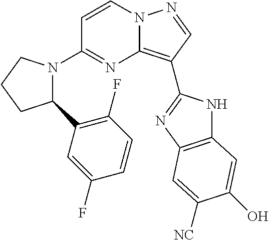 [SMILES: N#Cc1cc2nc(-c3cnn4ccc(N5CCC[C@@H]5c5cc(F)ccc5F)nc34)[nH]c2cc1O] | 457.5 |
| 150 | (R)-2-(5-(2-(2,5- difluorophenyl)pyrrolidin-1- yl)pyrazolo[1,5-a]pyrimidin-3- yl)-6-(2-hydroxyethoxy)-1H- benzo[d]imidazole-5-carbonitrile | 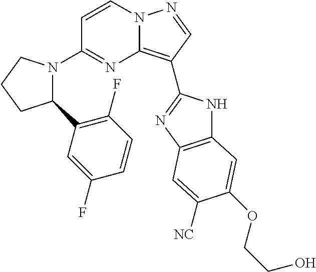 [SMILES: N#Cc1cc2nc(-c3cnn4ccc(N5CCC[C@@H]5c5cc(F)ccc5F)nc34)[nH]c2cc1OCCO] | 501.6 |
| 151 | (R)-6-bromo-2-(5-(2-(2,5- difluorophenyl)pyrrolidin-1- yl)pyrazolo[1,5-a]pyrimidin-3- yl)-1H-benzo[d]imidazole-5- carbonitrile | 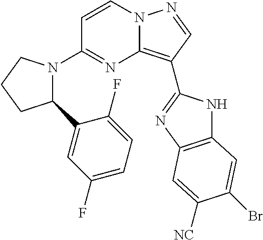 [SMILES: N#Cc1cc2nc(-c3cnn4ccc(N5CCC[C@@H]5c5cc(F)ccc5F)nc34)[nH]c2cc1Br] | 520.4 |
| 152 | methyl (R)-5-cyano-2-(5-(2-(2,5- difluorophenyl)pyrrolidin-1- yl)pyrazolo[1,5-a]pyrimidin-3- yl)-1H-benzo[d]imidazole-6- carboxylate | 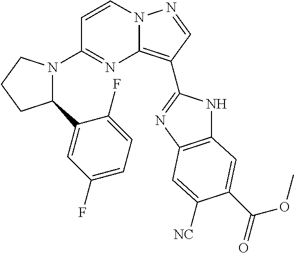 [SMILES: COC(=O)c1cc2[nH]c(-c3cnn4ccc(N5CCC[C@@H]5c5cc(F)ccc5F)nc34)nc2cc1C#N] | 499.6 |
| 153 | (R)-5-cyano-2-(5-(2-(2,5- difluorophenyl)pyrrolidin-1- yl)pyrazolo[1,5-a]pyrimidin-3- yl)-1H-benzo[d]imidazole-6- carboxylic acid | 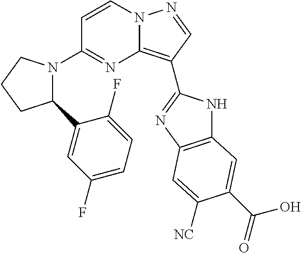 [SMILES: N#Cc1cc2nc(-c3cnn4ccc(N5CCC[C@@H]5c5cc(F)ccc5F)nc34)[nH]c2cc1C(=O)O] | 485.5 |
| 154 | (R)-5-cyano-2-(5-(2-(2,5- difluorophenyl)pyrrolidin-1- yl)pyrazolo[1,5-a]pyrimidin-3- yl)-1H-benzo[d]imidazole-6- carboxamide | 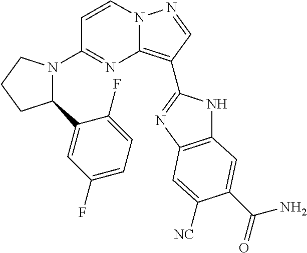 [SMILES: N#Cc1cc2nc(-c3cnn4ccc(N5CCC[C@@H]5c5cc(F)ccc5F)nc34)[nH]c2cc1C(N)=O] | 484.6 |
| 155 | methyl (R)-2-(5-(2-(2,5- difluorophenyl)pyrrolidin-1- yl)pyrazolo[1,5-a]pyrimidin-3- yl)-6-methoxy-1H- benzo[d]imidazole-5-carboxylate | 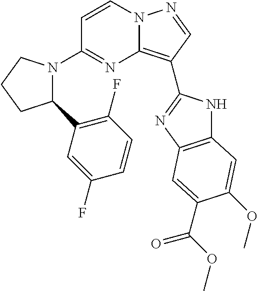 [SMILES: COC(=O)c1cc2nc(-c3cnn4ccc(N5CCC[C@@H]5c5cc(F)ccc5F)nc34)[nH]c2cc1OC] | 504.6 |
| 156 | (R)-6-(difluoromethoxy)-2-(5-(2- (2,5-difluorophenyl)pyrrolidin-1- yl)pyrazolo[1,5-a]pyrimidin-3- yl)-1H-benzo[d]imidazole-5- carbonitrile | 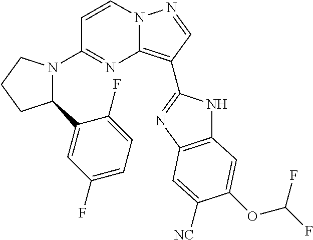 [SMILES: N#Cc1cc2nc(-c3cnn4ccc(N5CCC[C@@H]5c5cc(F)ccc5F)nc34)[nH]c2cc1OC(F)F] | 507.5 |
| 157 | (R)-2-(5-(2-(2,5- difluorophenyl)pyrrolidin-1- yl)pyrazolo[1,5-a]pyrimidin-3- yl)-5-(trifluoromethyl)-1H- benzo[d]imidazole-6-carbonitrile | 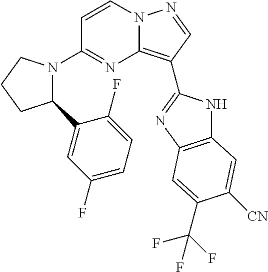 [SMILES: N#Cc1cc2[nH]c(-c3cnn4ccc(N5CCC[C@@H]5c5cc(F)ccc5F)nc34)nc2cc1C(F)(F)F] | 509.5 |
| 158 | methyl (R)-2-(5-(2-(2,5- difluorophenyl)pyrrolidin-1- yl)pyrazolo[1,5-a]pyrimidin-3- yl)-6-fluoro-1H- benzo[d]imidazole-7-carboxylate | 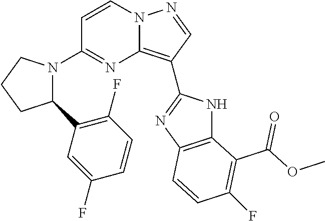 [SMILES: COC(=O)c1c(F)ccc2nc(-c3cnn4ccc(N5CCC[C@@H]5c5cc(F)ccc5F)nc34)[nH]c12] | 492.6 |
| 159 | (R)-2-(5-(2-(2,5- difluorophenyl)pyrrolidin-1- yl)pyrazolo[1,5-a]pyrimidin-3- yl)-6-methyl-1H- benzo[d]imidazole-5-carbonitrile | 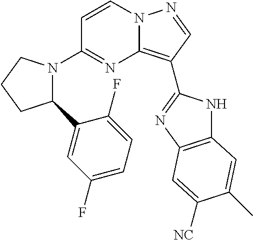 [SMILES: Cc1cc2[nH]c(-c3cnn4ccc(N5CCC[C@@H]5c5cc(F)ccc5F)nc34)nc2cc1C#N] | 455.6 |
| 160 | (R)-2-(5-(2-(2,5- difluorophenyl)pyrrolidin-1- yl)pyrazolo[1,5-a]pyrimidin-3- yl)-6-methoxy-N-methyl-1H- benzo[d]imidazole-5- carboxamide | 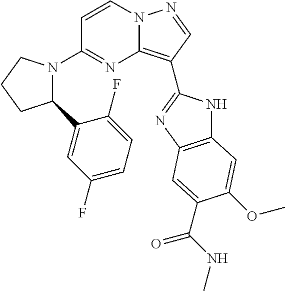 [SMILES: CNC(=O)c1cc2nc(-c3cnn4ccc(N5CCC[C@@H]5c5cc(F)ccc5F)nc34)[nH]c2cc1OC] | 503.6 |
| 161 | (R)-2-(5-(2-(2,5- difluorophenyl)pyrrolidin-1- yl)pyrazolo[1,5-a]pyrimidin-3- yl)-6-methoxy-N,N-dimethyl- 1H-benzo[d]imidazole-5- carboxamide | 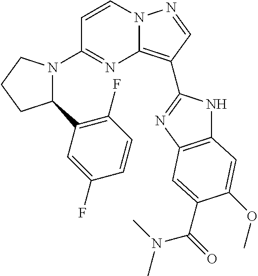 [SMILES: COc1cc2[nH]c(-c3cnn4ccc(N5CCC[C@@H]5c5cc(F)ccc5F)nc34)nc2cc1C(=O)N(C)C] | 517.6 |
| 162 | (R)-4-((2-(5-(2-(2,5- difluorophenyl)pyrrolidin-1- yl)pyrazolo[1,5-a]pyrimidin-3- yl)-1H-benzo[d]imidazol-5- yl)methyl)morpholine | 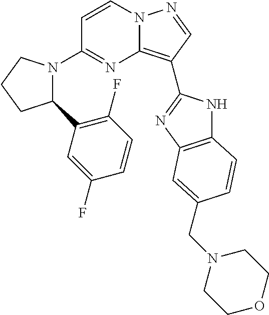 [SMILES: Fc1ccc(F)c([C@H]2CCCN2c2ccn3ncc(-c4nc5cc(CN6CCOCC6)ccc5[nH]4)c3n2)c1] | 515.7 |

*Remark: If there is an isomeride, such as a tautomer in the above compounds, the present invention also includes its isomeride, such as tautomer, and also includes their mixture.

### Example 125 Synthesis of Compound 125 and/or its Isomeride

### (R)-2-(5-(2-(2,5-difluorophenyl)pyrrolidin-1-yl)pyrazolo[1,5-a]pyrimidin-3-yl)-5-methoxy-1H-benzo[d]imidazole-6-carbonitrile

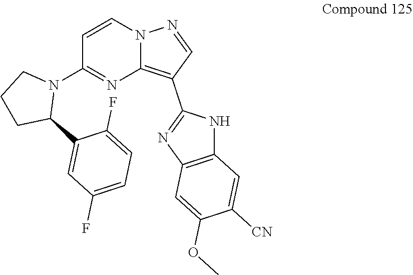 [SMILES: COc1cc2nc(-c3cnn4ccc(N5CCC[C@@H]5c5cc(F)ccc5F)nc34)[nH]c2cc1C#N]

### (R)-2-(5-(2-(2,5-difluorophenyl)pyrrolidin-1-yl)pyrazolo[1,5-a]pyrimidin-3-yl)-6-methoxy-1H-benzo[d]imidazole-5-carbonitrile

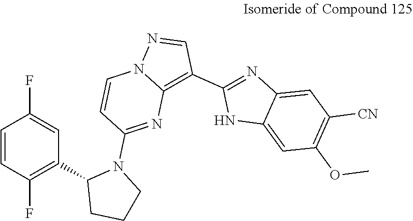 [SMILES: COc1cc2[nH]c(-c3cnn4ccc(N5CCC[C@@H]5c5cc(F)ccc5F)nc34)nc2cc1C#N]

### Step 1: Preparation of 4-amino-2-methoxy-5-nitrobenzonitrile

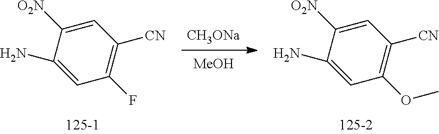

To a solution of CH₃ONa (14.6 g) in MeOH (300 mL) was added 4-amino-2-fluoro-5-nitrobenzonitrile (9.8 g) below 15° C. Then the solution was warmed to room temperature and stirred for 8 h. LCMS showed the reaction was completed, concentrated under reduced pressure to remove MeOH, the residue was added 1 L water and adjust pH to 4-5 with 2N HCl aqueous solution. Filtered and the solid was washed with water, dried under reduced pressure at 50° C. for 10 h to afford the product 4-amino-2-methoxy-5-nitrobenzonitrile (9.6 g) as a yellow solid.

### Step 2: Preparation of 4,5-diamino-2-methoxybenzonitrile

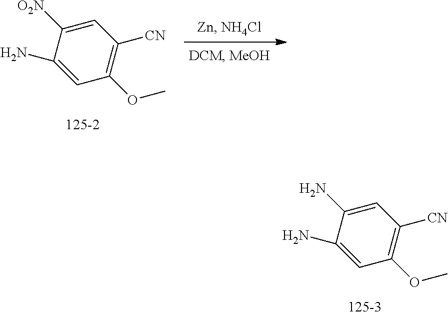

To a solution of 4-amino-2-methoxy-5-nitrobenzonitrile (9.6 g) in DCM/MeOH (1:1, 60 mL) was added saturated NH₄Cl(aq) (60 mL). Zinc powder (32.5 g) was added to the mixture, then the mixture was stirred at room temperature for 2 h. LCMS showed the reaction was completed. The reaction mixture was filtered and the filtrate was extracted with DCM (3*100 mL), combined the organic layers, washed with brine, concentrated under reduced pressure and the residue was purified by combiflash (PE:EA=50%:50%) to afford the product 4,5-diamino-2-methoxybenzonitrile (7.3 g) as a red solid.

### Step 3: Synthesis of (R)-2-(5-(2-(2,5-difluorophenyl)pyrrolidin-1-yl)pyrazolo[1,5-a]pyrimidin-3-yl)-5-methoxy-1H-benzo[d]imidazole-6-carbonitrile and/or Isomeride Thereof

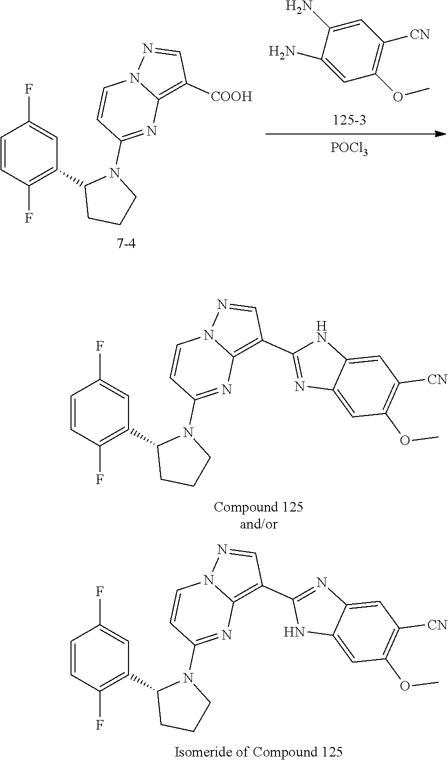

To a solution of (R)-5-(2-(2,5-difluorophenyl)pyrrolidin-1-yl)pyrazolo[1,5-a]pyrimidine-3-carboxylic acid (3.44 g) in POCl₃ (30 mL) was added 4,5-diamino-2-methoxybenzonitrile (1.96 g). The mixture was heater to 90° C. and stirred for 3 h. LCMS showed the reaction was completed. Cooled to room temperature and concentrated under reduced pressure to remove POCl₃, the residue was poured into water (300 mL) and precipitated, the precipitation was filtered, then the solid was added to 1N NaOH aqueous solution and stirred overnight, then the solid was filtered and washed with water, and the filter cake dried under vacuum at 60° C. for 10 h to afford final product (Compound 125 and/or the isomeride of Compound 125) as a yellow solid (3.98 g). MS: [M+H]⁺: 472.16.

1H NMR (500 MHz, DMSO-d6) δ 10.53-11.44 (m, 1H), 8.63 and 8.78 (br+br, 1H), 8.37 and 8.46 (s+s, 1H), 7.56 and 7.87 and 7.90 (s+s+s, 1H), 6.97 and 7.21-7.36 (m+m, 4H), 6.09 and 6.63 (br+br, 1H), 5.37 and 5.66 (br+br, 1H), 3.72 and 4.25 (br+br, 1H), 3.94-4.00 (m, 4H), 2.57 (br, 1H), 1.98-2.15 (m, 3H).

### Example 156 Synthesis of Compound 156 and/or its Isomeride

### (R)-6-(difluoromethoxy)-2-(5-(2-(2,5-difluorophenyl)pyrrolidin-1-yl)pyrazolo[1,5-a]pyrimidin-3-yl)-1H-benzo[d]imidazole-5-carbonitrile

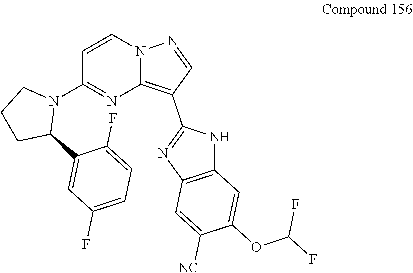 [SMILES: N#Cc1cc2nc(-c3cnn4ccc(N5CCC[C@@H]5c5cc(F)ccc5F)nc34)[nH]c2cc1OC(F)F]

### (R)-5-(difluoromethoxy)-2-(5-(2-(2,5-difluorophenyl)pyrrolidin-1-yl)pyrazolo[1,5-a]pyrimidin-3-yl)-1H-benzo[d]imidazole-6-carbonitrile

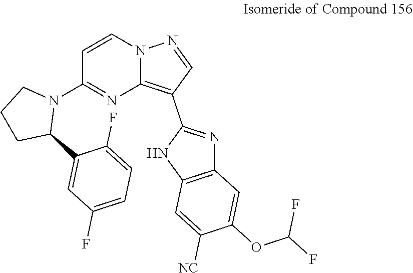 [SMILES: N#Cc1cc2[nH]c(-c3cnn4ccc(N5CCC[C@@H]5c5cc(F)ccc5F)nc34)nc2cc1OC(F)F]

### Step 1: Synthesis of 4-amino-2-hydroxy-5-nitrobenzonitrile

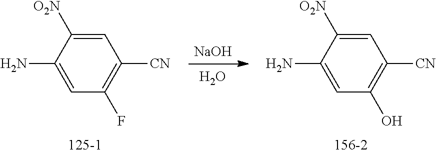

To a solution of NaOH (8.8 g) in water (100 mL) was added 4-amino-2-fluoro-5-nitrobenzonitrile (10 g) below 15° C., and the mixture was stirred for 8 h at 80° C. The reaction was monitored by LC-MS. After the 4-amino-2-fluoro-5-nitrobenzonitrile was consumed completely, the reaction mixture was adjusted to PH 6-7 using 6 N HCl below 20° C. The mixture was filtered and the filter cake was washed by water, dried under reduced pressure at 50° C. for 10 h to afford 4-amino-2-hydroxy-5-nitrobenzonitrile (9.0 g) as a yellow solid.

### Step 2: Synthesis of tert-butyl (4-cyano-5-hydroxy-2-nitrophenyl)carbamate

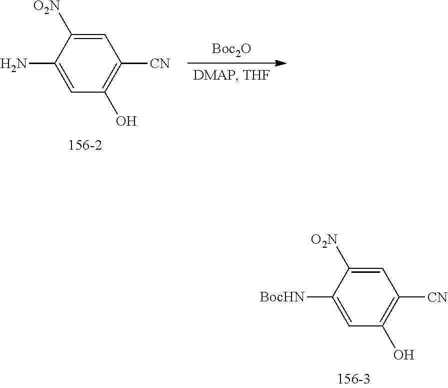

To a solution of 4-amino-2-hydroxy-5-nitrobenzonitrile (500 mg) in THF (15 mL) was added Boc₂O (670 mg) and DMAP (34 mg). The mixture was stirred for 4 h at room temperature, and TLC showed 4-amino-2-hydroxy-5-nitrobenzonitrile was completely. The mixture was evaporated under in vacuo, and the residue was diluted by EA. The organic phase was washed with 0.5 N HCl, water, brine and dried over Na₂SO₄. The solvent was evaporated in vacuo, and the residue was purified by silica gel column chromatography (EA/PE: 0˜20% in 30 min) to afford the desired product (646 mg) as a yellow solid.

### Step 3: Synthesis of tert-butyl (4-cyano-5-(difluoromethoxy)-2-nitrophenyl)carba-mate

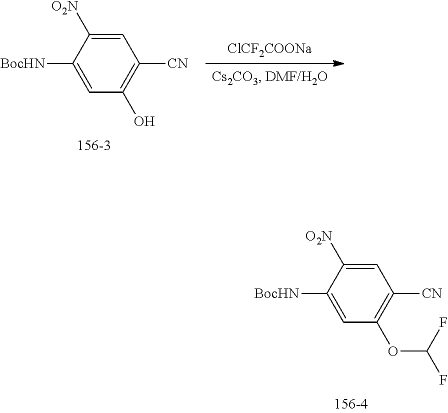

To a mixture of tert-butyl (4-cyano-5-hydroxy-2-nitrophenyl)carbamate (100 mg), ClCF₂COONa (109 mg) and Cs₂CO₃ (140 mg) was added DMF (3 mL) and water (0.3 mL). The mixture was stirred for 2 h at 90° C. After TLC showed tert-butyl (4-cyano-5-(difluoromethoxy)-2-nitrophenyl)carbamate was consumed completely, the reaction mixture diluted by EA. The organic phase was washed with water, brine and dried over Na₂SO₄. The solvent was evaporated in vacuo, and the residue was purified by silica gel column chromatography (EA/PE: O-20% in 30 min) to afford the desired product (77 mg) as a yellow solid.

### Step 4: tert-butyl (2-amino-4-cyano-5-(difluoromethoxy)phenyl)carbamate

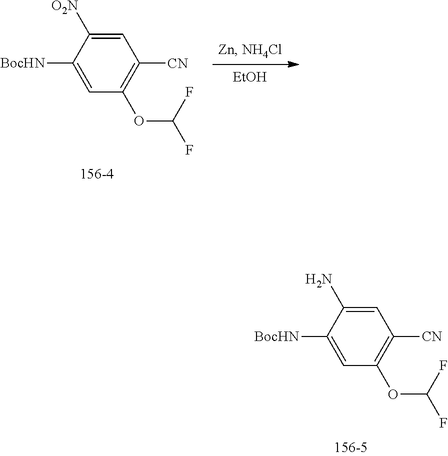

To a mixture of tert-butyl (4-cyano-5-(difluoromethoxy)-2-nitrophenyl)carbamate (77 mg), zinc powder (91 mg) and NH₄Cl (126 mg) was added EtOH (3 mL) and water (1 mL). The mixture was stirred for 12 h at 80° C. After TLC and LC-MS showed tert-butyl (4-cyano-5-(difluoromethoxy)-2-nitrophenyl)carbamate was consumed completely, the reaction mixture filtered. The filtrated was concentrated in vacuo, and the residue was diluted by water. The aqueous phase was extracted with DCM, and the combined organic phases were washed with brine, dried over Na₂SO₄ and concentrated in vacuo to afford crude product, which was purified by silica gel column chromatography (MeOH/DCM: 0˜5% in 30 min) to afford the desired product (56 mg) as a yellow solid.

### Step 5: 4,5-diamino-2-(difluoromethoxy)benzonitrile

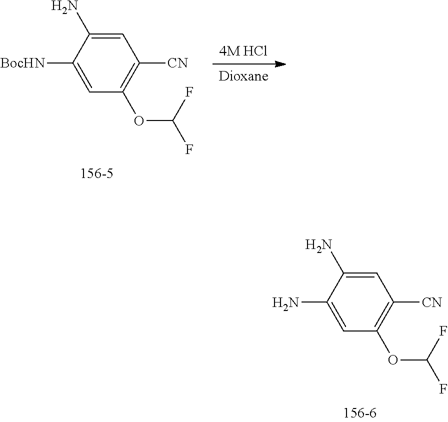

To tert-butyl (2-amino-4-cyano-5-(difluoromethoxy)phenyl)carbamate (56 mg) was added 4M HCl in dioxane (4 mL). The mixture was stirred for 4 h at room temperature. After LC-MS showed 4,5-diamino-2-(difluoromethoxy)benzonitrile was consumed completely, the reaction mixture was concentrated in vacuo, and the residue was diluted by aq. NaHCO₃. The aqueous phase was extracted with DCM, and the combined organic phases were washed with brine, dried over Na₂SO₄ and concentrated in vacuo to afford desired product (37 mg), which was used directly in next step.

### Step 6: Synthesis of (R)-6-(difluoromethoxy)-2-(5-(2-(2,5-difluorophenyl)pyrrolidin-1-yl)pyrazolo[1,5-a]pyrimidin-3-yl)-1H-benzo[d]imidazole-5-carbonitrile and/or isomeride thereof

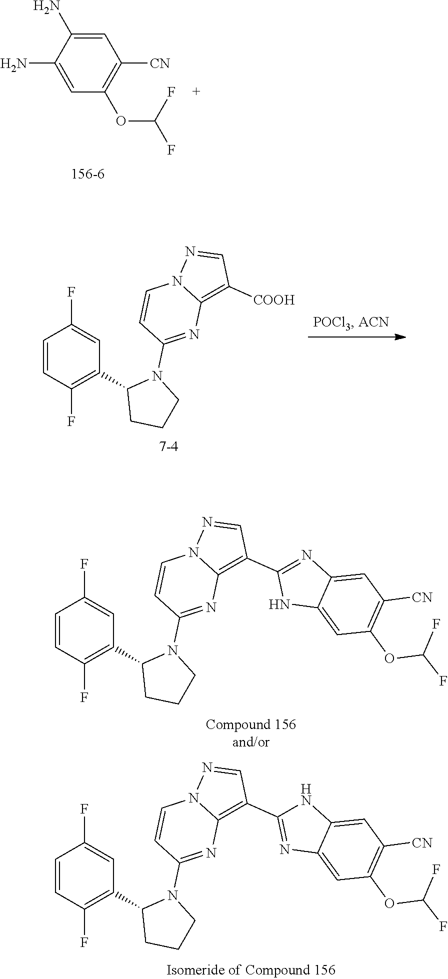 [SMILES: N#Cc1cc(N)c(N)cc1OC(F)F ; O=C(O)c1cnn2ccc(N3CCC[C@@H]3c3cc(F)ccc3F)nc12 ; N#Cc1cc2nc(-c3cnn4ccc(N5CCC[C@@H]5c5cc(F)ccc5F)nc34)[nH]c2cc1OC(F)F ; N#Cc1cc2[nH]c(-c3cnn4ccc(N5CCC[C@@H]5c5cc(F)ccc5F)nc34)nc2cc1OC(F)F]

To a solution of (R)-5-(2-(2,5-difluorophenyl)pyrrolidin-1-yl)pyrazolo[1,5-a]pyrimidine-3-carboxylic acid (64 mg) in CH₃CN (2 mL) was added POCl₃ (54 μL, 0.561 mmol) and 4,5-diamino-2-(difluoromethoxy)benzonitrile (37 mg). The mixture was stirred for 3 h at 90° C. After LC-MS showed 4,5-diamino-2-(difluoromethoxy)benzonitrile was consumed completely, the reaction mixture was concentrated in vacuo, and the residue was diluted by EA. The organic phase was washed with water, brine and dried over Na₂SO₄. The solvent was evaporated in vacuo, and the residue was purified by silica gel column chromatography (MeOH/DCM: 0˜8% in 30 min) to afford the desired product (Compound 156 and/or the isomeride of Compound 156) as yellow solid (18 mg). MS: [M+H]⁺ 508.18.

### Example 163 Synthesis of Compound 163 and/or its Isomeride

### (R)-2-(5-(2-(2,5-difluorophenyl)pyrrolidin-1-yl)pyrazolo[1,5-a]pyrimidin-3-yl)-6-(4-methylpiperazin-1-yl)-1H-benzo[d]imidazole-5-carbonitrile

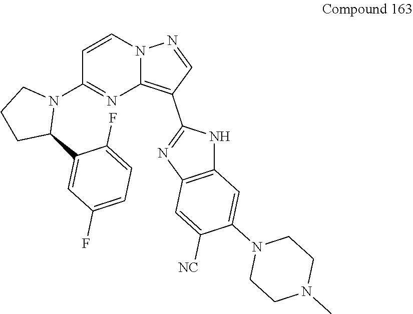 [SMILES: CN1CCN(c2cc3[nH]c(-c4cnn5ccc(N6CCC[C@@H]6c6cc(F)ccc6F)nc45)nc3cc2C#N)CC1]

### (R)-2-(5-(2-(2,5-difluorophenyl)pyrrolidin-1-yl)pyrazolo[1,5-a]pyrimidin-3-yl)-5-(4-methylpiperazin-1-yl)-1H-benzo[d]imidazole-6-carbonitrile

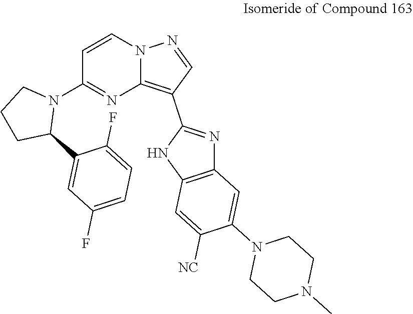 [SMILES: CN1CCN(c2cc3nc(-c4cnn5ccc(N6CCC[C@@H]6c6cc(F)ccc6F)nc45)[nH]c3cc2C#N)CC1]

### Step 1: Synthesis of 4-amino-2-(4-methylpiperazin-1-yl)-5-nitrobenzonitrile

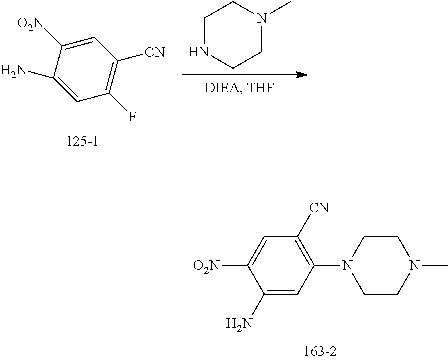

To a solution of 4-amino-2-fluoro-5-nitrobenzonitrile (18.1 g) in THF (300 mL) was added 1-methylpiperazine (12.1 g) and DIEA (25.8 g) below 15° C. Then the solution was warmed to room temperature and stirred for 3 h. LCMS showed the reaction was completed. The reaction mixture was poured into ice-water and extracted with EA (3*100 mL), combined the organic layers and washer with brine, dried over Na₂SO₄. Concentrated to afford the desired product 4-amino-2-(4-methylpiperazin-1-yl)-5-nitrobenzonitrile (21.5 g) as a brown solid.

### Step 2: Synthesis of 4,5-diamino-2-(4-methylpiperazin-1-yl)benzonitrile

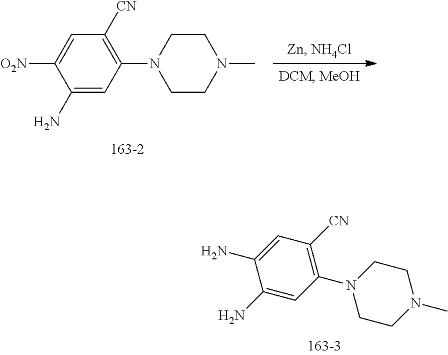

To a solution of 4-amino-2-(4-methylpiperazin-1-yl)-5-nitrobenzonitrile (13.1 g) in DCM/MeOH (1:1, 60 mL) was added NH₄Cl/H₂O (60 mL). The mixture was stirred, Zn (32.8 g) was added, then the solution was stirred at room temperature for 2 h. LCMS showed the reaction was completed. The reaction mixture was filtered and the filtrate was extracted with DCM (3*100 mL), combined the organic layers, washed with brine, Concentrated under reduced pressure and the residue was purified by combiflash (PE:EA=50%:50%) to afford the desired product 4,5-diamino-2-(4-methylpiperazin-1-yl)benzonitrile (8.7 g) as a brown solid.

### Step 3: Synthesis of (R)-2-(5-(2-(2,5-difluorophenyl)pyrrolidin-1-yl)pyrazolo[1,5-a]pyrimidin-3-yl)-6-(4-methylpiperazin-1-yl)-1H-benzo[d]imidazole-5-carbonitrile and/or Isomeride Thereof

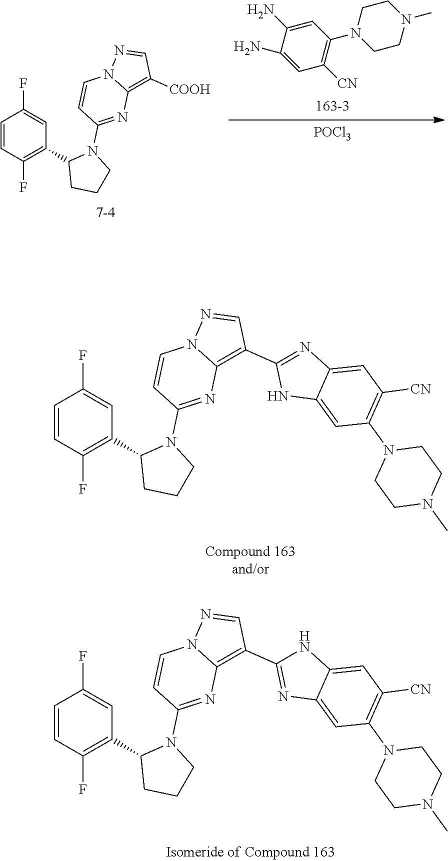

To a solution of (R)-5-(2-(2,5-difluorophenyl)pyrrolidin-1-yl)pyrazolo[1,5-a]pyrimidine-3-carboxylic acid (3.44 g) in POCl₃ (30 mL) was added 4,5-diamino-2-(4-methylpiperazin-1-yl)benzonitrile (2.77 g). The mixture was heater to 90° C. and stirred for 3 h. LCMS showed the reaction was completed. Cooled to room temperature and concentrated under reduced pressure to remove POCl₃, the residue was poured into water (300 mL) and filtered, the solid was washed with NaHCO₃ saturated solution and water, dried under reduce pressure at 60° C. for 10 h to afford the desired product (Compound 163 and/or the isomeride of Compound 163) (3.58 g) as a yellow solid. MS: [M+H]⁺ 540.81

Prepare the following examples (shown in Table 4) essentially as described for Example 163 using the corresponding starting materials. For example, prepare the following Example 164

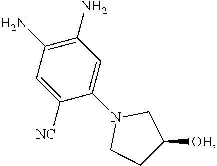 [SMILES: N#Cc1cc(N)c(N)cc1N1CC[C@H](O)C1]

(shown in Table 4) essentially as described for Example 163 using instead of

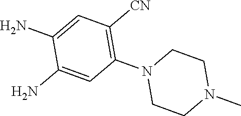 [SMILES: CN1CCN(c2cc(N)c(N)cc2C#N)CC1]

and other starting materials are either commercially available or made by known procedures in the reported literature or as illustrated.

TABLE 4

| | | | Physical Data |
|---|---|---|---|
| EX No. | Chemical Name | Structure | (MS) (M + H)⁺ |
| 164 | 2-(5-((R)-2-(2,5- difluorophenyl)pyrrolidin-1- yl)pyrazolo[1,5-a]pyrimidin-3-yl)- 6-((S)-3-hydroxypyrrolidin-1-yl)- 1H-benzo[d]imidazole-5- carbonitrile | 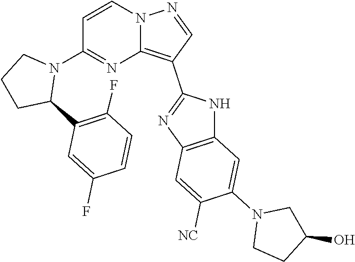 [SMILES: N#Cc1cc2nc(-c3cnn4ccc(N5CCC[C@@H]5c5cc(F)ccc5F)nc34)[nH]c2cc1N1CC[C@H](O)C1] | 526.6 |
| 165 | 6-((S)-2-cyanopyrrolidin-1-yl)-2- (5-((R)-2-(2,5- difluorophenyl)pyrrolidin-1- yl)pyrazolo[1,5-a]pyrimidin-3-yl)- 1H-benzo[d]imidazole-5- carbonitrile | 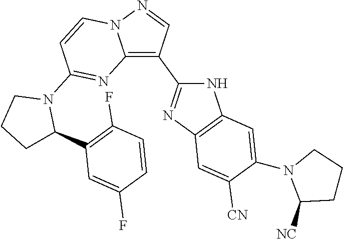 [SMILES: N#Cc1cc2nc(-c3cnn4ccc(N5CCC[C@@H]5c5cc(F)ccc5F)nc34)[nH]c2cc1N1CCC[C@H]1C#N] | 514.6 |
| 166 | methyl (5-cyano-2-(5-((R)-2-(2,5- difluorophenyl)pyrrolidin-1- yl)pyrazolo[1,5-a]pyrimidin-3-yl)- 1H-benzo[d]imidazol-6-yl)-L- prolinate | 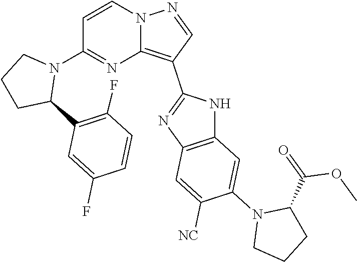 [SMILES: COC(=O)[C@@H]1CCCN1c1cc2[nH]c(-c3cnn4ccc(N5CCC[C@@H]5c5cc(F)ccc5F)nc34)nc2cc1C#N] | 568.7 |
| 167 | (5-cyano-2-(5-((R)-2-(2,5- difluorophenyl)pyrrolidin-1- yl)pyrazolo[1,5-a]pyrimidin-3-yl)- 1H-benzo[d]imidazol-6-yl)-L- proline | 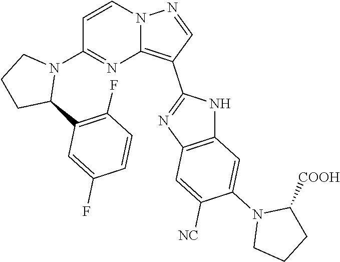 [SMILES: N#Cc1cc2nc(-c3cnn4ccc(N5CCC[C@@H]5c5cc(F)ccc5F)nc34)[nH]c2cc1N1CCC[C@H]1C(=O)O] | 554.7 |
| 168 | (R)-2-(5-(2-(2,5- difluorophenyl)pyrrolidin-1- yl)pyrazolo[1,5-a]pyrimidin-3-yl)- 6-((2- (dimethylamino)ethyl)(methyl) amino)-1H-benzo[d]imidazole-5- carbonitrile | 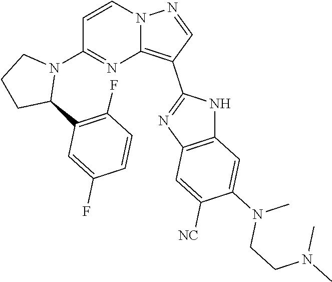 [SMILES: CN(C)CCN(C)c1cc2[nH]c(-c3cnn4ccc(N5CCC[C@@H]5c5cc(F)ccc5F)nc34)nc2cc1C#N] | 541.7 |
| 169 | (R)-2-(5-(2-(2,5- difluorophenyl)pyrrolidin-1- yl)pyrazolo[1,5-a]pyrimidin-3-yl)- 6-(2-methoxyethoxy)-1H- benzo[d]imidazole-5-carbonitrile | 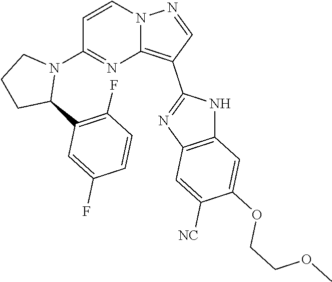 [SMILES: COCCOc1cc2[nH]c(-c3cnn4ccc(N5CCC[C@@H]5c5cc(F)ccc5F)nc34)nc2cc1C#N] | 515.6 |

*Remark: If there is an isomeride in the above compounds, the present invention also includes its isomeride and also includes their mixture.

### Example 170 Synthesis of Compound 170 and/or its Isomeride

### (R)-5-(2-(2,5-difluorophenyl)pyrrolidin-1-yl)-3-(6-(methylsulfonyl)-1H-benzo[d]imidazol-2-yl)pyrazolo[1,5-a]pyrimidine

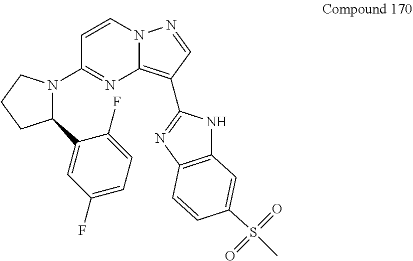 [SMILES: CS(=O)(=O)c1ccc2nc(-c3cnn4ccc(N5CCC[C@@H]5c5cc(F)ccc5F)nc34)[nH]c2c1]

### (R)-5-(2-(2,5-difluorophenyl)pyrrolidin-1-yl)-3-(5-(methylsulfonyl)-1H-benzo[d]imidazol-2-yl)pyrazolo[1,5-a]pyrimidine

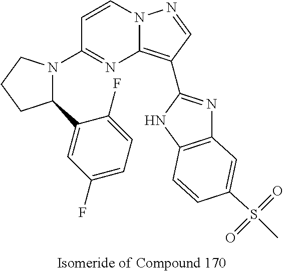 [SMILES: CS(=O)(=O)c1ccc2[nH]c(-c3cnn4ccc(N5CCC[C@@H]5c5cc(F)ccc5F)nc34)nc2c1]

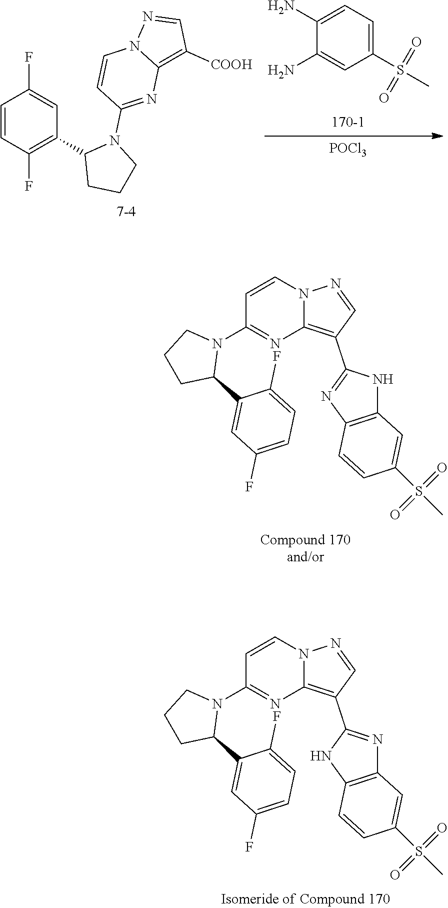

To a solution of (R)-5-(2-(2,5-difluorophenyl)pyrrolidin-1-yl)pyrazolo[1,5-a]pyrimidine-3-carboxylic acid (344 mg) in POCl₃ (5 mL) was added 4-(methylsulfonyl)benzene-1,2-diamine (223 mg). The mixture was heater to 90° C. and stirred for 3 h. LCMS showed the reaction was completed. Cooled to room temperature and concentrated under reduced pressure to remove POCl₃, the residue was poured into water (300 mL) and filtered, the solid was washed with NaHCO₃ saturated solution and water, dried under reduce pressure at 60° C. for 10 h to afford the desired product (Compound 170 and/or the isomeride of Compound 170) as a yellow solid (397 mg). LC-MS: [M+1-1]⁺495.66.

Prepare the following examples (shown in Table 5) essentially as described for Example 170 using the corresponding starting materials. For example, prepare the following Example 171 (shown in Table 5) essentially as described for Example 170 using

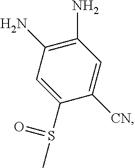

instead of

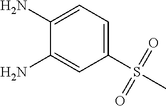

and other starting materials are either commercially available or made by known procedures in the reported literature or as illustrated.

TABLE 5

| | | | Physical Data |
|---|---|---|---|
| EX No. | Chemical Name | Structure | (MS) (M + H)⁺ |
| 171 | 2-(5-((R)-2-(2,5- difluorophenyl)pyrrolidin-1- yl)pyrazolo[1,5-a]pyrimidin-3-yl)- 5-(methylsulfinyl)-1H- benzo[d]imidazole-6-carbonitrile | 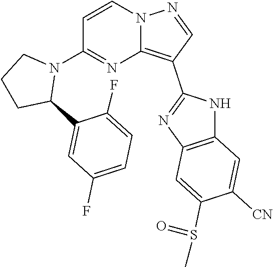 [SMILES: CS(=O)c1cc2nc(-c3cnn4ccc(N5CCC[C@@H]5c5cc(F)ccc5F)nc34)[nH]c2cc1C#N] | 503.6 |
| 172 | (R)-2-(5-(2-(2,5- difluorophenyl)pyrrolidin-1- yl)pyrazolo[1,5-a]pyrimidin-3-yl)- 5-(methylsulfonyl)-1H- benzo[d]imidazole-6-carbonitrile | 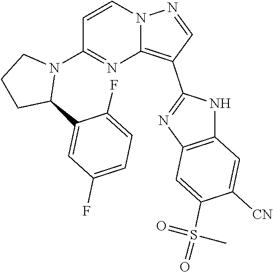 [SMILES: CS(=O)(=O)c1cc2nc(-c3cnn4ccc(N5CCC[C@@H]5c5cc(F)ccc5F)nc34)[nH]c2cc1C#N] | 519.6 |
| 173 | (R)-2-(5-(2-(2,5- difluorophenyl)pyrrolidin-1- yl)pyrazolo[1,5-a]pyrimidin-3-yl)- 5-(methylsulfonyl)-1H- benzo[d]imidazole-6-carboxamide | 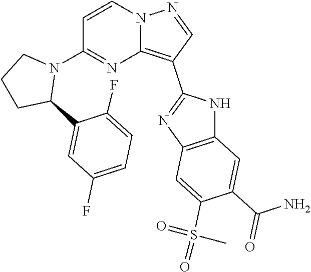 [SMILES: CS(=O)(=O)c1cc2nc(-c3cnn4ccc(N5CCC[C@@H]5c5cc(F)ccc5F)nc34)[nH]c2cc1C(N)=O] | 538.1 |

*Remark: If there is an isomeride in the above compounds, the present invention also includes its isomeride and also includes their mixture.

### Example 63 Synthesis of Compound 63

### 2-(5-((R)-2-(2,5-difluorophenyl)pyrrolidin-1-yl)pyrazolo[1,5-a]pyrimidin-3-yl)-6-((R)-hexahydropyrrolo[1,2-a]pyrazin-2(1H)-yl)benzo[d]thiazole

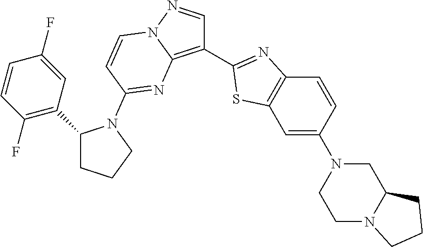 [SMILES: Fc1ccc(F)c([C@H]2CCCN2c2ccn3ncc(-c4nc5ccc(N6CCN7CCC[C@@H]7C6)cc5s4)c3n2)c1]

### Step 1: Synthesis of (R)-5-(hexahydropyrrolo[1,2-a]pyrazin-2(1H)-yl)-2-nitrobenzenethiol

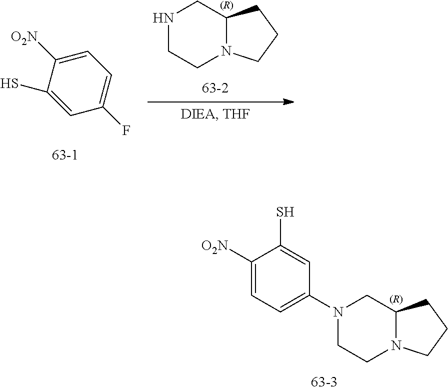

To a solution of 5-fluoro-2-nitrobenzenethiol (1.73 g) in THF (300 mL) was added (R)-octahydropyrrolo[1,2-a]pyrazine (1.51 g) and DIEA (2.58 g) below 15° C. Then the solution was warmed to room temperature and stirred for 3 h. LCMS showed the reaction was completed. The reaction mixture was poured into ice-water and extracted with EA (3*100 mL), combined the organic layers and washer with brine, dried over Na₂SO₄. Concentrated to afford the desired product (R)-5-(hexahydropyrrolo[1,2-a]pyrazin-2(1H)-yl)-2-nitrobenzenethiol (2.13 g) as a brown solid.

### Step 2: Synthesis of (R)-2-amino-5-(hexahydropyrrolo[1,2-a]pyrazin-2(1H)-yl)benzenethiol

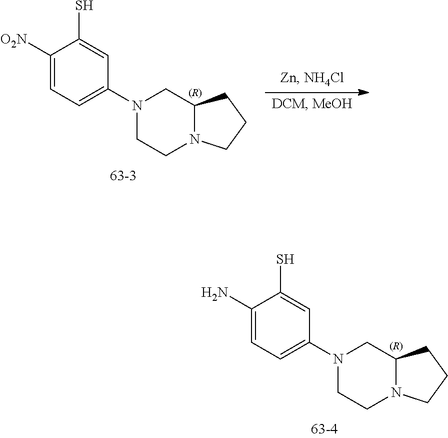

To a solution of (R)-5-(hexahydropyrrolo[1,2-a]pyrazin-2(1H)-yl)-2-nitrobenzenethiol (2.13 g) in DCM/MeOH (1:1, 30 mL) was added NH₄Cl/H₂O (30 mL). The mixture was stirred, Zn (4.9 g) was added, then the solution was stirred at room temperature for 2 h. LCMS showed the reaction was completed. The reaction mixture was filtered and the filtrate was extracted with DCM (3*100 mL), combined the organic layers, washed with brine, Concentrated under reduced pressure and the residue was purified by combiflash (PE:EA=50%:50%) to afford the desired product (R)-2-amino-5-(hexahydropyrrolo[1,2-a]pyrazin-2(1H)-yl)benzenethiol (1.37 g) as a brown solid.

### Step 3: Synthesis of 2-(5-((R)-2-(2,5-difluorophenyl)pyrrolidin-1-yl)pyrazolo[1,5-a]pyrimidin-3-yl)-64R)-hexahydropyrrolo[1,2-a]pyrazin-2(1H)-yl)benzo[d]thiazole

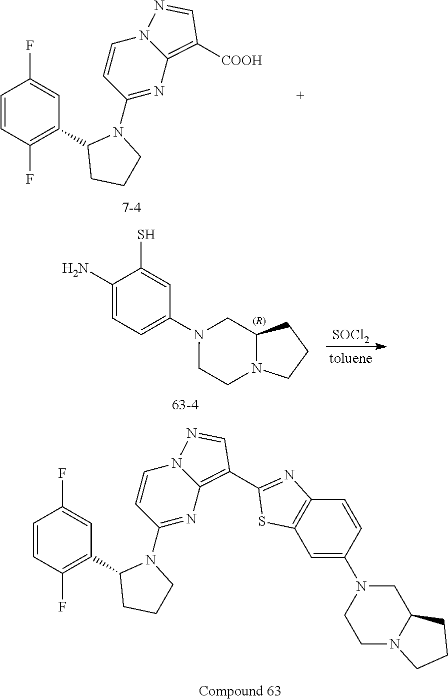

(R)-5-(2-(2,5-difluorophenyl)pyrrolidin-1-yl)pyrazolo[1,5-a]pyrimidine-3-carboxylic acid (3.44 g) was added to toluene, then with SOCl₂(2.38 g) was added, stirred at 80° C. for 2 h, excess SOCl₂ was distilled off, the residue was dissolve in toluene (30 mL) and then (R)-2-amino-5-(hexahydropyrrolo[1,2-a]pyrazin-2(1H)-yl)benzenethiol (2.49 g) was added at 0° C., followed by stirring at room temperature for 1 h.

LCMS showed the reaction was completed. The mixture was diluted with EtOAc (10 mL) and sat. aq NaHCO₃ (5 mL). The organic layer was separated and the aqueous layer extracted with EA (3*5 mL). The combined EtOAc extracts were washed with H₂O (3*5 mL), dried over Na₂SO₄ and concentrated under reduced pressure, the residue was purified by combiflash (DCM:MeOH=95%:5%) to afford the desired product 2-(5-((R)-2-(2,5-difluorophenyl)pyrrolidin-1-yl)pyrazolo[1,5-a]pyrimidin-3-yl)-6-((R)-hexahydropyrrolo[1,2-a]pyrazin-2(1H)-yl)benzo[d]thiazole (2.43 g) as a yellow solid. MS: [M+H]⁺ 558.81.

Prepare the following Example 75 (shown in Table 6) essentially as described for Example 63 using

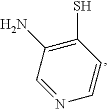

instead of

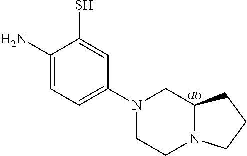

and other starting materials are either commercially available or made by known procedures in the reported literature or as illustrated.

TABLE 6

| | | | Physical Data |
|---|---|---|---|
| EX No. | Structure | Chemical Name | (MS) (M + H)⁺ |
| 75 | (R)-2-(5-(2-(2,5- difluorophenyl)pyrrolidin-1- yl)pyrazolo[1,5-a]pyrimidin-3- yl)thiazolo[4,5-c]pyridine | 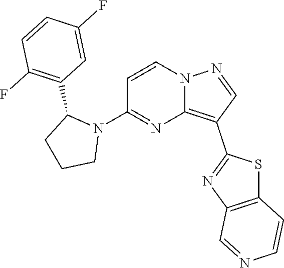 [SMILES: Fc1ccc(F)c([C@H]2CCCN2c2ccn3ncc(-c4nc5cnccc5s4)c3n2)c1] | 435.1 |

### Example 132 Synthesis of Compound 132

### 2-(5-((R)-2-(2,5-difluorophenyl)pyrrolidin-1-yl)pyrazolo[1,5-a]pyrimidin-3-yl)-5,6,7,8-tetrahydro-[1,2,4]triazolo[1,5-a]pyridin-6-01

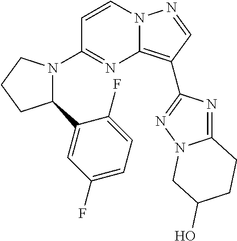 [SMILES: OC1CCc2nc(-c3cnn4ccc(N5CCC[C@@H]5c5cc(F)ccc5F)nc34)nn2C1]

### Step 1: Preparation of (5-oxotetrahydrofuran-3-yl)methyl methanesulfonate

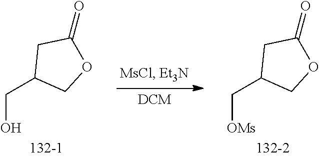 [SMILES: O=C1CC(CO)CO1 ; CS(=O)(=O)OCC1COC(=O)C1]

To a solution of 4-(hydroxymethyl)dihydrofuran-2(3H)-one (236.5 mg) in DCM (5 mL) was added Et₃N (658.7 mg) and MsCl (392.6 mg) at 0° C. for 1 h. The reaction was detected by TLC and LCMS. The mixture was added saturated NH₄Cl solution (3 mL) and DCM. The mixture was extracted by DCM (3*15 mL), the organic layer was dried by Na₂SO₄, then the mixture was concentrated in vacuo and the residue was purified by combi flash (DCM:MeOH=100%:O % to 95%:5%) to afford product (5-oxotetrahydrofuran-3-yl)methyl methanesulfonate (228.7 mg, 58%) as a yellow liquid.

### Step 2: Preparation of 1-amino-5-hydroxypiperidin-2-one

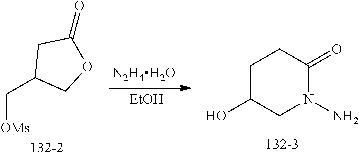

To a solution of (5-oxotetrahydrofuran-3-yl)methyl methanesulfonate (226.7 mg) in EtOH (5 mL) was added N₂H₄.H₂O (52.8 mg) at 80° C. for 6 h. The reaction was detected by TLC and LCMS. The mixture was concentrated in vacuo and the residue was purified by combi flash (DCM:MeOH=100%:O % to 95%:5%) to afford product 1-amino-5-hydroxypiperidin-2-one (56.3 mg, 90%) as a yellow solid.

### Step 3: Preparation of 2-(5-((R)-2-(2,5-difluorophenyl)pyrrolidin-1-yl)pyrazolo[1,5-a]pyrimidin-3-yl)-5,6,7,8-tetrahydro-[1,2,4]triazolo[1,5-a]pyridin-6-ol

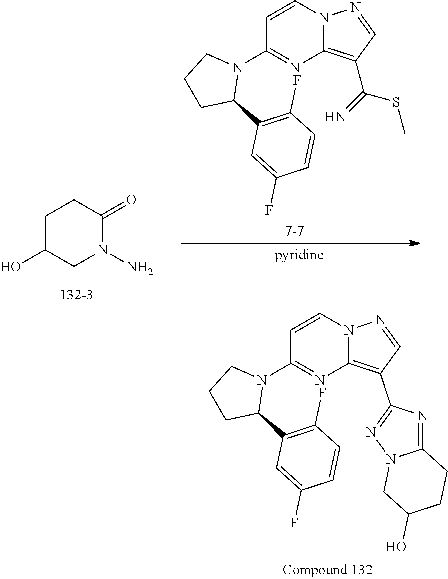 [SMILES: NN1CC(O)CCC1=O ; CSC(=N)c1cnn2ccc(N3CCC[C@@H]3c3cc(F)ccc3F)nc12 ; OC1CCc2nc(-c3cnn4ccc(N5CCC[C@@H]5c5cc(F)ccc5F)nc34)nn2C1]

To a solution of 1-amino-5-hydroxypiperidin-2-one (56.3 mg) in pyridine was added (R)-methyl 5-(2-(2,5-difluorophenyl)pyrrolidin-1-yl)pyrazolo[1,5-a]pyrimidine-3-carbimidothioate (148.7 mg) at 110° C. for 12 h. The reaction was detected by TLC and LCMS. The mixture was concentrated in vacuo and the residue was purified by combi flash (DCM:MeOH=100%:O % to 95%:5%) to yield 37.5 mg (20%, yield) of the title compound. MS(ES⁺): m/z=438.5 (M+H)⁺

¹H NMR (500 MHz, CD₃OD) δ 8.52-8.20 (m, 2H), 7.16 (s, 1H), 7.11-6.86 (m, 2H), 6.62 and 6.08 (1H, s+s), 5.65 and 5.30 (1H, s+s), 4.25-3.69 (m, 4H), 3.16 (s, 1H), 2.67-2.62 (m, 2H), 2.28-1.92 (m, 4H), 1.63-1.59 (m, 2H).

Prepare the following Example 187 (shown in Table 7) essentially as described for Example 132 using

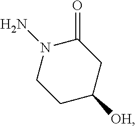

instead of

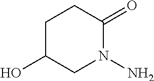

and other starting materials are either commercially available or made by known procedures in the reported literature or as illustrated.

TABLE 7

| | | | Physical Data |
|---|---|---|---|
| EX No. | Chemical Name | Structure | (MS) (M + H)⁺ |
| 187 | (S)-2-(5-((R)-2-(2,5- difluorophenyl)pyrrolidin-1- yl)pyrazolo[1,5-a]pyrimidin-3-yl)- 5,6,7,8-tetrahydro- [1,2,4]triazolo[1,5-a]pyridin-7-ol | 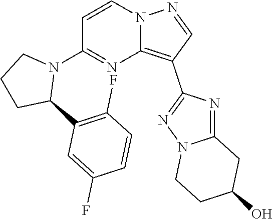 [SMILES: O[C@H]1CCn2nc(-c3cnn4ccc(N5CCC[C@@H]5c5cc(F)ccc5F)nc34)nc2C1] | 437.6 |

### Example 133 Synthesis of Compound 133

### (R)-2-(5-(2-(2,5-difluorophenyl)pyrrolidin-1-yl)pyrazolo[1,5-a]pyrimidin-3-yl)-1H-indole-5-carbonitrile

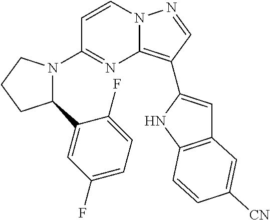 [SMILES: N#Cc1ccc2[nH]c(-c3cnn4ccc(N5CCC[C@@H]5c5cc(F)ccc5F)nc34)cc2c1]

### Step 1: Preparation of (R)-methyl 2-(5-(2-(2,5-difluorophenyl)pyrrolidin-1-yl)pyrazolo[1,5-a]pyrimidin-3-yl)-6-fluorobenzo[d]oxazole-5-carboxylate

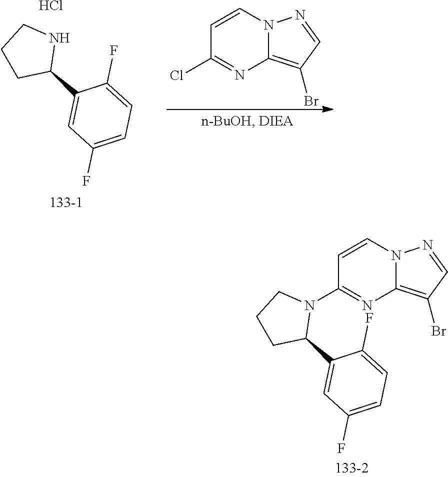 [SMILES: Cl ; Fc1ccc(F)c([C@H]2CCCN2)c1 ; Clc1ccn2ncc(Br)c2n1 ; Fc1ccc(F)c([C@H]2CCCN2c2ccn3ncc(Br)c3n2)c1]

To a solution of (R)-2-(2,5-difluorophenyl)pyrrolidine hydrochloride (2.2 g) in n-BuOH (30 mL) was added DIEA (4.82 g) and 3-bromo-5-chloropyrazolo[1,5-a]pyrimidine (2.61 g) at 100° C. for 3 h. The reaction was detected by TLC and LCMS. Then the mixture was concentrated in vacuo and the mixture was extracted by acetic ether (3×100 mL), the organic layer was dried by Na₂SO₄, then the mixture was concentrated in vacuo and the residue to afford product ((R)-3-bromo-5-(2-(2,5-difluorophenyl)pyrrolidin-1-yl)pyrazolo[1,5-a]pyrimidine (3.5 g, 92%) as a yellow solid.

### Step 2: Preparation of (R)-tert-butyl 5-cyano-2-(5-(2-(2,5-difluorophenyl)pyrrolidin-1-yl)pyrazolo[1,5-a]pyrimidin-3-yl)-1H-indole-1-carboxylate

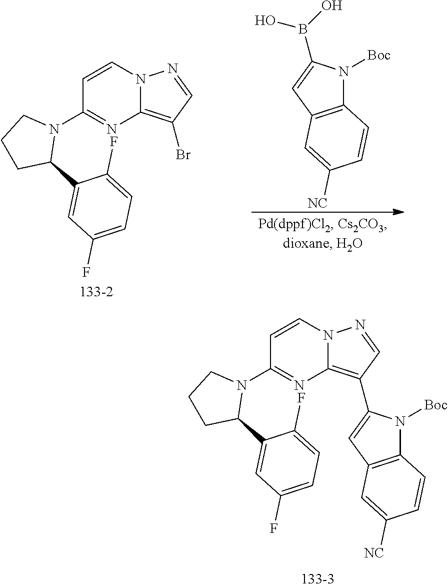 [SMILES: Fc1ccc(F)c([C@H]2CCCN2c2ccn3ncc(Br)c3n2)c1 ; CC(C)(C)OC(=O)n1c(B(O)O)cc2cc(C#N)ccc21 ; CC(C)(C)OC(=O)n1c(-c2cnn3ccc(N4CCC[C@@H]4c4cc(F)ccc4F)nc23)cc2cc(C#N)ccc21]

To a solution of ((R)-3-bromo-5-(2-(2,5-difluorophenyl)pyrrolidin-1-yl)pyrazolo[1,5-a]pyrimidine (162.8 mg) in dioxane (5 mL) was added Cs₂CO₃ (418.3 mg), H₂O (1 Pd(dppf)Cl₂ (62.5 mg) and (1-(tert-butoxycarbonyl)-5-cyano-1H-indol-2-yl)boronic acid (192.7 mg) at 80° C. for 6 h with N₂. The reaction was detected by TLC and LCMS. Then the mixture was concentrated in vacuo and the mixture was extracted by acetic ether (3×50 mL), the organic layer was dried by Na₂SO₄, then the mixture was concentrated in vacuo and the residue was purified by combi flash (PE:EA=100%:O % to 50%:50%) to afford crude product ((R)-tert-butyl 5-cyano-2-(5-(2-(2,5-difluorophenyl)pyrrolidin-1-yl)pyrazolo[1,5-a]pyrimidin-3-yl)-1H-indole-1-carboxylate (106.3 mg, 46%) as a yellow solid.

### Step 3: Preparation of (R)-2-(5-(2-(2,5-difluorophenyl)pyrrolidin-1-yl)pyrazolo[1,5-a]pyrimidin-3-yl)-1H-indole-5-carbonitrile (Compound 133)

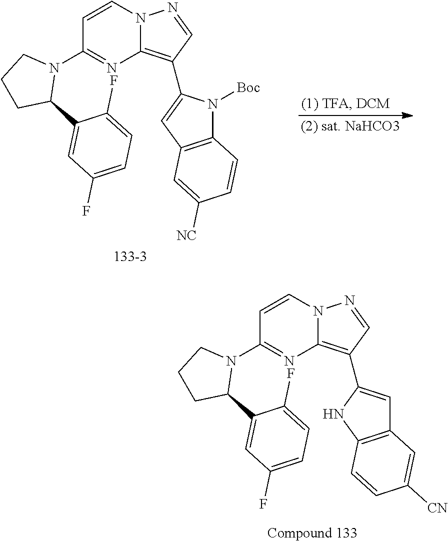 [SMILES: CC(C)(C)OC(=O)n1c(-c2cnn3ccc(N4CCC[C@@H]4c4cc(F)ccc4F)nc23)cc2cc(C#N)ccc21 ; N#Cc1ccc2[nH]c(-c3cnn4ccc(N5CCC[C@@H]5c5cc(F)ccc5F)nc34)cc2c1]

To a solution of ((R)-tert-butyl 5-cyano-2-(5-(2-(2,5-difluorophenyl)pyrrolidin-1-yl)pyrazolo[1,5-a]pyrimidin-3-yl)-1H-indole-1-carboxylate (102.8 mg) in DCM (2 mL) was added TFA (2 mL) at R.T for 12 h. The reaction was detected by TLC and LCMS. Then the mixture was concentrated in vacuo and the residue was adjusted pH=8, the residue was purified by combi flash (DCM:MeOH=100%:O % to 93%:7%) to yield 36.9 mg (45%, yield) of the title compound. MS(ES⁺): m/z=441.5 (M+H)⁺.

¹H NMR (500 MHz, CD₃OD) δ 8.60-8.78 (m, 2H), 7.52-7.77 (m, 3H), 7.18 (s, 1H), 7.10-6.85 (m, 3H), 6.61 and 6.07 (1H, s+s), 5.66 and 5.31 (1H, s+s), 4.22-3.66 (m, 2H), 2.53 (s, 1H), 2.29-1.93 (m, 3H).

Prepare the following examples (shown in Table 8) essentially as described for Example 133 using the corresponding starting materials. For example, prepare the following Example 183 (shown in Table 8) essentially as described for Example 133 using

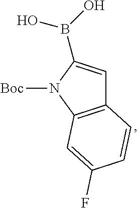 [SMILES: CC(C)(C)OC(=O)n1c(B(O)O)cc2ccc(F)cc21]

instead of

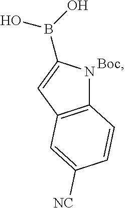 [SMILES: N#Cc1ccc2[nH]c(B(O)O)cc2c1]

and other starting materials are either commercially available or made by known procedures in the reported literature or as illustrated.

TABLE 8

| | | | Physical Data |
|---|---|---|---|
| EX No. | Chemical Name | Structure | (MS) (M + H)⁺ |
| 183 | (R)-5-(2-(2,5- difluorophenyl)pyrrolidin-1-yl)-3- (6-fluoro-1H-indol-2- yl)pyrazolo[1,5-a]pyrimidine | 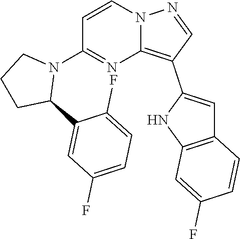 [SMILES: Fc1ccc(F)c([C@H]2CCCN2c2ccn3ncc(-c4cc5ccc(F)cc5[nH]4)c3n2)c1] | 433.5 |
| 184 | methyl (R)-2-(5-(2-(2,5- difluorophenyl)pyrrolidin-1- yl)pyrazolo[1,5-a]pyrimidin-3-yl)- 1H-indole-5-carboxylate | 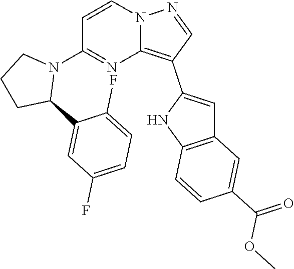 [SMILES: COC(=O)c1ccc2[nH]c(-c3cnn4ccc(N5CCC[C@@H]5c5cc(F)ccc5F)nc34)cc2c1] | 473.6 |
| 185 | (R)-2-(5-(2-(2,5- difluorophenyl)pyrrolidin-1- yl)pyrazolo[1,5-a]pyrimidin-3-yl)- 1H-indole-5-carboxylic acid | 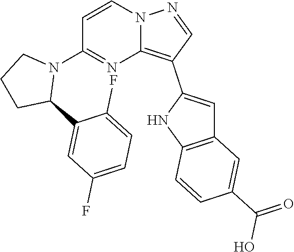 [SMILES: O=C(O)c1ccc2[nH]c(-c3cnn4ccc(N5CCC[C@@H]5c5cc(F)ccc5F)nc34)cc2c1] | 459.6 |
| 186 | (R)-2-(5-(2-(2,5- difluorophenyl)pyrrolidin-1- yl)pyrazolo[1,5-a]pyrimidin-3-yl)- 1H-indol-6-ol | 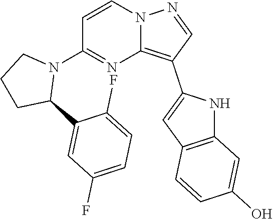 [SMILES: Oc1ccc2cc(-c3cnn4ccc(N5CCC[C@@H]5c5cc(F)ccc5F)nc34)[nH]c2c1] | 431.6 |

### Example 188 Synthesis of Compound 188

### (R)-2-(5-(2-(2,5-difluorophenyl)pyrrolidin-1-yl)pyrazolo[1,5-a]pyrimidin-3-yl)-3,4,6,7-tetrahydropyrano[3,4-d]imidazole

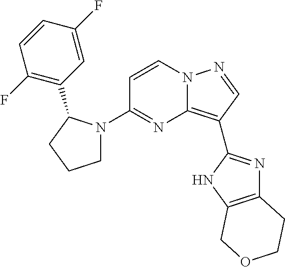 [SMILES: Fc1ccc(F)c([C@H]2CCCN2c2ccn3ncc(-c4nc5c([nH]4)COCC5)c3n2)c1]

### Step 1: Synthesis of (R)-5-(2-(2,5-difluorophenyl)pyrrolidin-1-yl)pyrazolo[1,5-a]pyrimidine-3-carbonitrile

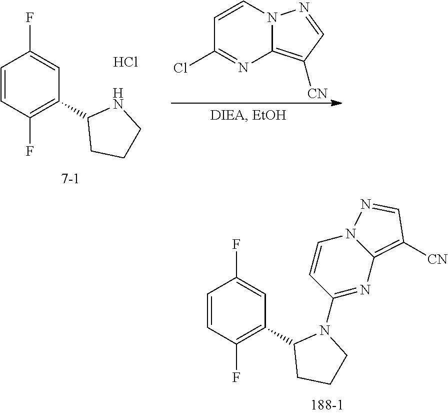

To a solution of ethyl 5-chloropyrazolo[1,5-a]pyrimidine-3-carbonitrile (17.8 g) in EtOH (400 mL) was added (R)-2-(2,5-difluorophenyl)pyrrolidine hydrochloride (26.2 g) and DIEA (25.8 g). The mixture was heated to 90° C. for 2 h.

TLC showed the reaction was completed, Concentrated under reduced pressure to remove EtOH and the residue was poured into cooled water and filtered, the solid was washed with 1N HCl and water, dried under reduce pressure at 60° C. for 10 h to afford the desired product (R)-5-(2-(2,5-difluorophenyl)pyrrolidin-1-yl)pyrazolo[1,5-a]pyrimidine-3-carbonitrile (29.9 g, 92%) as a white solid.

### Step 2: Synthesis of (R)-5-(2-(2,5-difluorophenyl)pyrrolidin-1-yl)-N-hydroxypyrazolo[1,5-a]pyrimidine-3-carboximidamide

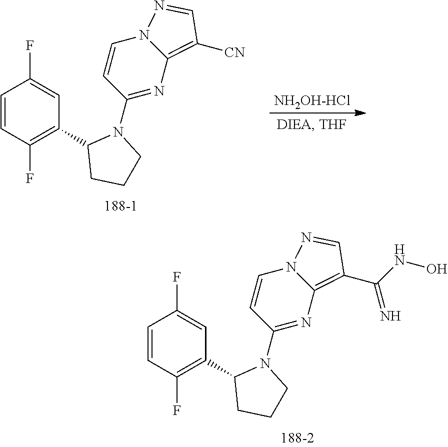

A mixture of (R)-5-(2-(2,5-difluorophenyl)pyrrolidin-1-yl)pyrazolo[1,5-a]pyrimidine-3-carbonitrile (16.2 g), NH₂OH—HCl (4.17 g), and DIEA (19.3 g) in THF (200 mL) was stirred overnight at 70° C. After cooling to room temperature, the mixture was concentrated under reduced pressure. The residue was taken up in water and the pH was adjusted to 2-3 with HCl (aqueous, 1 M). After washing the mixture with 3*40 mL EA, the pH of the aqueous layer was adjusted to 8-9 with NaOH (aqueous, 2 M) followed by extraction with 3*30 mL EA. The combined organic layers were dried over Na₂SO₄ and concentrated under reduced pressure to afford product (R)-5-(2-(2,5-difluorophenyl)pyrrolidin-1-yl)-N-hydroxypyrazolo[1,5-a]pyrimidine-3-carboximidamide (5.1 g) as a white solid.

### Step 3: Synthesis of (R)-5-(2-(2,5-difluorophenyl)pyrrolidin-1-yl)pyrazolo[1,5-a]pyrimidine-3-carboximidamide

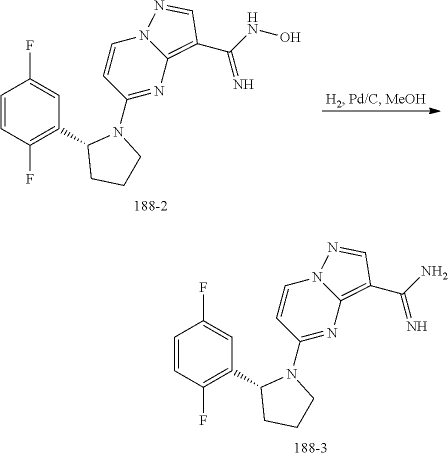 [SMILES: N=C(NO)c1cnn2ccc(N3CCC[C@@H]3c3cc(F)ccc3F)nc12 ; "H2, Pd/C, MeOH" ; N=C(N)c1cnn2ccc(N3CCC[C@@H]3c3cc(F)ccc3F)nc12]

A round-bottom flask, containing a solution of (R)-5-(2-(2,5-difluorophenyl)pyrrolidin-1-yl)-N-hydroxypyrazolo[1,5-a]pyrimidine-3-carboximidamide (5.0 g) in methanol (150 mL) was purged with nitrogen gas. To the solution was added Pd/C (1 g, 10%, 60% water) and the flask was then further purged with nitrogen gas. The atmosphere was then changed to hydrogen and the mixture was stirred overnight at 25° C. under a balloon. After purging the system with nitrogen, the solids were removed by filtration and the filtrate was concentrated under reduced pressure to afford desired product (R)-5-(2-(2,5-difluorophenyl)pyrrolidin-1-yl)pyrazolo[1,5-a]pyrimidine-3-carboximidamide (2.9 g) as a brown solid.

### Step 4: Synthesis of (R)-2-(5-(2-(2,5-difluorophenyl)pyrrolidin-1-yl)pyrazolo[1,5-a]pyrimidin-3-yl)-3,4,6,7-tetrahydropyrano[3,4-d]imidazole

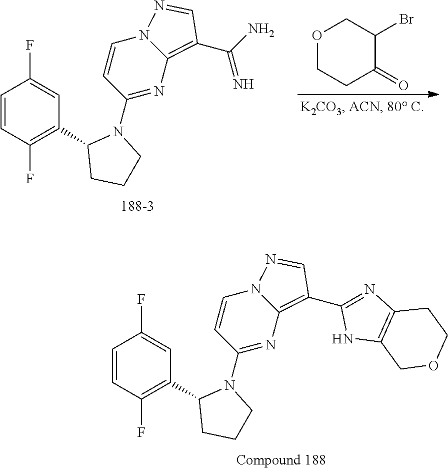 [SMILES: N=C(N)c1cnn2ccc(N3CCC[C@@H]3c3cc(F)ccc3F)nc12 ; O=C1CCOCC1Br ; Fc1ccc(F)c([C@H]2CCCN2c2ccn3ncc(-c4nc5c([nH]4)COCC5)c3n2)c1]

A mixture of (R)-5-(2-(2,5-difluorophenyl)pyrrolidin-1-yl)pyrazolo[1,5-a]pyrimidine-3-carboximidamide (685 mg),3-bromodihydro-2H-pyran-4(3H)-one (358 mg), and potassium carbonate (552 mg) in CH3CN (15 mL) was stirred overnight at 80° C. under nitrogen. After cooling to ambient temperature, the reaction mixture was concentrated under reduced pressure. The residue was dissolved in EA (50 mL) and washed with 2*10 mL of H₂O. The organic phase was dried over (Na₂SO₄) and concentrated under reduced pressure. The residue was purified using silica gel column chromatography, eluting with EA/PE (1/3) to afford desired product (R)-2-(5-(2-(2,5-difluorophenyl)pyrrolidin-1-yl)pyrazolo[1,5-a]pyrimidin-3-yl)-3,4,6,7-tetrahydropyrano[3,4-d]imidazole (295 mg) as a white solid. LC-MS: [M+H]⁺423.72.

### Example 189 Synthesis of Compound 189

### (R)-2-(5-(2-(2,5-difluorophenyl)pyrrolidin-1-yl)pyrazolo[1,5-a]pyrimidin-3-yl)-4,5,6,7-tetrahydrothiazolo[4,5-c]pyridine

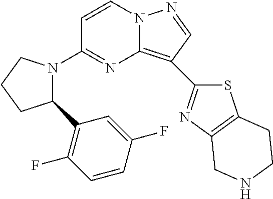 [SMILES: Fc1ccc(F)c([C@H]2CCCN2c2ccn3ncc(-c4nc5c(s4)CCNC5)c3n2)c1]

### Step 1: Synthesis of tert-butyl 3-(5-((R)-2-(2,5-difluorophenyl)pyrrolidin-1-yl)pyrazolo[1,5-a]pyrimidine-3-carboxamido)-4-hydroxypiperidine-1-carboxylate

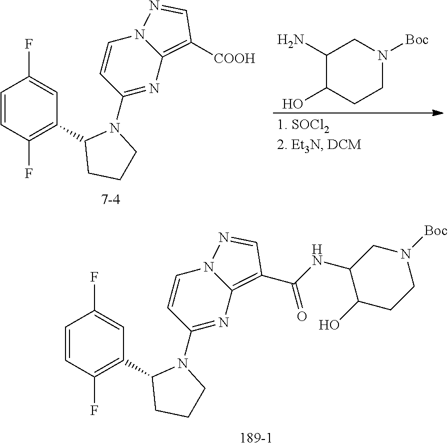

(R)-5-(2-(2,5-difluorophenyl)pyrrolidin-1-yl)pyrazolo[1,5-a]pyrimidine-3-carboxylic acid (3.44 g) was treated with SOCl₂(2.38 g) at 80° C. for 2 h, till an aliquot portion on quenching with a few drops of MeOH revealed the complete consumption of the acid and appearance of a new spot in the TLC. Excess SOCl₂ was distilled off, the residue was dissolve in DCM (30 mL) and then tert-butyl 3-amino-4-hydroxypiperidine-1-carboxylate (2.16 g), Et₃N (2.02 g) was added at 0° C., followed by stirring for 1 h.

100 mL ethylacetoacetate was added to the mixture and then washing with water. An organic layer dried over anhydrous magnesium sulfate. Filtered off and distilled off under reduced pressure, then the residue was purified by combiflash to afford desired product tert-butyl 3-(5-((R)-2-(2,5-difluorophenyl)pyrrolidin-1-yl)pyrazolo[1,5-a]pyrimidine-3-carboxamido)-4-hydroxypiperidine-1-carboxylate (2.77 g, 51%) as a yellow solid.

### Step 2: Synthesis of tert-butyl 3-(5-((R)-2-(2,5-difluorophenyl)pyrrolidin-1-yl)pyrazolo[1,5-a]pyrimidine-3-carboxamido)-4-oxopiperidine-1-carboxylate

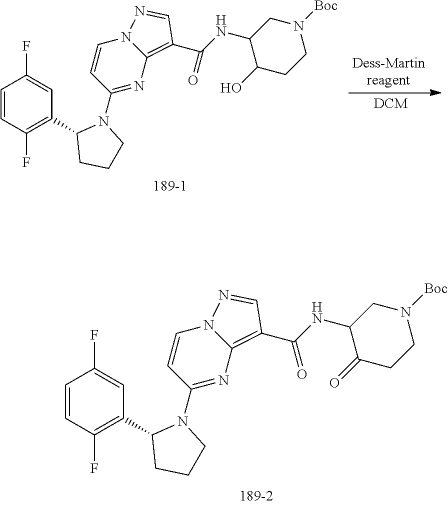 [SMILES: CC(C)(C)OC(=O)N1CCC(O)C(NC(=O)c2cnn3ccc(N4CCC[C@@H]4c4cc(F)ccc4F)nc23)C1 ; CC(C)(C)OC(=O)N1CCC(=O)C(NC(=O)c2cnn3ccc(N4CCC[C@@H]4c4cc(F)ccc4F)nc23)C1]

T-butyl 4-hydroxy-3-{[4-(trifluoromethyl)benzoyl]amino}piperidin-1-carboxylate (2.17 g) was dissolved in DCM (30 mL), and Dess-Martin periodinane (2.5 g) was dropwise added thereto. After stirring for 5 hours, ethylacetoacetate (50 mL) was dropwise added thereto, and the resulting solution was washed with water. An organic layer dried over anhydrous magnesium sulfate. The reaction solution was filtered off and distilled off under reduced pressure, then the residue was purified by combiflash (DCM:MeOH=95%:5%) to afford the desired product tert-butyl 3-(5-((R)-2-(2,5-difluorophenyl)pyrrolidin-1-yl)pyrazolo[1,5-a]pyrimidine-3-carboxamido)-4-oxopiperidine-1-carboxylate (1.6 g, 76%) as a yellow solid.

### Step 3: Synthesis of tert-butyl (R)-2-(5-(2-(2,5-difluorophenyl)pyrrolidin-1-yl)pyrazolo[1,5-a]pyrimidin-3-yl)-6,7-dihydrothiazolo[4,5-c]pyridine-5 (4H)-carboxylate

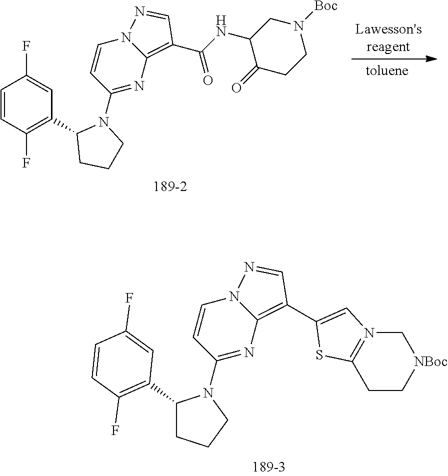

To a solution of tert-butyl 3-(5-((R)-2-(2,5-difluorophenyl)pyrrolidin-1-yl)pyrazolo[1,5-a]pyrimidine-3-carboxamido)-4-oxopiperidine-1-carboxylate (540 mg) in toluene was added Lawesson's reagent (485 mg), and the resulting solution was stirred under refluxing for 4 hours. LCMS show the reaction was completed, distilled off under reduced pressure to remove toluene. The residue was purified by combiflash (DCM:MeOH=95%:5%) to afford the product tert-butyl (R)-2-(5-(2-(2,5-difluorophenyl)pyrrolidin-1-yl)pyrazolo[1,5-a]pyrimidin-3-yl)-6,7-dihydrothiazolo[4,5-c]pyridine-5(4H)-carboxylate (242 mg) as a brown solid.

### Step 4: Synthesis of (R)-2-(5-(2-(2,5-difluorophenyl)pyrrolidin-1-yl)pyrazolo[1,5-a]pyrimidin-3-yl)-4,5,6,7-tetrahydrothiazolo[4,5-c]pyridine hydrochloride

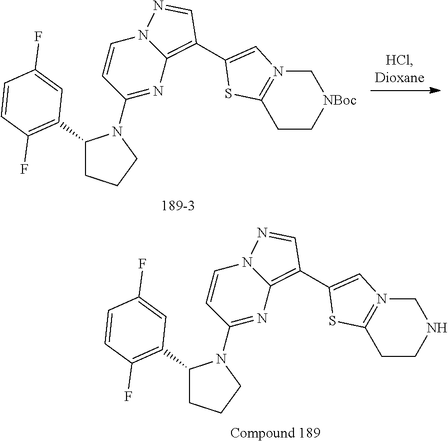

To a solution of tert-butyl (R)-2-(5-(2-(2,5-difluorophenyl)pyrrolidin-1-yl)pyrazolo[1,5-a]pyrimidin-3-yl)-6,7-dihydrothiazolo[4,5-c]pyridine-5(4H)-carboxylate (240 mg) in DCM (10 mL) was added 4N HCl/Dioxane (4 mL). The mixture was stirred for 3 h. LCMS showed the reaction was completed. Concentrated under reduced pressure to remove DCM and Dioxane to afford the product (R)-2-(5-(2-(2,5-difluorophenyl)pyrrolidin-1-yl)pyrazolo[1,5-a]pyrimidin-3-yl)-4,5,6,7-tetrahydrothiazolo[4,5-c]pyridine hydrochloride (148 mg) as a brown solid. MS: [M+H]⁺ 439.78.

Prepare the following examples (shown in Table 9) essentially as described for Example 189 using the corresponding starting materials.

TABLE 9

| | | | Physical Data |
|---|---|---|---|
| EX No. | Chemical Name | Structure | (MS) (M + H)⁺ |
| 190 | (R)-2-(5-(2-(2,5- difluorophenyl)pyrrolidin-1- yl)pyrazolo[1,5-a]pyrimidin-3- yl)-4,5,6,7- tetrahydrothiazolo[5,4-c]pyridine | 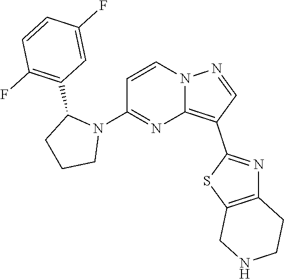 [SMILES: Fc1ccc(F)c([C@H]2CCCN2c2ccn3ncc(-c4nc5c(s4)CNCC5)c3n2)c1] | 438.6 |
| 191 | (R)-2-(5-(2-(2,5- difluorophenyl)pyrrolidin-1- yl)pyrazolo[1,5-a]pyrimidin-3- yl)-6,7-dihydrothiazolo[5,4-c] pyridine-5(4H)-carboxamide | 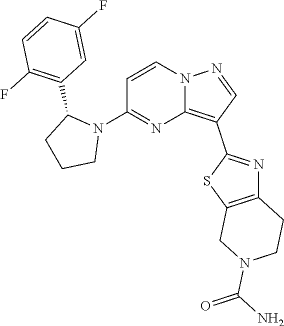 [SMILES: NC(=O)N1CCc2nc(-c3cnn4ccc(N5CCC[C@@H]5c5cc(F)ccc5F)nc34)sc2C1] | 481.6 |
| 192 | (R)-2-(5-(2-(2,5- difluorophenyl)pyrrolidin-1- yl)pyrazolo[1,5-a]pyrimidin-3- yl)-6,7-dihydro-4H-pyrano[4,3- d]thiazole | 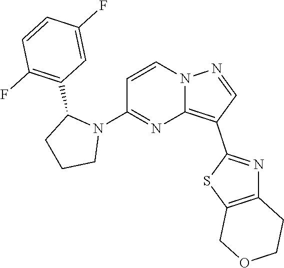 [SMILES: Fc1ccc(F)c([C@H]2CCCN2c2ccn3ncc(-c4nc5c(s4)COCC5)c3n2)c1] | 439.6 |

### Example 193 Synthesis of Compound 193

### (R)-5-(2-(2,5-difluorophenyl)pyrrolidin-1-yl)-3-(5,6-dimethoxy-1H-benzo[d]imidazol-2-yl)pyrazolo[1,5-a]pyrimidin-2-amine

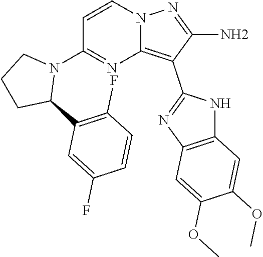 [SMILES: COc1cc2nc(-c3c(N)nn4ccc(N5CCC[C@@H]5c5cc(F)ccc5F)nc34)[nH]c2cc1OC]

### Step 1: Synthesis of ethyl (R)-2-amino-5-(2-(2,5-difluorophenyl)pyrrolidin-1-yl)pyrazolo[1,5-a]pyrimidine-3-carboxylate

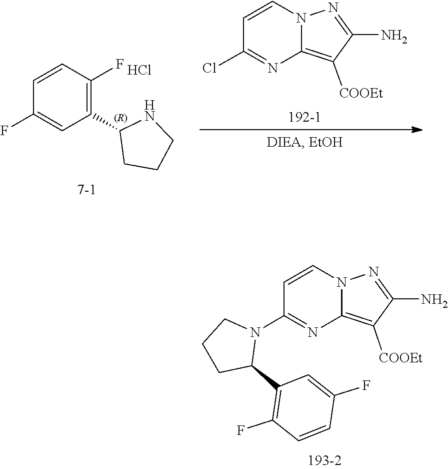

To a solution of (R)-2-(2,5-difluorophenyl)pyrrolidine hydrochloride (1.00 g) in EtOH (150 mL) was added DIEA (1.93 g) and ethyl 2-amino-5-chloropyrazolo[1,5-a]pyrimidine-3-carboxylate (1.13 g) at R.T and then heated to 80° C. for 3 h. The reaction was detected by TLC and LCMS. The reaction was cooled to room temperature, then the mixture was concentrated in vacuo. The reaction mixture was added into cooled water and stirring. The mixture was filtrated and the residue solid was stirring in 1N HCl solution, then the mixture was filtrated to afford crude product and drying at 50° C. for 16 h to afford ethyl (R)-2-amino-5-(2-(2,5-difluorophenyl)pyrrolidin-1-yl)pyrazolo[1,5-a]pyrimidine-3-carboxylate (1.51 g) as a light yellow solid.

### Step 2: Synthesis of (R)-2-amino-5-(2-(2,5-difluorophenyl)pyrrolidin-1-yl)pyrazolo[1,5-a]pyrimidine-3-carboxylic acid

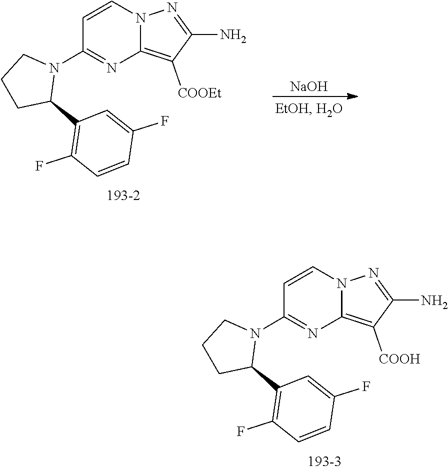 [SMILES: CCOC(=O)c1c(N)nn2ccc(N3CCC[C@@H]3c3cc(F)ccc3F)nc12 ; "NaOH" ; Nc1nn2ccc(N3CCC[C@@H]3c3cc(F)ccc3F)nc2c1C(=O)O]

To a solution of ethyl (R)-2-amino-5-(2-(2,5-difluorophenyl)pyrrolidin-1-yl)pyrazolo[1,5-a]pyrimidine-3-carboxylate (1.50 g) in EtOH (150 mL) and H₂O (150 mL) was added NaOH (467.9 mg) at 80° C. for 6 h. The reaction was detected by TLC and LCMS. The mixture was concentrated in vacuo and the residue was pour into 1N HCl solution. The mixture was filtrated and dried at 50° C. for 16 h to afford crude product (R)-2-amino-5-(2-(2,5-difluorophenyl)pyrrolidin-1-yl)pyrazolo[1,5-a]pyrimidine-3-carboxylic acid (1.30 g, 93%) as off-white solid.

### Step 3: Synthesis of (R)-5-(2-(2,5-difluorophenyl)pyrrolidin-1-yl)-3-(5,6-dimethoxy-1H-benzo[d]imidazol-2-yl)pyrazolo[1,5-a]pyrimidin-2-amine

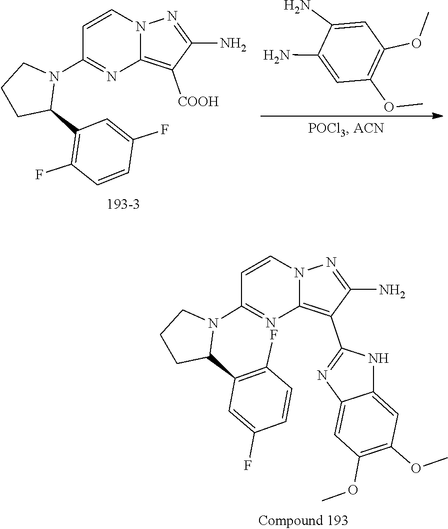

To a solution of (R)-2-amino-5-(2-(2,5-difluorophenyl)pyrrolidin-1-yl)pyrazolo[1,5-a]pyrimidine-3-carboxylic acid (1.30 g) in MeCN (150 mL) was added 4,5-dimethoxybenzene-1,2-diamine (669 mg) and POCl₃ (1.66 g) at 100° C. for 16 h. The reaction was detected by TLC and LCMS. The mixture was added MeCN (150 mL) and then filtered, the filter cake was adjusted PH=8 by 0.5N NaOH solution, then the mixture was filtrated to afford crude product, the filter cake was dried at 50° C. for 16 h to afford product (R)-5-(2-(2,5-difluorophenyl)pyrrolidin-1-yl)-3-(5,6-dimethoxy-1H-benzo[d]imidazol-2-yl)pyrazolo[1,5-a]pyrimidin-2-amine (1.2 g) as off-white solid. MS: [M+H]⁺ 492.81.

Prepare the following Example 194 (shown in Table 10) essentially as described for Example 193 using

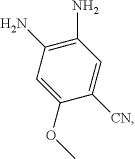

instead of

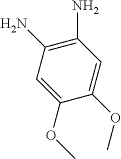

and other starting materials are either commercially available or made by known procedures in the reported literature or as illustrated.

TABLE 10

| EX | | | Physical Data |
|---|---|---|---|
| No. | Chemical Name | Structure | (MS) (M + H)⁺ |
| 194 | (R)-2-(2-amino-5-(2-(2,5- difluorophenyl)pyrrolidin-1- yl)pyrazolo[1,5-a]pyrimidin-3- yl)-5-methoxy-IH- benzo[d]imidazole-6- carbonitrile | 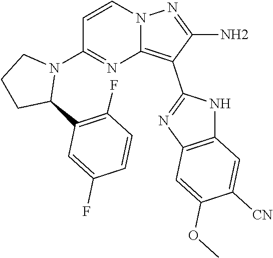 [SMILES: COc1cc2nc(-c3c(N)nn4ccc(N5CCC[C@@H]5c5cc(F)ccc5F)nc34)[nH]c2cc1C#N] | 486.6 |

*Remark: If there is an isomeride in the above compounds, the present invention also includes its isomeride and also includes their mixture.

### Example 195 Synthesis of Compound 195 and/or its Isomeride

### (R)-2-(5-(2-(2-fluorophenyl)pyrrolidin-1-yl)pyrazolo[1,5-a]pyrimidin-3-yl)-6-methoxy-1H-benzo[d]imidazole-5-carbonitrile

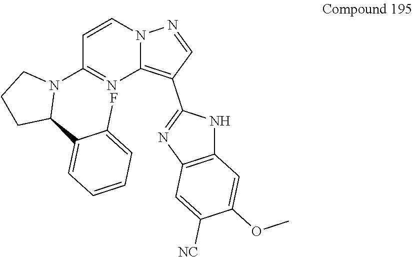 [SMILES: COc1cc2[nH]c(-c3cnn4ccc(N5CCC[C@@H]5c5ccccc5F)nc34)nc2cc1C#N]

### (R)-2-(5-(2-(2-fluorophenyl)pyrrolidin-1-yl)pyrazolo[1,5-a]pyrimidin-3-yl)-5-methoxy-1H-benzo[d]imidazole-6-carbonitrile

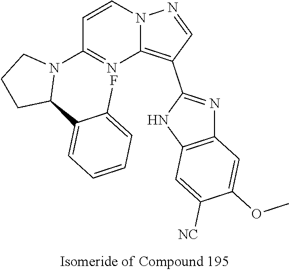 [SMILES: COc1cc2nc(-c3cnn4ccc(N5CCC[C@@H]5c5ccccc5F)nc34)[nH]c2cc1C#N]

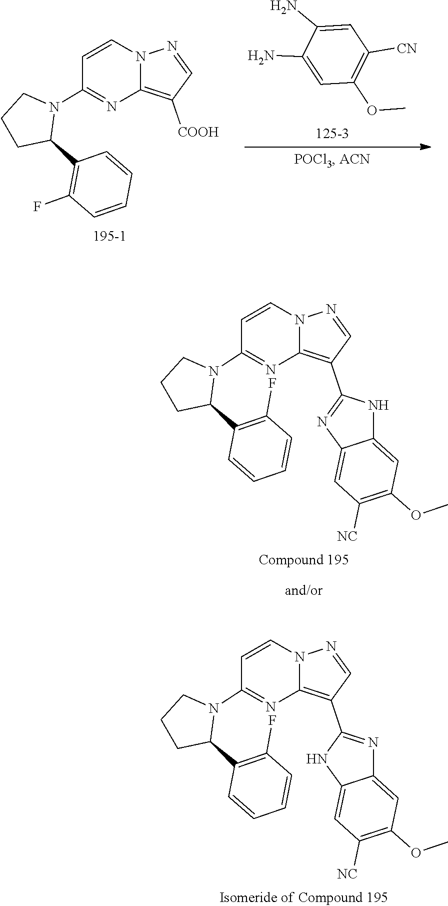 [SMILES: O=C(O)c1cnn2ccc(N3CCC[C@@H]3c3ccccc3F)nc12 ; COc1cc(N)c(N)cc1C#N ; COc1cc2[nH]c(-c3cnn4ccc(N5CCC[C@@H]5c5ccccc5F)nc34)nc2cc1C#N ; COc1cc2nc(-c3cnn4ccc(N5CCC[C@@H]5c5ccccc5F)nc34)[nH]c2cc1C#N]

To a solution of (R)-5-(2-(2-fluorophenyl)pyrrolidin-1-yl)pyrazolo[1,5-a]pyrimidine-3-carboxylic acid (13.0 g) in MeCN (150 mL) was added 4,5-diamino-2-methoxybenzonitrile (7.15 g) and POCl₃ (18.34 g) at 100° C. for 16 h. The reaction was detected by TLC and LCMS. The mixture was added MeCN (150 mL) and then filtrated, the filter cake was adjusted PH=8 by 0.5N NaOH solution, then the mixture was filtrated to afford crude product, the filter cake was dried to afford product (Compound 195 and/or the isomeride of Compound 195) as off-white solid (13.9 g). LC-MS: [M+H]⁺ 454.78.

### Example 196 Synthesis of Compound 196 and/or its Isomeride

### (R)-2-(5-(2-(3-fluorophenyl)pyrrolidin-1-yl)pyrazolo[1,5-a]pyrimidin-3-yl)-6-methoxy-1H-benzo[d]imidazole-5-carbonitrile

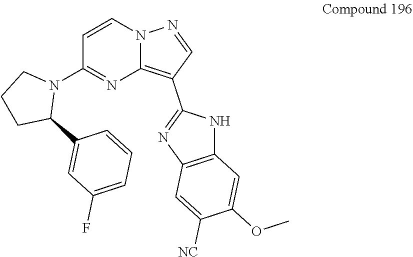 [SMILES: COc1cc2[nH]c(-c3cnn4ccc(N5CCC[C@@H]5c5cccc(F)c5)nc34)nc2cc1C#N]

### (R)-2-(5-(2-(3-fluorophenyl)pyrrolidin-1-yl)pyrazolo[1,5-a]pyrimidin-3-yl)-5-methoxy-1H-benzo[d]imidazole-6-carbonitrile

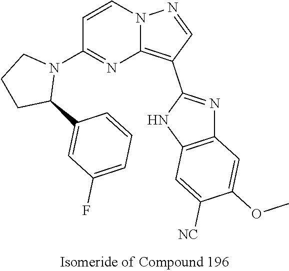 [SMILES: COc1cc2nc(-c3cnn4ccc(N5CCC[C@@H]5c5cccc(F)c5)nc34)[nH]c2cc1C#N]

### Step 1: Synthesis of (R)-2-(5-(2-(3-fluorophenyl)pyrrolidin-1-yl)pyrazolo[1,5-a]pyrimidin-3-yl)-6-methoxy-JH-benzo[d]imidazole-5-carbonitrile and/or Isomeride Thereof

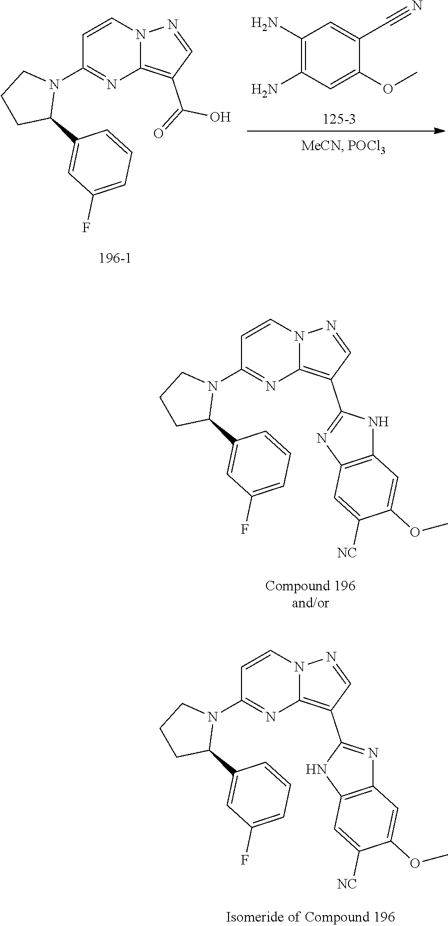 [SMILES: O=C(O)c1cnn2ccc(N3CCC[C@@H]3c3cccc(F)c3)nc12 ; COc1cc(N)c(N)cc1C#N ; COc1cc2[nH]c(-c3cnn4ccc(N5CCC[C@@H]5c5cccc(F)c5)nc34)nc2cc1C#N ; COc1cc2nc(-c3cnn4ccc(N5CCC[C@@H]5c5cccc(F)c5)nc34)[nH]c2cc1C#N]

To a solution of (R)-5-(2-(3-fluorophenyl)pyrrolidin-1-yl)pyrazolo[1,5-a]pyrimidine-3-carboxylic acid (13.0 g) in MeCN (150 mL) was added 4,5-diamino-2-methoxybenzonitrile (7.15 g) and POCl₃ (18.34 g) at 100° C. for 16 h. The reaction was detected by TLC and LCMS. The mixture was added MeCN (150 mL) and then filtrated, the filter cake was dried to afford the desired product (Compound 196 and/or the isomeride of Compound 196) as off-white solid (13.6 g). MS: [M+H]⁺ 454.78.

### Example 197 Synthesis of Compound 197

### (S)-2-(5-(2-(2,5-difluorophenyl)pyrrolidin-1-yl)pyrazolo[1,5-a]pyrimidin-3-yl)-6-methoxy-1H-benzo[d]imidazole-5-carbonitrile

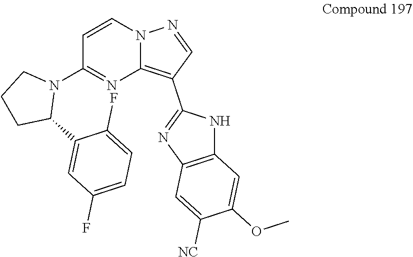 [SMILES: COc1cc2[nH]c(-c3cnn4ccc(N5CCC[C@H]5c5cc(F)ccc5F)nc34)nc2cc1C#N]

### (S)-2-(5-(2-(2,5-difluorophenyl)pyrrolidin-1-yl)pyrazolo[1,5-a]pyrimidin-3-yl)-5-methoxy-1H-benzo[d]imidazole-6-carbonitrile

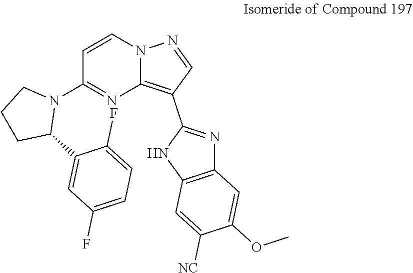 [SMILES: COc1cc2nc(-c3cnn4ccc(N5CCC[C@H]5c5cc(F)ccc5F)nc34)[nH]c2cc1C#N]

### Step 1: Synthesis of ethyl (S)-5-(2-(2,5-difluorophenyl)pyrrolidin-1-yl)pyrazolo[1,5-a]pyrimidine-3-carboxylate

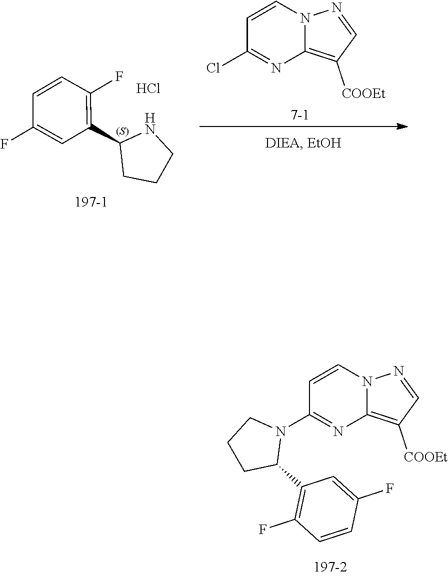

To a solution of (S)-2-(2,4-difluorophenyl)pyrrolidine hydrochloride (10.00 g) in EtOH (150 mL) was added DIEA (17.62 g) and ethyl 5-chloropyrazolo[1,5-a]pyrimidine-3-carboxylate (9.76 g) at R.T and then heated to 80° C. for 3 h. The reaction was detected by LCMS. The reaction was cooled to room temperature, then the mixture was concentrated in vacuo. The reaction mixture was added into cooled water and stirring. The mixture was filtrated and the residue solid was stirring in 1N HCl solution, then the mixture was filtrated to afford crude product and drying at 50° C. for 16 h to afford ethyl (S)-5-(2-(2,5-difluorophenyl)pyrrolidin-1-yl)pyrazolo[1,5-a]pyrimidine-3-carboxylate (15.20 g) as a light yellow solid.

### Step 2: Synthesis of (S)-5-(2-(2,5-difluorophenyl)pyrrolidin-1-yl)pyrazolo[1,5-a]pyrimidine-3-carboxylic Acid

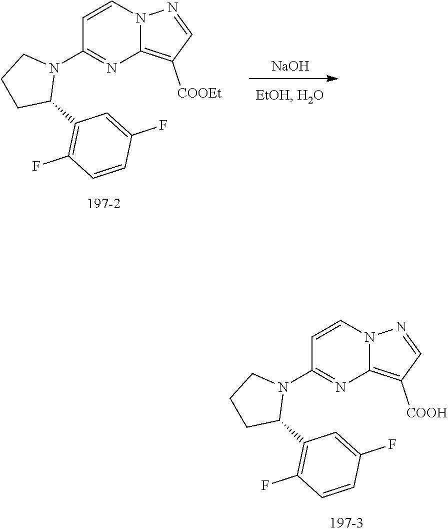 [SMILES: CCOC(=O)c1cnn2ccc(N3CCC[C@H]3c3cc(F)ccc3F)nc12 ; "NaOH" ; O=C(O)c1cnn2ccc(N3CCC[C@H]3c3cc(F)ccc3F)nc12]

To a solution of ethyl (S)-5-(2-(2,5-difluorophenyl)pyrrolidin-1-yl)pyrazolo[1,5-a]pyrimidine-3-carboxylate (15.2 g) in EtOH (150 mL) and H₂O (150 mL) was added NaOH (4.90 g) at 80° C. for 6 h. The reaction was detected by TLC and LCMS. The mixture was concentrated in vacuo and the residue was pour into 1N HCl solution. The mixture was filtrated and dried to afford product (S)-5-(2-(2,5-difluorophenyl)pyrrolidin-1-yl)pyrazolo[1,5-a]pyrimidine-3-carboxylic acid (13.0 g) as off-white solid.

### Step 3: Synthesis of (S)-2-(5-(2-(2,5-difluorophenyl)pyrrolidin-1-yl)pyrazolo[1,5-a]pyrimidin-3-yl)-6-methoxy-1H-benzo[d]imidazole-5-carbonitrile and/or isomeride thereof

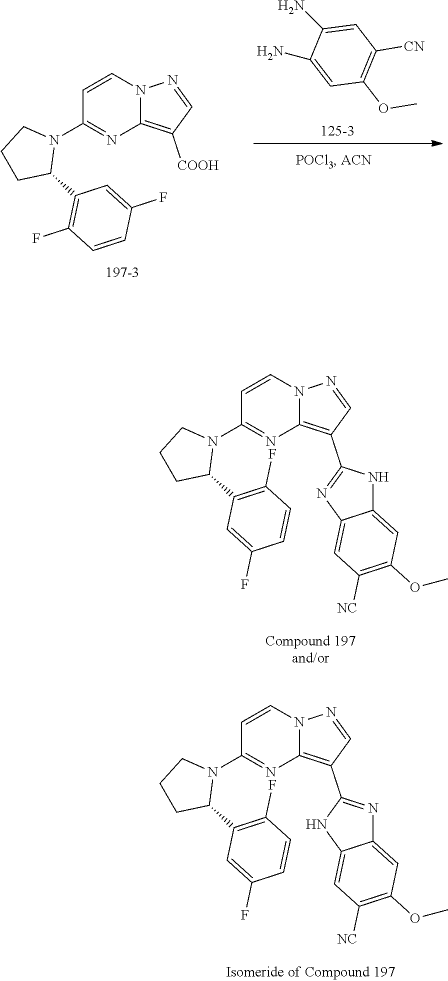 [SMILES: O=C(O)c1cnn2ccc(N3CCC[C@H]3c3cc(F)ccc3F)nc12 ; COc1cc(N)c(N)cc1C#N ; COc1cc2[nH]c(-c3cnn4ccc(N5CCC[C@H]5c5cc(F)ccc5F)nc34)nc2cc1C#N ; COc1cc2nc(-c3cnn4ccc(N5CCC[C@H]5c5cc(F)ccc5F)nc34)[nH]c2cc1C#N]

To a solution of (S)-5-(2-(2,5-difluorophenyl)pyrrolidin-1-yl)pyrazolo[1,5-a]pyrimidine-3-carboxylic acid (13.0 g) in MeCN (150 mL) was added 4,5-diamino-2-methoxybenzonitrile (6.77 g) and POCl₃(17.37 g) at 100° C. for 16 h. The reaction was detected by TLC and LCMS. The mixture was added MeCN (150 mL) and then filtrated, the filter cake was adjusted PH=8 by 0.5N NaOH solution, then the mixture was filtrated to afford crude product, the filter cake was dried to afford the desired product (Compound 197 and/or the isomeride of Compound 197) as off-white solid (13.5 g). MS: [M+H]⁺ 472.81.

### Example 198 Synthesis of Compound 198 and/or its Isomeride

### (R)-2-(5-(2-(4-fluorophenyl)pyrrolidin-1-yl)pyrazolo[1,5-a]pyrimidin-3-yl)-6-methoxy-1H-benzo[d]imidazole-5-carbonitrile

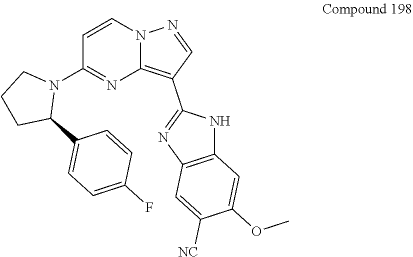 [SMILES: COc1cc2[nH]c(-c3cnn4ccc(N5CCC[C@@H]5c5ccc(F)cc5)nc34)nc2cc1C#N]

(R)-2-(5-(2-(4-fluorophenyl)pyrrolidin-1-yl)pyrazolo[1,5-a]pyrimidin-3-yl)-5-methoxy-1H-benzo[d]imidazole-6-carbonitrile

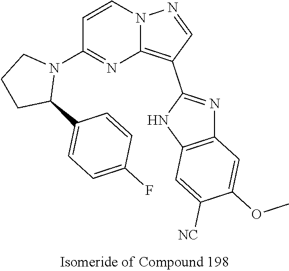 [SMILES: COc1cc2nc(-c3cnn4ccc(N5CCC[C@@H]5c5ccc(F)cc5)nc34)[nH]c2cc1C#N]

### Step 1: Synthesis of (R)-ethyl 5-(2-(4-fluorophenyl)pyrrolidin-1-yl)pyrazolo[1,5-a]pyrimidine-3-carboxylate

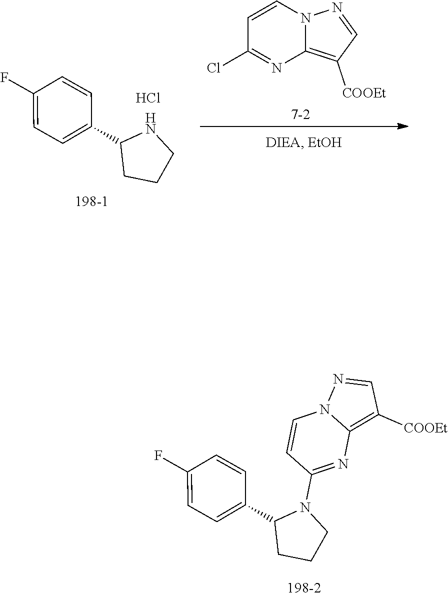

To a solution of (R)-2-(4-fluorophenyl)pyrrolidine hydrochloride (10.00 g) in EtOH (150 mL) was added DIEA (19.25 g) and ethyl 5-chloropyrazolo[1,5-a]pyrimidine-3-carboxylate (10.62 g) at R.T and then heated to 80° C. for 3 h. The reaction was detected by TLC and LCMS. The reaction was cooled to room temperature, then the mixture was concentrated in vacuo. The reaction mixture was added into cooled water and stirring. The mixture was filtrated and the residue solid was stirring in 1N HCl solution, then the mixture was filtrated to afford crude product and drying at 50° C. for 16 h to afford (R)-ethyl 5-(2-(4-fluorophenyl)pyrrolidin-1-yl)pyrazolo[1,5-a]Pyrimidine-3-carboxylate (15.20 g) as a light yellow solid.

### Step 2: Synthesis of (R)-5-(2-(4-fluorophenyl)pyrrolidin-1-yl)pyrazolo[1,5-a]pyrimidine-3-carboxylic Acid

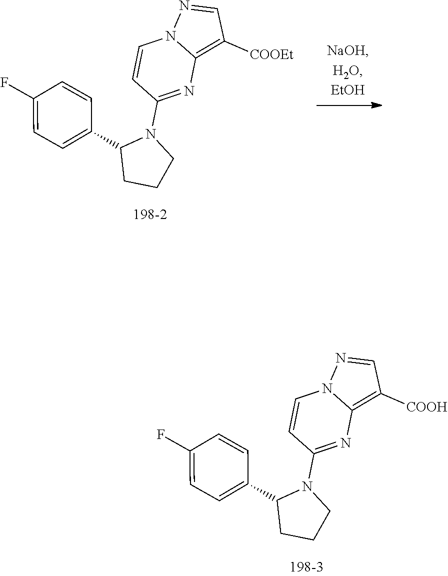

To a solution of (R)-ethyl 5-(2-(4-fluorophenyl)pyrrolidin-1-yl)pyrazolo[1,5-a]pyrimidine-3-carboxylate (15.2 g) in EtOH (150 mL) and H₂O (150 mL) was added NaOH (5.15 g) at 80° C. for 6 h. The reaction was detected by TLC and LCMS. The mixture was concentrated in vacuo and the residue was pour into 1N HCl solution. The mixture was filtrated and heated at 50° C. for 16 h to afford crude product (R)-5-(2-(4-fluorophenyl)pyrrolidin-1-yl)pyrazolo[1,5-a]pyrimidine-3-carboxylic acid (13.0 g) as off-white solid.

### Step 3: Synthesis of (R)-2-(5-(2-(4-fluorophenyl)pyrrolidin-1-yl)pyrazolo[1,5-a]pyrimidin-3-yl)-6-methoxy-1H-benzo[d]imidazole-5-carbonitrile and/or Isomeride Thereof

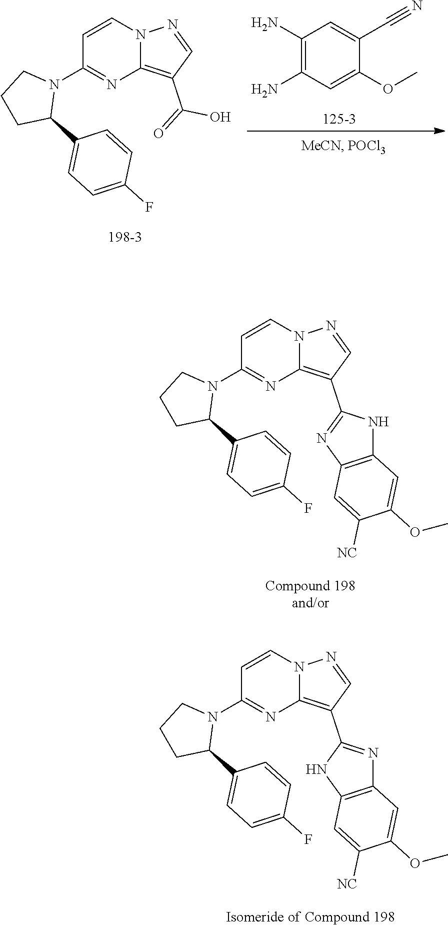 [SMILES: O=C(O)c1cnn2ccc(N3CCC[C@@H]3c3ccc(F)cc3)nc12 ; COc1cc(N)c(N)cc1C#N ; COc1cc2[nH]c(-c3cnn4ccc(N5CCC[C@@H]5c5ccc(F)cc5)nc34)nc2cc1C#N ; COc1cc2nc(-c3cnn4ccc(N5CCC[C@@H]5c5ccc(F)cc5)nc34)[nH]c2cc1C#N]

To a solution of (R)-5-(2-(4-fluorophenyl)pyrrolidin-1-yl)pyrazolo[1,5-a]pyrimidine-3-carboxylic acid (13.0 g) in MeCN (150 mL) was added 4,5-diamino-2-methoxybenzonitrile (7.15 g) and POCl₃ (18.34 g) at 100° C. for 16 h. The reaction was detected by TLC and LCMS. The mixture was added MeCN (150 mL) and then filtrated, the filter cake was adjusted PH=8 by 0.5N NaOH solution, then the mixture was filtrated to afford crude product, the filter cake was heated at 50° C. for 16 h to afford the desired product (Compound 198 and/or the isomeride of Compound 198) as off-white solid (13 g). MS: [M+H]⁺ 454.81.

### Example 199 Synthesis of Compound 199

### (R)-5-(2-(2,5-difluorophenyl)pyrrolidin-1-yl)-3-(6,7-dimethoxyimidazo[1,2-a]pyridin-2-yl)pyrazolo[1,5-a]pyrimidine

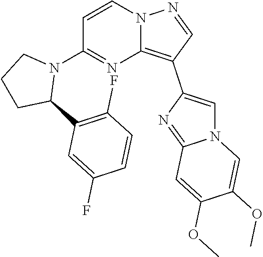 [SMILES: COc1cc2nc(-c3cnn4ccc(N5CCC[C@@H]5c5cc(F)ccc5F)nc34)cn2cc1OC]

### Step 1: Synthesis of (R)-5-(2-(2,5-difluorophenyl)pyrrolidin-1-yl)pyrazolo[1,5-a]pyrimidine-3-carbohydrazide

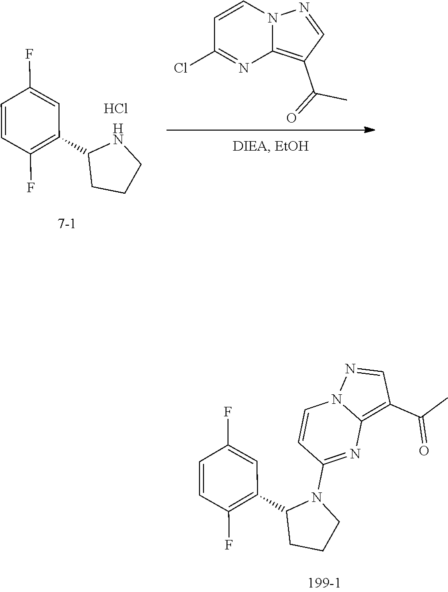

To a solution of (R)-2-(4-fluorophenyl)pyrrolidine hydrochloride (1.00 g) in EtOH (15 mL) was added DIEA (1.93 g) and 1-(5-chloropyrazolo[1,5-a]pyrimidin-3-yl)ethan-1-one (923.5 mg) at R.T and then heated to 80° C. for 3 h. The reaction was detected by TLC and LCMS. The reaction was cooled to room temperature, then the mixture was concentrated in vacuo. The reaction mixture was added into cooled water and stirring. The mixture was filtrated and the residue solid was stirring in 1N HCl solution, then the mixture was filtrated to afford crude product and drying at 50° C. for 16 h to afford (R)-1-(5-(2-(2,5-difluorophenyl)pyrrolidin-1-yl)pyrazolo[1,5-a]pyrimidin-3-yl)ethanone (1.56 g) as a light yellow solid.

### Step 2: Synthesis of (R)-2-chloro-1-(5-(2-(2,5-difluorophenyl)pyrrolidin-1-yl)pyrazolo[1,5-a]pyrimidin-3-yl)ethanone

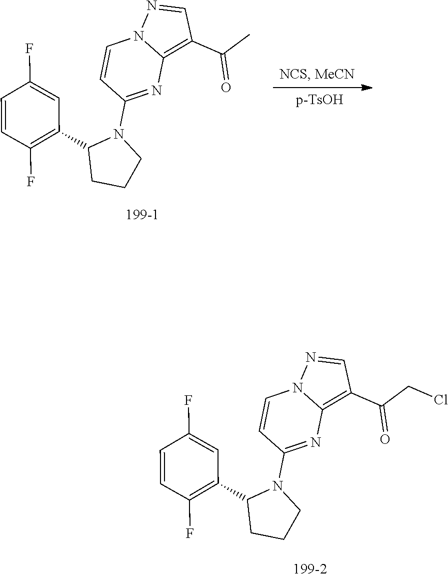

To a solution of (R)-1-(5-(2-(2,5-difluorophenyl)pyrrolidin-1-yl)pyrazolo[1,5-a]pyrimidin-3-yl)ethanone (1.50 g) in MeCN (15 mL) was added NCS (1.17 g) and p-TsOH (151.5 mg) at 90° C. for 6 h. The reaction was detected by TLC and LCMS. The mixture was concentrated in vacuo the residue was purified by combiflash (PE:EA=50%:50%) to afford (R)-2-chloro-1-(5-(2-(2,5-difluorophenyl)pyrrolidin-1-yl)pyrazolo[1,5-a]pyrimidin-3-yl)ethanone (906 mg) as a light yellow solid.

### Step 3: Synthesis of (R)-5-(2-(2,5-difluorophenyl)pyrrolidin-1-yl)-3-(6,7-dimethoxyimidazo[1,2-a]pyridin-2-yl)pyrazolo[1,5-a]pyrimidine

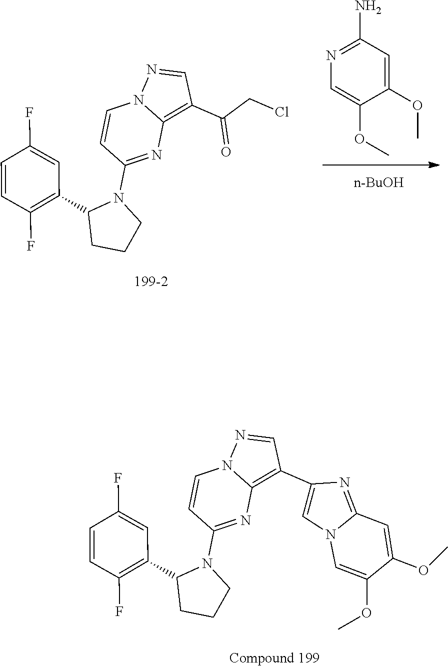

To a solution of (R)-2-chloro-1-(5-(2-(2,5-difluorophenyl)pyrrolidin-1-yl)pyrazolo[1,5-a]pyrimidin-3-yl)ethanone (900.00 mg) in n-BuOH (10 mL) was added 4,5-dimethoxypyridin-2-amine (1.11 g) at 130° C. for 6 h. The reaction was detected by TLC and LCMS. The mixture was concentrated in vacuo and the residue was purified by combiflash (DCM:MeOH=95%:5%) to afford (R)-5-(2-(2,5-difluorophenyl)pyrrolidin-1-yl)-3-(6,7-dimethoxyimidazo[1,2-a]pyridin-2-yl)pyrazolo[1,5-a]pyrimidine (906 mg) as a light yellow solid. MS: [M+H]⁺ 477.53.

### Example 200 Synthesis of Compound 200

### (R)-3-(5-(2-(2,5-difluorophenyl)pyrrolidin-1-yl)pyrazolo[1,5-a]pyrimidin-3-yl)-5,6-dihydro-8H-[1,2,4]triazolo[3,4-c][1,4]oxazine

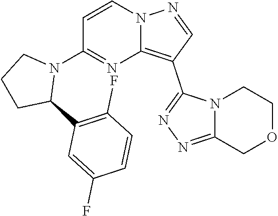 [SMILES: Fc1ccc(F)c([C@H]2CCCN2c2ccn3ncc(-c4nnc5n4CCOC5)c3n2)c1]

### Step 1: Synthesis of (R)-5-(2-(2,5-difluorophenyl)pyrrolidin-1-yl)pyrazolo[1,5-a]pyrimidine-3-carbohydrazide

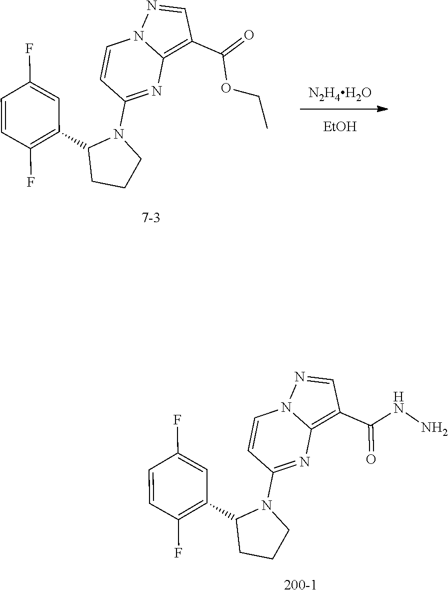

To a solution of ethyl (R)-5-(2-(2,5-difluorophenyl)pyrrolidin-1-yl)pyrazolo[1,5-a]pyrimidine-3-carboxylate (1.00 g) in EtOH (10 mL) was added N₂H₄.H₂O (437 mg) at R.T and then heated to 80° C. for 3 h. The reaction was detected by LCMS. The reaction was cooled to room temperature, then the mixture was concentrated in vacuo, the residue was purified by combiflash (PE:EA=50%:50%) to afford (R)-5-(2-(2,5-difluorophenyl)pyrrolidin-1-yl)pyrazolo[1,5-a]pyrimidine-3-carbohydrazide (1.01 g) as a light yellow solid.

### Step 2: Synthesis of (R)-3-(5-(2-(2,5-difluorophenyl)pyrrolidin-1-yl)pyrazolo[1,5-a]pyrimidin-3-yl)-5,6-dihydro-8H-[1,2,4]triazolo[3,4-c][1,4]oxazine

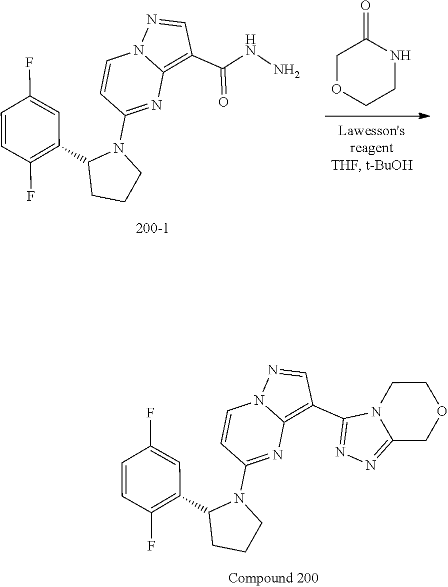

To a solution of (R)-5-(2-(2,5-difluorophenyl)pyrrolidin-1-yl)pyrazolo[1,5-a]pyrimidine-3-carbohydrazide (1.00 g) in THF (10 mL) was added Lawesson's reagent (3.39 g) and morpholin-3-one (459.8 mg) at 70° C. for 6 h. The reaction was detected by TLC and LCMS. The mixture was concentrated in vacuo and the residue was pour into 1N HCl solution. The mixture was added t-BuOH (10 mL) at 130° C. for 6 h. The reaction was cooled to room temperature, then the mixture was concentrated in vacuo the residue was purified by combiflash (DCM:MeOH=5%:95%) to afford (R)-3-(5-(2-(2,5-difluorophenyl)pyrrolidin-1-yl)pyrazolo[1,5-a]pyrimidin-3-yl)-5,6-dihydro-8H-[1,2,4]triazolo[3,4-c][1,4]oxazine (906 mg) as a light yellow solid. MS: [M+H]⁺ 424.48.

### Comparative compound A 5-[2-(2,5-Difluorophenyl)pyrrolidin-1-yl]-3-(5-methyl-1H-1,2,4-triazol-3-yl)pyrazolo[1,5-a]pyrimidine

Prepare the following Comparative compound A as described for Example 43 in WO2016097869.

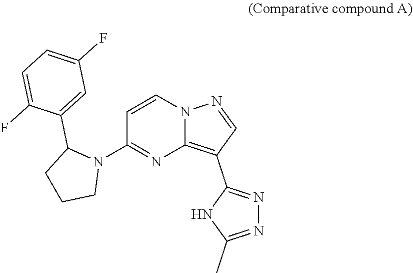 [SMILES: Cc1nnc(-c2cnn3ccc(N4CCCC4c4cc(F)ccc4F)nc23)[nH]1]

### Example 201 TrkA Kinase Assay

Mobility shift assay was used to determine the inhibitory activity of compounds against TrkA kinase. Assay procedures are as follows:

1. Reaction buffer:

1× Kinase base buffer (50 mM HEPES, pH 7.5; 0.0015% Brij-35)

Stop buffer (100 mM HEPES, pH 7.5; 0.015% Brij-35; 0.2% Coating Reagent #3; 50 mM EDTA)

2. Prepare compounds:

1) Dilute the compound to 50× of the final desired highest inhibitor concentration in reaction by 100% DMSO. Transfer 100 μl of this compound dilution to a well in a 96-well plate.

2) For all compounds, transfer the compound in tubes to one well on 96-well storage plate and serially dilute the compound by transferring 30 μl to 60 μl of 100% DMSO in the next well and so forth for a total of 10 concentrations.

3) Add 100 μl of 100% DMSO to two empty wells for no compound control and no enzyme control in the same 96-well plate. Mark the plate as source plate.

4) Prepare intermediate plate

Transfer 10 μl of compound from source plate to a new 96-well plate as the intermediate plate

Add 90 l of 1× kinase buffer to each well of the intermediate plate.

Mix the compounds in intermediate plate for 10 min on shaker.

3. Prepare assay plate

1) Transfer 5 μl of each well from the 96-well intermediate plate to a 384-well plate in duplicates. For example, A1 of the 96-well plate is transferred to A1 and A2 of the 384-well plate. A2 of the 96-well plate is transferred to A3 and A4 of the 384-well plate, and so on.

4. Kinase reaction

1) Prepare 2.5× enzyme solution

Add kinase in 1× kinase base buffer.

2) Prepare 2.5× peptide solution Add FAM-labeled peptide and ATP in the 1× kinase base buffer.

3) Assay plate already contains 5 μl of compound in 10% DMSO.

4) Transfer 2.5× enzyme solution to the assay plate

Add 10 μl of 2.5× enzyme solution to each well of the 384-well assay plate.

5) Incubate at room temperature for 10 min.

6) Transfer 2.5× peptide solution to the assay plate

Add 10 μl of 2.5× peptide solution to each well of the 384-well assay plate.

Kinase reaction conditions are shown as Table 11:

TABLE 11

| | Enzyme | ATP | | Peptide |
|---|---|---|---|---|
| Name | (nM) | (μM) | Peptide | Concentration(uM) |
| TRKA | 5 | 415 | P22 | 3 |

7) Kinase reaction and stop

Incubate at 28° C. for specified period of time.

Add 25 μl of stop buffer to stop reaction.

5. Caliper reading

Collect data on Caliper.

6. Curve fitting

1) Copy conversion data from Caliper program.

2) Convert conversion values to inhibition values.

Percent inhibition=(max-conversion)/(max-min)*100.

“max” stands for DMSO control; “min” stands for low control.

3) Fit the data in XLFit excel add-in version 5.4.0.8 to obtain IC₅₀ values.

Equation used is: Y=Bottom+(Top-Bottom)/(1+(IC₅₀/X)\hat{ }HillSlope)

The result is expressed with IC₅₀, shown as Table 11, Compounds of the present disclosure, as exemplified in the Examples, showed IC₅₀ values in the following ranges: “*” stands for “IC₅₀≤10 nM”; “*” stands for “10 nM<IC₅₀≤50 nM”; “***” stands for “IC₅₀>50 nM.

TABLE 12

| EX No. | Trk A Kinase IC₅₀(nm) |
|---|---|
| 1 | * |
| 2 | 0.43 |
| 3 | ** |
| 4 | 1.37 |
| 5 | 1.8 |
| 6 | * |
| 7 | 0.68 |
| 8 | ** |
| 9 | ** |
| 10 | ** |
| 11 | * |
| 12 | *** |
| 13 | * |
| 14 | *** |
| 15 | *** |
| 16 | *** |
| 17 | *** |
| 18 | ** |
| 19 | * |
| 20 | 47 |
| 21 | *** |
| 22 | ** |
| 23 | 2.3 |
| 24 | 11.8 |
| 25 | ** |
| 26 | 2.6 |
| 27 | *** |
| 28 | *** |
| 29 | 1.4 |
| 30 | 4.9 |
| 31 | 2.3 |
| 32 | ** |
| 33 | 2.41 |
| 34 | 0.71 |
| 35 | ** |
| 36 | 1.14 |
| 37 | 1.57 |
| 38 | 11.7 |
| 39 | 1.26 |
| 40 | 1.56 |
| 41 | 3.92 |
| 42 | 3.9 |
| 43 | 2.51 |
| 44 | 5.28 |
| 45 | 0.8 |
| 46 | 2.02 |
| 47 | 3.23 |
| 48 | * |
| 49 | 6.4 |
| 50 | 2.65 |
| 51 | 3 |
| 52 | 1.66 |
| 53 | 1.4 |
| 54 | 1.17 |
| 55 | ** |
| 56 | 1.2 |
| 57 | 0.6 |
| 58 | *** |
| 59 | * |
| 60 | ** |
| 61 | * |
| 62 | *** |
| 63 | *** |
| 64 | *** |
| 65 | 0.27 |
| 66 | * |
| 67 | 0.32 |
| 68 | * |
| 69 | * |
| 70 | 0.5 |
| 71 | 27.15 |
| 72 | 1.4 |
| 73 | 0.59 |
| 74 | 0.96 |
| 75 | * |
| 76 | 0.66 |
| 77 | 0.7 |
| 78 | 0.73 |
| 79 | 11.89 |
| 80 | 23.64 |
| 81 | 2.18 |
| 82 | ** |
| 83 | *** |
| 84 | 0.18 |
| 85 | 0.31 |
| 86 | 0.76 |
| 87 | 0.73 |
| 88 | 0.77 |
| 89 | ** |
| 90 | 0.44 |
| 91 | 0.26 |
| 92 | 20.67 |
| 93 | * |
| 94 | 0.79 |
| 95 | * |
| 96 | 0.7 |
| 97 | 1.9 |
| 98 | 8.8 |
| 99 | 0.5 |
| 100 | 1 |
| 101 | 0.4 |
| 102 | 7 |
| 103 | 1.2 |
| 104 | 13.5 |
| 105 | 32.9 |
| 106 | * |
| 107 | * |
| 108 | * |
| 109 | * |
| 110 | 1.9 |
| 111 | 5.3 |
| 112 | 3.2 |
| 113 | 6.7 |
| 114 | 3.8 |
| 115 | *** |
| 116 | ** |
| 117 | * |
| 118 | ** |
| 119 | * |
| 120 | * |
| 121 | ** |
| 122 | ** |
| 123 | * |
| 124 | ** |
| 125 | 1.1 |
| 126 | * |
| 127 | * |
| 128 | * |
| 129 | * |
| 130 | *** |
| 131 | ** |
| 132 | ** |
| 133 | ** |
| 134 | 1.1 |
| 135 | * |
| 136 | 1.9 |
| 137 | 2.65 |
| 138 | 0.48 |
| 139 | *** |
| 140 | * |
| 141 | 0.3 |
| 142 | * |
| 143 | * |
| 144 | 1.1 |
| 145 | * |
| 146 | * |
| 147 | * |
| 148 | 1.5 |
| 149 | * |
| 150 | * |
| 151 | * |
| 152 | * |
| 153 | * |
| 154 | * |
| 155 | 0.6 |
| 156 | 4.8 |
| 157 | 27 |
| 158 | * |
| 159 | * |
| 160 | 0.43 |
| 161 | 0.9 |
| 162 | 0.6 |
| 163 | * |
| 164 | * |
| 165 | * |
| 166 | 2.5 |
| 167 | 0.3 |
| 168 | * |
| 169 | * |
| 170 | * |
| 171 | 1 |
| 172 | 0.6 |
| 173 | * |
| 174 | * |
| 175 | * |
| 176 | ** |
| 177 | 1.2 |
| 178 | * |
| 179 | ** |
| 180 | *** |
| 181 | ** |
| 182 | ** |
| 183 | 20.38 |
| 184 | * |
| 185 | * |
| 186 | ** |
| 187 | 6.6 |
| 188 | * |
| 189 | * |
| 190 | * |
| 191 | ** |
| 192 | ** |
| 193 | * |
| 194 | * |
| 195 | 2.3 |
| 196 | 2.6 |
| 197 | ** |
| 198 | *** |
| 199 | ** |
| 200 | ** |

### Example 202 Ba/F3-TPM3-NTRK1 and Ba/F3-ETV6-NTRK3 cells proliferation assay

1. Cell Culture

Cell line: Ba/F3 cells with TPM3-NTRK1 or ETV6-NTRK3 fusion mutation gene stably overexpressed named Ba/F3-TPM3-NTRK1 and Ba/F3-ETV6-NTRK3.

A. Culture Medium

RPMI 1640 and 10% FBS and 1% PS and 2 ug/mL puromycin.

B. Cell Recovery

a) The medium was preheated in a 37° C. water bath in advance.

b) Remove the cryogenic vials from the liquid nitrogen tank, quickly put it into a 37° C. water bath, and completely melt it in 1 min.

c) Transfer the cell suspension to a 15 mL centrifuge tube containing 8 mL medium, centrifuge 1000 rpm, 5 min.

d) Discard the supernatant, resuspend the cells in 1 mL medium, and transfer to a 75 cm² flask containing 15 mL medium, culture the cells in an incubator at 37° C., 5% CO₂.

C. Cell Passage

a) The medium was preheated in a 37° C. water bath in advance.

b) Collect cell to a 15 mL centrifuged tube, centrifuge at 1000 rpm for 5 min. Discard the supernatant, count, and make the cell density at 1.49×10⁴ cells/mL, then place it in a 37° C., 5% CO₂ incubator.

2. Compound Preparation

a) The test compound (20 mM stock solution) was diluted to 60 μM with 100% DMSO as starting concentration then 3-fold serial diluted with “9+0” concentrations. in a 96-well dilution plate (Thermo, Cat. No. 249944);

b) The above compound solution was diluted 1:20 times with culture medium to prepare a 10 fold working solution;

3. Cell Plating

a) Take cells in log phase growth, centrifuge at 1000 rpm for 5 min, then resuspend the cells with culture medium, then count cells;

b) Cells were seeded to 96-well cell culture plate with density at 2000 cells/well;

4. Compound Treatment

a) Compounds prepared at step 2 were added to cell plate with 15 μL per well, the final concentrations were 300, 100, 33.33, 11.11, 3.70, 1.23, 0.41, 0.14, 0.05 and 0 nM, and the final concentration of DMSO was 0.5%. The blank control well was a culture medium (0.5% DMSO);

c) The cells were incubated for an additional 72 h in the incubator.

5. Detection

a) Remove the 96-well cell culture plate and add 50 μl of CTG reagent (CellTiter Glo kit, promega, Cat #G7573).

b) Plate was shaked for 2 min and then let it cool for 30 min at room temperature.

c) The Luminescence signal value was read using a PerkinElmer reader.

Experimental Data Analysis

Data were analyzed using GraphPad Prism 6.0 software to obtain a fitted curve of compound activity.

Fit the Compound IC50 from non-linear regression equation:

Y=Bottom+(Top-Bottom)/(1+10\hat{ }((Log IC50-X)×HillSlope));

X: The log of the concentration of the compound; Y: Luminescence value.

TABLE 13

| | TPM3-NTRK1 | ETV6-NTRK3 | NTRK1-G595R |
|---|---|---|---|
| EX No. | IC₅₀ (nM) | IC₅₀ (nM) | IC₅₀ (nM) |
| LOXO-101 | — | — | 3073.1 |
| Compound 4 | 3.55 | 2.78 | — |
| Compound 11 | 13.64 | 5.785 | — |
| Compound 7 | 8.17 | 5.68 | 107 |
| Compound 8 | 2.209 | 1.548 | — |
| Compound 9 | 2.05 | 2.11 | — |
| Compound 10 | 1.16 | 1.2 | — |
| Compound 11 | 13.64 | 5.785 | — |
| Compound 12 | 7.713 | 2.716 | — |
| Compound 13 | 0.98 | 1.83 | — |
| Compound 14 | 12.52 | 10.03 | — |
| Compound 16 | 9.182 | 4.212 | — |
| Compound 17 | 4.1 | 3.56 | — |
| Compound 18 | 3.813 | 2.833 | — |
| Compound 22 | 2.929 | 2.454 | — |
| Compound 23 | 20.87 | — | — |
| Compound 30 | 13.78 | — | — |
| Compound 31 | 3.88 | — | — |
| Compound 34 | 3.33 | — | — |
| Compound 39 | 7.42 | — | — |
| Compound 94 | 1.38 | — | 62.9 |
| Compound 101 | 0.28 | — | 164.6 |
| Compound 125 | 1.38 | 2.21 | 46.6 |
| Compound 123 | 1.9 | — | 116.8 |
| Compound 126 | 1.04 | — | 105.9 |
| Compound 141 | 30.08 | 20.77 | — |
| Compound 144 | 3.39 | — | 221.5 |
| Compound 148 | 2.19 | 1.56 | 81.8 |
| Compound 155 | 1.2 | 0.53 | 18.2 |
| Compound 156 | 3.92 | 5.65 | 182.9 |
| Compound 160 | 1.06 | 2.05 | 15.6 |
| Compound 162 | 1.33 | — | 118.8 |
| Compound 166 | 4.67 | 7.73 | — |
| Compound 167 | 13.24 | 19.6 | — |
| Compound 169 | 2.91 | — | 102.7 |
| Compound 171 | 3.41 | 2.63 | 215.7 |
| Compound 172 | 2.08 | 2.49 | 256.9 |
| Compound 173 | 2.19 | — | 89.8 |

Note:
“—” stands for “not tested”.

### Example 203 Liver Microsomes Metabolic Stability Assay

Pooled human liver microsomes (Cat. 452117) were purchased from Corning. Pooled male rat liver microsomes (Cat. R1000) and pooled male mouse liver microsomes (Cat. M1000) were purchased from XENOTECH. Microsomes were stored at −80° C.

1) Make a master solution containing phosphate buffer, ultra-pure H₂O and MgCl₂ solution according to Table 14.

TABLE 14
Preparation of master solution

| | Stock | | | | Final | |
|---|---|---|---|---|---|---|
| Buffer | Concentration | | Volume | | Concentration | |
| Phosphate buffer | 200 | mM | 200 | μL | 100 | mM |
| Ultra-pure H₂O | — | | 106 | μL | — | |
| MgCl₂ solution | 50 | mM | 40 | μL | 5 | mM |

2) Two separated experiments were performed as follows.

a) With NADPH: 10 μL of 20 mg/mL liver microsomes and 40 μL of 10 mM NADPH were added to the incubations. The final concentrations of microsomes and NADPH were 0.5 mg/mL and 1 mM, respectively.

b) Without NADPH: 10 μL of 20 mg/mL liver microsomes and 40 μL of ultra-pure H₂O were added to the incubations. The final concentration of microsomes was 0.5 mg/mL.

3) The reaction was started with the addition of 4 μL test compounds solutions or control compound solution (verapamil) at the final concentration of 2 μM and carried out at 37° C.

4) Aliquots of 50 μL were taken from the reaction solution at 0, 15, 30, 45 and 60 min. The reaction solutions were stopped by the addition of 4 volumes of cold acetonitrile with 1S (100 nM alprazolam, 200 nM caffeine, 200 nM labetalol and 2 μM ketoprofen). Samples were centrifuged at 3,220 g for 40 minutes. Aliquot of 100 μL of the supernatant was mixed with 100 μL of ultra-pure H₂O and then used for LC-MS/MS analysis. All experiments were performed in duplicate.

The slope value, k, was determined by linear regression of the natural logarithm of the remaining percentage of the parent drug vs. incubation time curve.

The in vitro half-life (in vitro t_{1/2}) was determined from the slope value:

in vitro t_{1/2}=—(0.693/k)

Conversion of the in vitro t_{1/2} (in min) into the in vitro intrinsic clearance (in vitro CLᵢₙₜ, in μL/min/mg proteins) was done using the following equation (mean of duplicate determinations):

$$in vitro {CL}_{int} = \frac{0.693}{{t}_{1 \text{/} 2}} \times \frac{volume of incubation ( μ L )}{amount of proteins ( mg )}$$

The control compound (verapamil) was included in the assay. Any value of the compounds that was not within the specified limits will be rejected and the experiment will be repeated.

The results of metabolic stability in different species of liver microsomes are shown in Table 15.

TABLE 15

| | CLᵢₙₜ(μL/min/mg proteins) | | | t_{1/2} (min) | | |
|---|---|---|---|---|---|---|
| Compound | Human | Rat | Mouse | Human | Rat | Mouse |
| Comparative | 107.97 | 78.48 | 214.08 | 12.84 | 17.66 | 6.47 |
| compound A | | | | | | |
| Compound 7 | 11.14 | 25.26 | 19.38 | 124.43 | 54.86 | 71.52 |
| Compound 76 | 21.20 | 22.20 | 15.60 | 65.37 | 62.43 | 88.84 |
| Compound 36 | 4.40 | 19.20 | 4.60 | 315.00 | 72.19 | 301.30 |
| Compound 71 | 17.00 | 12.20 | 10.80 | 81.53 | 113.61 | 128.33 |
| Compound 40 | 3.20 | 4.60 | 12.40 | 433.13 | 301.30 | 111.77 |
| Compound 26 | 23.03 | 40.53 | 25.91 | 60.17 | 34.2 | 53.5 |
| Compound 65 | 12.60 | 17.20 | 24.00- | 110.00 | 80.58 | 57.75 |
| Compound 17 | 0.27 | 0.00 | 2.23 | ∞ | 622.12 | ∞ |
| Compound 112 | 16.80 | 34.0 | 27.40 | 82.50 | 40.77 | 50.58 |
| Compound 96 | 37.00 | 29.40 | 32.00 | 37.46 | 47.14 | 43.31 |
| Compound 92 | 20.40 | 10.20 | 5.60 | 67.94 | 135.88 | 247.50 |
| Compound 90 | 5.60 | 1.40 | 0.80 | 247.50 | 990.00 | 1732.50 |
| Compound 80 | 2.40 | 7.00 | 8.40 | 577.50 | 198.00 | 165.00 |
| Compound 79 | 10.20 | 10.80 | 10.00 | 135.88 | 128.33 | 138.60 |
| Compound 4 | 20.4 | 29.31 | 96.4 | 67.941 | 47.29 | 14.378 |
| Compound 11 | 4.27 | 5.23 | 61.91 | — | — | — |
| Compound 23 | 9.36 | 50 | 48.59 | 148.09 | 27.72 | 28.52 |
| Compound 30 | 16.77 | 69.6 | 35.64 | 82.65 | 19.9 | 38.9 |
| Compound 31 | 16.38 | 69.07 | 37 | 84.62 | 20.07 | 37.4 |
| Compound 39 | 17.2 | 80.2 | 41.4 | 80.58 | 17.28 | 33.47 |
| Compound 101 | 49.4 | 105.4 | 102.8 | 28.05 | 13.15 | 13.48 |
| Compound 125 | 17.2 | 15 | 16.4 | 80.58 | 92.4 | 84.51 |
| Compound 123 | 94.09 | 60.8 | 39.05 | 14.73 | 22.8 | 35.5 |
| Compound 126 | 42.2 | 21 | 33.4 | 32.84 | 66 | 41.49 |
| Compound 148 | 1.2 | 3.6 | 14 | 1155 | 385 | 99 |
| Compound 160 | 56.2 | 92.4 | 104.8 | 24.66 | 15 | 13.225 |
| Compound 169 | 55.8 | 78.4 | 102.4 | 24.84 | 17.68 | 13.53 |
| Compound 171 | 45.4 | 58.2 | 71.2 | 30.52 | 23.81 | 19.46 |
| Compound 172 | 41.6 | 44.8 | 32.4 | 33.31 | 30.94 | 42.77 |
| Compound 173 | 46.6 | 45.2 | 97.6 | 29.742 | 30.66 | 14.2 |

Note:
“—” stands for “not tested”.

As a result, it has been confirmed that the exemplified compounds of the present invention have drastically improved metabolic stability in Human/Rat/Mouse liver microsomes compared with the Comparative compound A. This improved stability indicated superior pharmacokinetic properties and better clinical output in human.

### Example 204 Plasma Protein Binding Measurements

The plasma protein binding was measured as the following procedure.

1) Preparation of 100 mM sodium phosphate and 150 mM NaCl buffer (PBS)

A basic solution was prepared by dissolving 14.2 g/L Na₂HPO₄ and 8.77 g/L NaCl in deionized water and the solution could be stored at 4° C. for up to 7 days. An acidic solution was prepared by dissolving 12.0 g/L NaH₂PO₄ and 8.77 g/L NaCl in deionized water and the solution could be stored at 4° C. for up to 7 days. The basic solution was titrated with the acidic solution to pH 7.4 and store at 4° C. for up to 7 days. pH was checked on the day of experiment and was adjusted if outside specification of 7.4±0.1.

2) Preparation of plasma

Frozen plasma was thawed immediately at room temperature.

The plasma was centrifuged at 3,220 g for 10 minutes to remove clots and supernatant was collected into a fresh tube. The pH of the plasma was checked and recorded.

Note: a). Only use plasma that has been thawed no more than twice since arrival. b). Only use plasma within the range of pH 7 to pH 8.

3) Preparation of working solutions

The working solutions of test compounds and control compound ketoconazole were prepared in DMSO at the concentration of 200 μM. And then 3 μL of working solution was removed to mix with 597 μL of human, rat or mouse plasma to achieve a final concentration of 1 μM (0.5% DMSO). Plasma samples were vortexed thoroughly.

4) Preparation of dialysis membranes

The dialysis membranes were soaked in ultrapure water for 60 minutes to separate strips, then in 20% ethanol for 20 minutes, finally in dialysis buffer for 20 minutes.

5) Procedure for equilibrium dialysis

The dialysis apparatus was assembled according to the manufacturer's instruction. Each cell was filled with 120 μL of plasma sample and dialyzed against equal volume of dialysis buffer (PBS). The assay was performed in duplicate. The dialysis plate was sealed and incubated in an incubator at 37° C. with 5% CO₂ at 100 rpm for 6 hours. At the end of incubation, seal was removed and 50 μL of samples from both buffer and plasma chambers were transferred to wells of a 96-well plate.

6) Procedure for sample analysis

50 μL of blank plasma was added to each buffer samples and an equal volume of PBS was supplemented to the collected plasma sample. 300 μL of room temperature quench solution (acetonitrile containing internal standards (1S, 100 nM Alprazolam, 500 nM Labetalol and 2 μM Ketoprofen)) was added to precipitate protein. Samples in plate were vortexed for 5 minutes and centrifuged at 3,220 g for 30 minutes at 4° C. And then 100 μL of the supernatant was transferred to a new 96-well plate with 100 μL or 200 μL water (depends on the LC-MS signal response and peak shape) for LC-MS/MS analysis.

Calculate the percentages of test compound and control compound bound as follows:

% Free=(Peak Area Ratio_{buffer chamber}/Peak Area Ratio_{plasma chamber})*100

% Bound=100−% Free

% Recovery=(Peak Area Ratio_{buffer chamber}+Peak Area Ratio_{plasma chamber})/Peak Area Ratio total sample*100

Peak Area Ratio_{buffer chamber} means the conc for free fraction

Peak Area Ratio_{plasma chamber} means the conc for both free and bound fraction

Peak Area Ratio_{total sample} means the conc for starting sample before incubation

Plasma protein binding results of control compound and test Compounds in different species are shown in Table 16.

TABLE 16

| | Mean % Bound | | | Mean % Recovery | | |
|---|---|---|---|---|---|---|
| Compound | Human | Rat | Mouse | Human | Rat | Mouse |
| Comparative | 96.77 | 92.71 | 95.12 | 93.81 | 93.78 | 89.38 |
| Compound A | | | | | | |
| Compound 4 | 93.09 | 89.99 | 95.57 | — | — | — |
| Compound 11 | 71.63 | 71.07 | 75.65 | 89.30 | 87.45 | 85.49 |
| Compound 7 | 90.12 | 84..80 | 90.71 | 96.26 | 90.80 | 89.38 |
| Compound 23 | 93.96 | 93.01 | 96.03 | — | — | — |
| Compound 24 | 87.40 | 83.61 | 91.83 | 85.93 | 82.52 | 86.51 |
| Compound 30 | 86.10 | 82.59 | 91.13 | 90.71 | 92.49 | 89.95 |
| Compound 31 | 87.88 | 86.90 | 90.02 | 96.84 | 98.92 | 94.25 |
| Compound 34 | 91.32 | 89.11 | 90.82 | 91.73 | 92.37 | 98.60 |
| Compound 39 | 88.90 | 88.45 | 93.24 | 97.40 | 89.69 | 91.34 |

Note:
“—” stands for “not tested”.

Generally, only the unbound fraction can have a biological effect or be metabolized. Therefore, the degree of binding to plasma proteins significantly influences the pharmacokinetic and pharmacodynamics properties of a drug.

As shown in Table 16, the Comparative compound A reflected a high degree of plasma protein binding, therefore the efficacy of the drug might be reduced. Unexpectedly, the exemplified compounds of the present invention have lower degree of plasma protein binding compared with the Comparative compound A. It indicated the present invention had superior pharmacokinetic and pharmacodynamics properties in human.

### Example 205 Cytochrome P450 Measurements

The cytochrome P450 was measured as the following procedure:

1) A master solution containing phosphate buffer, ultra-pure H₂O, MgCl₂ solution and human liver microsomes was made according to Table 17, and then 1 μL of 2 mM of compound solution or 1 μL of DMSO (as without inhibitor control) was added to the above master solution. The final concentration of test compounds or control compounds was 10 μM.

TABLE 17

| | Stock | | | | Final | |
|---|---|---|---|---|---|---|
| Reagent | Concentration | | Volume | | Concentration | |
| MgCl₂ solution | 50 | mM | 20 | μL | 5 | mM |
| Phosphate buffer | 200 | mM | 100 | μL | 100 | mM |
| Ultra-pure H₂O | — | | 56 | μL | — | |
| Human liver microsomes | 20 | mg/mL | 2 | μL | 0.2 | mg/mL |

2) For CYP1A2 inhibition, 1 μL of specific drug substrate (Phenacetin: 8 mM) was added at the final concentration of 40 μM to the above solution.

3) For CYP2C8 inhibition, 1 μL of specific drug substrate (Paclitaxel: 1 mM) was added at the final concentration of 5 μM to the above solution.

4) For CYP2C9 inhibition, 1 μL of specific drug substrate (Tolbutamide: 40 mM) was added at the final concentration of 200 μM to the above solution.

5) For CYP2C19 inhibition, 1 μL of specific drug substrate ((s)-Mephenytoin: 10 mM) was added at the final concentration of 50 μM to the above solution.

6) For CYP3A4 inhibition, 1 μL of specific drug substrate (Midazolam: 1 mM) was added at the final concentration of 5 μM to the above solution.

7) For CYP3A4 inhibition, 1 μL of specific drug substrate (Testosterone: 10 mM) was added at the final concentration of 50 μM to the above solution.

8) The mixture was pre-warmed at 37° C. for 5 min. The reaction was started by the addition of 20 μL of 10 mM NADPH solution at the final concentration of 1 mM and carried out at 37° C.

9) The reaction was stopped by addition of 300 μL of cold quench solution (methanol containing internal standards (1S, 500 nM Labetalol, 100 nM Alprazolam and 2 μM Ketoprofen) at the designated time points (Phenacetin: 20 min; Paclitaxel: 10 min; Tolbutamide: 20 min; (s)-Mephenytoin: 20 min; Midazolam: 5 min; Testosterone: 10 min). Samples were vortexed for 5 minutes and centrifuged at 3220 g for 40 minutes at 4° C. And then 100 μL of the supernatant was transferred to a new 96-well plate with 100 μL or 200 μL water (depends on the LC-MS signal response and peak shape) for LC-MS/MS analysis.

All experiments were performed in duplicates.

Inhibition percentages for compounds against CYP1A2, CYP2C8, CYP2C9, CYP2C19, and

CYP3A4 shown in Table 18 (the unit is %).

TABLE 18

| | CYP1A2 | CYP2C8 | CYP2C9 | CYP2C19 | CYP3A4 | CYP3A4 |
|---|---|---|---|---|---|---|
| Compound | (Phenacetin) | (Paclitaxel) | (Tolbutamide) | ((s)-Mephenytoin) | (Midazolam) | (Testosterone) |
| Comparative | 28.99 | 19.34 | 25.39 | 12.98 | 70.08 | 38.29 |
| compound A | | | | | | |
| Compound 4 | 1.53 | 18.22 | 5.96 | 7.23 | 20.03 | 22.78 |
| Compound 7 | 8.54 | 1.37 | 22.66 | 6.79 | 6.97 | 5.7 |
| Compound 47 | 4.38 | 32.82 | 50.37 | 20.34 | 22.4 | 29.53 |

As a result, it has been confirmed that the exemplified compounds of the present invention have low inhibition against CYP1A2, CYP2C8, CYP2C9, CYP2C19 and CYP3A4. Especially for CYP3A4, which is a main isoform for drug metabolism, compounds in the present invention have much less inhibition compared with the Comparative compound A.

1. A compound of Formula I, or an isomeride, a stereoisomer, tautomer, pharmaceutically acceptable salt, prodrug, chelate, non-covalent complex, or solvate thereof, 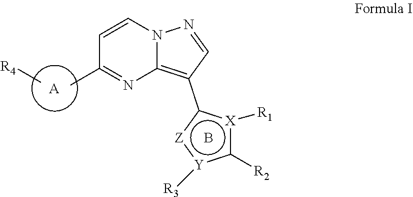
wherein,
ring A is C₅₋₆ heterocyclic ring, wherein the C₅₋₆ heterocyclic ring optionally comprises 1, 2 or 3 hetero atoms independently selected from N, S, or O;
ring B is 5-membered aromatic heterocycle;
X and Z are each independently selected from C, N, O, or S;
Y is C or N;
R₁ is absent, H, or —C₁₋₈ alkyl;
R₂ is H, —C₀₋₄ alkyl-COOR₁₀, —C₀₋₄ alkyl-NH—COOR₁₀, —C₀₋₄ alkyl-O(CO)R₁₀, —C₀₋₄ alkyl-O(CO)—C₁₋₄ alkyl-NHCO—R₁₀, —C₁₋₄ alkyl-NH₂, —C₀₋₄ alkyl-OH, —C₁₋₄ alkyl-C₃₋₁₀ carbocyclic ring, or —C₀₋₄ alkyl-C₃₋₁₀ heterocyclic ring, —C₀₋₄ alkyl-C₆₋₁₀ aryl ring, or —C₀₋₄ alkyl-C₅₋₁₀ heteroaryl ring, wherein the —C₀₋₄ alkyl-COOR₁₀, —C₀₋₄ alkyl-NH—COOR₁₀, —C₀₋₄ alkyl-O(CO)R₁₀, —C₀₋₄ alkyl-O(CO)—C₁₋₄ alkyl-NHCO—R₁₀, —C₁₋₄ alkyl-NH₂, —C₀₋₄ alkyl-OH, —C₀₋₄ alkyl-C₃₋₁₀ carbocyclic ring, —C₀₋₄ alkyl-C₃₋₁₀ heterocyclic ring, —C₀₋₄ alkyl-C₆₋₁₀ aryl ring, or —C₀₋₄ alkyl-C₅₋₁₀ heteroaryl ring is optionally substituted with —C₁₋₈ alkyl, —C₂₋₈ alkynyl, —C₁₋₈ haloalkyl, —C₁₋₈ alkyl-OH, halogen, OH, CN, NH₂, —C₀₋₄ alkyl-COOR₁₀, —C₆₋₁₀ aryl ring, —O—C₆₋₁₀ aryl ring, substituted or unsubstituted —C₃₋₁₀ carbocyclic ring, or substituted or unsubstituted —C₃₋₁₀ heterocyclic ring;
R₃ is absent, C₃₋₁₀ heterocyclic ring; or
R₂ and R₃ together with the atoms to which they are attached to form a 5- to 6-membered carbocyclic ring, heterocyclic ring, aryl ring, or heteroaryl ring, wherein the 5- to 6-membered carbocyclic ring, heterocyclic ring, aryl ring, or heteroaryl ring is optionally substituted with halogen, OH, CN, NH₂, —CONHOH, —CONH₂, —C₀₋₄ alkyl-COOR₁₀, —C₀₋₄ alkyl-O(CO)OR₁₀, —C₁₋₈alkoxy, —C₁₋₈ haloalkoxy, —C₁₋₈ alkoxy-C₁₋₈ alkoxy, —C₁₋₈ alkylthio, —C₁₋₈ haloalkylthio, —C₁₋₈alkyl, —C₁₋₈ haloalkyl, —C₀₋₄ alkyl-OH, —O—CH₂—CN, —C₀₋₄ alkyl-O—C₃₋₁₀ heterocyclic ring, substituted or unsubstituted —C₃₋₁₀ carbocyclic ring or substituted or unsubstituted —C₃₋₁₀ heterocyclic ring, or the 5- to 6-membered carbocyclic ring, heterocyclic ring, aryl ring, or heteroaryl ring forms a ring structure with other substituted or unsubstituted carbocyclic ring, substituted or unsubstituted heterocyclic ring, substituted or unsubstituted aryl ring, or substituted or unsubstituted heteroaryl ring;
R₄ is (i) phenyl optionally substituted with one or more substituents independently selected from halogen, —C₁₋₄ alkyl, —C₁₋₄ haloalkyl, C₁₋₄alkoxyl, or (ii) a C₅₋₆ heteroaryl ring having a heteroatom selected from N, S, or O, wherein the C₅₋₆ heteroaryl ring is optionally substituted with one or more halogen atoms;
R₁₀ is H, or —C₁₋₈ alkyl;
wherein the heterocyclic ring or the heteroaryl ring optionally has 1, 2 or 3 heteroatoms independently selected from N, S, O or B.

2. The compound of claim 1, wherein ring A is

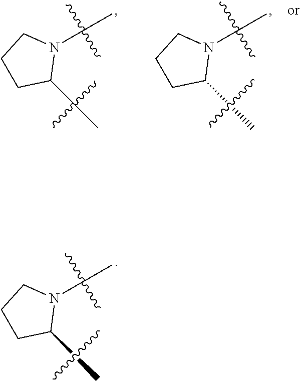

3. The compound of claim 1, wherein X is independently selected from O, S or N.

4. The compound of claim 1, wherein Y is C.

5. The compound of claim 1, wherein Z is N.

6. The compound of claim 1, wherein R₄ is

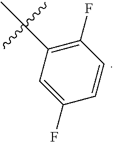 [SMILES: *c1cc(F)ccc1F]

7. The compound of claim 1, wherein the compound is formula II or a stereoisomer, tautomer, pharmaceutically acceptable salt, prodrug, chelate, non-covalent complex, or solvate thereof, 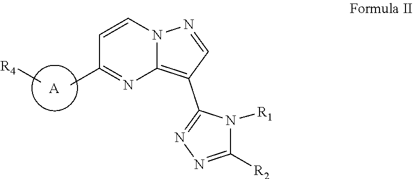
wherein,
ring A is C₅-6 heterocyclic ring, wherein the C₅₋₆ heterocyclic ring optionally comprises 1, 2 or 3 hetero atoms independently selected from N, S, or O;
R₁ is H, or —C₁₋₈ alkyl;
R₂ is H, —C₀₋₄ alkyl-COOR₁₀, —C₀₋₄ alkyl-NH—COOR₁₀, —C₀₋₄ alkyl-O(CO)R₁₀, —C₀₋₄ alkyl-O(CO)—C₁₋₄ alkyl-NHCO—R₁₀, —C₁₋₄ alkyl-NH₂, —C₀₋₄ alkyl-OH, —C₁₋₄ alkyl-C₃₋₁₀ carbocyclic ring, or —C₀₋₄ alkyl-C₃₋₁₀ heterocyclic ring, —C₀₋₄ alkyl-C₆₋₁₀ aryl ring, or —C₀₋₄ alkyl-C₅₋₁₀ heteroaryl ring, wherein the —C₀₋₄ alkyl-COOR₁₀, —C₀₋₄ alkyl-NH—COOR₁₀, —C₀₋₄ alkyl-O(CO)R₁₀, —C₀₋₄ alkyl-O(CO)—C₁₋₄ alkyl-NHCO—R₁₀, —C₁₋₄ alkyl-NH₂, —C₀₋₄ alkyl-OH, —C₁₋₄ alkyl-C₃₋₁₀ carbocyclic ring, —C₀₋₄ alkyl-C₃₋₁₀ heterocyclic ring, —C₀₋₄ alkyl-C₆₋₁₀ aryl ring, or —C₀₋₄ alkyl-C₅₋₁₀ heteroaryl ring is optionally substituted with —C₁₋₈alkyl, —C₂₋₈ alkynyl, —C₁₋₈ haloalkyl, —C₁₋₈ alkyl-OH, halogen, OH, CN, NH₂, —C₀₋₄ alkyl-COOR₁₀, —C₆₋₁₀ aryl ring, —O—C₆₋₁₀ aryl ring, substituted or unsubstituted —C₃₋₁₀ carbocyclic ring or substituted or unsubstituted —C₃₋₁₀ heterocyclic ring;
R₄ is (i) phenyl optionally substituted with one or more substituents independently selected from halogen, —C₁₋₄ alkyl, —C₁₋₄ haloalkyl, C₁₋₄ alkoxyl, or (ii) a C₅₋₆ heteroaryl ring having a ring heteroatom selected from N, S, or O, wherein the C₅₋₆ heteroaryl ring is optionally substituted with one or more halogen atoms;
R₁₀ is H, or —C₁₋₈ alkyl;
wherein the heterocyclic ring or the heteroaryl ring optionally has 1, 2 or 3 heteroatoms independently selected from N, S, O or B.

8. The compound of claim 7, wherein ring A is

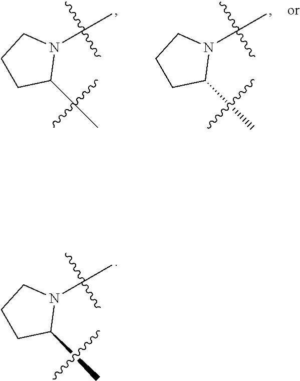

9. The compound of claim 7, wherein R₁ is independently selected from H or CH₃.

10. The compound of claim 7, wherein R₄ is

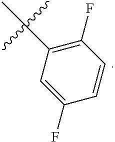 [SMILES: *c1cc(F)ccc1F]

11. The compound of claim 7, wherein R₂ is independently selected from

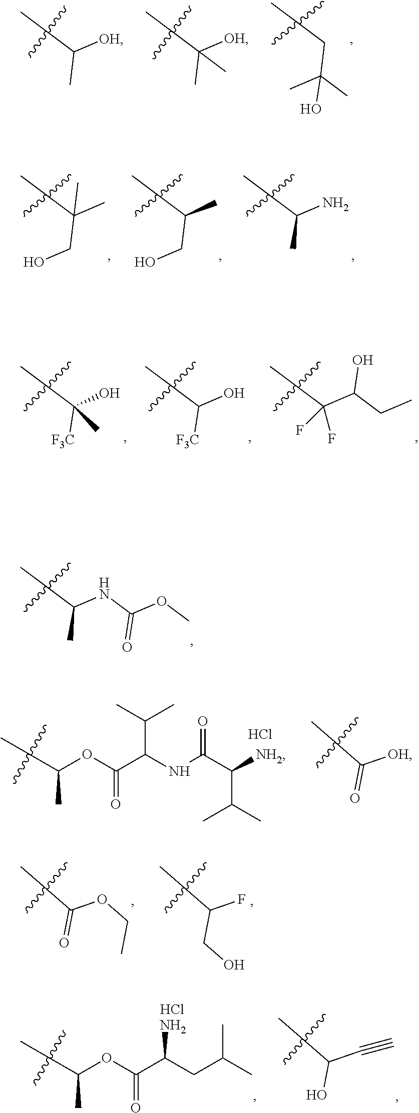

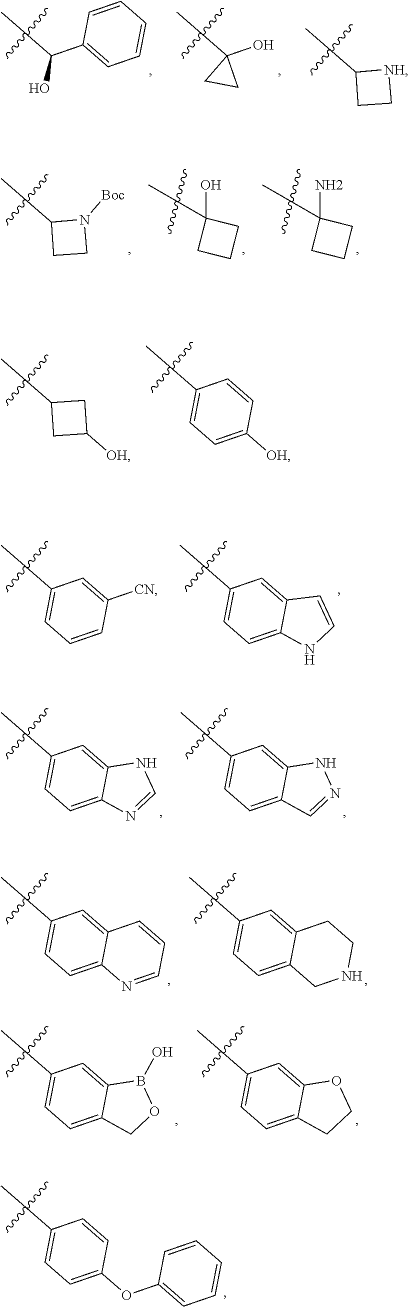

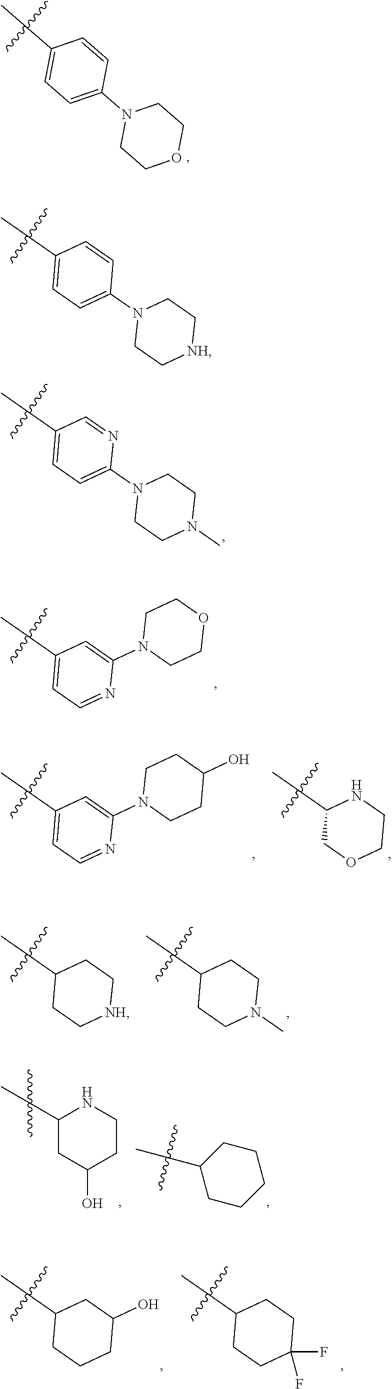

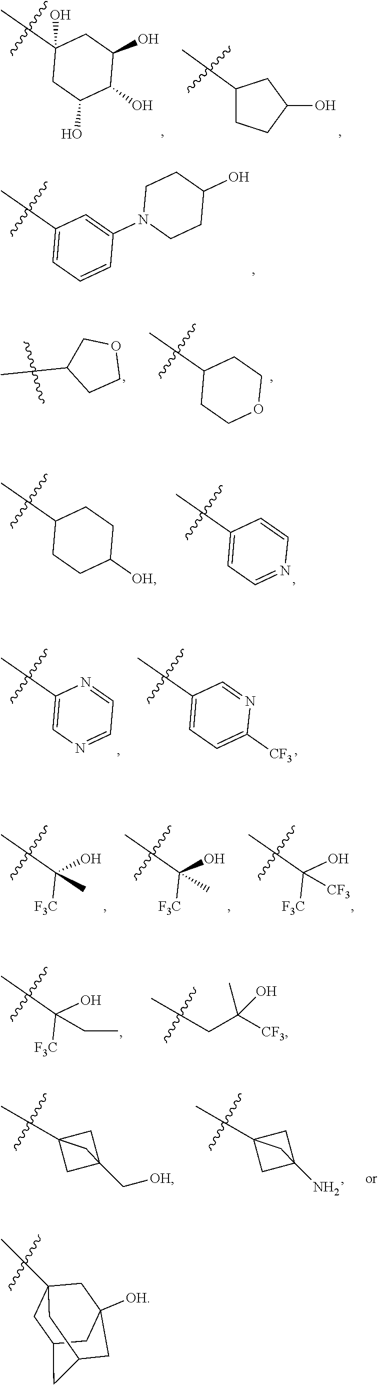

12. The compound of claim 7, wherein R₂ is

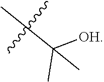 [SMILES: *C(C)(C)O]

13. The compound of claim 1, wherein the compound is of formula III or an isomeride, a stereoisomer, tautomer, pharmaceutically acceptable salt, prodrug, chelate, non-covalent complex, or solvate thereof: 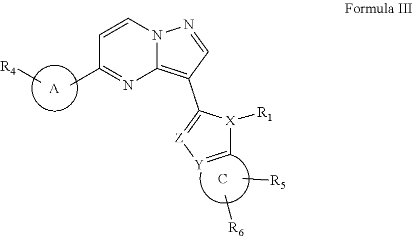
wherein,
ring A is C₅₋₆ heterocyclic ring, wherein the C₅₋₆ heterocyclic ring optionally comprises 1, 2 or 3 hetero atoms independently selected from N, S, or O;
ring C is a 5- to 6-membered carbocyclic ring, heterocyclic ring, aryl ring, or heteroaryl ring;
X and Z are each independently selected from C, N, O, or S;
Y is C or N;
R₁ is absent, H, or —C₁₋₈ alkyl;
R₄ is (i) phenyl optionally substituted with one or more substituents independently selected from halogen, —C₁₋₄ alkyl, —C₁₋₄ haloalkyl, C₁₋₄ alkoxyl, or (ii) a C₅₋₆ heteroaryl ring having a ring heteroatom selected from N, S, or O, wherein the C₅₋₆ heteroaryl ring is optionally substituted with one or more halogen atoms;
R₅ and R₆ are each independently selected from H, OH, NH₂, CN, —COOH, —CONHOH, —CONH₂, halogen, —C₁₋₈ alkyl, —C₀₋₄ alkyl-COOR₁₀, —C₀₋₄ alkyl-O(CO)OR₁₀, —C₁₋₈ alkoxy, —C₁₋₈ haloalkoxy, —C₁₋₈ alkoxy-C₁₋₈ alkoxy, —C₁₋₈ alkylthio, —C₁₋₈ haloalkylthio, —C₁₋₈ alkyl, —C₁₋₈ haloalkyl, —C₀₋₄ alkyl-OH, —O—CH₂—CN, —C₀₋₄ alkyl-O—C₃₋₁₀ heterocyclic ring, substituted or unsubstituted carbocyclic ring or substituted or unsubstituted —C₃₋₁₀ heterocyclic ring;
or R₅ and R₆ together with the atoms to which they are attached to form a 5 to 12-membered carbocyclic ring, heterocyclic ring, aryl ring, or heteroaryl ring, wherein the 5 to 12-membered carbocyclic ring, heterocyclic ring, aryl ring, or heteroaryl ring is optionally substituted with halogen;
R₁₀ is H, or —C₁₋₈ alkyl;
wherein the heterocyclic ring or the heteroaryl ring optionally has 1, 2 or 3 heteroatoms independently selected from N, S, or O.

14. The compound of claim 13, wherein ring A is

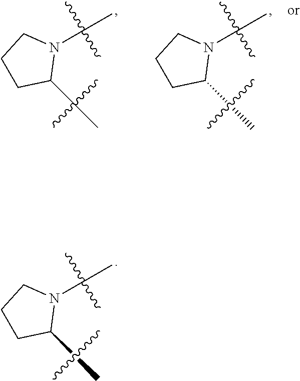

15. The compound of claim 13, wherein ring C is 6-membered aromatic ring.

16. The compound of claim 13, wherein ring C is phenyl, pyridyl, pyridazinyl, or pyrimidinyl.

17. The compound of claim 13, wherein ring C is phenyl.

18. The compound of claim 13, wherein X is selected from O, S, or N.

19. The compound of claim 13, wherein X is N.

20. The compound of claim 13, wherein Y is C.

21. The compound of claim 13, wherein Z is N.

22. The compound of claim 13, wherein R₁ is absent, H, or CH₃.

23. The compound of claim 13, wherein R₄ is

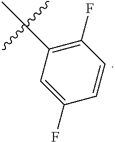 [SMILES: *c1cc(F)ccc1F]

24. The compound of claim 13, wherein R₅ and R₆ are each independently selected from H, OH, NH₂, F, Cl, Br, —CN, —CF₃, —OCF₃, CH₃, —O—CH₃, —S— CH₃, —CH₂OH, —COOH,

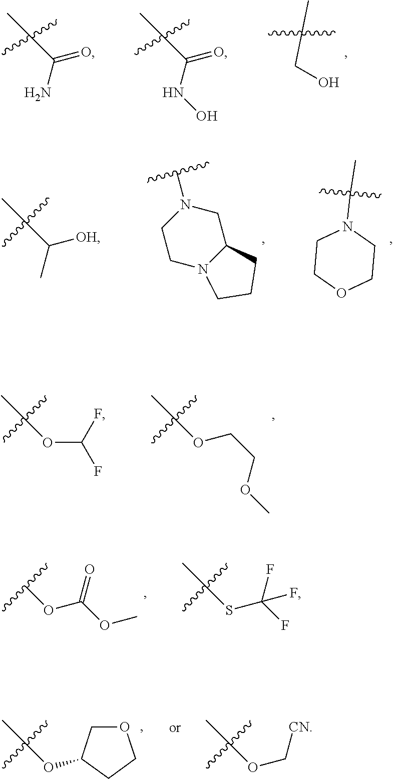

25. The compound of claim 13, wherein R₅ and R₆ are both —O—CH₃.

26. The compound of claim 13, wherein R₅ and R₆ together with the atoms to which they are attached from

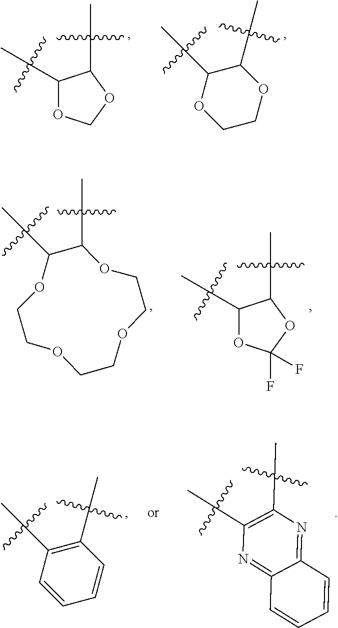 [SMILES: *C1OCOC1* ; *C1OCCOC1* ; *C1OCCOCCOCCOC1* ; *C1OC(F)(F)OC1* ; *c1ccccc1* ; *c1nc2ccccc2nc1*]

27. The compound of claim 1, wherein the compound is Formula IV or an isomeride, a stereoisomer, tautomer, pharmaceutically acceptable salt, prodrug, chelate, non-covalent complex, or solvate thereof, 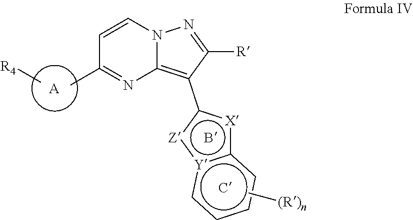
wherein,
Ring A is C₅₋₆ heterocyclic ring, wherein the C₅₋₆ heterocyclic ring optionally comprises 1, 2 or 3 hetero atoms independently selected from N, S, or O;
R₄ is (i) phenyl optionally substituted with one or more substituents independently selected from halogen, —C₁₋₄ alkyl, —C₁₋₄ haloalkyl, —C₁₋₄ alkoxyl, or (ii) a C₅₋₆ heteroaryl ring having a ring heteroatom selected from N, S, or O, wherein the C₅₋₆ heteroaryl ring is optionally substituted with one or more halogen atoms;
R′ is H, NH₂, or —C₁₋₄ alkyl;
Ring B′ is a 5-membered aromatic heterocyclic ring optionally comprising 1, 2 or 3 hetero atoms independently selected from N, S, or O;
Ring C′ is a phenyl, 6-membered heterocyclic ring, or 6-membered heteroaryl ring;
X′ and Z′ are each independently selected from C, N, O, or S;
Y′ is C or N;
R″ is —C(O)—C₁₋₄ alkyl, —SO—C₁₋₄ alkyl, —SO₂—C₁₋₄ alkyl, —NR₇(CH₂)ₘNR₈R₉, —(CH₂)ₘC₄₋₁₀ heterocyclyl; or NH₂, —C(O)OH, —C(O)NH₂, —C₁₋₄ alkyl, —C₁₋₄ alkoxyl, —C(O)—C₁₋₄ alkyl, —C(O)O—C₁₋₄ alkyl, —OC(O)O—C₁₋₄ alkyl, —S—C₁₋₄ alkyl, —SO—C₁₋₄ alkyl, —SO₂—C₁₋₄ alkyl, —OC₄₋₆heterocyclyl, —NR₇(CH₂)ₘNR₈R₉, —(CH₂)ₘC₄₋₁₀ heterocyclyl optionally substituted with one or more substituents independently selected from OH, CN, NH₂, —C(O)OH, halogen, —C₁₋₄ alkyl or —C₁₋₄ alkoxyl; or
any two R″ together with the atoms to which they are attached form a 5- to 12-membered ring;
R₇, R₈ and R₉ are each independently selected from H, or —C₁₋₄ alkyl;
m and n are each independently selected from 0, 1, 2, 3 or 4.

28. The compound of claim 27, wherein Ring A is

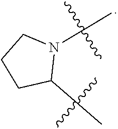 [SMILES: *C1CCCN1*]

29. The compound of claim 27, wherein R′ is selected from H.

30. The compound of claim 27, wherein R₄ is

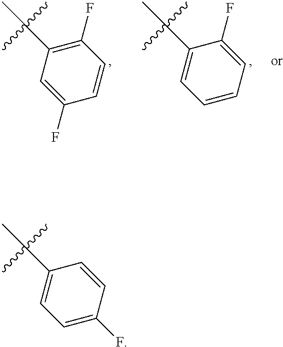 [SMILES: *c1cc(F)ccc1F ; *c1ccccc1F ; *c1ccc(F)cc1]

31. The compound of claim 27, wherein Ring B′ is selected from imidazole, oxazole, thiazole, triazole or pyrrole.

32. The compound of claim 27, wherein Ring B′ is selected from

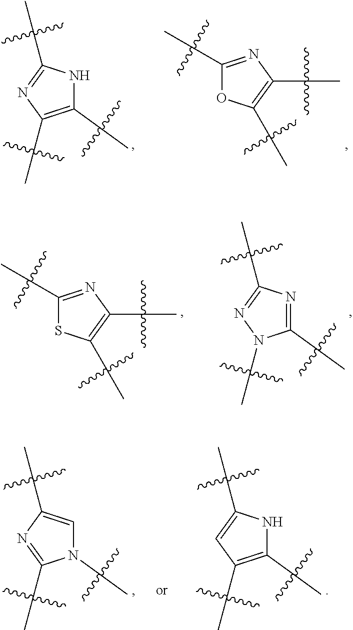 [SMILES: *c1nc(*)c(*)[nH]1 ; *c1nc(*)c(*)o1 ; *c1nc(*)c(*)s1 ; *c1nc(*)n(*)n1 ; *c1cn(*)c(*)n1 ; *c1cc(*)c(*)[nH]1]

33. The compound of claim 27, wherein Ring C′ is selected from phenyl, pyridine, pyrazine, pyrimidine, pyridazine, piperidine, or tetrahydropyran.

34. The compound of claim 27, wherein Ring C′ is selected from

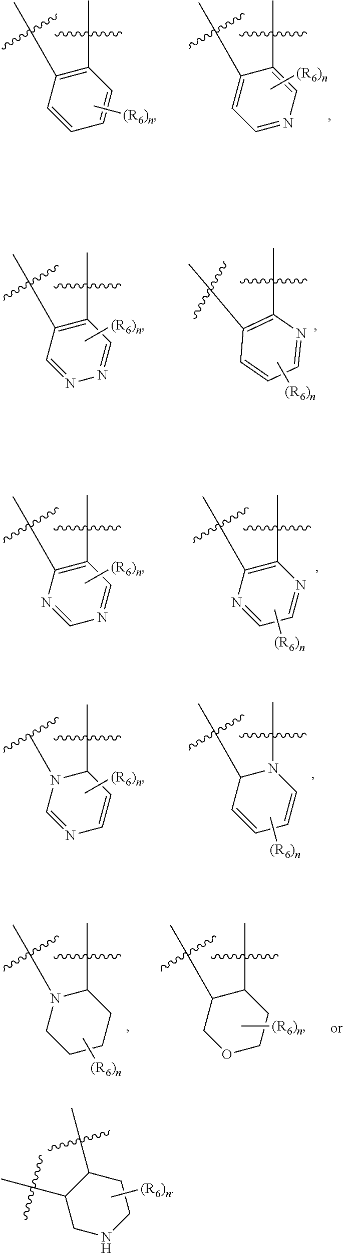

35. The compound of claim 27, wherein R″ is selected from

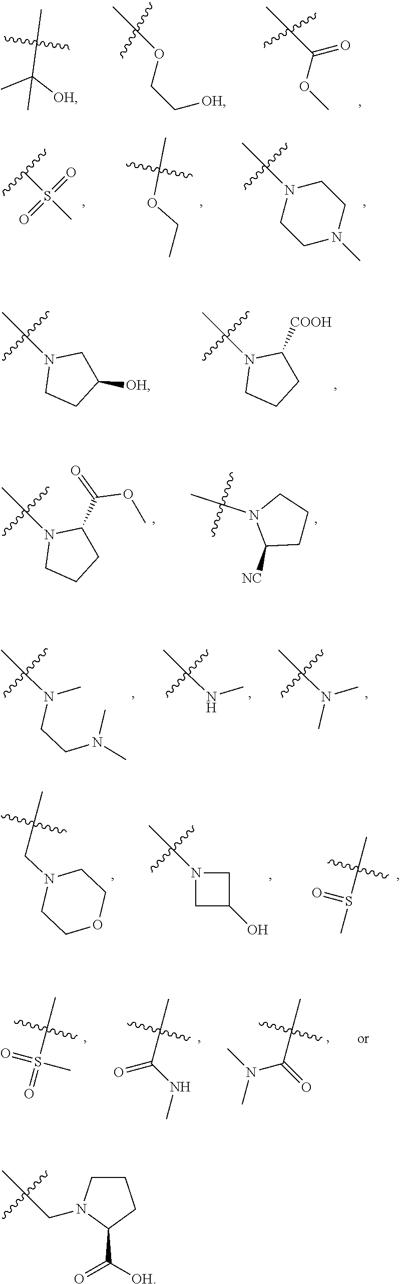

36. The compound of claim 27, wherein two R″ with the atoms to which they are attached form

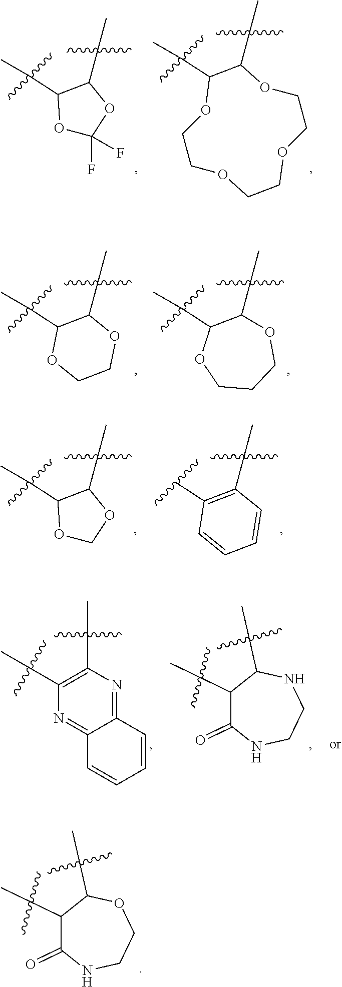

37. The compound of claim 1, or an isomeride, pharmaceutically acceptable salt or solvate thereof, wherein the compound is:
1) (R)-4-(4-(5-(5-(2-(2,5-difluorophenyl)pyrrolidin-1-yl)pyrazolo[1,5-a]pyrimidin-3-yl)-4H-1,2,4-triazol-3-yl)phenyl)morpholine;
2) (R)-1-(4-(5-(5-(2-(2,5-difluorophenyl)pyrrolidin-1-yl)pyrazolo[1,5-a]pyrimidin-3-yl)-4H-1,2,4-triazol-3-yl)pyridin-2-yl)piperidin-4-ol;
3) 5-((R)-2-(2,5-difluorophenyl)pyrrolidin-1-yl)-3-(5-(tetrahydrofuran-3-yl)-4H-1,2,4-triazol-3-yl)pyrazolo[1,5-a]pyrimidine;
4) (S)-1-(5-(5-((R)-2-(2,5-difluorophenyl)pyrrolidin-1-yl)pyrazolo[1,5-a]pyrimidin-3-yl)-4H-1,2,4-triazol-3-yl)ethan-1-ol;
5) (1S,4s)-4-(5-(5-((R)-2-(2,5-difluorophenyl)pyrrolidin-1-yl)pyrazolo[1,5-a]pyrimidin-3-yl)-4H-1,2,4-triazol-3-yl)cyclohexan-1-ol;
6) (R)-4-(4-(5-(5-(2-(2,5-difluorophenyl)pyrrolidin-1-yl)pyrazolo[1,5-a]pyrimidin-3-yl)-4H-1,2,4-triazol-3-yl)pyridin-2-yl)morpholine;
7) (R)-2-(5-(5-(2-(2,5-difluorophenyl)pyrrolidin-1-yl)pyrazolo[1,5-a]pyrimidin-3-yl)-4H-1,2,4-triazol-3-yl)propan-2-ol;
8) (R)-5-(2-(2,5-difluorophenyl)pyrrolidin-1-yl)-3-(5-(pyridin-4-yl)-4H-1,2,4-triazol-3-yl)pyrazolo[1,5-a]pyrimidine;
9) (R)-4-(5-(5-(2-(2,5-difluorophenyl)pyrrolidin-1-yl)pyrazolo[1,5-a]pyrimidin-3-yl)-4H-1,2,4-triazol-3-yl)phenol;
10) (R)-5-(2-(2,5-difluorophenyl)pyrrolidin-1-yl)-3-(5-(pyrazin-2-yl)-4H-1,2,4-triazol-3-yl)pyrazolo[1,5-a]pyrimidine;
11) (S)-1-(5-(5-((R)-2-(2,5-difluorophenyl)pyrrolidin-1-yl)pyrazolo[1,5-a]pyrimidin-3-yl)-4H-1,2,4-triazol-3-yl)ethan-1-amine;
12) methyl ((S)-1-(5-(5-((R)-2-(2,5-difluorophenyl)pyrrolidin-1-yl)pyrazolo[1,5-a]pyrimidin-3-yl)-4H-1,2,4-triazol-3-yl)ethyl)carbamate;
13) (R)-3-(5-(5-(2-(2,5-difluorophenyl)pyrrolidin-1-yl)pyrazolo[1,5-a]pyrimidin-3-yl)-4H-1,2,4-triazol-3-yl)benzonitrile;
14) (R)-5-(2-(2,5-difluorophenyl)pyrrolidin-1-yl)-3-(5-(6-(trifluoromethyl)pyridin-3-yl)-4H-1,2,4-triazol-3-yl)pyrazolo[1,5-a]pyrimidine;
15) 3-(5-(azetidin-2-yl)-4H-1,2,4-triazol-3-yl)-5-((R)-2-(2,5-difluorophenyl)pyrrolidin-1-yl)pyrazolo[1,5-a]pyrimidine;
16) ethyl (R)-5-(5-(2-(2,5-difluorophenyl)pyrrolidin-1-yl)pyrazolo[1,5-a]pyrimidin-3-yl)-4H-1,2,4-triazole-3-carboxylate;
17) (R)-5-(5-(2-(2,5-difluorophenyl)pyrrolidin-1-yl)pyrazolo[1,5-a]pyrimidin-3-yl)-4H-1,2,4-triazole-3-carboxylic acid;
18) (3S)-3-(5-(5-((R)-2-(2,5-difluorophenyl)pyrrolidin-1-yl)pyrazolo[1,5-a]pyrimidin-3-yl)-4H-1,2,4-triazol-3-yl)cyclohexan-1-ol;
19) (3S)-3-(5-(5-((R)-2-(2,5-difluorophenyl)pyrrolidin-1-yl)pyrazolo[1,5-a]pyrimidin-3-yl)-4H-1,2,4-triazol-3-yl)cyclopentan-1-ol;
20) tert-butyl 2-(5-(5-((R)-2-(2,5-difluorophenyl)pyrrolidin-1-yl)pyrazolo[1,5-a]pyrimidin-3-yl)-4H-1,2,4-triazol-3-yl)azetidine-1-carboxylate;
21) (R)-1-(4-(5-(5-(2-(2,5-difluorophenyl)pyrrolidin-1-yl)pyrazolo[1,5-a]pyrimidin-3-yl)-4-methyl-4H-1,2,4-triazol-3-yl)pyridin-2-yl)piperidin-4-ol;
22) (R)-5-(2-(2,5-difluorophenyl)pyrrolidin-1-yl)-3-(5-(piperidin-4-yl)-4H-1,2,4-triazol-3-yl)pyrazolo[1,5-a]pyrimidine;
23) (R)-1-(5-(5-(2-(2,5-difluorophenyl)pyrrolidin-1-yl)pyrazolo[1,5-a]pyrimidin-3-yl)-4H-1,2,4-triazol-3-yl)cyclobutan-1-ol;
24) (R)-1-(5-(5-(2-(2,5-difluorophenyl)pyrrolidin-1-yl)pyrazolo[1,5-a]pyrimidin-3-yl)-4H-1,2,4-triazol-3-yl)cyclobutan-1-amine;
25) (S)-2-(5-(5-((R)-2-(2,5-difluorophenyl)pyrrolidin-1-yl)pyrazolo[1,5-a]pyrimidin-3-yl)-4H-1,2,4-triazol-3-yl)-1,1,1-trifluoropropan-2-ol;
26) (R)-2-(5-(5-((R)-2-(2,5-difluorophenyl)pyrrolidin-1-yl)pyrazolo[1,5-a]pyrimidin-3-yl)-4H-1,2,4-triazol-3-yl)-1,1,1-trifluoropropan-2-ol;
27) (R)-2-(5-(5-(2-(2,5-difluorophenyl)pyrrolidin-1-yl)pyrazolo[1,5-a]pyrimidin-3-yl)-4H-1,2,4-triazol-3-yl)-1,1,1,3,3,3-hexafluoropropan-2-ol;
28) 2-(5-(5-((R)-2-(2,5-difluorophenyl)pyrrolidin-1-yl)pyrazolo[1,5-a]pyrimidin-3-yl)-4H-1,2,4-triazol-3-yl)-1,1,1-trifluorobutan-2-ol;
29) 3-(5-(5-((R)-2-(2,5-difluorophenyl)pyrrolidin-1-yl)pyrazolo[1,5-a]pyrimidin-3-yl)-4H-1,2,4-triazol-3-yl)-1,1,1-trifluoro-2-methylpropan-2-ol;
30) (R)-1-(5-(5-(2-(2,5-difluorophenyl)pyrrolidin-1-yl)pyrazolo[1,5-a]pyrimidin-3-yl)-4H-1,2,4-triazol-3-yl)-2-methylpropan-2-ol;
31) (R)-3-(5-(5-(2-(2,5-difluorophenyl)pyrrolidin-1-yl)pyrazolo[1,5-a]pyrimidin-3-yl)-4H-1,2,4-triazol-3-yl)cyclobutan-1-ol;
32) (R)-5-(2-(2,5-difluorophenyl)pyrrolidin-1-yl)-3-(5-(tetrahydro-2H-pyran-4-yl)-4H-1,2,4-triazol-3-yl)pyrazolo[1,5-a]pyrimidine;
33) (R)-2-(5-(5-(2-(2,5-difluorophenyl)pyrrolidin-1-yl)pyrazolo[1,5-a]pyrimidin-3-yl)-4H-1,2,4-triazol-3-yl)-2-methylpropan-1-ol;
34) (R)-1-(5-(5-((R)-2-(2,5-difluorophenyl)pyrrolidin-1-yl)pyrazolo[1,5-a]pyrimidin-3-yl)-4H-1,2,4-triazol-3-yl)ethan-1-ol;
35) 2-(5-(5-((R)-2-(2,5-difluorophenyl)pyrrolidin-1-yl)pyrazolo[1,5-a]pyrimidin-3-yl)-4H-1,2,4-triazol-3-yl)piperidin-4-ol;
36) (R)-6-(5-(5-(2-(2,5-difluorophenyl)pyrrolidin-1-yl)pyrazolo[1,5-a]pyrimidin-3-yl)-4H-1,2,4-triazol-3-yl)-1,2,3,4-tetrahydroisoquinoline;
37) (1R,3r)-3-(5-(5-((R)-2-(2,5-difluorophenyl)pyrrolidin-1-yl)pyrazolo[1,5-a]pyrimidin-3-yl)-4H-1,2,4-triazol-3-yl)adamantan-1-ol;
38) (R)-5-(2-(2,5-difluorophenyl)pyrrolidin-1-yl)-3-(5-(1-methylpiperidin-4-yl)-4H-1,2,4-triazol-3-yl)pyrazolo[1,5-a]pyrimidine;
39) (R)-2-(5-(5-((R)-2-(2,5-difluorophenyl)pyrrolidin-1-yl)pyrazolo[1,5-a]pyrimidin-3-yl)-4H-1,2,4-triazol-3-yl)propan-1-ol;
40) (R)-5-(2-(2,5-difluorophenyl)pyrrolidin-1-yl)-3-(5-(4-(piperazin-1-yl)phenyl)-4H-1,2,4-triazol-3-yl)pyrazolo[1,5-a]pyrimidine;
41) (R)-3-(5-(4,4-difluorocyclohexyl)-4H-1,2,4-triazol-3-yl)-5-(2-(2,5-difluorophenyl)pyrrolidin-1-yl)pyrazolo[1,5-a]pyrimidine;
42) (R)-(5-(5-((R)-2-(2,5-difluorophenyl)pyrrolidin-1-yl)pyrazolo[1,5-a]pyrimidin-3-yl)-4H-1,2,4-triazol-3-yl)(phenyl)methanol;
43) (R)-(3-(5-(5-(2-(2,5-difluorophenyl)pyrrolidin-1-yl)pyrazolo[1,5-a]pyrimidin-3-yl)-4H-1,2,4-triazol-3-yl)bicyclo[1.1.1]pentan-1-yl)methanol;
44) (R)-3-(5-(5-(2-(2,5-difluorophenyl)pyrrolidin-1-yl)pyrazolo[1,5-a]pyrimidin-3-yl)-4H-1,2,4-triazol-3-yl)bicyclo[1.1.1]pentan-1-amine;
45) (R)-6-(5-(5-(2-(2,5-difluorophenyl)pyrrolidin-1-yl)pyrazolo[1,5-a]pyrimidin-3-yl)-4H-1,2,4-triazol-3-yl)benzo[c][1,2]oxaborol-1(3H)-ol;
46) 1-(5-(5-((R)-2-(2,5-difluorophenyl)pyrrolidin-1-yl)pyrazolo[1,5-a]pyrimidin-3-yl)-4H-1,2,4-triazol-3-yl)-1,1-difluorobutan-2-ol;
47) 1-(5-(5-((R)-2-(2,5-difluorophenyl)pyrrolidin-1-yl)pyrazolo[1,5-a]pyrimidin-3-yl)-4H-1,2,4-triazol-3-yl)-2,2,2-trifluoroethan-1-ol;
48) 1-(5-(5-((R)-2-(2,5-difluorophenyl)pyrrolidin-1-yl)pyrazolo[1,5-a]pyrimidin-3-yl)-4H-1,2,4-triazol-3-yl)prop-2-yn-1-ol;
49) 3-(5-(5-((R)-2-(2,5-difluorophenyl)pyrrolidin-1-yl)pyrazolo[1,5-a]pyrimidin-3-yl)-4H-1,2,4-triazol-3-yl)morpholine;
50) (R)-3-(5-(1H-indol-5-yl)-4H-1,2,4-triazol-3-yl)-5-(2-(2,5-difluorophenyl)pyrrolidin-1-yl)pyrazolo[1,5-a]pyrimidine;
51) (S)-1-(5-(5-((R)-2-(2,5-difluorophenyl)pyrrolidin-1-yl)pyrazolo[1,5-a]pyrimidin-3-yl)-4H-1,2,4-triazol-3-yl)ethyl L-leucinate hydrochloride;
52) 2-(5-(5-((R)-2-(2,5-difluorophenyl)pyrrolidin-1-yl)pyrazolo[1,5-a]pyrimidin-3-yl)-4H-1,2,4-triazol-3-yl)-2-fluoroethan-1-ol;
53) (R)-1-(5-(5-(2-(2,5-difluorophenyl)pyrrolidin-1-yl)pyrazolo[1,5-a]pyrimidin-3-yl)-4H-1,2,4-triazol-3-yl)cyclopropan-1-ol;
54) (R)-5-(2-(2,5-difluorophenyl)pyrrolidin-1-yl)-3-(5-(6-(4-methylpiperazin-1-yl)pyridin-3-yl)-4H-1,2,4-triazol-3-yl)pyrazolo[1,5-a]pyrimidine;
55) (S)-1-(5-(5-((R)-2-(2,5-difluorophenyl)pyrrolidin-1-yl)pyrazolo[1,5-a]pyrimidin-3-yl)-4H-1,2,4-triazol-3-yl)ethyl L-valylvalinate hydrochloride;
56) (R)-6-(5-(5-(2-(2,5-difluorophenyl)pyrrolidin-1-yl)pyrazolo[1,5-a]pyrimidin-3-yl)-4H-1,2,4-triazol-3-yl)quinoline;
57) (R)-3-(5-(1H-benzo[d]imidazol-6-yl)-4H-1,2,4-triazol-3-yl)-5-(2-(2,5-difluorophenyl)pyrrolidin-1-yl)pyrazolo[1,5-a]pyrimidine;
58) (R)-5-(2-(2,5-difluorophenyl)pyrrolidin-1-yl)-3-(5-(4-phenoxyphenyl)-4H-1,2,4-triazol-3-yl)pyrazolo[1,5-a]pyrimidine;
59) (R)-3-(5-(1H-indazol-6-yl)-4H-1,2,4-triazol-3-yl)-5-(2-(2,5-difluorophenyl)pyrrolidin-1-yl)pyrazolo[1,5-a]pyrimidine;
60) (1R,2S,3R,5S)-5-(5-(5-((R)-2-(2,5-difluorophenyl)pyrrolidin-1-yl)pyrazolo[1,5-a]pyrimidin-3-yl)-4H-1,2,4-triazol-3-yl)cyclohexane-1,2,3,5-tetraol;
61) (R)-5-(2-(2,5-difluorophenyl)pyrrolidin-1-yl)-3-(5-(2,3-dihydrobenzofuran-6-yl)-4H-1,2,4-triazol-3-yl)pyrazolo[1,5-a]pyrimidine;
62) 5-((R)-2-(2,5-difluorophenyl)pyrrolidin-1-yl)-3-(6-((R)-hexahydropyrrolo[1,2-a]pyrazin-2(1H)-yl)-1H-benzo[d]imidazol-2-yl)pyrazolo[1,5-a]pyrimidine;
63) 2-(5-((R)-2-(2,5-difluorophenyl)pyrrolidin-1-yl)pyrazolo[1,5-a]pyrimidin-3-yl)-6-((R)-hexahydropyrrolo[1,2-a]pyrazin-2(1H)-yl)benzo[d]thiazole;
64) (R)-4-(2-(5-(2-(2,5-difluorophenyl)pyrrolidin-1-yl)pyrazolo[1,5-a]pyrimidin-3-yl)-1H-imidazo[4,5-c]pyridin-6-yl)morpholine;
65) (R)-2-(5-(2-(2,5-difluorophenyl)pyrrolidin-1-yl)pyrazolo[1,5-a]pyrimidin-3-yl)-1H-imidazo[4,5-c]pyridine;
66) 1-(2-(5-((R)-2-(2,5-difluorophenyl)pyrrolidin-1-yl)pyrazolo[1,5-a]pyrimidin-3-yl)-5-methoxy-1H-benzo[d]imidazol-6-yl)ethan-1-ol;
67) (R)-(2-(5-(2-(2,5-difluorophenyl)pyrrolidin-1-yl)pyrazolo[1,5-a]pyrimidin-3-yl)-5-methoxy-1H-benzo[d]imidazol-6-yl)methanol;
68) 1-(2-(5-((R)-2-(2,5-difluorophenyl)pyrrolidin-1-yl)pyrazolo[1,5-a]pyrimidin-3-yl)benzo[d]oxazol-6-yl)ethan-1-ol;
69) (R)-(2-(5-(2-(2,5-difluorophenyl)pyrrolidin-1-yl)pyrazolo[1,5-a]pyrimidin-3-yl)benzo[d]oxazol-6-yl)methanol;
70) 1-(2-(5-((R)-2-(2,5-difluorophenyl)pyrrolidin-1-yl)pyrazolo[1,5-a]pyrimidin-3-yl)-3H-imidazo[4,5-c]pyridin-6-yl)ethan-1-ol;
71) (R)-5-(2-(2,5-difluorophenyl)pyrrolidin-1-yl)-3-(5-(trifluoromethoxy)-1H-benzo[d]imidazol-2-yl)pyrazolo[1,5-a]pyrimidine;
72) (R)-2-(5-(2-(2,5-difluorophenyl)pyrrolidin-1-yl)pyrazolo[1,5-a]pyrimidin-3-yl)-6-(trifluoromethyl)-3H-imidazo[4,5-c]pyridine;
73) (R)-2-(5-(2-(2,5-difluorophenyl)pyrrolidin-1-yl)pyrazolo[1,5-a]pyrimidin-3-yl)oxazolo[4,5-c]pyridine;
74) (R)-5-(2-(2,5-difluorophenyl)pyrrolidin-1-yl)-3-(6-fluoro-1H-benzo[d]imidazol-2-yl)pyrazolo[1,5-a]pyrimidine;
75) (R)-2-(5-(2-(2,5-difluorophenyl)pyrrolidin-1-yl)pyrazolo[1,5-a]pyrimidin-3-yl)thiazolo[4,5-c]pyridine;
76) (R)-2-(5-(2-(2,5-difluorophenyl)pyrrolidin-1-yl)pyrazolo[1,5-a]pyrimidin-3-yl)-1H-imidazo[4,5-d]pyridazine;
77) (R)-5-(2-(2,5-difluorophenyl)pyrrolidin-1-yl)-3-(6-methoxy-1H-benzo[d]imidazol-2-yl)pyrazolo[1,5-a]pyrimidine;
78) (R)-5-(2-(2,5-difluorophenyl)pyrrolidin-1-yl)-3-(5,6-dimethoxy-1H-benzo[d]imidazol-2-yl)pyrazolo[1,5-a]pyrimidine;
79) (R)-6-(5-(2-(2,5-difluorophenyl)pyrrolidin-1-yl)pyrazolo[1,5-a]pyrimidin-3-yl)-2,2-difluoro-5H-[1,3]dioxolo[4′,5′:4,5]benzo[1,2-d]imidazole;
80) (R)-2-(5-(2-(2,5-difluorophenyl)pyrrolidin-1-yl)pyrazolo[1,5-a]pyrimidin-3-yl)-6-(trifluoromethoxy)benzo[d]oxazole;
81) (R)-3-(6-(difluoromethoxy)-1H-benzo[d]imidazol-2-yl)-5-(2-(2,5-difluorophenyl)pyrrolidin-1-yl)pyrazolo[1,5-a]pyrimidine;
82) (R)-2-(5-(2-(2,5-difluorophenyl)pyrrolidin-1-yl)pyrazolo[1,5-a]pyrimidin-3-yl)-6,7,9,10,12,13-hexahydro-1H-[1,4,7,10]tetraoxacyclododecino[2′,3′:4,5]benzo[1,2-d]imidazole;
83) (R)-2-(5-(2-(2,5-difluorophenyl)pyrrolidin-1-yl)pyrazolo[1,5-a]pyrimidin-3-yl)-1-methyl-6,7-dihydro-1H-[1,4]dioxino[2′,3′:4,5]benzo[1,2-d]imidazole;
84) (R)-2-(5-(2-(2,5-difluorophenyl)pyrrolidin-1-yl)pyrazolo[1,5-a]pyrimidin-3-yl)-5-methoxy-3H-imidazo[4,5-b]pyridine;
85) (R)-2-(5-(2-(2,5-difluorophenyl)pyrrolidin-1-yl)pyrazolo[1,5-a]pyrimidin-3-yl)-6-methoxy-1H-imidazo[4,5-c]pyridine;
86) (R)-2-(5-(2-(2,5-difluorophenyl)pyrrolidin-1-yl)pyrazolo[1,5-a]pyrimidin-3-yl)-6-methyl-1H-imidazo[4,5-c]pyridine;
87) (R)-8-(5-(2-(2,5-difluorophenyl)pyrrolidin-1-yl)pyrazolo[1,5-a]pyrimidin-3-yl)-7H-purin-6-amine;
88) (R)-8-(5-(2-(2,5-difluorophenyl)pyrrolidin-1-yl)pyrazolo[1,5-a]pyrimidin-3-yl)-7H-purin-6-ol;
89) (R)-2-(5-(2-(2,5-difluorophenyl)pyrrolidin-1-yl)pyrazolo[1,5-a]pyrimidin-3-yl)-N-hydroxy-5-methoxy-1H-benzo[d]imidazole-6-carboxamide;
90) (R)-2-(5-(2-(2,5-difluorophenyl)pyrrolidin-1-yl)pyrazolo[1,5-a]pyrimidin-3-yl)-5-methoxy-1H-benzo[d]imidazole-6-carboxylic acid;
91) (R)-2-(5-(2-(2,5-difluorophenyl)pyrrolidin-1-yl)pyrazolo[1,5-a]pyrimidin-3-yl)-5-methoxy-1H-benzo[d]imidazole-6-carboxamide;
92) (R)-3-(5-chloro-6-(trifluoromethoxy)-1H-benzo[d]imidazol-2-yl)-5-(2-(2,5-difluorophenyl)pyrrolidin-1-yl)pyrazolo[1,5-a]pyrimidine;
93) 1-(2-(5-((R)-2-(2,5-difluorophenyl)pyrrolidin-1-yl)pyrazolo[1,5-a]pyrimidin-3-yl)-6-fluorobenzo[d]oxazol-5-yl)ethan-1-ol;
94) (R)-(2-(5-(2-(2,5-difluorophenyl)pyrrolidin-1-yl)pyrazolo[1,5-a]pyrimidin-3-yl)-6-fluorobenzo[d]oxazol-5-yl)methanol;
95) (R)-(2-(5-(2-(2,5-difluorophenyl)pyrrolidin-1-yl)pyrazolo[1,5-a]pyrimidin-3-yl)-3H-imidazo[4,5-c]pyridin-6-yl)methanol;
96) (R)-2-(5-(2-(2,5-difluorophenyl)pyrrolidin-1-yl)pyrazolo[1,5-a]pyrimidin-3-yl)-6,7-dihydro-1H-[1,4]dioxino[2¹,3′:4,5]benzo[1,2-d]imidazole;
97) (R)-3-(7-chloro-1H-benzo[d]imidazol-2-yl)-5-(2-(2,5-difluorophenyl)pyrrolidin-1-yl)pyrazolo[1,5-a]pyrimidine;
98) (R)-3-(7-chloro-5-fluoro-1H-benzo[d]imidazol-2-yl)-5-(2-(2,5-difluorophenyl)pyrrolidin-1-yl)pyrazolo[1,5-a]pyrimidine;
99) (R)-2-(5-(2-(2,5-difluorophenyl)pyrrolidin-1-yl)pyrazolo[1,5-a]pyrimidin-3-yl)-7-methyl-1H-imidazo[4,5-c]pyridine;
100) (R)-2-(5-(2-(2,5-difluorophenyl)pyrrolidin-1-yl)pyrazolo[1,5-a]pyrimidin-3-yl)-4-methoxybenzo[d]oxazole;
101) (R)-3-(5,6-bis(2-methoxyethoxy)-1H-benzo[d]imidazol-2-yl)-5-(2-(2,5-difluorophenyl)pyrrolidin-1-yl)pyrazolo[1,5-a]pyrimidine;
102) (R)-6,7-dichloro-2-(5-(2-(2,5-difluorophenyl)pyrrolidin-1-yl)pyrazolo[1,5-a]pyrimidin-3-yl)-1H-imidazo[4,5-b]pyridine;
103) (R)-2-(5-(2-(2,5-difluorophenyl)pyrrolidin-1-yl)pyrazolo[1,5-a]pyrimidin-3-yl)-4-methyl-3H-imidazo[4,5-c]pyridine;
104) (R)-3-(4,7-dichloro-1H-benzo[d]imidazol-2-yl)-5-(2-(2,5-difluorophenyl)pyrrolidin-1-yl)pyrazolo[1,5-a]pyrimidine;
105) (R)-3-(5,6-dichloro-1H-benzo[d]imidazol-2-yl)-5-(2-(2,5-difluorophenyl)pyrrolidin-1-yl)pyrazolo[1,5-a]pyrimidine;
106) (R)-2-(5-(2-(2,5-difluorophenyl)pyrrolidin-1-yl)pyrazolo[1,5-a]pyrimidin-3-yl)-5-methyl-3H-imidazo[4,5-b]pyridine;
107) (R)-2-(5-(2-(2,5-difluorophenyl)pyrrolidin-1-yl)pyrazolo[1,5-a]pyrimidin-3-yl)-1H-benzo[d]imidazole-6-carbonitrile;
108) (R)-2-(5-(2-(2,5-difluorophenyl)pyrrolidin-1-yl)pyrazolo[1,5-a]pyrimidin-3-yl)-5-fluoro-3H-imidazo[4,5-b]pyridine;
109) (R)-3-(5,6-bis(difluoromethoxy)-1H-benzo[d]imidazol-2-yl)-5-(2-(2,5-difluorophenyl)pyrrolidin-1-yl)pyrazolo[1,5-a]pyrimidine;
110) (R)-2-(5-(2-(2,5-difluorophenyl)pyrrolidin-1-yl)pyrazolo[1,5-a]pyrimidin-3-yl)-6-(trifluoromethyl)-1H-imidazo[4,5-b]pyridine;
111) (R)-2-(5-(2-(2,5-difluorophenyl)pyrrolidin-1-yl)pyrazolo[1,5-a]pyrimidin-3-yl)-5,7-difluorobenzo[d]oxazole;
112) (R)-3-(5-chloro-6-methoxy-1H-benzo[d]imidazol-2-yl)-5-(2-(2,5-difluorophenyl)pyrrolidin-1-yl)pyrazolo[1,5-a]pyrimidine;
113) (R)-5-(2-(2,5-difluorophenyl)pyrrolidin-1-yl)-3-(7-(trifluoromethoxy)-1H-benzo[d]imidazol-2-yl)pyrazolo[1,5-a]pyrimidine;
114) (R)-2-(5-(2-(2,5-difluorophenyl)pyrrolidin-1-yl)pyrazolo[1,5-a]pyrimidin-3-yl)-5-(trifluoromethyl)-3H-imidazo[4,5-b]pyridine;
115) (R)-2-(5-(2-(2,5-difluorophenyl)pyrrolidin-1-yl)pyrazolo[1,5-a]pyrimidin-3-yl)-1H-benzo[d]imidazole-5,6-diyl dimethyl bis(carbonate);
116) (R)-5-(2-(2,5-difluorophenyl)pyrrolidin-1-yl)-3-(6-((trifluoromethyl)thio)-1H-benzo[d]imidazol-2-yl)pyrazolo[1,5-a]pyrimidine;
117) (R)-2-(5-(2-(2,5-difluorophenyl)pyrrolidin-1-yl)pyrazolo[1,5-a]pyrimidin-3-yl)-1H-benzo[d]imidazole-5,6-diol;
118) 5-((R)-2-(2,5-difluorophenyl)pyrrolidin-1-yl)-3-(5-(((R)-tetrahydrofuran-3-yl)oxy)-6-(((S)-tetrahydrofuran-3-yl)oxy)-1H-benzo[d]imidazol-2-yl)pyrazolo[1,5-a]pyrimidine;
119) (R)-2,2′-((2-(5-(2-(2,5-difluorophenyl)pyrrolidin-1-yl)pyrazolo[1,5-a]pyrimidin-3-yl)-1H-benzo[d]imidazole-5,6-diyl)bis(oxy))diacetonitrile;
120) (R)-2-(5-(2-(2,5-difluorophenyl)pyrrolidin-1-yl)pyrazolo[1,5-a]pyrimidin-3-yl)-5,6-dimethoxybenzo[d]oxazole;
121) (R)-2-(5-(2-(2,5-difluorophenyl)pyrrolidin-1-yl)pyrazolo[1,5-a]pyrimidin-3-yl)-1H-imidazo[4,5-b]quinoxaline;
122) (R)-2-(5-(2-(2,5-difluorophenyl)pyrrolidin-1-yl)pyrazolo[1,5-a]pyrimidin-3-yl)-7-methyl-3H-imidazo[4,5-b]pyridine;
123) (R)-2-(5-(2-(2,5-difluorophenyl)pyrrolidin-1-yl)pyrazolo[1,5-a]pyrimidin-3-yl)-5-fluoro-1H-benzo[d]imidazole-6-carbonitrile;
124) (R)-2-(5-(2-(2,5-difluorophenyl)pyrrolidin-1-yl)pyrazolo[1,5-a]pyrimidin-3-yl)-1-methyl-1H-imidazo[4,5-c]pyridine;
125) (R)-2-(5-(2-(2,5-difluorophenyl)pyrrolidin-1-yl)pyrazolo[1,5-a]pyrimidin-3-yl)-5-methoxy-1H-benzo[d]imidazole-6-carbonitrile;
126) (R)-2-(5-(2-(2,5-difluorophenyl)pyrrolidin-1-yl)pyrazolo[1,5-a]pyrimidin-3-yl)-5-(methylthio)-1H-benzo[d]imidazole-6-carbonitrile;
127) (R)-5-(2-(2,5-difluorophenyl)pyrrolidin-1-yl)-3-(7-fluoro-6-methoxy-1H-benzo[d]imidazol-2-yl)pyrazolo[1,5-a]pyrimidine;
128) (R)-2-(5-(2-(2,5-difluorophenyl)pyrrolidin-1-yl)pyrazolo[1,5-a]pyrimidin-3-yl)-1H-imidazo[4,5-b]pyrazine;
129) (R)-6-bromo-2-(5-(2-(2,5-difluorophenyl)pyrrolidin-1-yl)pyrazolo[1,5-a]pyrimidin-3-yl)-1H-imidazo[4,5-b]pyrazine;
130) (R)-2-(5-(2-(2,5-difluorophenyl)pyrrolidin-1-yl)pyrazolo[1,5-a]pyrimidin-3-yl)-1H-imidazo[4,5-b]phenazine;
131) (R)-6-(5-(2-(2,5-difluorophenyl)pyrrolidin-1-yl)pyrazolo[1,5-a]pyrimidin-3-yl)-[1,3]dioxolo[4′,5′:4,5]benzo[1,2-d]oxazole;
132) 2-(5-((R)-2-(2,5-difluorophenyl)pyrrolidin-1-yl)pyrazolo[1,5-a]pyrimidin-3-yl)-5,6,7,8-tetrahydro-[1,2,4]triazolo[1,5-a]pyridin-6-ol;
133) (R)-2-(5-(2-(2,5-difluorophenyl)pyrrolidin-1-yl)pyrazolo[1,5-a]pyrimidin-3-yl)-1H-indole-5-carbonitrile;
134) (R)-2-(5-(2-(2,5-difluorophenyl)pyrrolidin-1-yl)pyrazolo[1,5-a]pyrimidin-3-yl)-7,8-dihydro-1H,6H-[1,4]dioxepino[2′,3′:4,5]benzo[1,2-d]imidazole;
135) (R)-(2-(5-(2-(2,5-difluorophenyl)pyrrolidin-1-yl)pyrazolo[1,5-a]pyrimidin-3-yl)-3H-imidazo[4,5-c]pyridin-6-yl)methanol;
136) (R)-3-(5,6-difluoro-1H-benzo[d]imidazol-2-yl)-5-(2-(2,5-difluorophenyl)pyrrolidin-1-yl)pyrazolo[1,5-a]pyrimidine;
137) methyl (R)-2-(5-(2-(2,5-difluorophenyl)pyrrolidin-1-yl)pyrazolo[1,5-a]pyrimidin-3-yl)-4,5-difluoro-1H-benzo[d]imidazole-6-carboxylate;
138) (R)-2-(5-(2-(2,5-difluorophenyl)pyrrolidin-1-yl)pyrazolo[1,5-a]pyrimidin-3-yl)-4,5-difluoro-1H-benzo[d]imidazole-6-carboxylic acid;
139) (R)-5-(2-(2,5-difluorophenyl)pyrrolidin-1-yl)-3-(5-fluoro-6-(trifluoromethyl)-1H-benzo[d]imidazol-2-yl)pyrazolo[1,5-a]pyrimidine;
140) (R)-2-(5-(2-(2,5-difluorophenyl)pyrrolidin-1-yl)pyrazolo[1,5-a]pyrimidin-3-yl)-6-ethoxy-1H-benzo[d]imidazole-5-carbonitrile;
141) (R)-2-(5-(2-(2,5-difluorophenyl)pyrrolidin-1-yl)pyrazolo[1,5-a]pyrimidin-3-yl)-6-fluoro-1H-benzo[d]imidazole-5-carboxylic acid;
142) (R)-2-(5-(2-(2,5-difluorophenyl)pyrrolidin-1-yl)pyrazolo[1,5-a]pyrimidin-3-yl)-6-(methylamino)-1H-benzo[d]imidazole-5-carbonitrile;
143) (R)-2-(5-(2-(2,5-difluorophenyl)pyrrolidin-1-yl)pyrazolo[1,5-a]pyrimidin-3-yl)-6-morpholino-1H-benzo[d]imidazole-5-carbonitrile;
144) (R)-2-(5-(2-(2,5-difluorophenyl)pyrrolidin-1-yl)pyrazolo[1,5-a]pyrimidin-3-yl)-6-(dimethylamino)-1H-benzo[d]imidazole-5-carbonitrile;
145) (R)-2-(5-(2-(2,5-difluorophenyl)pyrrolidin-1-yl)pyrazolo[1,5-a]pyrimidin-3-yl)-6-(3-hydroxyazetidin-1-yl)-1H-benzo[d]imidazole-5-carbonitrile;
146) (R)-2-(5-(2-(2,5-difluorophenyl)pyrrolidin-1-yl)pyrazolo[1,5-a]pyrimidin-3-yl)-5,6,7,8-tetrahydroimidazo[4′,5′:4,5]benzo[1,2-e][1,4]diazepin-9(3H)-one;
147) (R)-2-(5-(2-(2,5-difluorophenyl)pyrrolidin-1-yl)pyrazolo[1,5-a]pyrimidin-3-yl)-7,8-dihydro-3H-imidazo[4′,5′:4,5]benzo[1,2-f][1,4]oxazepin-9(6H)-one;
148) (R)-2-(5-(2-(2,5-difluorophenyl)pyrrolidin-1-yl)pyrazolo[1,5-a]pyrimidin-3-yl)-1H-benzo[d]imidazole-5,6-dicarbonitrile;
149) (R)-2-(5-(2-(2,5-difluorophenyl)pyrrolidin-1-yl)pyrazolo[1,5-a]pyrimidin-3-yl)-6-hydroxy-1H-benzo[d]imidazole-5-carbonitrile;
150) (R)-2-(5-(2-(2,5-difluorophenyl)pyrrolidin-1-yl)pyrazolo[1,5-a]pyrimidin-3-yl)-6-(2-hydroxyethoxy)-1H-benzo[d]imidazole-5-carbonitrile;
151) (R)-6-bromo-2-(5-(2-(2,5-difluorophenyl)pyrrolidin-1-yl)pyrazolo[1,5-a]pyrimidin-3-yl)-1H-benzo[d]imidazole-5-carbonitrile;
152) methyl (R)-5-cyano-2-(5-(2-(2,5-difluorophenyl)pyrrolidin-1-yl)pyrazolo[1,5-a]pyrimidin-3-yl)-1H-benzo[d]imidazole-6-carboxylate;
153) (R)-5-cyano-2-(5-(2-(2,5-difluorophenyl)pyrrolidin-1-yl)pyrazolo[1,5-a]pyrimidin-3-yl)-1H-benzo[d]imidazole-6-carboxylic acid;
154) (R)-5-cyano-2-(5-(2-(2,5-difluorophenyl)pyrrolidin-1-yl)pyrazolo[1,5-a]pyrimidin-3-yl)-1H-benzo[d]imidazole-6-carboxamide;
155) methyl (R)-2-(5-(2-(2,5-difluorophenyl)pyrrolidin-1-yl)pyrazolo[1,5-a]pyrimidin-3-yl)-6-methoxy-1H-benzo[d]imidazole-5-carboxylate;
156) (R)-6-(difluoromethoxy)-2-(5-(2-(2,5-difluorophenyl)pyrrolidin-1-yl)pyrazolo[1,5-a]pyrimidin-3-yl)-1H-benzo[d]imidazole-5-carbonitrile;
157) (R)-2-(5-(2-(2,5-difluorophenyl)pyrrolidin-1-yl)pyrazolo[1,5-a]pyrimidin-3-yl)-5-(trifluoromethyl)-1H-benzo[d]imidazole-6-carbonitrile;
158) methyl (R)-2-(5-(2-(2,5-difluorophenyl)pyrrolidin-1-yl)pyrazolo[1,5-a]pyrimidin-3-yl)-6-fluoro-1H-benzo[d]imidazole-7-carboxylate;
159) (R)-2-(5-(2-(2,5-difluorophenyl)pyrrolidin-1-yl)pyrazolo[1,5-a]pyrimidin-3-yl)-6-methyl-1H-benzo[d]imidazole-5-carbonitrile;
160) (R)-2-(5-(2-(2,5-difluorophenyl)pyrrolidin-1-yl)pyrazolo[1,5-a]pyrimidin-3-yl)-6-methoxy-N-methyl-1H-benzo[d]imidazole-5-carboxamide;
161) (R)-2-(5-(2-(2,5-difluorophenyl)pyrrolidin-1-yl)pyrazolo[1,5-a]pyrimidin-3-yl)-6-methoxy-N,N-dimethyl-1H-benzo[d]imidazole-5-carboxamide;
162) (R)-4-((2-(5-(2-(2,5-difluorophenyl)pyrrolidin-1-yl)pyrazolo[1,5-a]pyrimidin-3-yl)-1H-benzo[d]imidazol-5-yl)methyl)morpholine;
163) (R)-2-(5-(2-(2,5-difluorophenyl)pyrrolidin-1-yl)pyrazolo[1,5-a]pyrimidin-3-yl)-6-(4-methylpiperazin-1-yl)-1H-benzo[d]imidazole-5-carbonitrile;
164) 2-(5-((R)-2-(2,5-difluorophenyl)pyrrolidin-1-yl)pyrazolo[1,5-a]pyrimidin-3-yl)-6-((S)-3-hydroxypyrrolidin-1-yl)-1H-benzo[d]imidazole-5-carbonitrile;
165) 6-((S)-2-cyanopyrrolidin-1-yl)-2-(5-((R)-2-(2,5-difluorophenyl)pyrrolidin-1-yl)pyrazolo[1,5-a]pyrimidin-3-yl)-1H-benzo[d]imidazole-5-carbonitrile;
166) methyl (5-cyano-2-(5-((R)-2-(2,5-difluorophenyl)pyrrolidin-1-yl)pyrazolo[1,5-a]pyrimidin-3-yl)-1H-benzo[d]imidazol-6-yl)-L-prolinate;
167) (5-cyano-2-(5-((R)-2-(2,5-difluorophenyl)pyrrolidin-1-yl)pyrazolo[1,5-a]pyrimidin-3-yl)-1H-benzo[d]imidazol-6-yl)-L-proline;
168) (R)-2-(5-(2-(2,5-difluorophenyl)pyrrolidin-1-yl)pyrazolo[1,5-a]pyrimidin-3-yl)-6-((2-(dimethylamino)ethyl)(methyl)amino)-1H-benzo[d]imidazole-5-carbonitrile;
169) (R)-2-(5-(2-(2,5-difluorophenyl)pyrrolidin-1-yl)pyrazolo[1,5-a]pyrimidin-3-yl)-6-(2-methoxyethoxy)-1H-benzo[d]imidazole-5-carbonitrile;
170) (R)-5-(2-(2,5-difluorophenyl)pyrrolidin-1-yl)-3-(6-(methylsulfonyl)-1H-benzo[d]imidazol-2-yl)pyrazolo[1,5-a]pyrimidine;
171) 2-(5-((R)-2-(2,5-difluorophenyl)pyrrolidin-1-yl)pyrazolo[1,5-a]pyrimidin-3-yl)-5-(methylsulfinyl)-1H-benzo[d]imidazole-6-carbonitrile;
172) (R)-2-(5-(2-(2,5-difluorophenyl)pyrrolidin-1-yl)pyrazolo[1,5-a]pyrimidin-3-yl)-5-(methylsulfonyl)-1H-benzo[d]imidazole-6-carbonitrile;
173) (R)-2-(5-(2-(2,5-difluorophenyl)pyrrolidin-1-yl)pyrazolo[1,5-a]pyrimidin-3-yl)-5-(methylsulfonyl)-1H-benzo[d]imidazole-6-carboxamide;
174) (R)-2-(5-(2-(2,5-difluorophenyl)pyrrolidin-1-yl)pyrazolo[1,5-a]pyrimidin-3-yl)-6-methoxybenzo[d]oxazole-5-carbonitrile;
175) methyl (R)-2-(5-(2-(2,5-difluorophenyl)pyrrolidin-1-yl)pyrazolo[1,5-a]pyrimidin-3-yl)-4-fluorobenzo[d]oxazole-7-carboxylate;
176) (R)-2-(5-(2-(2,5-difluorophenyl)pyrrolidin-1-yl)pyrazolo[1,5-a]pyrimidin-3-yl)-6-(trifluoromethoxy)benzo[d]oxazole-5-carbonitrile;
177) (R)-2-(5-(2-(2,5-difluorophenyl)pyrrolidin-1-yl)pyrazolo[1,5-a]pyrimidin-3-yl)-6-hydroxybenzo[d]oxazole-5-carbonitrile;
178) methyl (R)-2-(5-(2-(2,5-difluorophenyl)pyrrolidin-1-yl)pyrazolo[1,5-a]pyrimidin-3-yl)-5-methoxybenzo[d]oxazole-6-carboxylate;
179) (R)-6-(difluoromethoxy)-2-(5-(2-(2,5-difluorophenyl)pyrrolidin-1-yl)pyrazolo[1,5-a]pyrimidin-3-yl)-5-methylbenzo[d]oxazole;
180) ((2-(5-((R)-2-(2,5-difluorophenyl)pyrrolidin-1-yl)pyrazolo[1,5-a]pyrimidin-3-yl)-6-methoxybenzo[d]oxazol-5-yl)methyl)-L-proline;
181) (R)-2-(5-(2-(2,5-difluorophenyl)pyrrolidin-1-yl)pyrazolo[1,5-a]pyrimidin-3-yl)-5,8-dimethoxy-[1,2,4]triazolo[1,5-c]pyrimidine;
182) (R)-2-(5-(2-(2,5-difluorophenyl)pyrrolidin-1-yl)pyrazolo[1,5-a]pyrimidin-3-yl)-6,7-dimethoxy-[1,2,4]triazolo[1,5-a]pyridine;
183) (R)-5-(2-(2,5-difluorophenyl)pyrrolidin-1-yl)-3-(6-fluoro-1H-indol-2-yl)pyrazolo[1,5-a]pyrimidine;
184) methyl (R)-2-(5-(2-(2,5-difluorophenyl)pyrrolidin-1-yl)pyrazolo[1,5-a]pyrimidin-3-yl)-1H-indole-5-carboxylate;
185) (R)-2-(5-(2-(2,5-difluorophenyl)pyrrolidin-1-yl)pyrazolo[1,5-a]pyrimidin-3-yl)-1H-indole-5-carboxylic acid;
186) (R)-2-(5-(2-(2,5-difluorophenyl)pyrrolidin-1-yl)pyrazolo[1,5-a]pyrimidin-3-yl)-1H-indol-6-ol;
187) (S)-2-(5-((R)-2-(2,5-difluorophenyl)pyrrolidin-1-yl)pyrazolo[1,5-a]pyrimidin-3-yl)-5,6,7,8-tetrahydro-[1,2,4]triazolo[1,5-a]pyridin-7-ol;
188) (R)-2-(5-(2-(2,5-difluorophenyl)pyrrolidin-1-yl)pyrazolo[1,5-a]pyrimidin-3-yl)-3,4,6,7-tetrahydropyrano[3,4-d]imidazole;
189) (R)-2-(5-(2-(2,5-difluorophenyl)pyrrolidin-1-yl)pyrazolo[1,5-a]pyrimidin-3-yl)-4,5,6,7-tetrahydrothiazolo[4,5-c]pyridine;
190) (R)-2-(5-(2-(2,5-difluorophenyl)pyrrolidin-1-yl)pyrazolo[1,5-a]pyrimidin-3-yl)-4,5,6,7-tetrahydrothiazolo[5,4-c]pyridine;
191) (R)-2-(5-(2-(2,5-difluorophenyl)pyrrolidin-1-yl)pyrazolo[1,5-a]pyrimidin-3-yl)-6,7-dihydrothiazolo[5,4-c]pyridine-5(4H)-carboxamide;
192) (R)-2-(5-(2-(2,5-difluorophenyl)pyrrolidin-1-yl)pyrazolo[1,5-a]pyrimidin-3-yl)-6,7-dihydro-4H-pyrano[4,3-d]thiazole;
193) (R)-5-(2-(2,5-difluorophenyl)pyrrolidin-1-yl)-3-(5,6-dimethoxy-1H-benzo[d]imidazol-2-yl)pyrazolo[1,5-a]pyrimidin-2-amine;
194) (R)-2-(2-amino-5-(2-(2,5-difluorophenyl)pyrrolidin-1-yl)pyrazolo[1,5-a]pyrimidin-3-yl)-5-methoxy-1H-benzo[d]imidazole-6-carbonitrile;
195) (R)-2-(5-(2-(2-fluorophenyl)pyrrolidin-1-yl)pyrazolo[1,5-a]pyrimidin-3-yl)-6-methoxy-1H-benzo[d]imidazole-5-carbonitrile;
196) (R)-2-(5-(2-(3-fluorophenyl)pyrrolidin-1-yl)pyrazolo[1,5-a]pyrimidin-3-yl)-6-methoxy-1H-benzo[d]imidazole-5-carbonitrile;
197) (S)-2-(5-(2-(2,5-difluorophenyl)pyrrolidin-1-yl)pyrazolo[1,5-a]pyrimidin-3-yl)-6-methoxy-1H-benzo[d]imidazole-5-carbonitrile;
198) (R)-2-(5-(2-(4-fluorophenyl)pyrrolidin-1-yl)pyrazolo[1,5-a]pyrimidin-3-yl)-6-methoxy-1H-benzo[d]imidazole-5-carbonitrile;
199) (R)-5-(2-(2,5-difluorophenyl)pyrrolidin-1-yl)-3-(6,7-dimethoxyimidazo[1,2-a]pyridin-2-yl)pyrazolo[1,5-a]pyrimidine; or
200) (R)-3-(5-(2-(2,5-difluorophenyl)pyrrolidin-1-yl)pyrazolo[1,5-a]pyrimidin-3-yl)-5,6-dihydro-8H-[1,2,4]triazolo[3,4-c][1,4]oxazine.

38. A pharmaceutical composition comprising a compound of claim 1, or an isomeride, a pharmaceutically acceptable salt or a stereoisomer thereof, and at least one pharmaceutically acceptable carrier or excipient.

39. A method of inhibiting various different forms of Trk, including wildtype TrkA, TrkB and TrkC, the TrkA G595R, the TrkA G667C, the TrkA A608D, the TrkA F589L, and TrkC G623R, said method comprising administering to a patient a compound claim 1, or a pharmaceutically acceptable salt or an isomeride thereof.

40. A method of treating a disease associated with inhibition of Trk, including wildtype TrkA, TrkB and TrkC, the TrkA G595R, the TrkA G667C, the TrkA A608D, the TrkA F589L, and TrkC G623R, said method comprising administering to a patient in need thereof a therapeutically effective amount of a compound of claim 1, or a pharmaceutically acceptable salt or isomeride thereof.

41. The method of claim 40, wherein the disease is mammary analogue secretory carcinoma (MASC) of the salivary glands, infantile fibrosarcoma, spitz tumors, colon cancer, gastric cancer, thyroid cancer, lung cancer, leukemia, pancreatic cancer, melanoma, brain cancer, renal cancer, prostate cancer, ovarian cancer or breast cancer.

42. The method of claim 41, wherein the thyroid cancer is papillary thyroid cancer, the brain cancer is pontine glioma, the renal cancer is congenital mesoblastic nephroma, the breast cancer is secretory breast carcinoma.

43. (canceled)

44. (canceled)

45. (canceled)

46. (canceled)

47. (canceled)

48. (canceled)

49. A method of enhancing, stimulating and/or increasing the immune response in a patient, said method comprising administering to the patient in need thereof a therapeutically effective amount of a compound of claim 1, or a pharmaceutically acceptable salt or an isomeride thereof.
